(19) United States
(12) Patent Application Publication
(10) Pub. No.: US 2011/0009392 A1
(43) Pub. Date: Jan. 13, 2011

(54) GAMMA SECRETASE MODULATORS

(75) Inventors: Zhaoning Zhu, Plainsboro, NJ (US); William J. Greenlee, Teaneck, NJ (US); John P. Caldwell, Ringwood, NJ (US); Robert D. Mazzola, JR., Stewartsville, NJ (US); Brian McKittrick, New Vernon, NJ (US); Chad E. Bennett, Metuchen, NJ (US); Xianhai Huang, Warren, NJ (US); Hubert B. Josien, Jersey City, NJ (US); Duane A. Burnett, Bernardsville, NJ (US)

Correspondence Address: MERCK;PATENT DEPARTMENT (K-6-1, 1990), 2000 GALLOPING HILL ROAD, KENILWORTH, NJ, 07033-0530, US

(73) Assignee: Schering Corporation

(21) Appl. No.: 12/671,806

(22) PCT Filed: Aug. 4, 2008
(86) PCT No.: PCT/US08/09369

§ 371 Date: Sep. 13, 2010

Related U.S. Application Data
(60) Provisional application No. 60/954,178, filed on Aug. 6, 2007.

(51) Int. Cl. A61K 31/55; C07D 487/04; C07D 235/02; C07D 498/04; C07D 491/04; A61K 31/4188; A61K 31/519; A61K 31/535; A61K 31/4353; A61K 31/435; A61K 31/47; A61K 31/505; A61K 31/397; A61K 31/4406; A61K 38/16; A61P 25/28

(52) U.S. Cl. 514/221; 544/281; 548/303.1; 544/105; 540/568; 514/393; 514/259.1; 514/230.5; 514/319; 514/297; 514/311; 514/275; 514/210.02; 514/356; 514/1.1

(57) ABSTRACT

In its many embodiments, the present invention provides a novel class of heterocyclic compounds as modulators of gamma secretase, methods of preparing such compounds, pharmaceutical compositions containing one or more such compounds, methods of preparing pharmaceutical formulations comprising one or more such compounds, and methods of treatment, prevention, inhibition, or amelioration of one or more diseases associated with the central nervous system using such compounds or pharmaceutical compositions.

### REFERENCE TO RELATED APPLICATION

This application claims the benefit of U.S. Provisional Application No. 60/954,178 filed Aug. 6, 2007.

### FIELD OF THE INVENTION

The present invention relates to certain heterocyclic compounds useful as gamma secretase modulators (including inhibitors, antagonists and the like), pharmaceutical compositions containing the compounds, and methods of treatment using the compounds and compositions to treat various diseases including central nervous system disorders such as, for example, neurodegenerative diseases such as Alzheimer's disease and other diseases relating to the deposition of amyloid protein. They are especially useful for reducing Amyloid beta (hereinafter referred to as Aβ) production which is effective in the treatment of diseases caused by Aβ such as, for example, Alzheimers and Down Syndrome.

### BACKGROUND OF THE INVENTION

Alzheimer's disease is a disease characterized by degeneration and loss of neurons and also by the formation of senile plaques and neurofibrillary change. Presently, treatment of Alzheimer's disease is limited to symptomatic therapies with a symptom-improving agent represented by an acetylcholinesterase inhibitor, and the basic remedy which prevents progress of the disease has not been developed. A method of controlling the cause of onset of pathologic conditions needs to be developed for creation of the basic remedy of Alzheimer's disease.

Aβ protein, which is a metabolite of amyloid precursor protein (hereinafter referred to as APP), is considered to be greatly involved in degeneration and loss of neurons as well as onset of demential conditions (for example, see Klein W L, et al Proceeding National Academy of Science USA, Sep. 2, 2003, 100(18), p. 10417-22, suggest a molecular basis for reversible memory loss).

Nitsch R M, and 16 others, Antibodies against β-amyloid slow cognitive decline in Alzheimer's disease, Neuron, May 22, 2003, 38(4), p. 547-554) suggest that the main components of Aβ protein are Aβ40 consisting of 40 amino acids and Aβ42 having two additional amino acids at the C-terminal. The Aβ40 and Aβ42 tend to aggregate (for example, see Jarrell J T et al, The carboxy terminus of the β amyloid protein is critical for the seeding of amyloid formation: implications for the pathogenesis of Alzheimer's disease, Biochemistry, May 11, 1993, 32(18), p. 4693-4697) and constitute main components of senile plaques (for example, (Glenner G G, et al, Alzheimer's disease: initial report of the purification and characterization of a novel cerebrovascular amyloid protein, Biochemical and Biophysical Research Communications, May 16, 1984, 120(3), p. 885-90. See also Masters C L, et al, Amyloid plaque core protein in Alzheimer disease and Down syndrome, Proceeding National Academy of Science USA, June 1985, 82(12), p. 4245-4249.).

Furthermore, it is known that mutations of APP and presenelin genes, which is observed in familial Alzheimer's disease, increase production of Aβ40 and Aβ42 (for example, see Gouras G K, et al, Intraneuronal Aβ42 accumulation in human brain, American Journal of Pathology, January 2000, 156(1), p. 15-20. Also, see Scheuner D, et al, Nature Medicine, August 1996, 2(8), p. 864-870; and Forman M S, et al, Differential effects of the Swedish mutant amyloid precursor protein on β-amyloid accumulation and secretion in neurons and nonneuronal cells, Journal of Biological Chemistry, Dec. 19, 1997, 272(51), p. 32247-32253.). Therefore, compounds which reduce production of Aβ40 and Aβ42 are expected as an agent for controlling progress of Alzheimer's disease or for preventing the disease.

These Aβs are produced when APP is cleaved by beta secretase and subsequently clipped by gamma secretase. In consideration of this, creation of inhibitors of γ secretase and β secretase has been attempted for the purpose of reducing production of Aβs. Many of these secretase inhibitors already known are peptides or peptidomimetics such as L-685,458. L-685,458, an aspartyl protease transition state mimic, is a potent inhibitor of amyloid β-protein precursor γ-secretase activity, Biochemistry, Aug. 1, 2000, 39(30), p. 8698-8704).

Also of interest in connection with the present invention are: US 2007/0117798 (Eisai, published May 24, 2007); US 2007/0117839 (Eisai, published May 24, 2007); US 2006/0004013 (Eisai, published Jan. 5, 2006); WO 2005/110422 (Boehringer Ingelheim, published Nov. 24, 2005); WO 2006/045554 (Cellzone AG, published May 4, 2006); WO 2004/110350 (Neurogenetics, published Dec. 23, 2004); WO 2004/071431 (Myriad Genetics, published Aug. 26, 2004); US 2005/0042284 (Myriad Genetics, published Feb. 23, 2005) and WO 2006/001877 (Myriad Genetics, published Jan. 5, 2006).

There is a need for new compounds, formulations, treatments and therapies to treat diseases and disorders associated with Aβ. It is, therefore, an object of this invention to provide compounds useful in the treatment or prevention or amelioration of such diseases and disorders.

### SUMMARY OF THE INVENTION

In its many embodiments, the present invention provides a novel class of heterocyclic compounds as gamma secretase modulators (including inhibitors, antagonists and the like), methods of preparing such compounds, pharmaceutical compositions comprising one or more such compounds, methods of preparing pharmaceutical formulations comprising one or more such compounds, and methods of treatment, prevention, inhibition or amelioration of one or more diseases associated with the Aβ using such compounds or pharmaceutical compositions.

The compounds of this invention (Formula (I)) can be useful as gamma secretase modulators and can be useful in the treatment and prevention of diseases such as, for example, Alzheimers disease, mild cognitive impairment (MCI), Downs Syndrome, Glaucoma (Guo et. al., Proc. Natl. Acad. Sci. USA 104, 13444-13449 (2007)), Cerebral amyloid angiopathy, stroke or dementia (Frangione et al., Amyloid: J. Protein folding Disord. 8, suppl. 1, 36-42 (2001), Microgliosis and brain inflammation (M P Lamber, Proc. Natl. Acad. Sci. USA 95, 6448-53 (1998)), Olfactory function loss (Getchell, et. al. Neurobiology of Aging, 663-673, 24; 2003).

Thus, this invention provides compounds of the formula:

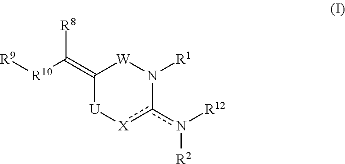

or a pharmaceutically acceptable salt, ester, solvate or prodrug thereof, wherein R¹, R², R⁸, R⁹, R¹⁰, R¹², U, W and X are each independently selected and are as defined below.

The compounds of Formula (I) can be useful as gamma secretase modulators and can be useful in the treatment and prevention of diseases such as, for example, central nervous system disorders such as Alzheimers disease and Downs Syndrome.

This invention also provides compounds of formula (I).

This invention also provides pharmaceutically acceptable salts of the compounds of formula (I).

This invention also provides pharmaceutically acceptable esters of the compounds of formula (I).

This invention also provides solvates of the compounds of formula (I).

This invention also provides compounds of formula (I) in pure and isolated form.

This invention also provides compounds of formula (I) in pure form.

This invention also provides compounds of formula (I) in isolated form.

This invention also provides compounds Y1, Y2, Y3, A9-A14, B1-B15, C3-C5, D4, E4, E6-E9, F7-F19, F20d-F20h, F21d-F21h, F22d-F22h, F23c-F23h, F24c-F24h, F25a-F25h, F26a-F26h, F27a-F27h, F28a-F28h, F29a-F29h, F30a-F30h, F31a-F31h, F32a-F32h, F33a-F33h, J1, J2, K7, K8b-K8h, K9a-K9h, K10a-K10h, K11a-K11h, K12a-K12h, K13a-K13h, K14a-K14h, K15a-K15h, K16a-K16h, K17a-K17h, K18a-K18h, K19a-K19h, K20a-K20h, K21a-K21h, K22a-K22h, and X1-X11.

This invention also provides compounds (R)-A9, (R)-B7, F7-F13, J1, (S)-A9, (S)-B7, F14-F19, J2, A10, B8, B15 and D3.

This invention also provides pharmaceutical compositions comprising an effective amount of one or more (e.g., one) compounds of formula (I), or a pharmaceutically acceptable salt, ester or solvate thereof, and a pharmaceutically acceptable carrier.

This invention also provides pharmaceutical compositions comprising an effective amount of one or more (e.g., one) compounds of formula (I), or a pharmaceutically acceptable salt, ester or solvate thereof, and an effective amount of one or more (e.g., one) other pharmaceutically active ingredients (e.g., drugs), and a pharmaceutically acceptable carrier.

This invention also provides a method for modulating (including inhibiting, antagonizing and the like) gamma-secretase, comprising administering an effective (i.e., therapeutically effective) amount of one or more compounds of formula (I) to a patient in need of treatment.

This invention also provides a method for modulating (including inhibiting, antagonizing and the like) gamma-secretase, comprising administering an effective (i.e., therapeutically effective) amount of a compound of formula (I) to a patient in need of treatment.

This invention also provides a method of treating one or more neurodegenerative diseases, comprising administering an effective (i.e., therapeutically effective) amount of one or more compounds of formula (I) to a patient in need of treatment.

This invention also provides a method of treating one or more neurodegenerative diseases, comprising administering an effective (i.e., therapeutically effective) amount of a compound of formula (I) to a patient in need of treatment.

This invention also provides a method of inhibiting the deposition of amyloid protein (e.g., amyloid beta protein) in, on or around neurological tissue (e.g., the brain), comprising administering an effective (i.e., therapeutically effective) amount of one or more compounds of formula (I) to a patient in need of treatment.

This invention also provides a method of inhibiting the deposition of amyloid protein (e.g., amyloid beta protein) in, on or around neurological tissue (e.g., the brain), comprising administering an effective (i.e., therapeutically effective) amount of a compound of formula (I) to a patient in need of treatment.

This invention also provides a method of treating Alzheimer's disease, comprising administering an effective (i.e., therapeutically effective) amount of one or more compounds of formula (I) to a patient in need of treatment.

This invention also provides a method of treating Alzheimer's disease, comprising administering an effective (i.e., therapeutically effective) amount of a compound of formula (I) to a patient in need of treatment.

This invention also provides a method of treating Alzheimer's disease, comprising administering an effective (i.e., therapeutically effective) amount of one or more compounds of formula (I), in combination with an effective (i.e., therapeutically effective) amount of one or more cholinesterase inhibitors (such as, for example, (±)-2,3-dihydro-5,6-dimethoxy-2-[[1-(phenylmethyl)-4-piperidinyl]methyl]-1H-inden-1-one hydrochloride, i.e., donepezil hydrochloride, available as the Aricept® brand of donepezil hydrochloride), to a patient in need of treatment.

This invention also provides a method of treating Alzheimer's disease, comprising administering an effective (i.e., therapeutically effective) amount of one or more compounds of formula (I), in combination with an effective (i.e., therapeutically effective) amount of one or more compounds selected from the group consisting of Aβ antibody inhibitors, gamma secretase inhibitors and beta secretase inhibitors.

This invention also provides combinations comprising an effective (i.e., therapeutically effective) amount of one or more compounds of formula (I), in combination with an effective (i.e., therapeutically effective) amount of one or more compounds selected from the group consisting of cholinesterase inhibitors (such as, for example, (±)-2,3-dihydro-5,6-dimethoxy-2-[[1-(phenylmethyl)-4-piperidinyl]methyl]-1H-inden-1-one hydrochloride, i.e., donepezil hydrochloride, available as the Aricept® brand of donepezil hydrochloride), Aβ antibody inhibitors, gamma secretase inhibitors and beta secretase inhibitors.

This invention also provides a method of treating Alzheimer's disease, comprising administering an effective (i.e., therapeutically effective) amount of a compound of formula (I), in combination with an effective (i.e., therapeutically effective) amount of one or more (e.g., one) cholinesterase inhibitors (such as, for example, (±)-2,3-dihydro-5,6-dimethoxy-2-[[1-(phenylmethyl)-4-piperidinyl]methyl]-1H-inden-1-one hydrochloride, i.e., donepezil hydrochloride, available as the Aricept® brand of donepezil hydrochloride), to a patient in need of treatment.

This invention also provides a method of treating Downs syndrome, comprising administering an effective (i.e., therapeutically effective) amount of one or more compounds of formula (I) to a patient in need of treatment.

This invention also provides a method of treating Downs syndrome, comprising administering an effective (i.e., therapeutically effective) amount of a compound of formula (I) to a patient in need of treatment.

This invention also provides a method of treating Downs syndrome, comprising administering an effective (i.e., therapeutically effective) amount of one or more compounds of formula (I), in combination with an effective (i.e., therapeutically effective) amount of one or more cholinesterase inhibitors (such as, for example, (±)-2,3-dihydro-5,6-dimethoxy-2-[[1-(phenylmethyl)-4-piperidinyl]methyl]-1H-inden-1-one hydrochloride, i.e., donepezil hydrochloride, available as the Aricept® brand of donepezil hydrochloride), to a patient in need of treatment.

This invention also provides a method of treating Downs syndrome, comprising administering an effective (i.e., therapeutically effective) amount of a compound of formula (I), in combination with an effective (i.e., therapeutically effective) amount of one or more (e.g., one) cholinesterase inhibitors (such as, for example, (±)-2,3-dihydro-5,6-dimethoxy-2-[[1-(phenylmethyl)-4-piperidinyl]methyl]-1H-inden-1-one hydrochloride, i.e., donepezil hydrochloride, available as the Aricept® brand of donepezil hydrochloride), to a patient in need of treatment.

This invention also provides combination therapies for (1) modulating gamma-secretase, or (2) treating one or more neurodegenerative diseases, or (3) inhibiting the deposition of amyloid protein (e.g., amyloid beta protein) in, on or around neurological tissue (e.g., the brain), or (4) treating Alzheimer's disease. The combination therapies are directed to methods comprising the administration of an effective amount of one or more (e.g. one) compounds of formula (I) and the administration of an effective amount of one or more (e.g., one) other pharmaceutical active ingredients (e.g., drugs).

This invention also provides a method of treating mild cognitive impairment, comprising administering an effective amount of one or more (e.g., one) compounds of formula (I) to a patient in need of treatment.

This invention also provides a method of treating glaucoma, comprising administering an effective amount of one or more (e.g., one) compounds of formula (I) to a patient in need of treatment.

This invention also provides a method of treating cerebral amyloid angiopathy, comprising administering an effective amount of one or more (e.g., one) compounds of formula (I) to a patient in need of treatment.

This invention also provides a method of treating stroke, comprising administering an effective amount of one or more (e.g., one) compounds of formula (I) to a patient in need of treatment.

This invention also provides a method of treating dementia, comprising administering an effective amount of one or more (e.g., one) compounds of formula (I) to a patient in need of treatment.

This invention also provides a method of treating microgliosis, comprising administering an effective amount of one or more (e.g., one) compounds of formula (I) to a patient in need of treatment.

This invention also provides a method of treating brain inflammation, comprising administering an effective amount of one or more (e.g., one) compounds of formula (I) to a patient in need of treatment.

This invention also provides a method of treating olfactory function loss, comprising administering an effective amount of one or more (e.g., one) compounds of formula (I) to a patient in need of treatment.

This invention also provides pharmaceutical compositions comprising a combination of an effective amount of one or more (e.g., one) compounds of formula (I), in combination with an effective amount of one or more compounds selected from the group consisting of cholinesterase inhibitors, Aβ antibody inhibitors, gamma secretase inhibitors and beta secretase inhibitors. The pharmaceutical compositions also comprise a pharmaceutically acceptable carrier.

This invention also provides a kit comprising, in separate containers, in a single package, pharmaceutical compositions for use in combination, wherein one container comprises an effective amount of a compound of formula (I) in a pharmaceutically acceptable carrier, and another container (i.e., a second container) comprises an effective amount of another pharmaceutically active ingredient, the combined quantities of the compound of formula (I) and the other pharmaceutically active ingredient being effective to: (a) treat Alzheimer's disease, or (b) inhibit the deposition of amyloid protein in, on or around neurological tissue (e.g., the brain), or (c) treat neurodegenerative diseases, or (d) modulate the activity of gamma-secretase.

This invention also provides a kit comprising, in separate containers, in a single package, pharmaceutical compositions for use in combination, wherein one container comprises an effective amount of a compound of formula (I) in a pharmaceutically acceptable carrier, and another container (i.e., a second container) comprises an effective amount of another pharmaceutically active ingredient (as described below), the combined quantities of the compound of formula (I) and the other pharmaceutically active ingredient being effective to: (a) treat Alzheimer's disease, or (b) inhibit the deposition of amyloid protein (e.g., amyloid beta protein) in, on or around neurological tissue (e.g., the brain), or (c) treat neurodegenerative diseases, or (d) modulate the activity of gamma-secretase.

This invention also provides any one of the methods disclosed above and below wherein the compound is selected from the group consisting of compounds of the formula: Y1, Y2, Y3, A9-A14, B1-B15, C3-C5, D4, E4, E6-E9, F7-F19, F20d-F20h, F21d-F21h, F22d-F22h, F23c-F23h, F24c-F24h, F25a-F25h, F26a-F26h, F27a-F27h, F28a-F28h, F29a-F29h, F30a-F30h, F31a-F31h, F32a-F32h, F33a-F33h, J1, J2, K7, K8b-K8h, K9a-K9h, K10a-K10h, K11a-K11h, K12a-K12h, K13a-K13h, K14a-K14h, K15a-K15h, K16a-K16h, K17a-K17h, K18a-K18h, K19a-K19h, K20a-K20h, K21a-K21h, K22a-K22h, and X1-X11.

This invention also provides any one of the pharmaceutical compositions disclosed above and below wherein the compound is selected from the group consisting of the compounds of formula: Y1, Y2, Y3, A9-A14, B1-B15, C3-C5, D4, E4, E6-E9, F7-F19, F20d-F20h, F21d-F21h, F22d-F22h, F23c-F23h, F24c-F24h, F25a-F25h, F26a-F26h, F27a-F27h, F28a-F28h, F29a-F29h, F30a-F30h, F31a-F31h, F32a-F32h, F33a-F33h, J1, J2, K7, K8b-K8h, K9a-K9h, K10a-K10h, K11a-K11h, K12a-K12h, K13a-K13h, K14a-K14h, K15a-K15h, K16a-K16h, K17a-K17h, K18a-K18h, K19a-K19h, K20a-K20h, K21a-K21h, K22a-K22h, and X1-X11.

### DETAILED DESCRIPTION

In one embodiment, the present invention discloses compounds which are represented by structural Formula (I), or a pharmaceutically acceptable salt, solvate, ester or prodrug thereof, wherein the various moieties are described above.

In another embodiment, the present application discloses a compound, or pharmaceutically acceptable salts, solvates, esters or prodrugs of said compound, said compound having the general structure shown in Formula:

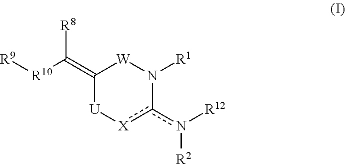

wherein:
either (i) R¹ and R² are joined together to form a 5-14 membered heteroaryl, 5-8 membered heterocyclyl or 5-8 membered heterocyclenyl moiety, with each of said heteroaryl, heterocyclyl or heterocyclenyl moiety being unsubstituted or optionally independently being substituted with 1-5 substituents which can be the same or different, each substituent being independently selected from the group consisting of the moieties shown below; or (ii) R² and R⁶ are joined together to form a 5-14 membered heteroaryl, 5-8 membered heterocyclyl or 5-8 membered heterocyclenyl moiety, with each of said heteroaryl, heterocyclyl or heterocyclenyl moiety being unsubstituted or optionally independently being substituted with 1-5 substituents which can be the same or different, each substituent being independently selected from the group consisting of the moieties shown below; or (iii) R⁶ and R⁵ are joined together to form a 5-14 membered heteroaryl, 5-8 membered heterocyclyl or 5-8 membered heterocyclenyl moiety, with each of said heteroaryl, heterocyclyl or heterocyclenyl moiety being unsubstituted or optionally independently being substituted with 1-5 substituents which can be the same or different, each substituent being independently selected from the group consisting of the moieties shown below; or (iv) R² and R¹⁴ are joined together to form a 5-14 membered heteroaryl, 5-8 membered heterocyclyl or 5-8 membered heterocyclenyl moiety, with each of said heteroaryl, heterocyclyl or heterocyclenyl moiety being unsubstituted or optionally independently being substituted with 1-5 substituents which can be the same or different, each substituent being independently selected from the group consisting of the moieties shown below; or (v) R³ and R¹⁴ are joined together to form a 5-14 membered heteroaryl, 5-8 membered heterocyclyl or 5-8 membered heterocyclenyl moiety, with each of said heteroaryl, heterocyclyl or heterocyclenyl moiety being unsubstituted or optionally independently being substituted with 1-5 substituents which can be the same or different, each substituent being independently selected from the group consisting of the moieties shown below; or (vi) R³ and R⁶ are joined together to form a 5-14 membered aryl, 5-8 membered cycloalkyl, 5-8 membered cycloalkenyl, 5-14 membered heteroaryl, 5-8 membered heterocyclyl or 5-8 membered heterocyclenyl moiety, with each of said aryl, cycloalkyl, cycloalkenyl, heteroaryl, heterocyclyl or heterocyclenyl moiety being unsubstituted or optionally independently being substituted with 1-5 substituents which can be the same or different, each substituent being independently selected from the group consisting of the moieties shown below; or (vii) R¹⁴ and R⁵ are joined together to form a 5-14 membered heteroaryl, 5-8 membered heterocyclyl or 5-8 membered heterocyclenyl moiety, with each of said heteroaryl, heterocyclyl or heterocyclenyl moiety being unsubstituted or optionally independently being substituted with 1-5 substituents which can be the same or different, each substituent being independently selected from the group consisting of the moieties shown below;
W is —S(O)—, —S(O)₂— or —C(O)—;
U is a bond, —C(O)—, —O—, —N(R⁵)— or —C(R³)(R⁴)—;
X is —N(R¹⁴)— or —C(R⁶)(R⁷)—;
each dashed line of

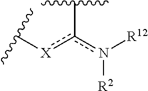

along with its adjoining single bond, together represents an optional double bond with the proviso that only one such double bond [symbol] is present at any given time, and further such that when the nitrogen of N(R²)(R¹²) is double bonded to the adjacent carbon between said nitrogen and X by the optional double bond, then R¹² is absent;

R¹ (when R¹ is not joined to R²) is selected from the group consisting of H, alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl-, wherein each of said alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl- can be unsubstituted or optionally independently substituted with 1-5 substituents which can be the same or different, each substituent being independently selected from the group consisting of the moieties shown below;

R² (when R² is not joined to R¹, R⁶ or R¹⁴) is selected from the group consisting of H, alkyl, alkenyl-, alkynyl, cycloalkyl, cycloalkylalkyl, cycloalkenyl, heterocyclyl, heterocyclylalkyl, aryl, arylalkyl, heteroaryl, heteroarylalkyl, —CN, —C(O)R¹⁵, —C(O)OR¹⁵, —C(O)N(R¹⁵)(R¹⁶), —S(O)N(R¹⁵)(R¹⁶), —S(O)₂N(R¹⁵)(R¹⁶), —S(O)R¹⁵, —S(O)₂R¹⁵, —C(═NOR¹⁵)R¹⁶ and —P(O)(OR¹⁵)(OR¹⁶);

R⁵ (when R⁵ is not joined to R⁶ or R¹⁴) is selected from the group consisting of H, alkyl, alkenyl, alkynyl, cycloalkyl, cycloalkylalkyl, cycloalkenyl, heterocyclyl, heterocyclylalkyl, aryl, arylalkyl, heteroaryl, heteroarylalkyl, —CN, —C(O)R¹⁵, —C(O)OR¹⁵, —C(O)N(R¹⁵)(R¹⁶), —S(O)N(R¹⁵)(R¹⁶), —S(O)₂N(R¹⁵)(R¹⁶), —S(O)R¹⁵, —S(O)₂R¹⁵, —C(═NOR¹⁵)R¹⁶ and —P(O)(OR¹⁵)(OR¹⁶);

R¹² is independently selected from the group consisting of H, alkyl, alkenyl, alkynyl, cycloalkyl, cycloalkylalkyl, cycloalkenyl, heterocyclyl, heterocyclylalkyl, aryl, arylalkyl, heteroaryl, heteroarylalkyl, —CN, —C(O)R¹⁵, —C(O)OR¹⁵, —C(O)N(R¹⁵)(R¹⁶), —S(O)N(R¹⁵)(R¹⁶), —S(O)₂N(R¹⁵)(R¹⁶), —S(O)R¹⁵, —S(O)₂R¹⁵, —C(═NOR¹⁵)R¹⁶ and —P(O)(OR¹⁵)(OR¹⁶);

each R¹⁴ (when R¹⁴ is not joined to R², R³ or R⁵) can be the same or different, each being independently selected from the group consisting of H, alkyl, alkenyl, alkynyl, cycloalkyl, cycloalkylalkyl, cycloalkenyl, heterocyclyl, heterocyclylalkyl, aryl, arylalkyl, heteroaryl, heteroarylalkyl, —CN, —C(O)R¹⁵, —C(O)OR¹⁵, —C(O)N(R¹⁵)(R¹⁶), —S(O)N(R¹⁵)(R¹⁶), —S(O)₂N(R¹⁵)(R¹⁶), —S(O)R¹⁵, —S(O)₂R¹⁵, —C(═NOR¹⁵)R¹⁶, and —P(O)(OR¹⁵)(OR¹⁶);

R³ (when R³ is not joined to R⁶ or R¹⁴) is independently selected from the group consisting of H, alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl-, wherein each of said alkyl-, alkenyl, alkynyl-, aryl-, arylalkyl-, alkylaryl-, cycloalkyl-, cycloalkylalkyl-, heteroaryl-, heteroarylalkyl-, heterocyclyl- and heterocyclyalkyl- can be unsubstituted or optionally independently substituted with 1-5 substituents which can be the same or different, each substituent being independently selected from the group consisting of consisting of the moieties shown below;

R⁴ is independently selected from the group consisting of H, alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl-, wherein each of said alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl- can be unsubstituted or optionally independently substituted with 1-5 substituents which can be the same or different, each substituent being independently selected from the group consisting of consisting of the moieties shown below;

R⁶ (when R⁶ is not joined to R², R³ or R⁵) is selected from the group consisting of H, alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl-, wherein each of said alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl- can be unsubstituted or optionally independently substituted with 1-5 substituents which can be the same or different, each substituent being independently selected from the group consisting of consisting of the moieties shown below;

R⁷ is selected from the group consisting of H, alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl-, wherein each of said alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl- can be unsubstituted or optionally independently substituted with 1-5 substituents which can be the same or different, each substituent being independently selected from the group consisting of consisting of the moieties shown below;

R⁸ is selected from the group consisting of H, alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl-, with each of said alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl- being unsubstituted or optionally independently substituted with 1-3 substituents which can be the same or different, each substituent being independently selected from the group consisting of the moieties shown below;

R⁹ is selected from the group consisting of alkyl-, alkenyl- alkynyl-, aryl-, arylalkyl-, alkylaryl-, cycloalkyl-, cycloalkylalkyl-, heteroaryl-, heteroarylalkyl-, heterocyclyl- and heterocyclyalkyl-, wherein each of said alkyl-, alkenyl- alkynyl-, aryl-, arylalkyl-, alkylaryl-, cycloalkyl-, cycloalkylalkyl-, heteroaryl-, heteroarylalkyl-, heterocyclyl- and heterocyclyalkyl- can be unsubstituted or optionally independently substituted with 1-3 substituents which can be the same or different, each substituent being independently selected from the group consisting of the moieties shown below,

R¹⁰ is selected from the group consisting of a bond, alkyl-, alkenyl- alkynyl-, aryl-, arylalkyl-, alkylaryl-, cycloalkyl-, cycloalkylalkyl-, heteroaryl-, heteroarylalkyl-, heterocyclyl-, heterocyclyalkyl- and the moieties:

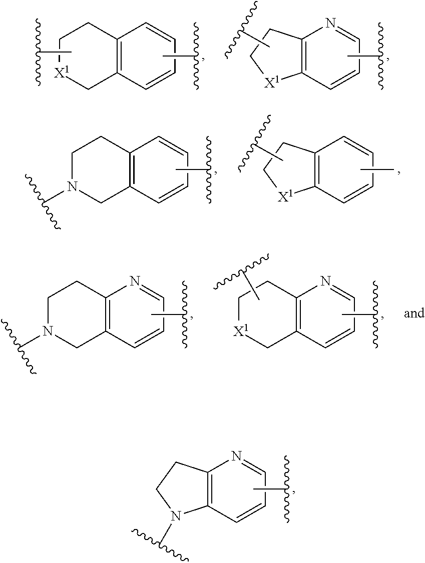

where X¹ is O, N(R¹⁴) or S;

wherein each of said alkyl-, alkenyl- alkynyl-, aryl-, arylalkyl-, alkylaryl-, cycloalkyl-, cycloalkylalkyl-, heteroaryl-, heteroarylalkyl-, heterocyclyl- heterocyclyalkyl- and the above-noted moieties for R¹⁰ can be unsubstituted or optionally independently substituted with 1-3 substituents which can be the same or different, each being independently selected from the group consisting of the moieties shown below; and

R¹⁵, R¹⁶ and R¹⁷ are independently selected from the group consisting of H, alkyl, alkenyl, alkynyl, cycloalkyl, cycloalkylalkyl, heterocyclyl, heterocyclylalkyl, aryl, arylalkyl, heteroaryl, heteroarylalkyl, arylcycloalkyl, arylheterocyclyl, R¹⁸-alkyl, R¹⁸-cycloalkyl, R¹⁸-cycloalkylalkyl, R¹⁸-heterocyclyl, R¹⁸-heterocyclylalkyl, R¹⁸-aryl, R¹⁸-arylalkyl, R¹⁸-heteroaryl and R¹⁸-heteroarylalkyl;

R¹⁸ is 1-5 substituents independently selected from the group consisting of alkyl, alkenyl, alkynyl, aryl, arylalkyl, arylalkenyl, arylalkynyl, —NO₂, halo, heteroaryl, HO-alkyoxyalkyl, —CF₃, —CN, alkyl-CN, —C(O)R¹⁹, —C(O)OH, —C(O)OR¹⁹, —C(O)NHR²⁰, —C(O)NH₂, —C(O)NH₂—C(O)N(alkyl)₂, —C(O)N(alkyl)(aryl), —C(O)N(alkyl)(heteroaryl), —SR¹⁹, —S(O)₂R²⁰, —S(O)NH₂, —S(O)NH(alkyl), —S(O)N(alkyl)(alkyl), —S(O)NH(aryl), —S(O)₂NH₂, —S(O)₂NHR¹⁹, —S(O)₂NH(heterocyclyl), —S(O)₂N(alkyl)₂, —S(O)₂N(alkyl)(aryl), —OCF₃, —OH, —OR²⁰, —O-heterocyclyl, —O-cycloalkylalkyl, —O-heterocyclylalkyl, —NH₂, —NHR²⁰, —N(alkyl)₂, —N(arylalkyl)₂, —N(arylalkyl)-(heteroarylalkyl), —NHC(O)R²⁰, —NHC(O)NH₂, —NHC(O)NH(alkyl), —NHC(O)N(alkyl)(alkyl), —N(alkyl)C(O)NH(alkyl), —N(alkyl)C(O)N(alkyl)(alkyl), —NHS(O)₂R²⁰, —NHS(O)₂NH(alkyl), —NHS(O)₂N(alkyl)(alkyl), —N(alkyl)S(O)₂NH(alkyl) and —N(alkyl)S(O)₂N(alkyl)(alkyl);

or, alternately, two R¹⁸ moieties on adjacent carbons can be linked together to form:

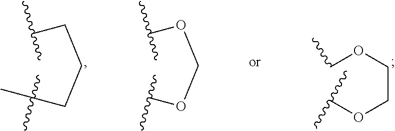

R¹⁹ is alkyl, cycloalkyl, aryl, arylalkyl or heteroarylalkyl;

R²⁰ is alkyl, cycloalkyl, aryl, halo substituted aryl, arylalkyl, heteroaryl or heteroarylalkyl;

wherein each of the alkyl, cycloalkyl, cycloalkylalkyl, heterocyclyl, heterocyclylalkyl, aryl, arylalkyl, alkylaryl, heteroaryl, heteroarylalkyl, alkenyl and alkynyl groups in R¹, R², R³, R⁴, R⁵, R⁶, R⁷, R⁸, R⁹, R¹⁰, R¹² and R¹⁴, the 5-14 membered heteroaryl, 5-8 membered heterocyclyl or 5-8 membered heterocyclenyl moiety formed from the joining of R¹ and R², R² and R⁶, R⁶ and R⁵, R² and R¹⁴, R³ and R¹⁴, R¹⁴ and R⁵ or R³ and R⁶, or the 5-14 membered aryl, 5-8 membered cycloalkyl or 5-8 membered cycloalkenyl moiety formed from the joining of R³ and R⁶ are independently unsubstituted or substituted by 1 to 5 R²¹ groups independently selected from the group consisting of alkyl, alkenyl, alkynyl, cycloalkyl, cycloalkylalkyl, cycloalkenyl, heterocyclyl, heterocyclylalkyl, aryl, arylalkyl, heteroaryl, heteroarylalkyl, halo, —CN, —OR¹⁵, —C(O)R¹⁵, —C(O)OR¹⁵, —C(O)N(R¹⁵)(R¹⁶), —SR¹⁵, —S(O)N(R¹⁵)(R¹⁶), —CH(R¹⁵)(R¹⁶), —S(O)₂N(R¹⁵)(R¹⁶), —C(═NOR¹⁵)R¹⁶, —P(O)(OR¹⁵)(OR¹⁶), —N(R¹⁵)(R¹⁶), -alkyl-N(R¹⁵)(R¹⁶), —N(R¹⁵)C(O)R¹⁶, —CH₂—N(R¹⁵)C(O)R¹⁶, —CH₂—N(R¹⁵)C(O)N(R¹⁶)(R¹⁷), —CH₂—R¹⁵; —CH₂N(R¹⁵)(R¹⁶), —N(R¹⁵)S(O)R¹⁶, —N(R¹⁵)S(O)₂R¹⁶, —CH₂—N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —CH₂—N(R¹⁵)C(O)N(R¹⁶)(R¹⁷), —N(R¹⁵)C(O)OR¹⁶, —CH₂—N(R¹⁵)C(O)OR¹⁶, —S(O)R¹⁵, ═NOR¹⁵, —N₃, —NO₂ and —S(O)₂R¹⁵;

wherein each of the alkyl, cycloalkenyl, cycloalkyl, cycloalkylalkyl, heterocyclyl, heterocyclylalkyl, aryl, arylalkyl, heteroaryl, heteroarylalkyl, alkenyl and alkynyl groups in R²¹ are independently unsubstituted or substituted by 1 to 5 R²² groups independently selected from the group consisting of alkyl, cycloalkyl, cycloalkenyl, heterocyclyl, aryl, heteroaryl, halo, —CF₃, —CN, —OR¹⁵, —C(O)R¹⁵, —C(O)OR¹⁵, -alkyl-C(O)OR¹⁵, C(O)N(R¹⁵)(R¹⁶), —SR¹⁵, —S(O)N(R¹⁵)(R¹⁶), —S(O)₂N(R¹⁵)(R¹⁶), —C(═NOR¹⁵)R¹⁶, —P(O)(OR¹⁵)(OR¹⁶), —N(R¹⁵)(R¹⁶), -alkyl-N(R¹⁵)(R¹⁶), —N(R¹⁵)C(O)R¹⁶, —CH₂—N(R¹⁵)C(O)R¹⁶, —N(R¹⁵)S(O)R¹⁶, —N(R¹⁵)S(O)₂R¹⁶, —CH₂—N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —CH₂—N(R¹⁵)C(O)N(R¹⁶)(R¹⁷), —N(R¹⁵)C(O)OR¹⁶, —CH₂—N(R¹⁵)C(O)OR¹⁶, —N₃, ═NOR¹⁵, —NO₂, —S(O)R¹⁵ and —S(O)₂R¹⁵.

The statement above: “either (i) R¹ and R² are joined together to form a 5-14 membered heteroaryl, 5-8 membered heterocyclyl or 5-8 membered heterocyclenyl moiety, with each of said heteroaryl, heterocyclyl or heterocyclenyl moiety being unsubstituted or optionally independently being substituted with 1-5 substituents which can be the same or different, each substituent being independently selected from the group consisting of the moieties shown below; or (ii) R² and R⁶ are joined together to form a 5-14 membered heteroaryl, 5-8 membered heterocyclyl or 5-8 membered heterocyclenyl moiety, with each of said heteroaryl, heterocyclyl or heterocyclenyl moiety being unsubstituted or optionally independently being substituted with 1-5 substituents which can be the same or different, each substituent being independently selected from the group consisting of the moieties shown below; or (iii) R⁶ and R⁵ are joined together to form a 5-14 membered heteroaryl, 5-8 membered heterocyclyl or 5-8 membered heterocyclenyl moiety, with each of said heteroaryl, heterocyclyl or heterocyclenyl moiety being unsubstituted or optionally independently being substituted with 1-5 substituents which can be the same or different, each substituent being independently selected from the group consisting of the moieties shown below; or (iv) R² and R¹⁴ are joined together to form a 5-14 membered heteroaryl, 5-8 membered heterocyclyl or 5-8 membered heterocyclenyl moiety, with each of said heteroaryl, heterocyclyl or heterocyclenyl moiety being unsubstituted or optionally independently being substituted with 1-5 substituents which can be the same or different, each substituent being independently selected from the group consisting of the moieties shown below; or (v) R³ and R¹⁴ are joined together to form a 5-14 membered heteroaryl, 5-8 membered heterocyclyl or 5-8 membered heterocyclenyl moiety, with each of said heteroaryl, heterocyclyl or heterocyclenyl moiety being unsubstituted or optionally independently being substituted with 1-5 substituents which can be the same or different, each substituent being independently selected from the group consisting of the moieties shown below; or (vi) R³ and R⁶ are joined together to form a 5-14 membered aryl, 5-8 membered cycloalkyl, 5-8 membered cycloalkenyl, 5-14 membered heteroaryl, 5-8 membered heterocyclyl or 5-8 membered heterocyclenyl moiety, with each of said aryl, cycloalkyl, cycloalkenyl, heteroaryl, heterocyclyl or heterocyclenyl moiety being unsubstituted or optionally independently being substituted with 1-5 substituents which can be the same or different, each substituent being independently selected from the group consisting of the moieties shown below; or (vii) R¹⁴ and R⁵ are joined together to form a 5-14 membered heteroaryl, 5-8 membered heterocyclyl or 5-8 membered heterocyclenyl moiety, with each of said heteroaryl, heterocyclyl or heterocyclenyl moiety being unsubstituted or optionally independently being substituted with 1-5 substituents which can be the same or different, each substituent being independently selected from the group consisting of the moieties shown below;” means that the occurrences of (i), (ii), (iii), (iv), (v), (vi) and (vii) are mutually exclusive and that only one of (i), (ii), (iii), (iv), (v) (vi) and (vii) can be present at any given time.

It should be understood that when R¹ and R² are joined together to form a 5-14 membered heteroaryl, 5-8 membered heterocyclyl or 5-8 membered heterocyclenyl moiety, each of said heteroaryl, heterocyclyl or heterocyclenyl moiety independently may optionally additionally be fused with an aryl or heteroaryl ring, wherein the ring moiety resulting from the fusion may be unsubstituted or optionally independently substituted with 1-5 substituents which can be the same or different, each substituent being independently selected from the group consisting of the moieties shown above.

It should be understood that when R² and R⁶ are joined together to form a 5-14 membered heteroaryl, 5-8 membered heterocyclyl or 5-8 membered heterocyclenyl moiety, each of said heteroaryl, heterocyclyl or heterocyclenyl moiety independently may optionally additionally be fused with an aryl or heteroaryl ring, wherein the ring moiety resulting from the fusion may be unsubstituted or optionally independently substituted with 1-5 substituents which can be the same or different, each substituent being independently selected from the group consisting of the moieties shown above.

It should be understood that when R⁶ and R⁵ are joined together to form a 5-14 membered heteroaryl, 5-8 membered heterocyclyl or 5-8 membered heterocyclenyl moiety, each of said heteroaryl, heterocyclyl or heterocyclenyl moiety independently may optionally additionally be fused with an aryl or heteroaryl ring, wherein the ring moiety resulting from the fusion may be unsubstituted or optionally independently substituted with 1-5 substituents which can be the same or different, each substituent being independently selected from the group consisting of the moieties shown above.

It should be understood that when R² and R¹⁴ are joined together to form a 5-14 membered heteroaryl, 5-8 membered heterocyclyl or 5-8 membered heterocyclenyl moiety, each of said heteroaryl, heterocyclyl or heterocyclenyl moiety independently may optionally additionally be fused with an aryl or heteroaryl ring, wherein the ring moiety resulting from the fusion may be unsubstituted or optionally independently substituted with 1-5 substituents which can be the same or different, each substituent being independently selected from the group consisting of the moieties shown above.

It should be understood that when R³ and R¹⁴ are joined together to form a 5-14 membered heteroaryl, 5-8 membered heterocyclyl or 5-8 membered heterocyclenyl moiety, each of said heteroaryl, heterocyclyl or heterocyclenyl moiety independently may optionally additionally be fused with an aryl or heteroaryl ring, wherein the ring moiety resulting from the fusion may be unsubstituted or optionally independently substituted with 1-5 substituents which can be the same or different, each substituent being independently selected from the group consisting of the moieties shown above.

It should be understood that when R³ and R⁶ are joined together to form a 5-14 membered aryl, 5-8 membered cycloalkyl, 5-8 membered cycloalkenyl, 5-14 membered heteroaryl, 5-8 membered heterocyclyl or 5-8 membered heterocyclenyl moiety, each of said aryl, cycloalkyl, cycloalkenyl, heteroaryl, heterocyclyl or heterocyclenyl moiety independently may optionally additionally be fused with an aryl or heteroaryl ring, wherein the ring moiety resulting from the fusion may be unsubstituted or optionally independently substituted with 1-5 substituents which can be the same or different, each substituent being independently selected from the group consisting of the moieties shown above.

It should be understood that when R¹⁴ and R⁵ are joined together to form a 5-14 membered heteroaryl, 5-8 membered heterocyclyl or 5-8 membered heterocyclenyl moiety, each of said heteroaryl, heterocyclyl or heterocyclenyl moiety independently may optionally additionally be fused with an aryl or heteroaryl ring, wherein the ring moiety resulting from the fusion may be unsubstituted or optionally independently substituted with 1-5 substituents which can be the same or different, each substituent being independently selected from the group consisting of the moieties shown above.

Thus, in one embodiment, the present application discloses a compound of the Formula (I):

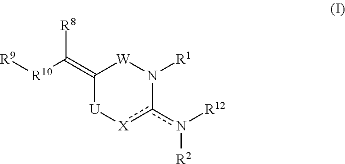

or a pharmaceutically acceptable salt, solvate, ester or prodrug thereof, wherein:

either
(i) R¹ and R² are joined together to form a 5-14 membered heteroaryl, 5-8 membered heterocyclyl or 5-8 membered heterocyclenyl moiety, wherein: (a) said heteroaryl moiety is optionally substituted with 1 to 5 independently selected R²¹ groups, (b) said heterocyclyl moiety is optionally substituted with 1 to 5 independently selected R²¹ groups, (c) said heterocyclenyl moiety is optionally substituted with 1 to 5 independently selected R²¹ groups, and (d) said heteroaryl, heterocyclyl or heterocyclenyl moiety is optionally fused with an aryl or heteroaryl ring, and the ring moiety resulting from the fusion is optionally substituted with 1 to 5 independently selected R²¹ groups; or
(ii) R² and R⁶ are joined together to form a 5-14 membered heteroaryl,

5-8 membered heterocyclyl or 5-8 membered heterocyclenyl moiety, wherein: (a) said heteroaryl moiety is optionally substituted with 1 to 5 independently selected R²¹ groups, (b) said heterocyclyl moiety is optionally substituted with 1 to 5 independently selected R²¹ groups, (c) said heterocyclenyl moiety is optionally substituted with 1 to 5 independently selected R²¹ groups, and (d) said heteroaryl, heterocyclyl or heterocyclenyl moiety is optionally fused with an aryl or heteroaryl ring, and the ring moiety resulting from the fusion is optionally substituted with 1 to 5 independently selected R²¹ groups; or
(iii) R⁵ and R⁶ are joined together to form a 5-14 membered heteroaryl, 5-8 membered heterocyclyl or 5-8 membered heterocyclenyl moiety, wherein: (a) said heteroaryl moiety is optionally substituted with 1 to 5 independently selected R²¹ groups, (b) said heterocyclyl moiety is optionally substituted with 1 to 5 independently selected R²¹ groups, (c) said heterocyclenyl moiety is optionally substituted with 1 to 5 independently selected R²¹ groups, and (d) said heteroaryl, heterocyclyl or heterocyclenyl moiety is optionally fused with an aryl or heteroaryl ring, and the ring moiety resulting from the fusion is optionally substituted with 1 to 5 independently selected R²¹ groups; or
(iv) R² and R¹⁴ are joined together to form a 5-14 membered heteroaryl, 5-8 membered heterocyclyl or 5-8 membered heterocyclenyl moiety, wherein: (a) said heteroaryl moiety is optionally substituted with 1 to 5 independently selected R²¹ groups, (b) said heterocyclyl moiety is optionally substituted with 1 to 5 independently selected R²¹ groups, (c) said heterocyclenyl moiety is optionally substituted with 1 to 5 independently selected R²¹ groups, and (d) said heteroaryl, heterocyclyl or heterocyclenyl moiety is optionally fused with an aryl or heteroaryl ring, and the ring moiety resulting from the fusion is optionally substituted with 1 to 5 independently selected R²¹ groups; or
(v) R³ and R¹⁴ are joined together to form a 5-14 membered heteroaryl, 5-8 membered heterocyclyl or 5-8 membered heterocyclenyl moiety, wherein: (a) said heteroaryl moiety is optionally substituted with 1 to 5 independently selected R²¹ groups, (b) said heterocyclyl moiety is optionally substituted with 1 to 5 independently selected R²¹ groups, (c) said heterocyclenyl moiety is optionally substituted with 1 to 5 independently selected R²¹ groups, and (d) said heteroaryl, heterocyclyl or heterocyclenyl moiety is optionally fused with an aryl or heteroaryl ring, and the ring moiety resulting from the fusion is optionally substituted with 1 to 5 independently selected R²¹ groups; or
(vi) R³ and R⁶ are joined together to form a 5-14 membered aryl, 5-8 membered cycloalkyl, 5-8 membered cycloalkenyl, 5-14 membered heteroaryl, 5-8 membered heterocyclyl or 5-8 membered heterocyclenyl moiety, wherein: (a) said aryl moiety is optionally substituted with 1 to 5 independently selected R²¹ groups, (b) said cycloalkyl moiety is optionally substituted with 1 to 5 independently selected R²¹ groups, (c) said cycloalkenyl moiety is optionally substituted with 1 to 5 independently selected R²¹ groups, (d) said heteroaryl moiety is optionally substituted with 1 to 5 independently selected R²¹ groups, (e) said heterocyclyl moiety is optionally substituted with 1 to 5 independently selected R²¹ groups, (f) said heterocyclenyl moiety is optionally substituted with 1 to 5 independently selected R²¹ groups, and (g) said aryl, cycloalkyl, cycloalkenyl, heteroaryl, heterocyclyl or heterocyclenyl moiety is optionally fused with an aryl or heteroaryl ring, and the ring moiety resulting from the fusion is optionally substituted with 1 to 5 independently selected R²¹ groups; or
(vii) R⁵ and R¹⁴ are joined together to form a 5-14 membered heteroaryl, 5-8 membered heterocyclyl or 5-8 membered heterocyclenyl moiety, wherein: (a) said heteroaryl moiety is optionally substituted with 1 to 5 independently selected R²¹ groups, (b) said heterocyclyl moiety is optionally substituted with 1 to 5 independently selected R²¹ groups, (c) said heterocyclenyl moiety is optionally substituted with 1 to 5 independently selected R²¹ groups, and (d) said heteroaryl, heterocyclyl or heterocyclenyl moiety is optionally fused with an aryl or heteroaryl ring, and the ring moiety resulting from the fusion is optionally substituted with 1 to 5 independently selected R²¹ groups;

W is —S(O)—, —S(O)₂— or —C(O)—;

U is a bond, —C(O)—, —O—, —N(R⁵)— or —C(R³)(R⁴)—;

X is —N(R¹⁴)— or —C(R⁶)(R⁷)—;

The dashed lines [symbol] in formula (I) represent optional bonds provided that: (a) only one optional bond can be present (i.e, either there can be an optional bond between X and the adjacent ring carbon, or there can be an optional bond between the nitrogen and the ring carbon), and (b) when the optional bond between the nitrogen (of the NR² moiety) and the ring carbon is present then R¹² is absent (i.e., there is no R¹² moiety bound to the nitrogen);

R¹ (when R¹ is not joined to R²) is independently selected from the group consisting of H, alkyl, alkenyl, alkynyl, aryl, arylalkyl-, alkylaryl-, cycloalkyl, cycloalkylalkyl-, heteroaryl, heteroarylalkyl-, heterocyclyl and heterocyclyalkyl-, wherein each of said alkyl, alkenyl, alkynyl, aryl, arylalkyl-, alkylaryl-, cycloalkyl, cycloalkylalkyl-, heteroaryl, heteroarylalkyl-, heterocyclyl and heterocyclyalkyl-R¹ group is optionally substituted with 1-5 independently selected R²¹ substituents;

R² (when R² is not joined to R¹, R⁶ or R¹⁴) is independently selected from the group consisting of H, alkyl, alkenyl, alkynyl, cycloalkyl, cycloalkylalkyl-, cycloalkenyl, heterocyclyl, heterocyclylalkyl-, aryl, arylalkyl-, heteroaryl, heteroarylalkyl-, —CN, —C(O)R¹⁵, —C(O)OR¹⁵, —C(O)N(R¹⁵)(R¹⁶), —S(O)N(R¹⁵)(R¹⁶), —S(O)₂N(R¹⁵)(R¹⁶), —S(O)R¹⁵, —S(O)₂R¹⁵, —C(═NOR¹⁵)R¹⁶ and —P(O)(OR¹⁵)(OR¹⁶), and wherein each of said alkyl, alkenyl, alkynyl, cycloalkyl, cycloalkylalkyl-, cycloalkenyl, heterocyclyl, heterocyclylalkyl-, aryl, arylalkyl-, heteroaryl, and heteroarylalkyl-R² group is optionally substituted with 1-5 independently selected R²¹ groups;

R³ (when R³ is not joined to R⁶ or R¹⁴) is independently selected from the group consisting of H, alkyl, alkenyl, alkynyl, aryl, arylalkyl-, alkylaryl-, cycloalkyl, cycloalkylalkyl-, heteroaryl, heteroarylalkyl-, heterocyclyl and heterocyclyalkyl-, wherein each of said alkyl-, alkenyl, alkynyl-, aryl-, arylalkyl-, alkylaryl-, cycloalkyl-, cycloalkylalkyl-, heteroaryl-, heteroarylalkyl-, heterocyclyl- and heterocyclyalkyl-R³ group is optionally substituted with 1-5 independently selected R²¹ groups;

R⁴ is independently selected from the group consisting of H, alkyl, alkenyl, alkynyl, aryl, arylalkyl-, alkylaryl-, cycloalkyl-, cycloalkylalkyl-, heteroaryl-, heteroarylalkyl-, heterocyclyl- and heterocyclyalkyl-, and wherein each of said alkyl, alkenyl, alkynyl, aryl, arylalkyl-, alkylaryl-, cycloalkyl, cycloalkylalkyl-, heteroaryl, heteroarylalkyl-, heterocyclyl and heterocyclyalkyl-R⁴ group is optionally substituted with 1-5 independently selected R²¹ substituents;

R⁵ (when R⁵ is not joined to R⁶ or R¹⁴) is independently selected from the group consisting of H, alkyl, alkenyl, alkynyl, cycloalkyl, cycloalkylalkyl, cycloalkenyl, heterocyclyl, heterocyclylalkyl, aryl, arylalkyl, heteroaryl, heteroarylalkyl, —CN, —C(O)R¹⁵, —C(O)OR¹⁵, —C(O)N(R¹⁵)(R¹⁶), —S(O)N(R¹⁵)(R¹⁶), —S(O)₂N(R¹⁵)(R¹⁶), —S(O)R¹⁵, —S(O)₂R¹⁵, —C(═NOR¹⁵)R¹⁶ and —P(O)(OR¹⁵)(OR¹⁶), and wherein each of said alkyl, alkenyl, alkynyl, cycloalkyl, cycloalkylalkyl, cycloalkenyl, heterocyclyl, heterocyclylalkyl, aryl, arylalkyl, heteroaryl, and heteroarylalkyl R⁵ groups is optionally substituted with 1-5 independently selected R²¹ groups;

R⁶ (when R⁶ is not joined to R², R³ or R⁵) is independently selected from the group consisting of H, alkyl, alkenyl, alkynyl, aryl, arylalkyl-, alkylaryl-, cycloalkyl, cycloalkylalkyl-, heteroaryl, heteroarylalkyl-, heterocyclyl- and heterocyclyalkyl-, wherein each of said alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl-R⁶ group is optionally substituted with 1-5 independently selected R²¹;

R⁷ is independently selected from the group consisting of H, alkyl, alkenyl, alkynyl, aryl, arylalkyl-, alkylaryl-, cycloalkyl, cycloalkylalkyl-, heteroaryl, heteroarylalkyl-, heterocyclyl and heterocyclyalkyl-, and wherein each of said alkyl, alkenyl, alkynyl, aryl, arylalkyl-, alkylaryl-, cycloalkyl, cycloalkylalkyl-, heteroaryl, heteroarylalkyl-, heterocyclyl and heterocyclyalkyl-R⁷ group is optionally substituted with 1-5 independently selected R²¹ substituents;

R⁸ is independently selected from the group consisting of H, alkyl, alkenyl, alkynyl, aryl, arylalkyl-, alkylaryl-, cycloalkyl, cycloalkylalkyl-, heteroaryl, heteroarylalkyl-, heterocyclyl and heterocyclyalkyl-, and wherein each of said alkyl, alkenyl, alkynyl, aryl, arylalkyl-, alkylaryl-, cycloalkyl, cycloalkylalkyl-, heteroaryl, heteroarylalkyl-, heterocyclyl and heterocyclyalkyl-R⁸ group is optionally substituted with 1-3 independently selected R²¹ substituents;

R⁹ is independently selected from the group consisting of alkyl, alkenyl alkynyl, aryl, arylalkyl-, alkylaryl-, cycloalkyl, cycloalkylalkyl-, heteroaryl, heteroarylalkyl-, heterocyclyl and heterocyclyalkyl-, and wherein each of said alkyl, alkenyl, alkynyl, aryl, arylalkyl-, alkylaryl-, cycloalkyl, cycloalkylalkyl-, heteroaryl, heteroarylalkyl-, heterocyclyl and heterocyclyalkyl-R⁹ group is optionally substituted with 1-3 independently selected R²¹ groups,

R¹⁰ is independently selected from the group consisting of: a bond, alkyl, alkenyl, alkynyl, aryl, arylalkyl-, alkylaryl-, cycloalkyl, cycloalkylalkyl-, heteroaryl, heteroarylalkyl-, heterocyclyl, heterocyclyalkyl-,

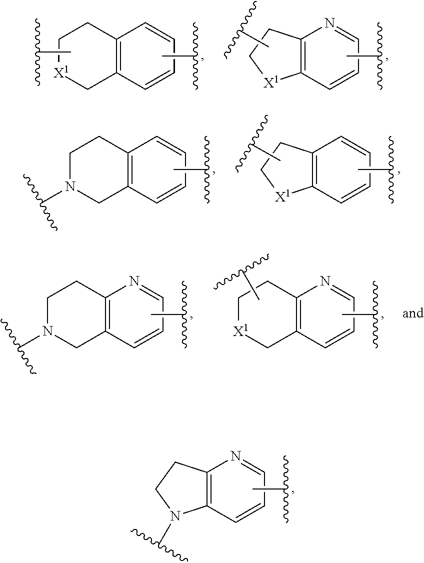

wherein X¹ is O, N(R¹⁴) or S; and wherein each of said R¹⁰ moieties (except for the bond) is optionally substituted with 1-3 independently selected R²¹ substituents;

R¹² is independently selected from the group consisting of H, alkyl, alkenyl, alkynyl, cycloalkyl, cycloalkylalkyl-, cycloalkenyl, heterocyclyl, heterocyclylalkyl-, aryl, arylalkyl-, heteroaryl, heteroarylalkyl-, —CN, —C(O)R¹⁶, —C(O)OR¹⁶, —C(O)N(R¹⁵)(R¹⁶), —S(O)N(R¹⁵)(R¹⁶), —S(O)₂N(R¹⁵)(R¹⁶), —S(O)R¹⁵, —S(O)₂R¹⁵, —C(═NOR¹⁵)R¹⁶ and —P(O)(OR¹⁵)(OR¹⁶), and wherein each of said alkyl, alkenyl, alkynyl, cycloalkyl, cycloalkylalkyl-, cycloalkenyl, heterocyclyl, heterocyclylalkyl-, aryl, arylalkyl-, heteroaryl, and heteroarylalkyl-R¹² group is optionally substituted with 1 to 5 independently selected R²¹ groups;

R¹⁴ (when R¹⁴ is not joined to R², R³ or R⁵) is independently selected from the group consisting of H, alkyl, alkenyl, alkynyl, cycloalkyl, cycloalkylalkyl-, cycloalkenyl, heterocyclyl, heterocyclylalkyl-, aryl, arylalkyl-, heteroaryl, heteroarylalkyl-, —CN, —C(O)R¹⁵, —C(O)OR¹⁵, —C(O)N(R¹⁵)(R¹⁶), —S(O)N(R¹⁵)(R¹⁶), —S(O)₂N(R¹⁵)(R¹⁶), —S(O)R¹⁵, —S(O)₂R¹⁵, —C(═NOR¹⁵)R¹⁶, and —P(O)(OR¹⁵)(OR¹⁶), and wherein each of said alkyl, alkenyl, alkynyl, cycloalkyl, cycloalkenyl, heterocyclyl, heterocyclylalkyl-, aryl, arylalkyl-, heteroaryl, and heteroarylalkyl-R¹⁴ group is optionally substituted with 1 to 5 independently selected R²¹ groups;

R¹⁵, R¹⁶ and R¹⁷ are each independently selected from the group consisting of H, alkyl, alkenyl, alkynyl, cycloalkyl, cycloalkylalkyl-, heterocyclyl, heterocyclylalkyl-, aryl, arylalkyl-, heteroaryl, heteroarylalkyl-, arylcycloalkyl-, arylheterocyclyl-, (R¹⁸)ₙ-alkyl-, (R¹⁸)ₙ-cycloalkyl-, (R¹⁸)ₙ-cycloalkylalkyl-, (R¹⁸)ₙ-heterocyclyl-, (R¹⁸)ₙ-heterocyclylalkyl-, (R¹⁸)ₙ-aryl-, (R¹⁸)ₙ-arylalkyl-, (R¹⁸)ₙ-heteroaryl- and (R¹⁸)ₙ-heteroarylalkyl, wherein n is 1 to 5;

Each R¹⁸ is independently selected from the group consisting of alkyl, alkenyl, alkynyl, aryl, arylalkyl-, arylalkenyl-, arylalkynyl-, —NO₂, halo, heteroaryl, HO-alkyoxyalkyl-, —CF₃, —CN, -alkyl-CN, —C(O)R¹⁹, —C(O)OH, —C(O)OR¹⁹, —C(O)NHR²⁰, —C(O)NH₂, —C(O)NH₂—C(O)N(alkyl)₂, —C(O)N(alkyl)(aryl), —C(O)N(alkyl)(heteroaryl), —SR¹⁹, —S(O)₂R²⁰, —S(O)NH₂, —S(O)NH(alkyl), —S(O)N(alkyl)(alkyl), —S(O)NH(aryl), —S(O)₂NH₂, —S(O)₂NHR¹⁹, —S(O)₂NH(heterocyclyl), —S(O)₂N(alkyl)₂, —S(O)₂N(alkyl)(aryl), —OCF₃, —OH, —OR²⁰, —O-heterocyclyl, —O-cycloalkylalkyl, —O-heterocyclylalkyl, —NH₂, —NHR²⁰, —N(alkyl)₂, —N(arylalkyl)₂, —N(arylalkyl)-(heteroarylalkyl), —NHC(O)R²⁰, —NHC(O)NH₂, —NHC(O)NH(alkyl), —NHC(O)N(alkyl)(alkyl), —N(alkyl)C(O)NH(alkyl), —N(alkyl)C(O)N(alkyl)(alkyl), —NHS(O)₂R²⁰, —NHS(O)₂NH(alkyl), —NHS(O)₂N(alkyl)(alkyl), —N(alkyl)S(O)₂NH(alkyl) and —N(alkyl)S(O)₂N(alkyl)(alkyl);

or, alternately, two R¹⁸ moieties on adjacent carbons can be linked together to form:

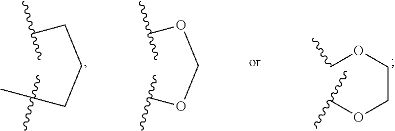

R¹⁹ is independently selected from the group consisting of: alkyl, cycloalkyl, aryl, arylalkyl- and heteroarylalkyl-;

R²⁰ is independently selected from the group consisting of: alkyl, cycloalkyl, aryl, halo substituted aryl, arylalkyl-, heteroaryl or heteroarylalkyl;

each R²¹ is independently selected from the group consisting of: alkyl, alkenyl, alkynyl, cycloalkyl, cycloalkylalkyl-, cycloalkenyl, heterocyclyl, heterocyclylalkyl-, aryl, arylalkyl-, heteroaryl, heteroarylalkyl, halo, —CN, —OR¹⁵, —C(O)R¹⁵, —C(O)OR¹⁵, —C(O)N(R¹⁵)(R¹⁶), —SR¹⁵, —S(O)N(R¹⁵)(R¹⁶), —CH(R¹⁵)(R¹⁶), —S(O)₂N(R¹⁵)(R¹⁶), —C(═NOR¹⁵)R¹⁶, —P(O)(OR¹⁵)(OR¹⁶), —N(R¹⁵)(R¹⁶), -alkyl-N(R¹⁵)(R¹⁶), —N(R¹⁵)C(O)R¹⁶, —CH₂—N(R¹⁵)C(O)R¹⁶, —CH₂—N(R¹⁵)C(O)N(R¹⁶)(R¹⁷), —CH₂—R¹⁵; —CH₂N(R¹⁵)(R¹⁶), —N(R¹⁵)S(O)R¹⁶, —N(R¹⁵)S(O)₂R¹⁶, —CH₂—N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —CH₂—N(R¹⁵)C(O)N(R¹⁶)(R¹⁷), —N(R¹⁵)C(O)OR¹⁶, —CH₂—N(R¹⁵)C(O)OR¹⁶, —S(O)R¹⁵, ═NOR¹⁵, —N₃, —NO₂ and —S(O)₂R¹⁵; and wherein each of the alkyl, cycloalkenyl, cycloalkyl, cycloalkylalkyl, heterocyclyl, heterocyclylalkyl, aryl, arylalkyl, heteroaryl, heteroarylalkyl, alkenyl and alkynyl R²¹ groups is optionally substituted with 1 to 5 independently selected R²² groups (those skilled in the art will appreciate that the optional R²² substitution on one R²¹ group is independent of the optional R²² substitution on any other R²¹ group, and when there is more than one optional R²² substituent on the same R²¹ group each optional R²² substitutent is independently selected);

Each R²² is independently selected from the group consisting of: alkyl, cycloalkyl, cycloalkenyl, heterocyclyl, aryl, heteroaryl, halo, —CF₃, —CN, —OR¹⁵, —C(O)R¹⁵, —C(O)OR¹⁵, -alkyl-C(O)OR¹⁵, C(O)N(R¹⁵)(R¹⁶), —SR'⁵, —S(O)N(R¹⁵)(R¹⁶), —S(O)₂N(R¹⁵)(R¹⁶), —C(═NOR¹⁵)R¹⁶, —P(O)(OR¹⁵)(OR¹⁶), —N(R¹⁵)(R¹⁶), -alkyl-N(R¹⁵)(R¹⁶), —N(R¹⁵)C(O)R¹⁶, —CH₂—N(R¹⁵)C(O)R¹⁶, —N(R¹⁵)S(O)R¹⁶, —N(R¹⁵)S(O)₂R¹⁶, —CH₂—N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —CH₂—N(R¹⁵)C(O)N(R¹⁶)(R¹⁷), —N(R¹⁵)C(O)OR¹⁶, —CH₂—N(R¹⁵)C(O)OR¹⁶, —N₃, ═NOR¹⁵, —NO₂, —S(O)R¹⁵ and —S(O)₂R¹⁵.

In another embodiment, the present application discloses a compound, or pharmaceutically acceptable salts, solvates, esters or prodrugs of said compound, said compound having the general structure shown in Formula:

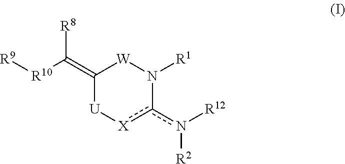

wherein:
R¹ and R² are joined together to form a 5-14 membered heteroaryl, 5-8 membered heterocyclyl or 5-8 membered heterocyclenyl moiety, with each of said heteroaryl, heterocyclyl or heterocyclenyl moiety being unsubstituted or optionally independently being substituted with 1-5 substituents which can be the same or different, each substituent being independently selected from the group consisting of the moieties shown below;
W is —S(O)—, —S(O)₂— or —C(O)—;
U is a bond, —C(O)—, —O—, —N(R⁵)— or —C(R³)(R⁴)—;
X is —N(R¹⁴)— or —C(R⁶)(R⁷)—;
each dashed line of

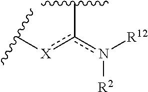

along with its adjoining single bond, together represents an optional double bond with the proviso that only one such double bond [symbol] is present at any given time, and further such that when the nitrogen of N(R²)(R¹²) is double bonded to the adjacent carbon between said nitrogen and X by the optional double bond, then R¹² is absent;

R⁵ is selected from the group consisting of H, alkyl, alkenyl, alkynyl, cycloalkyl, cycloalkylalkyl, cycloalkenyl, heterocyclyl, heterocyclylalkyl, aryl, arylalkyl, heteroaryl, heteroarylalkyl, —CN, —C(O)R¹⁵, —C(O)OR¹⁵, —C(O)N(R¹⁵)(R¹⁶), —S(O)N(R¹⁵)(R¹⁶), —S(O)₂N(R¹⁵)(R¹⁶), —S(O)R¹⁵, —S(O)₂R¹⁵, —C(═NOR¹⁵)R¹⁶ and —P(O)(OR¹⁵)(OR¹⁶);

R¹² is independently selected from the group consisting of H, alkyl, alkenyl, alkynyl, cycloalkyl, cycloalkylalkyl, cycloalkenyl, heterocyclyl, heterocyclylalkyl, aryl, arylalkyl, heteroaryl, heteroarylalkyl, —CN, —C(O)R¹⁵, —C(O)OR¹⁵, —C(O)N(R¹⁵)(R¹⁶), —S(O)N(R¹⁵)(R¹⁶), —S(O)₂N(R¹⁵)(R¹⁶), —S(O)R¹⁵, —S(O)₂R¹⁵, —C(═NOR¹⁵)R¹⁶ and —P(O)(OR¹⁵)(OR¹⁶);

each R¹⁴ can be the same or different, each being independently selected from the group consisting of H, alkyl, alkenyl, alkynyl, cycloalkyl, cycloalkylalkyl, cycloalkenyl, heterocyclyl, heterocyclylalkyl, aryl, arylalkyl, heteroaryl, heteroarylalkyl, —CN, —C(O)R¹⁵, —C(O)OR¹⁵, —C(O)N(R¹⁵)(R¹⁶), —S(O)N(R¹⁵)(R¹⁶), —S(O)₂N(R¹⁵)(R¹⁶), —S(O)R¹⁵, —S(O)₂R¹⁵, —C(═NOR¹⁵)R¹⁶, and —P(O)(OR¹⁵)(OR¹⁶);

R³ is independently selected from the group consisting of H, alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl-, wherein each of said alkyl-, alkenyl- alkynyl-, aryl-, arylalkyl-, alkylaryl-, cycloalkyl-, cycloalkylalkyl-, heteroaryl-, heteroarylalkyl-, heterocyclyl- and heterocyclyalkyl- can be unsubstituted or optionally independently substituted with 1-5 substituents which can be the same or different, each substituent being independently selected from the group consisting of consisting of the moieties shown below;

R⁴ is independently selected from the group consisting of H, alkyl-, alkenyl-alkynyl-, aryl-, arylalkyl-, alkylaryl-, cycloalkyl-, cycloalkylalkyl-, heteroaryl-, heteroarylalkyl-, heterocyclyl- and heterocyclyalkyl-, wherein each of said alkyl-, alkenyl- alkynyl-, aryl-, arylalkyl-, alkylaryl-, cycloalkyl-, cycloalkylalkyl-, heteroaryl-, heteroarylalkyl-, heterocyclyl- and heterocyclyalkyl- can be unsubstituted or optionally independently substituted with 1-5 substituents which can be the same or different, each substituent being independently selected from the group consisting of consisting of the moieties shown below;

R⁶ is selected from the group consisting of H, alkyl-, alkenyl- alkynyl-, aryl-, arylalkyl-, alkylaryl-, cycloalkyl-, cycloalkylalkyl-, heteroaryl-, heteroarylalkyl-, heterocyclyl- and heterocyclyalkyl-, wherein each of said alkyl-, alkenyl- alkynyl-, aryl-, arylalkyl-, alkylaryl-, cycloalkyl-, cycloalkylalkyl-, heteroaryl-, heteroarylalkyl-, heterocyclyl- and heterocyclyalkyl- can be unsubstituted or optionally independently substituted with 1-5 substituents which can be the same or different, each substituent being independently selected from the group consisting of consisting of the moieties shown below;

R⁷ is selected from the group consisting of H, alkyl-, alkenyl- alkynyl-, aryl-, arylalkyl-, alkylaryl-, cycloalkyl-, cycloalkylalkyl-, heteroaryl-, heteroarylalkyl-, heterocyclyl- and heterocyclyalkyl-, wherein each of said alkyl-, alkenyl- alkynyl-, aryl-, arylalkyl-, alkylaryl-, cycloalkyl-, cycloalkylalkyl-, heteroaryl-, heteroarylalkyl-, heterocyclyl- and heterocyclyalkyl- can be unsubstituted or optionally independently substituted with 1-5 substituents which can be the same or different, each substituent being independently selected from the group consisting of consisting of the moieties shown below;

R⁸ is selected from the group consisting of H, alkyl-, alkenyl- alkynyl-, aryl-, arylalkyl-, alkylaryl-, cycloalkyl-, cycloalkylalkyl-, heteroaryl-, heteroarylalkyl-, heterocyclyl- and heterocyclyalkyl-, with each of said alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl- being unsubstituted or optionally independently substituted with 1-3 substituents which can be the same or different, each substituent being independently selected from the group consisting of the moieties shown below;

R⁹ is selected from the group consisting of alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl-, wherein each of said alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl- can be unsubstituted or optionally independently substituted with 1-3 substituents which can be the same or different, each substituent being independently selected from the group consisting of the moieties shown below,

R¹⁰ is selected from the group consisting of a bond, alkyl-, alkenyl-, alkynyl-, aryl-, arylalkyl-, alkylaryl-, cycloalkyl-, cycloalkylalkyl-, heteroaryl-, heteroarylalkyl-, heterocyclyl-, heterocyclyalkyl- and the moieties:

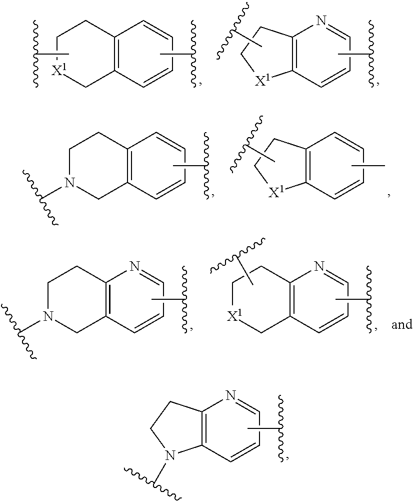

where X¹ is O, N(R¹⁴) or S;

wherein each of said alkyl-, alkenyl-, alkynyl-, aryl-, arylalkyl-, alkylaryl-, cycloalkyl-, cycloalkylalkyl-, heteroaryl-, heteroarylalkyl-, heterocyclyl- heterocyclyalkyl- and the above-noted moieties for R¹⁰ can be unsubstituted or optionally independently substituted with 1-3 substituents which can be the same or different, each being independently selected from the group consisting of the moieties shown below; and

R¹⁵, R¹⁶ and R¹⁷ are independently selected from the group consisting of H, alkyl, alkenyl, alkynyl, cycloalkyl, cycloalkylalkyl, heterocyclyl, heterocyclylalkyl, aryl, arylalkyl, heteroaryl, heteroarylalkyl, arylcycloalkyl, arylheterocyclyl, R¹⁸-alkyl, R¹⁸-cycloalkyl, R¹⁸-cycloalkylalkyl, R¹⁸-heterocyclyl, R¹⁸-heterocyclylalkyl, R¹⁸-aryl, R¹⁸-arylalkyl, R¹⁸-heteroaryl and R¹⁸-heteroarylalkyl;

R¹⁸ is 1-5 substituents independently selected from the group consisting of alkyl, alkenyl, alkynyl, aryl, arylalkyl, arylalkenyl, arylalkynyl, —NO₂, halo, heteroaryl, HO-alkyoxyalkyl, —CF₃, —CN, alkyl-CN, —C(O)R¹⁹, —C(O)OR¹⁹, —C(O)NHR²⁰, —C(O)NH₂, —C(O)NH₂—C(O)N(alkyl)₂, —C(O)N(alkyl)(aryl), —C(O)N(alkyl)(heteroaryl), —SR¹⁹, —S(O)₂R²⁰, —S(O)NH₂, —S(O)NH(alkyl), —S(O)N(alkyl)(alkyl), —S(O)NH(aryl), —S(O)₂NH₂, —S(O)₂NHR¹⁹, —S(O)₂NH(heterocyclyl), —S(O)₂N(alkyl)₂, —S(O)₂N(alkyl)(aryl), —OCF₃, —OH, —OR²⁰, —O-heterocyclyl, —O-cycloalkylalkyl, —O-heterocyclylalkyl, —NH₂, —NHR²⁰, —N(alkyl)₂, —N(arylalkyl)₂, —N(arylalkyl)-(heteroarylalkyl), —NHC(O)R²⁰, —NHC(O)NH₂, —NHC(O)NH(alkyl), —NHC(O)N(alkyl)(alkyl), —N(alkyl)C(O)NH(alkyl), —N(alkyl)C(O)N(alkyl)(alkyl), —NHS(O)₂R²⁰, —NHS(O)₂NH(alkyl), —NHS(O)₂N(alkyl)(alkyl), —N(alkyl)S(O)₂NH(alkyl) and —N(alkyl)S(O)₂N(alkyl)(alkyl);

or, alternately, two R¹⁸ moieties on adjacent carbons can be linked together to form:

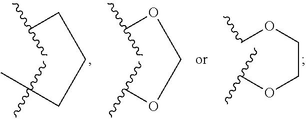

R¹⁹ is alkyl, cycloalkyl, aryl, arylalkyl or heteroarylalkyl;

R²⁰ is alkyl, cycloalkyl, aryl, halo substituted aryl, arylalkyl, heteroaryl or heteroarylalkyl;

wherein each of the alkyl, cycloalkyl, cycloalkylalkyl, heterocyclyl, heterocyclylalkyl, aryl, arylalkyl, alkylaryl, heteroaryl, heteroarylalkyl, alkenyl and alkynyl groups in R³, R⁴, R⁵, R⁶, R⁷, R⁸, R⁹, R¹⁰, R¹² and R¹⁴, the 5-14 membered heteroaryl, 5-8 membered heterocyclyl or 5-8 membered heterocyclenyl moiety formed from the joining of R¹ and R² are independently unsubstituted or substituted by 1 to 5 R²¹ groups independently selected from the group consisting of alkyl, alkenyl, alkynyl, cycloalkyl, cycloalkylalkyl, cycloalkenyl, heterocyclyl, heterocyclylalkyl, aryl, arylalkyl, heteroaryl, heteroarylalkyl, halo, —CN, —OR¹⁵, —C(O)R¹⁵, —C(O)OR¹⁵, —C(O)N(R¹⁵)(R¹⁶), —SR¹⁵, —S(O)N(R¹⁵)(R¹⁶), —CH(R¹⁵)(R¹⁶), —S(O)₂N(R¹⁵)(R¹⁶), —C(═NOR¹⁵)R¹⁶, —P(O)(OR¹⁵)(OR¹⁶), —N(R¹⁵)(R¹⁶), -alkyl-N(R¹⁵)(R¹⁶), —N(R¹⁵)C(O)R¹⁶, —CH₂—N(R¹⁵)C(O)R¹⁶, —CH₂—N(R¹⁵)C(O)N(R¹⁶)(R¹⁷), —CH₂—R¹⁵; —CH₂N(R¹⁵)(R¹⁶), —N(R¹⁵)S(O)R¹⁶, —N(R¹⁵)S(O)₂R¹⁶, —CH₂—N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —CH₂—N(R¹⁵)C(O)N(R¹⁶)(R¹⁷), —N(R¹⁵)C(O)OR¹⁶, —CH₂—N(R¹⁵)C(O)OR¹⁶, —S(O)R¹⁵, ═NOR¹⁵, —N₃, —NO₂ and —S(O)₂R¹⁵;

wherein each of the alkyl, cycloalkenyl, cycloalkyl, cycloalkylalkyl, heterocyclyl, heterocyclylalkyl, aryl, arylalkyl, heteroaryl, heteroarylalkyl, alkenyl and alkynyl groups in R²¹ are independently unsubstituted or substituted by 1 to 5 R²² groups independently selected from the group consisting of alkyl, cycloalkyl, cycloalkenyl, heterocyclyl, aryl, heteroaryl, halo, —CF₃, —CN, —OR¹⁵, —C(O)R¹⁵, —C(O)OR¹⁵, -alkyl-C(O)OR¹⁵, C(O)N(R¹⁵)(R¹⁶), —SR¹⁵, —S(O)N(R¹⁵)(R¹⁶), —S(O)₂N(R¹⁵)(R¹⁶), —C(═NOR¹⁵)R¹⁶, —P(O)(OR¹⁵)(OR¹⁶), —N(R¹⁵)(R¹⁶), -alkyl-N(R¹⁵)(R¹⁶), —N(R¹⁵)C(O)R¹⁶, —CH₂—N(R¹⁵)C(O)R¹⁶, —N(R¹⁵)S(O)R¹⁶, —N(R¹⁵)S(O)₂R¹⁶, —CH₂—N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —CH₂—N(R¹⁵)C(O)N(R¹⁶)(R¹⁷), —N(R¹⁵)C(O)OR¹⁶, —CH₂—N(R¹⁵)C(O)OR¹⁶, —N₃, ═NOR¹⁵, —NO₂, —S(O)R¹⁵ and —S(O)₂R¹⁵.

In another embodiment, the present application discloses a compound, or pharmaceutically acceptable salts, solvates, esters or prodrugs of said compound, said compound having the general structure shown in Formula:

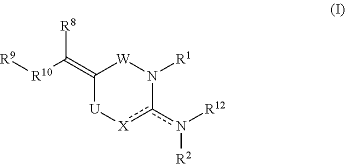

wherein:
R² and R⁶ are joined together to form a 5-14 membered heteroaryl, 5-8 membered heterocyclyl or 5-8 membered heterocyclenyl moiety, with each of said heteroaryl, heterocyclyl or heterocyclenyl moiety being unsubstituted or optionally independently being substituted with 1-5 substituents which can be the same or different, each substituent being independently selected from the group consisting of the moieties shown below;
W is —S(O)—, —S(O)₂— or —C(O)—;
U is a bond, —C(O)—, —O—, —N(R⁵)— or —C(R³)(R⁴)—;
X is —C(R⁶)(R⁷)—;
each dashed line of

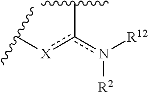

along with its adjoining single bond, together represents an optional double bond with the proviso that only one such double bond [symbol] is present at any given time, and further such that when the nitrogen of N(R²)(R¹²) is double bonded to the adjacent carbon between said nitrogen and X by the optional double bond, then R¹² is absent;

R¹ is selected from the group consisting of H, alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl-, wherein each of said alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl- can be unsubstituted or optionally independently substituted with 1-5 substituents which can be the same or different, each substituent being independently selected from the group consisting of the moieties shown below;

R⁵ is selected from the group consisting of H, alkyl, alkenyl, alkynyl, cycloalkyl, cycloalkylalkyl, cycloalkenyl, heterocyclyl, heterocyclylalkyl, aryl, arylalkyl, heteroaryl, heteroarylalkyl, —CN, —C(O)R¹⁵, —C(O)OR¹⁵, —C(O)N(R¹⁵)(R¹⁶), —S(O)N(R¹⁵)(R¹⁶), —S(O)₂N(R¹⁵)(R¹⁶), —S(O)R¹⁵, —S(O)₂R¹⁵, —C(═NOR¹⁵)R¹⁶ and —P(O)(OR¹⁵)(OR¹⁶);

R¹² is independently selected from the group consisting of H, alkyl, alkenyl, alkynyl, cycloalkyl, cycloalkylalkyl, cycloalkenyl, heterocyclyl, heterocyclylalkyl, aryl, arylalkyl, heteroaryl, heteroarylalkyl, —CN, —C(O)R¹⁵, —C(O)OR¹⁵, —C(O)N(R¹⁵)(R¹⁶), —S(O)N(R¹⁵)(R¹⁶), —S(O)₂N(R¹⁵)(R¹⁶), —S(O)R¹⁵, —S(O)₂R¹⁵, —C(═NOR¹⁵)R¹⁶ and —P(O)(OR¹⁵)(OR¹⁶);

each R¹⁴ can be the same or different, each being independently selected from the group consisting of H, alkyl, alkenyl, alkynyl, cycloalkyl, cycloalkylalkyl, cycloalkenyl, heterocyclyl, heterocyclylalkyl, aryl, arylalkyl, heteroaryl, heteroarylalkyl, —CN, —C(O)R¹⁵, —C(O)OR¹⁵, —C(O)N(R¹⁵)(R¹⁶), —S(O)N(R¹⁵)(R¹⁶), —S(O)₂N(R¹⁵)(R¹⁶), —S(O)R¹⁵, —S(O)₂R¹⁵, —C(═NOR¹⁵)R¹⁶, and —P(O)(OR¹⁵)(OR¹⁶);

R³ is independently selected from the group consisting of H, alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl-, wherein each of said alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl- can be unsubstituted or optionally independently substituted with 1-5 substituents which can be the same or different, each substituent being independently selected from the group consisting of consisting of the moieties shown below;

R⁴ is independently selected from the group consisting of H, alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl-, wherein each of said alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl- can be unsubstituted or optionally independently substituted with 1-5 substituents which can be the same or different, each substituent being independently selected from the group consisting of consisting of the moieties shown below;

R⁷ is selected from the group consisting of H, alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl-, wherein each of said alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl- can be unsubstituted or optionally independently substituted with 1-5 substituents which can be the same or different, each substituent being independently selected from the group consisting of consisting of the moieties shown below;

R⁸ is selected from the group consisting of H, alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl-, with each of said alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl- being unsubstituted or optionally independently substituted with 1-3 substituents which can be the same or different, each substituent being independently selected from the group consisting of the moieties shown below;

R⁹ is selected from the group consisting of alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl-, wherein each of said alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl- can be unsubstituted or optionally independently substituted with 1-3 substituents which can be the same or different, each substituent being independently selected from the group consisting of the moieties shown below,

R¹⁰ is selected from the group consisting of a bond, alkyl-, alkenyl-, alkynyl-, aryl-, arylalkyl-, alkylaryl-, cycloalkyl-, cycloalkylalkyl-, heteroaryl-, heteroarylalkyl-, heterocyclyl-, heterocyclyalkyl- and the moieties:

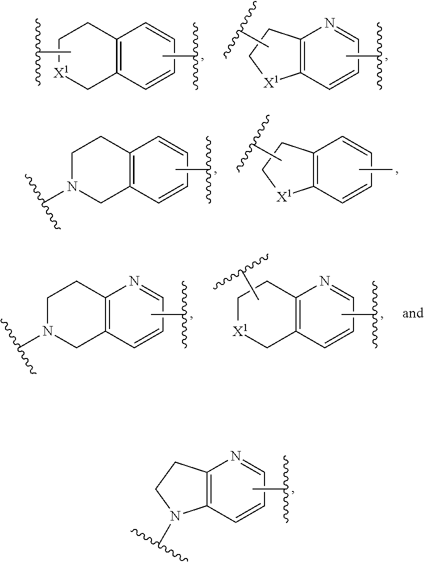

where X¹ is O, N(R¹⁴) or S;

wherein each of said alkyl-, alkenyl-, alkynyl-, aryl-, arylalkyl-, alkylaryl-, cycloalkyl-, cycloalkylalkyl-, heteroaryl-, heteroarylalkyl-, heterocyclyl- heterocyclyalkyl- and the above-noted moieties for R¹⁰ can be unsubstituted or optionally independently substituted with 1-3 substituents which can be the same or different, each being independently selected from the group consisting of the moieties shown below; and

R¹⁵, R¹⁶ and R¹⁷ are independently selected from the group consisting of H, alkyl, alkenyl, alkynyl, cycloalkyl, cycloalkylalkyl, heterocyclyl, heterocyclylalkyl, aryl, arylalkyl, heteroaryl, heteroarylalkyl, arylcycloalkyl, arylheterocyclyl, R¹⁸-alkyl, R¹⁸-cycloalkyl, R¹⁸-cycloalkylalkyl, R¹⁸-heterocyclyl, R¹⁸-heterocyclylalkyl, R¹⁸-aryl, R¹⁸-arylalkyl, R¹⁸-heteroaryl and R¹⁸-heteroarylalkyl;

R¹⁸ is 1-5 substituents independently selected from the group consisting of alkyl, alkenyl, alkynyl, aryl, arylalkyl, arylalkenyl, arylalkynyl, —NO₂, halo, heteroaryl, HO-alkyoxyalkyl, —CF₃, —CN, alkyl-CN, —C(O)R¹⁹, —C(O)OH, —C(O)OR¹⁹, —C(O)NHR²⁰, —C(O)NH₂, —C(O)NH₂—C(O)N(alkyl)₂, —C(O)N(alkyl)(aryl), —C(O)N(alkyl)(heteroaryl), —SR¹⁹, —S(O)₂R²⁰, —S(O)NH₂, —S(O)NH(alkyl), —S(O)N(alkyl)(alkyl), —S(O)NH(aryl), —S(O)₂NH₂, —S(O)₂NHR¹⁹, —S(O)₂NH(heterocyclyl), —S(O)₂N(alkyl)₂, —S(O)₂N(alkyl)(aryl), —OCF₃, —OH, —OR²⁰, —O-heterocyclyl, —O-cycloalkylalkyl, —O-heterocyclylalkyl, —NH₂, —NHR²⁰, —N(alkyl)₂, —N(arylalkyl)₂, —N(arylalkyl)-(heteroarylalkyl), —NHC(O)R²⁰, —NHC(O)NH₂, —NHC(O)NH(alkyl), —NHC(O)N(alkyl)(alkyl), —N(alkyl)C(O)NH(alkyl), —N(alkyl)C(O)N(alkyl)(alkyl), —NHS(O)₂R²⁰, —NHS(O)₂NH(alkyl), —NHS(O)₂N(alkyl)(alkyl), —N(alkyl)S(O)₂NH(alkyl) and —N(alkyl)S(O)₂N(alkyl)(alkyl);

or, alternately, two R¹⁸ moieties on adjacent carbons can be linked together to form:

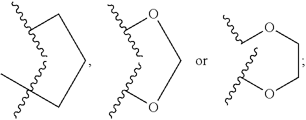

R¹⁹ is alkyl, cycloalkyl, aryl, arylalkyl or heteroarylalkyl;

R²⁰ is alkyl, cycloalkyl, aryl, halo substituted aryl, arylalkyl, heteroaryl or heteroarylalkyl;

wherein each of the alkyl, cycloalkyl, cycloalkylalkyl, heterocyclyl, heterocyclylalkyl, aryl, arylalkyl, alkylaryl, heteroaryl, heteroarylalkyl, alkenyl and alkynyl groups in R¹, R³, R⁴, R⁵, R⁷, R⁸, R⁹, R¹⁰, R¹² and R¹⁴, the 5-14 membered heteroaryl, 5-8 membered heterocyclyl or 5-8 membered heterocyclenyl moiety formed from the joining of R² and R⁶ are independently unsubstituted or substituted by 1 to 5 R²¹ groups independently selected from the group consisting of alkyl, alkenyl, alkynyl, cycloalkyl, cycloalkylalkyl, cycloalkenyl, heterocyclyl, heterocyclylalkyl, aryl, arylalkyl, heteroaryl, heteroarylalkyl, halo, —CN, —OR¹⁵, —C(O)R¹⁵, —C(O)OR¹⁵, —C(O)N(R¹⁵)(R¹⁶), —SR¹⁵, —S(O)N(R¹⁵)(R¹⁶), —CH(R¹⁵)(R¹⁶), —S(O)₂N(R¹⁵)(R¹⁶), —C(═NOR¹⁵)R¹⁶, —P(O)(OR¹⁵)(OR¹⁶), —N(R¹⁵)(R¹⁶), -alkyl-N(R¹⁵)(R¹⁶), —N(R¹⁵)C(O)R¹⁶, —CH₂—N(R¹⁵)C(O)R¹⁶, —CH₂—N(R¹⁵)C(O)N(R¹⁶)(R¹⁷), —CH₂—R¹⁵; —CH₂N(R¹⁵)(R¹⁶), —N(R¹⁵)S(O)R¹⁶, —N(R¹⁵)S(O)₂R¹⁶, —CH₂—N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —CH₂—N(R¹⁵)C(O)N(R¹⁶)(R¹⁷), —N(R¹⁵)C(O)OR¹⁶, —CH₂—N(R¹⁵)C(O)OR¹⁶, —S(O)R¹⁵, ═NOR¹⁵, —N₃, —NO₂ and —S(O)₂R¹⁵; and

wherein each of the alkyl, cycloalkenyl, cycloalkyl, cycloalkylalkyl, heterocyclyl, heterocyclylalkyl, aryl, arylalkyl, heteroaryl, heteroarylalkyl, alkenyl and alkynyl groups in R²¹ are independently unsubstituted or substituted by 1 to 5 R²² groups independently selected from the group consisting of alkyl, cycloalkyl, cycloalkenyl, heterocyclyl, aryl, heteroaryl, halo, —CF₃, —CN, —OR¹⁵, —C(O)R¹⁵, —C(O)OR¹⁵, -alkyl-C(O)OR¹⁵, C(O)N(R¹⁵)(R¹⁶), —S(O)N(R¹⁵)(R¹⁶), —S(O)₂N(R¹⁵)(R¹⁶), —C(═NOR¹⁵)R¹⁶, —P(O)(OR¹⁵)(OR¹⁶), —N(R¹⁵)(R¹⁶), -alkyl-N(R¹⁵)(R¹⁶), —N(R¹⁵)R¹⁶, —CH₂—N(R¹⁵)C(O)R¹⁶, —N(R¹⁵)S(O)R¹⁶, —N(R¹⁵)S(O)₂R¹⁶, —CH₂—N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —CH₂—N(R¹⁵)C(O)N(R¹⁶)(R¹⁷), —N(R¹⁵)C(O)OR¹⁶, —CH₂—N(R¹⁵)C(O)OR¹⁶, —N₃, ═NOR¹⁵, —NO₂, —S(O)R¹⁵ and —S(O)₂R¹⁵.

In another embodiment, the present application discloses a compound, or pharmaceutically acceptable salts, solvates, esters or prodrugs of said compound, said compound having the general structure shown in Formula:

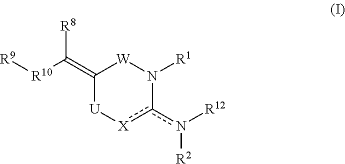

wherein:
R⁶ and R⁵ are joined together to form a 5-14 membered heteroaryl, 5-8 membered heterocyclyl or 5-8 membered heterocyclenyl moiety, with each of said heteroaryl, heterocyclyl or heterocyclenyl moiety being unsubstituted or optionally independently being substituted with 1-5 substituents which can be the same or different, each substituent being independently selected from the group consisting of the moieties shown below;
W is —S(O)—, —S(O)₂— or —C(O)—;
U is —N(R⁵)—;
X is —C(R⁶)(R⁷)—;
each dashed line of

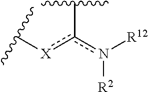

along with its adjoining single bond, together represents an optional double bond with the proviso that only one such double bond [symbol] is present at any given time, and further such that when the nitrogen of N(R²)(R¹²) is double bonded to the adjacent carbon between said nitrogen and X by the optional double bond, then R¹² is absent;

R¹ is selected from the group consisting of H, alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl-, wherein each of said alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl- can be unsubstituted or optionally independently substituted with 1-5 substituents which can be the same or different, each substituent being independently selected from the group consisting of the moieties shown below;

R² is selected from the group consisting of H, alkyl, alkenyl, alkynyl, cycloalkyl, cycloalkylalkyl, cycloalkenyl, heterocyclyl, heterocyclylalkyl, aryl, arylalkyl, heteroaryl, heteroarylalkyl, —CN, —C(O)R¹⁵, —C(O)OR¹⁵, —C(O)N(R¹⁵)(R¹⁶), —S(O)N(R¹⁵)(R¹⁶), —S(O)₂N(R¹⁵)(R¹⁶), —S(O)R¹⁵, —S(O)₂R¹⁵, —C(═NOR¹⁵)R¹⁶ and —P(O)(OR¹⁵)(OR¹⁶);

R¹² is independently selected from the group consisting of H, alkyl, alkenyl, alkynyl, cycloalkyl, cycloalkylalkyl, cycloalkenyl, heterocyclyl, heterocyclylalkyl, aryl, arylalkyl, heteroaryl, heteroarylalkyl, —CN, —C(O)R¹⁵, —C(O)OR¹⁵, —C(O)N(R¹⁵)(R¹⁶), —S(O)N(R¹⁵)(R¹⁶), —S(O)₂N(R¹⁵)(R¹⁶)^{, —S}(O)R¹⁵, —S(O)₂R¹⁵, —C(═NOR¹⁵)R¹⁶ and —P(O)(OR¹⁵)(OR¹⁶);

each R¹⁴ can be the same or different, each being independently selected from the group consisting of H, alkyl, alkenyl, alkynyl, cycloalkyl, cycloalkylalkyl, cycloalkenyl, heterocyclyl, heterocyclylalkyl, aryl, arylalkyl, heteroaryl, heteroarylalkyl, —CN, —C(O)R¹⁵, —C(O)OR¹⁵, —C(O)N(R¹⁵)(R¹⁶), —S(O)N(R¹⁵)(R¹⁶), —S(O)₂N(R¹⁵)(R¹⁶), —S(O)R¹⁵, —S(O)₂R¹⁵, —C(═NOR¹⁵)R¹⁶, and —P(O)(OR¹⁵)(OR¹⁶);

R³ is independently selected from the group consisting of H, alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl-, wherein each of said alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl- can be unsubstituted or optionally independently substituted with 1-5 substituents which can be the same or different, each substituent being independently selected from the group consisting of consisting of the moieties shown below;

R⁴ is independently selected from the group consisting of H, alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl-, wherein each of said alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl- can be unsubstituted or optionally independently substituted with 1-5 substituents which can be the same or different, each substituent being independently selected from the group consisting of consisting of the moieties shown below;

R⁷ is selected from the group consisting of H, alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl-, wherein each of said alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl- can be unsubstituted or optionally independently substituted with 1-5 substituents which can be the same or different, each substituent being independently selected from the group consisting of consisting of the moieties shown below;

R⁸ is selected from the group consisting of H, alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl-, with each of said alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl- being unsubstituted or optionally independently substituted with 1-3 substituents which can be the same or different, each substituent being independently selected from the group consisting of the moieties shown below;

R⁹ is selected from the group consisting of alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl-, wherein each of said alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl- can be unsubstituted or optionally independently substituted with 1-3 substituents which can be the same or different, each substituent being independently selected from the group consisting of the moieties shown below,

R¹⁰ is selected from the group consisting of a bond, alkyl-, alkenyl-, alkynyl-, aryl-, arylalkyl-, alkylaryl-, cycloalkyl-, cycloalkylalkyl-, heteroaryl-, heteroarylalkyl-, heterocyclyl-, heterocyclyalkyl- and the moieties:

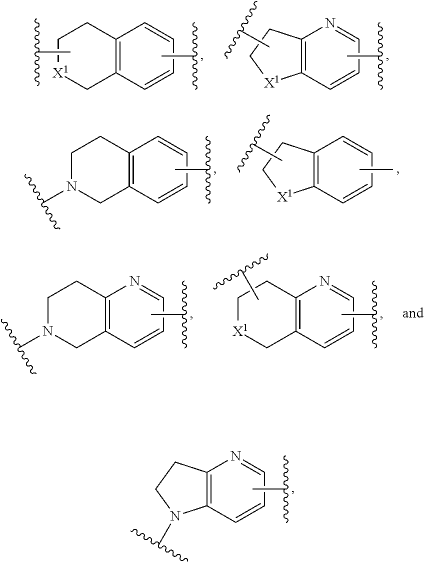

where X¹ is O, N(R¹⁴) or S;

wherein each of said alkyl-, alkenyl-, alkynyl-, aryl-, arylalkyl-, alkylaryl-, cycloalkyl-, cycloalkylalkyl-, heteroaryl-, heteroarylalkyl-, heterocyclyl- heterocyclyalkyl- and the above-noted moieties for R¹⁰ can be unsubstituted or optionally independently substituted with 1-3 substituents which can be the same or different, each being independently selected from the group consisting of the moieties shown below; and

R¹⁵, R¹⁶ and R¹⁷ are independently selected from the group consisting of H, alkyl, alkenyl, alkynyl, cycloalkyl, cycloalkylalkyl, heterocyclyl, heterocyclylalkyl, aryl, arylalkyl, heteroaryl, heteroarylalkyl, arylcycloalkyl, arylheterocyclyl, R¹⁸-alkyl, R¹⁸-cycloalkyl, R¹⁸-cycloalkylalkyl, R¹⁸-heterocyclyl, R¹⁶-heterocyclylalkyl, R¹⁸-aryl, R¹⁸-arylalkyl, R¹⁸-heteroaryl and R¹⁸-heteroarylalkyl;

R¹⁸ is 1-5 substituents independently selected from the group consisting of alkyl, alkenyl, alkynyl, aryl, arylalkyl, arylalkenyl, arylalkynyl, —NO₂, halo, heteroaryl, HO-alkyoxyalkyl, —CF₃, —CN, alkyl-CN, —C(O)R¹⁹, —C(O)OH, —C(O)OR¹⁹, —C(O)NHR²⁰, —C(O)NH₂, —C(O)NH₂—C(O)N(alkyl)₂, —C(O)N(alkyl)(aryl), —C(O)N(alkyl)(heteroaryl), —SR¹⁹, —S(O)₂R²⁰, —S(O)NH₂, —S(O)NH(alkyl), —S(O)N(alkyl)(alkyl), —S(O)NH(aryl), —S(O)₂NH₂, —S(O)₂NHR¹⁹, —S(O)₂NH(heterocyclyl), —S(O)₂N(alkyl)₂, —S(O)₂N(alkyl)(aryl), —OCF₃, —OH, —OR²⁰, —O-heterocyclyl, —O-cycloalkylalkyl, —O-heterocyclylalkyl, —NH₂, —NHR²⁰, —N(alkyl)₂, —N(arylalkyl)₂, —N(arylalkyl)-(heteroarylalkyl), —NHC(O)R²⁰, —NHC(O)NH₂, —NHC(O)NH(alkyl), —NHC(O)N(alkyl)(alkyl), —N(alkyl)C(O)NH(alkyl), —N(alkyl)C(O)N(alkyl)(alkyl), —NHS(O)₂R²⁰, —NHS(O)₂NH(alkyl), —NHS(O)₂N(alkyl)(alkyl), —N(alkyl)S(O)₂NH(alkyl) and —N(alkyl)S(O)₂N(alkyl)(alkyl);

or, alternately, two R¹⁸ moieties on adjacent carbons can be linked together to form:

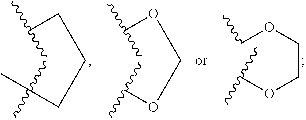

R¹⁹ is alkyl, cycloalkyl, aryl, arylalkyl or heteroarylalkyl;

R²⁰ is alkyl, cycloalkyl, aryl, halo substituted aryl, arylalkyl, heteroaryl or heteroarylalkyl;

wherein each of the alkyl, cycloalkyl, cycloalkylalkyl, heterocyclyl, heterocyclylalkyl, aryl, arylalkyl, alkylaryl, heteroaryl, heteroarylalkyl, alkenyl and alkynyl groups in R¹, R², R³, R⁴, R⁷, R⁸, R⁹, R¹⁰, R¹² and R¹⁴, the 5-14 membered heteroaryl, 5-8 membered heterocyclyl or 5-8 membered heterocyclenyl moiety formed from the joining of R⁶ and R⁵ are independently unsubstituted or substituted by 1 to 5 R²¹ groups independently selected from the group consisting of alkyl, alkenyl, alkynyl, cycloalkyl, cycloalkylalkyl, cycloalkenyl, heterocyclyl, heterocyclylalkyl, aryl, arylalkyl, heteroaryl, heteroarylalkyl, halo, —CN, —OR¹⁵, —C(O)R¹⁵, —C(O)OR¹⁵, —C(O)N(R¹⁵)(R¹⁶), —SR¹⁵, —S(O)N(R¹⁵)(R¹⁶), —CH(R¹⁵)(R¹⁶), —S(O)₂N(R¹⁵)(R¹⁶), —C(═NOR¹⁵)R¹⁶, —P(O)(OR¹⁵)(OR¹⁶), —N(R¹⁵)(R¹⁶), -alkyl-N(R¹⁵)(R¹⁶), —N(R¹⁵)C(O)R¹⁶, —CH₂—N(R¹⁵)C(O)R¹⁶, —CH₂—N(R¹⁵)C(O)N(R¹⁶)(R¹⁷), —CH₂—R¹⁵; —CH₂N(R¹⁵)(R¹⁶), —N(R¹⁵)S(O)R¹⁶, —N(R¹⁵)S(O)₂R¹⁶, —CH₂—N(R¹⁵)S(O)₂R¹⁵, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —CH₂—N(R¹⁵)C(O)N(R¹⁶)(R¹⁷), —N(R¹⁵)C(O)OR¹⁶, —CH₂—N(R¹⁵)C(O)OR¹⁶, —S(O)R¹⁵, ═NOR¹⁵, —N₃, —NO₂ and —S(O)₂R¹⁵; and

wherein each of the alkyl, cycloalkenyl, cycloalkyl, cycloalkylalkyl, heterocyclyl, heterocyclylalkyl, aryl, arylalkyl, heteroaryl, heteroarylalkyl, alkenyl and alkynyl groups in R²¹ are independently unsubstituted or substituted by 1 to 5 R²² groups independently selected from the group consisting of alkyl, cycloalkyl, cycloalkenyl, heterocyclyl, aryl, heteroaryl, halo, —CF₃, —CN, —OR¹⁵, —C(O)R¹⁵, —C(O)OR¹⁵, -alkyl-C(O)OR¹⁵, C(O)N(R¹⁵)(R¹⁶), —SR¹⁵, —S(O)N(R¹⁵)(R¹⁶), —S(O)₂N(R¹⁵)(R¹⁶), —C(═NOR¹⁵)R¹⁶, —P(O)(OR¹⁵)(OR¹⁶), —N(R¹⁵)(R¹⁶), -alkyl-N(R¹⁵)(R¹⁶), —N(R¹⁵)C(O)R¹⁶, —CH₂—N(R¹⁵)C(O)R¹⁶, —N(R¹⁵)S(O)R¹⁶, —N(R¹⁵)S(O)₂R¹⁶, —CH₂—N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —CH₂—N(R¹⁵)C(O)N(R¹⁶)(R¹⁷), —N(R¹⁵)C(O)OR¹⁶, —CH₂—N(R¹⁵)C(O)OR¹⁶, —N₃, ═NOR¹⁵, —NO₂, —S(O)R¹⁵ and —S(O)₂R¹⁵.

In another embodiment, the present application discloses a compound, or pharmaceutically acceptable salts, solvates, esters or prodrugs of said compound, said compound having the general structure shown in Formula:

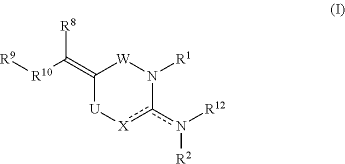

wherein:
R² and R¹⁴ are joined together to form a 5-14 membered heteroaryl, 5-8 membered heterocyclyl or 5-8 membered heterocyclenyl moiety, with each of said heteroaryl, heterocyclyl or heterocyclenyl moiety being unsubstituted or optionally independently being substituted with 1-5 substituents which can be the same or different, each substituent being independently selected from the group consisting of the moieties shown below;
W is —S(O)—, —S(O)₂— or —C(O)—;
U is a bond, —C(O)—, —O—, —N(R⁵)— or —C(R³)(R⁴)—;
X is —N(R¹⁴)—;
each dashed line of

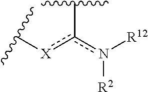

along with its adjoining single bond, together represents an optional double bond with the proviso that only one such double bond [symbol] is present at any given time, and further such that when the nitrogen of N(R²)(R¹²) is double bonded to the adjacent carbon between said nitrogen and X by the optional double bond, then R¹² is absent;

R¹ is selected from the group consisting of H, alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl-, wherein each of said alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl- can be unsubstituted or optionally independently substituted with 1-5 substituents which can be the same or different, each substituent being independently selected from the group consisting of the moieties shown below;

R⁵ is selected from the group consisting of H, alkyl, alkenyl, alkynyl, cycloalkyl, cycloalkylalkyl, cycloalkenyl, heterocyclyl, heterocyclylalkyl, aryl, arylalkyl, heteroaryl, heteroarylalkyl, —CN, —C(O)R¹⁵, —C(O)OR¹⁵, —C(O)N(R¹⁵)(R¹⁶), —S(O)N(R¹⁵)(R¹⁶), —S(O)₂N(R¹⁵)(R¹⁶), —S(O)R¹⁵, —S(O)₂R¹⁵, —C(═NOR¹⁵)R¹⁶ and —P(O)(OR¹⁵)(OR¹⁶);

R¹² is independently selected from the group consisting of H, alkyl, alkenyl, alkynyl, cycloalkyl, cycloalkylalkyl, cycloalkenyl, heterocyclyl, heterocyclylalkyl, aryl, arylalkyl, heteroaryl, heteroarylalkyl, —CN, —C(O)R¹⁵, —C(O)OR¹⁵, —C(O)N(R¹⁵)(R¹⁶), —S(O)N(R¹⁵)(R¹⁶), —S(O)₂N(R¹⁵)(R¹⁶), —S(O)R¹⁵, —S(O)₂R¹⁵, —C(═NOR¹⁵)R¹⁶ and —P(O)(OR¹⁵)(OR¹⁶);

each R¹⁴ (when R¹⁴ is not joined to R²) can be the same or different, each being independently selected from the group consisting of H, alkyl, alkenyl, alkynyl, cycloalkyl, cycloalkylalkyl, cycloalkenyl, heterocyclyl, heterocyclylalkyl, aryl, arylalkyl, heteroaryl, heteroarylalkyl, —CN, —C(O)R¹⁵, —C(O)OR¹⁵, —C(O)N(R¹⁵)(R¹⁶), —S(O)N(R¹⁵)(R¹⁶), —S(O)₂N(R¹⁵)(R¹⁶), —S(O)R¹⁵, —S(O)₂R¹⁵, —C(═NOR¹⁵)R¹⁶, and —P(O)(OR¹⁵)(OR¹⁶);

R³ is independently selected from the group consisting of H, alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl-, wherein each of said alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl- can be unsubstituted or optionally independently substituted with 1-5 substituents which can be the same or different, each substituent being independently selected from the group consisting of consisting of the moieties shown below;

R⁴ is independently selected from the group consisting of H, alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl-, wherein each of said alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl- can be unsubstituted or optionally independently substituted with 1-5 substituents which can be the same or different, each substituent being independently selected from the group consisting of consisting of the moieties shown below;

R⁶ is selected from the group consisting of H, alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl-, wherein each of said alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl- can be unsubstituted or optionally independently substituted with 1-5 substituents which can be the same or different, each substituent being independently selected from the group consisting of consisting of the moieties shown below;

R⁷ is selected from the group consisting of H, alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl-, wherein each of said alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl- can be unsubstituted or optionally independently substituted with 1-5 substituents which can be the same or different, each substituent being independently selected from the group consisting of consisting of the moieties shown below;

R⁸ is selected from the group consisting of H, alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl-, with each of said alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl- being unsubstituted or optionally independently substituted with 1-3 substituents which can be the same or different, each substituent being independently selected from the group consisting of the moieties shown below;

R⁹ is selected from the group consisting of alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl-, wherein each of said alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl- can be unsubstituted or optionally independently substituted with 1-3 substituents which can be the same or different, each substituent being independently selected from the group consisting of the moieties shown below,

R¹⁰ is selected from the group consisting of a bond, alkyl-, alkenyl-, alkynyl-, aryl-, arylalkyl-, alkylaryl-, cycloalkyl-, cycloalkylalkyl-, heteroaryl-, heteroarylalkyl-, heterocyclyl-, heterocyclyalkyl- and the moieties:

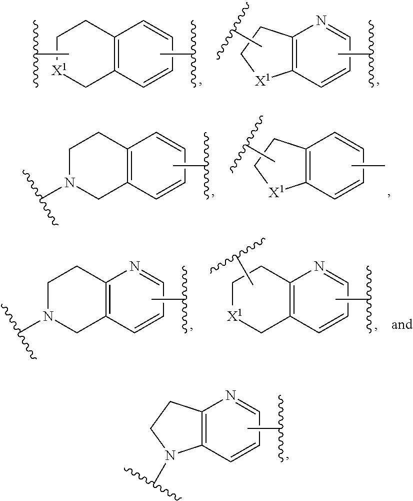

where X¹ is O, N(R¹⁴) or S;

wherein each of said alkyl-, alkenyl-, alkynyl-, aryl-, arylalkyl-, alkylaryl-, cycloalkyl-, cycloalkylalkyl-, heteroaryl-, heteroarylalkyl-, heterocyclyl- heterocyclyalkyl- and the above-noted moieties for R¹⁰ can be unsubstituted or optionally independently substituted with 1-3 substituents which can be the same or different, each being independently selected from the group consisting of the moieties shown below; and

R¹⁵, R¹⁶ and R¹⁷ are independently selected from the group consisting of H, alkyl, alkenyl, alkynyl, cycloalkyl, cycloalkylalkyl, heterocyclyl, heterocyclylalkyl, aryl, arylalkyl, heteroaryl, heteroarylalkyl, arylcycloalkyl, arylheterocyclyl, R¹⁸-alkyl, R¹⁸-cycloalkyl, R¹⁸-cycloalkylalkyl, R¹⁸-heterocyclyl, R¹⁸-heterocyclylalkyl, R¹⁸-aryl, R¹⁸-arylalkyl, R¹⁸-heteroaryl and R¹⁸-heteroarylalkyl;

R¹⁸ is 1-5 substituents independently selected from the group consisting of alkyl, alkenyl, alkynyl, aryl, arylalkyl, arylalkenyl, arylalkynyl, —NO₂, halo, heteroaryl, HO-alkyoxyalkyl, —CF₃, —CN, alkyl-CN, —C(O)R¹⁹, —C(O)OH, —C(O)OR¹⁹, —C(O)NHR²⁰, —C(O)NH₂, —C(O)NH₂—C(O)N(alkyl)₂, —C(O)N(alkyl)(aryl), —C(O)N(alkyl)(heteroaryl), —SR¹⁹, —S(O)₂R²⁰, —S(O)NH₂, —S(O)NH(alkyl), —S(O)N(alkyl)(alkyl), —S(O)NH(aryl), —S(O)₂NH₂, —S(O)₂NHR¹⁹, —S(O)₂NH(heterocyclyl), —S(O)₂N(alkyl)₂, —S(O)₂N(alkyl)(aryl), —OCF₃, —OH, —OR²⁰, —O-heterocyclyl, —O-cycloalkylalkyl, —O-heterocyclylalkyl, —NH₂, —NHR²⁰, —N(alkyl)₂, —N(arylalkyl)₂, —N(arylalkyl)-(heteroarylalkyl); —NHC(O)R²⁰, —NHC(O)NH₂, —NHC(O)NH(alkyl), —NHC(O)N(alkyl)(alkyl), —N(alkyl)C(O)NH(alkyl), —N(alkyl)C(O)N(alkyl)(alkyl), —NHS(O)₂R²⁰, —NHS(O)₂NH(alkyl), —NHS(O)₂N(alkyl)(alkyl), —N(alkyl)S(O)₂NH(alkyl) and —N(alkyl)S(O)₂N(alkyl)(alkyl);

or, alternately, two R¹⁸ moieties on adjacent carbons can be linked together to form:

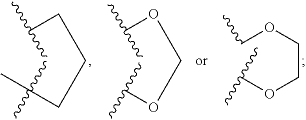

R¹⁹ is alkyl, cycloalkyl, aryl, arylalkyl or heteroarylalkyl;

R²⁰ is alkyl, cycloalkyl, aryl, halo substituted aryl, arylalkyl, heteroaryl or heteroarylalkyl;

wherein each of the alkyl, cycloalkyl, cycloalkylalkyl, heterocyclyl, heterocyclylalkyl, aryl, arylalkyl, alkylaryl, heteroaryl, heteroarylalkyl, alkenyl and alkynyl groups in R¹, R³, R⁴, R⁵, R⁶, R⁷, R⁸, R⁹, R¹⁰, R¹² and R¹⁴, the 5-14 membered heteroaryl, 5-8 membered heterocyclyl or 5-8 membered heterocyclenyl moiety formed from the joining of R² and R¹⁴ are independently unsubstituted or substituted by 1 to 5 R²¹ groups independently selected from the group consisting of alkyl, alkenyl, alkynyl, cycloalkyl, cycloalkylalkyl, cycloalkenyl, heterocyclyl, heterocyclylalkyl, aryl, arylalkyl, heteroaryl, heteroarylalkyl, halo, —CN, —OR¹⁵, —C(O)R¹⁵, —C(O)OR¹⁵, —C(O)N(R¹⁵)(R¹⁶), —SR¹⁵, —S(O)N(R¹⁵)(R¹⁶), —CH(R¹⁵)(R¹⁶), —S(O)₂N(R¹⁵)(R¹⁶), —C(═NOR¹⁵)R¹⁶, —P(O)(OR¹⁵)(OR¹⁶), —N(R¹⁵)(R¹⁶), -alkyl-N(R¹⁵)(R¹⁶), —N(R¹⁵)C(O)R¹⁶, —CH₂—N(R¹⁵)C(O)R¹⁶, —CH₂—N(R¹⁵)C(O)N(R¹⁶)(R¹⁷), —CH₂—R¹⁵; —CH₂N(R¹⁵)(R¹⁶), —N(R¹⁵)S(O)R¹⁶, —N(R¹⁵)S(O)₂R¹⁶, —CH₂—N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —CH₂—N(R¹⁵)C(O)N(R¹⁶)(R¹⁷), —N(R¹⁵)C(O)OR¹⁶, —CH₂—N(R¹⁵)C(O)OR¹⁶, —S(O)R¹⁵, ═NOR¹⁵, —N₃, —NO₂ and —S(O)₂R¹⁵; and

wherein each of the alkyl, cycloalkenyl, cycloalkyl, cycloalkylalkyl, heterocyclyl, heterocyclylalkyl, aryl, arylalkyl, heteroaryl, heteroarylalkyl, alkenyl and alkynyl groups in R²¹ are independently unsubstituted or substituted by 1 to 5 R²² groups independently selected from the group consisting of alkyl, cycloalkyl, cycloalkenyl, heterocyclyl, aryl, heteroaryl, halo, —CF₃, —CN, —OR¹⁵, —C(O)R¹⁵, —C(O)OR¹⁵, -alkyl-C(O)OR¹⁵, C(O)N(R¹⁵)(R¹⁶), —SR¹⁵, —S(O)N(R¹⁵)(R¹⁶), —S(O)₂N(R¹⁵)(R¹⁶), —C(═NOR¹⁵)R¹⁶, —P(O)(OR¹⁵)(OR¹⁶), —N(R¹⁵)(R¹⁶), -alkyl-N(R¹⁵)(R¹⁶), —N(R¹⁵)C(O)R¹⁶, —CH₂—N(R¹⁵)C(O)R¹⁶, —N(R¹⁵)S(O)R¹⁶, —N(R¹⁵)S(O)₂R¹⁶, —CH₂—N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —CH₂—N(R¹⁵)C(O)N(R¹⁶)(R¹⁷), —N(R¹⁵)C(O)OR¹⁶, —CH₂—N(R¹⁵)C(O)OR¹⁶, —N₃, ═NOR¹⁵, —NO₂, —S(O)R¹⁵ and —S(O)₂R¹⁵.

In another embodiment, the present application discloses a compound, or pharmaceutically acceptable salts, solvates, esters or prodrugs of said compound, said compound having the general structure shown in Formula:

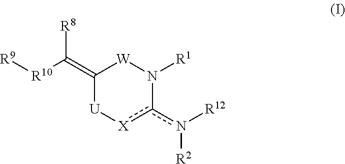

wherein:
R³ and R¹⁴ are joined together to form a 5-14 membered heteroaryl, 5-8 membered heterocyclyl or 5-8 membered heterocyclenyl moiety, with each of said heteroaryl, heterocyclyl or heterocyclenyl moiety being unsubstituted or optionally independently being substituted with 1-5 substituents which can be the same or different, each substituent being independently selected from the group consisting of the moieties shown below;
W is —S(O)—, —S(O)₂— or —C(O)—;
U is —C(R³)(R⁴)—;
X is —N(R¹⁴)—;
each dashed line of

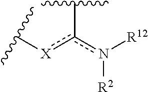

along with its adjoining single bond, together represents an optional double bond with the proviso that only one such double bond [symbol] is present at any given time, and further such that when the nitrogen of N(R²)(R¹²) is double bonded to the adjacent carbon between said nitrogen and X by the optional double bond, then R¹² is absent;

R¹ is selected from the group consisting of H, alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl-, wherein each of said alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl- can be unsubstituted or optionally independently substituted with 1-5 substituents which can be the same or different, each substituent being independently selected from the group consisting of the moieties shown below;

R² is selected from the group consisting of H, alkyl, alkenyl, alkynyl, cycloalkyl, cycloalkylalkyl, cycloalkenyl, heterocyclyl, heterocyclylalkyl, aryl, arylalkyl, heteroaryl, heteroarylalkyl, —CN, —C(O)R¹⁵, —C(O)OR¹⁵, —C(O)N(R¹⁵)(R¹⁶), —S(O)N(R¹⁵)(R¹⁶), —S(O)₂N(R¹⁵)(R¹⁶), —S(O)R¹⁵, —S(O)₂R¹⁵, —C(═NOR¹⁵)R¹⁶ and —P(O)(OR¹⁵)(OR¹⁶);

R⁵ is selected from the group consisting of H, alkyl, alkenyl, alkynyl, cycloalkyl, cycloalkylalkyl, cycloalkenyl, heterocyclyl, heterocyclylalkyl, aryl, arylalkyl, heteroaryl, heteroarylalkyl, —CN, —C(O)R¹⁵, —C(O)OR¹⁵, —C(O)N(R¹⁵)(R¹⁶), —S(O)N(R¹⁵)(R¹⁶), —S(O)₂N(R¹⁵)(R¹⁶), —S(O)R¹⁵, —S(O)₂R¹⁵, —C(═NOR¹⁵)R¹⁶ and —P(O)(OR¹⁵)(OR¹⁶);

R¹² is independently selected from the group consisting of H, alkyl, alkenyl; alkynyl, cycloalkyl, cycloalkylalkyl, cycloalkenyl, heterocyclyl, heterocyclylalkyl, aryl, arylalkyl, heteroaryl, heteroarylalkyl, —CN, —C(O)R¹⁵, —C(O)OR¹⁵, —C(O)N(R¹⁵)(R¹⁶), —S(O)N(R¹⁵)(R¹⁶), —S(O)₂N(R¹⁵)(R¹⁶), —S(O)R¹⁵, —S(O)₂R¹⁵, —C(═NOR¹⁵)R¹⁶ and —P(O)(OR¹⁵)(OR¹⁶);

each R¹⁴ (when R¹⁴ is not joined to R³) can be the same or different, each being independently selected from the group consisting of H, alkyl, alkenyl, alkynyl, cycloalkyl, cycloalkylalkyl, cycloalkenyl, heterocyclyl, heterocyclylalkyl, aryl, arylalkyl, heteroaryl, heteroarylalkyl, —CN, —C(O)R¹⁵, —C(O)OR¹⁵, —C(O)N(R¹⁵)(R¹⁶), —S(O)N(R¹⁵)(R¹⁶), —S(O)₂N(R¹⁵)(R¹⁶), —S(O)R¹⁵, —S(O)₂R¹⁵, —C(═NOR¹⁵)R¹⁶, and —P(O)(OR¹⁵)(OR¹⁶);

R⁴ is independently selected from the group consisting of H, alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl-, wherein each of said alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl- can be unsubstituted or optionally independently substituted with 1-5 substituents which can be the same or different, each substituent being independently selected from the group consisting of consisting of the moieties shown below;

R⁶ is selected from the group consisting of H, alkyl-, alkenyl- and alkynyl-, aryl-, arylalkyl-, alkylaryl-, cycloalkyl-, cycloalkylalkyl-, heteroaryl-, heteroarylalkyl-, heterocyclyl- and heterocyclyalkyl-, wherein each of said alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl- can be unsubstituted or optionally independently substituted with 1-5 substituents which can be the same or different, each substituent being independently selected from the group consisting of consisting of the moieties shown below;

R⁷ is selected from the group consisting of H, alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl-, wherein each of said alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl- can be unsubstituted or optionally independently substituted with 1-5 substituents which can be the same or different, each substituent being independently selected from the group consisting of consisting of the moieties shown below;

R⁸ is selected from the group consisting of H, alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl-, with each of said alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl- being unsubstituted or optionally independently substituted with 1-3 substituents which can be the same or different, each substituent being independently selected from the group consisting of the moieties shown below;

R⁹ is selected from the group consisting of alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl-, wherein each of said alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl- can be unsubstituted or optionally independently substituted with 1-3 substituents which can be the same or different, each substituent being independently selected from the group consisting of the moieties shown below,

R¹⁰ is selected from the group consisting of a bond, alkyl-, alkenyl-, alkynyl-, aryl-, arylalkyl-, alkylaryl-, cycloalkyl-, cycloalkylalkyl-, heteroaryl-, heteroarylalkyl-, heterocyclyl-, heterocyclyalkyl- and the moieties:

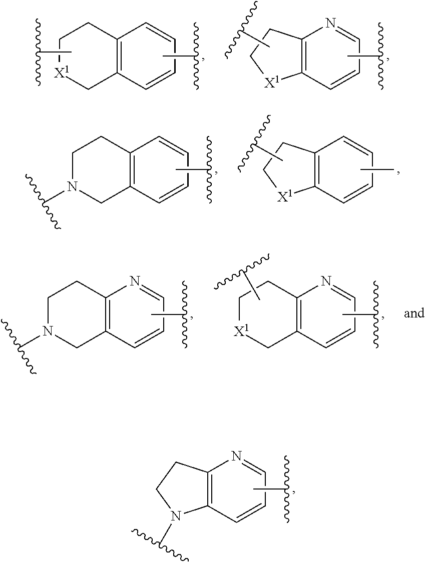

where X¹ is O, N(R¹⁴) or S;

wherein each of said alkyl-, alkenyl-, alkynyl-, aryl-, arylalkyl-, alkylaryl-, cycloalkyl-, cycloalkylalkyl-, heteroaryl-, heteroarylalkyl-, heterocyclyl- heterocyclyalkyl- and the above-noted moieties for R¹⁰ can be unsubstituted or optionally independently substituted with 1-3 substituents which can be the same or different, each being independently selected from the group consisting of the moieties shown below; and

R¹⁵, R¹⁶ and R¹⁷ are independently selected from the group consisting of H, alkyl, alkenyl, alkynyl, cycloalkyl, cycloalkylalkyl, heterocyclyl, heterocyclylalkyl, aryl, arylalkyl, heteroaryl, heteroarylalkyl, arylcycloalkyl, arylheterocyclyl, R¹⁸-alkyl, R¹⁸-cycloalkyl, R¹⁸-cycloalkylalkyl, R¹⁸-heterocyclyl, R¹⁸-heterocyclylalkyl, R¹⁸-aryl, R¹⁸-arylalkyl, R¹⁸-heteroaryl and R¹⁸-heteroarylalkyl;

R¹⁸ is 1-5 substituents independently selected from the group consisting of alkyl, alkenyl, alkynyl, aryl, arylalkyl, arylalkenyl, arylalkynyl, —NO₂, halo, heteroaryl, HO-alkyoxyalkyl, —CF₃, —CN, alkyl-CN, —C(O)R¹⁹, —C(O)OH, —C(O)OR¹⁹, —C(O)NHR²⁰, —C(O)NH₂, —C(O)NH₂—C(O)N(alkyl)₂, —C(O)N(alkyl)(aryl), —C(O)N(alkyl)(heteroaryl), —SR¹⁹, —S(O)₂R²⁰, —S(O)NH₂, —S(O)NH(alkyl), —S(O)N(alkyl)(alkyl), —S(O)NH(aryl), —S(O)₂NH₂, —S(O)₂NHR¹⁹, —S(O)₂NH(heterocyclyl), —S(O)₂N(alkyl)₂, —S(O)₂N(alkyl)(aryl), —OCF₃, —OH, —OR²⁰, —O-heterocyclyl, —O-cycloalkylalkyl, —O-heterocyclylalkyl, —NH₂, —NHR²⁰, —N(alkyl)₂, —N(arylalkyl)₂, —N(arylalkyl)-(heteroarylalkyl), —NHC(O)R²⁰, —NHC(O)NH₂, —NHC(O)NH(alkyl), —NHC(O)N(alkyl)(alkyl), —N(alkyl)C(O)NH(alkyl), —N(alkyl)C(O)N(alkyl)(alkyl), —NHS(O)₂R²⁰, —NHS(O)₂NH(alkyl), —NHS(O)₂N(alkyl)(alkyl), —N(alkyl)S(O)₂NH(alkyl) and —N(alkyl)S(O)₂N(alkyl)(alkyl);

or, alternately, two R¹⁸ moieties on adjacent carbons can be linked together to form:

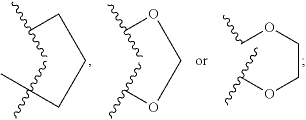

R¹⁹ is alkyl, cycloalkyl, aryl, arylalkyl or heteroarylalkyl;

R²⁰ is alkyl, cycloalkyl, aryl, halo substituted aryl, arylalkyl, heteroaryl or heteroarylalkyl;

wherein each of the alkyl, cycloalkyl, cycloalkylalkyl, heterocyclyl, heterocyclylalkyl, aryl, arylalkyl, alkylaryl, heteroaryl, heteroarylalkyl, alkenyl and alkynyl groups in R¹, R², R⁴, R⁵, R⁶, R⁷, R⁸, R⁹, R¹⁰, R¹² and R¹⁴, the 5-14 membered heteroaryl, 5-8 membered heterocyclyl or 5-8 membered heterocyclenyl moiety formed from the joining of R³ and R¹⁴ are independently unsubstituted or substituted by 1 to 5 R²¹ groups independently selected from the group consisting of alkyl, alkenyl, alkynyl, cycloalkyl, cycloalkylalkyl, cycloalkenyl, heterocyclyl, heterocyclylalkyl, aryl, arylalkyl, heteroaryl, heteroarylalkyl, halo, —CN, —OR¹⁵, —C(O)R¹⁵, —C(O)OR¹⁵, —C(O)N(R¹⁵)(R¹⁶), —SR¹⁵, —S(O)N(R¹⁵)(R¹⁶), —CH(R¹⁵)(R¹⁶), —S(O)₂N(R¹⁵)(R¹⁶), —C(═NOR¹⁵)R¹⁶, —P(O)(OR¹⁵)(OR¹⁶), —N(R¹⁵)(R¹⁶), -alkyl-N(R¹⁵)(R¹⁶), —N(R¹⁵)C(O)R¹⁶, —CH₂—N(R¹⁵)C(O)R¹⁶, —CH₂—N(R¹⁵)C(O)N(R¹⁶)(R¹⁷), —CH₂—R¹⁵; —CH₂N(R¹⁵)(R¹⁶), —N(R¹⁵)S(O)R¹⁶, —N(R¹⁵)S(O)₂R¹⁶, —CH₂—N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —CH₂—N(R¹⁵)C(O)N(R¹⁶)(R¹⁷), —N(R¹⁵)C(O)OR¹⁶, —CH₂—N(R¹⁵)C(O)OR¹⁶, —S(O)R¹⁵, ═NOR¹⁵, —N₃, —NO₂ and —S(O)₂R¹⁵; and

wherein each of the alkyl, cycloalkenyl, cycloalkyl, cycloalkylalkyl, heterocyclyl, heterocyclylalkyl, aryl, arylalkyl, heteroaryl, heteroarylalkyl, alkenyl and alkynyl groups in R²¹ are independently unsubstituted or substituted by 1 to 5 R²² groups independently selected from the group consisting of alkyl, cycloalkyl, cycloalkenyl, heterocyclyl, aryl, heteroaryl, halo, —CF₃, —CN, —OR¹⁵, —C(O)R¹⁵, —C(O)OR¹⁵, -alkyl-C(O)OR¹⁵, C(O)N(R¹⁵)(R¹⁶), —SR¹⁵, —S(O)N(R¹⁵)(R¹⁶), —S(O)₂N(R¹⁵)(R¹⁶), —C(═NOR¹⁵)R¹⁶, —P(O)(OR¹⁵)(OR¹⁶), —N(R¹⁵)(R¹⁶), -alkyl-N(R¹⁵)(R¹⁶), —N(R¹⁶)C(O)R¹⁶, —CH₂—N(R¹⁵)C(O)R¹⁶, —N(R¹⁵)S(O)R¹⁶, —N(R¹⁶)S(O)₂R¹⁶, —CH₂—N(R¹⁵)S(O)₂R¹⁶, —N(R¹⁶)S(O)₂N(R¹⁶)(R¹⁷), —N(R¹⁵)S(O)N(R¹⁶)(R¹⁷), —N(R¹⁵)C(O)N(R¹⁶)(R¹⁷), —CH₂—N(R¹⁵)C(O)N(R¹⁶)(R¹⁷), —N(R¹⁵)C(O)OR¹⁶, —CH₂—N(R¹⁵)C(O)OR¹⁶, —N₃, ═NOR¹⁵, —NO₂, —S(O)R¹⁵ and —S(O)₂R¹⁵.

In another embodiment, the present application discloses a compound, or pharmaceutically acceptable salts, solvates, esters or prodrugs of said compound, said compound having the general structure shown in Formula:

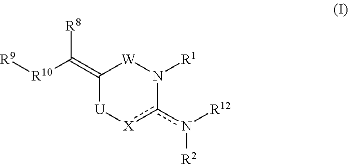

wherein:
R³ and R⁶ are joined together to form a 5-14 membered aryl, 5-8 membered cycloalkyl, 5-8 membered cycloalkenyl, 5-14 membered heteroaryl, 5-8 membered heterocyclyl or 5-8 membered heterocyclenyl moiety, with each of said aryl, cycloalkyl, cycloalkenyl, heteroaryl, heterocyclyl or heterocyclenyl moiety being unsubstituted or optionally independently being substituted with 1-5 substituents which can be the same or different, each substituent being independently selected from the group consisting of the moieties shown below;
W is —S(O)—, —S(O)₂— or —C(O)—;
U is —C(R³)(R⁴)—;
X is —C(R⁶)(R⁷)—;
each dashed line of

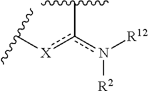

along with its adjoining single bond, together represents an optional double bond with the proviso that only one such double bond [symbol] is present at any given time, and further such that when the nitrogen of N(R²)(R¹²) is double bonded to the adjacent carbon between said nitrogen and X by the optional double bond, then R¹² is absent;

R¹ is selected from the group consisting of H, alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl-, wherein each of said alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl- can be unsubstituted or optionally independently substituted with 1-5 substituents which can be the same or different, each substituent being independently selected from the group consisting of the moieties shown below;

R² is selected from the group consisting of H, alkyl, alkenyl, alkynyl, cycloalkyl, cycloalkylalkyl, cycloalkenyl, heterocyclyl, heterocyclylalkyl, aryl, arylalkyl, heteroaryl, heteroarylalkyl, —CN, —C(O)R¹⁵, —C(O)OR¹⁵, —C(O)N(R¹⁵)(R¹⁶), —S(O)N(R¹⁵)(R¹⁶) S(O)₂N(R¹⁵)(R¹⁶), —S(O)R¹⁵, —S(O)₂R¹⁵, —C(═NOR¹⁵)R¹⁶ and —P(O)(OR¹⁵)(OR¹⁶);

R⁵ is selected from the group consisting of H, alkyl, alkenyl, alkynyl, cycloalkyl, cycloalkylalkyl, cycloalkenyl, heterocyclyl, heterocyclylalkyl, aryl, arylalkyl, heteroaryl, heteroarylalkyl, —CN, —C(O)R¹⁵, —C(O)OR¹⁵, —C(O)N(R¹⁵)(R¹⁶), —S(O)N(R¹⁵)(R¹⁶), —S(O)₂N(R¹⁵)(R¹⁶), —S(O)R¹⁵, —S(O)₂R¹⁵, —C(═NOR¹⁵)R¹⁶ and —P(O)(OR¹⁵)(OR¹⁶);

R¹² is independently selected from the group consisting of H, alkyl, alkenyl, alkynyl, cycloalkyl, cycloalkylalkyl, cycloalkenyl, heterocyclyl, heterocyclylalkyl, aryl, arylalkyl, heteroaryl, heteroarylalkyl, —CN, —C(O)R¹⁵, —C(O)OR¹⁵, —C(O)N(R¹⁵)(R¹⁶), —S(O)N(R¹⁵)(R¹⁶), —S(O)₂N(R¹⁵)(R¹⁶), —S(O)R¹⁵, —S(O)₂R¹⁵, —C(═NOR¹⁵)R¹⁶ and —P(O)(OR¹⁵)(OR¹⁶);

each R¹⁴ can be the same or different, each being independently selected from the group consisting of H, alkyl, alkenyl, alkynyl, cycloalkyl, cycloalkylalkyl, cycloalkenyl, heterocyclyl, heterocyclylalkyl, aryl, arylalkyl, heteroaryl, heteroarylalkyl, —CN, —C(O)R¹⁵, —C(O)OR¹⁶, —C(O)N(R¹⁵)(R¹⁶), —S(O)N(R¹⁵)(R¹⁶), —S(O)₂N(R¹⁵)(R¹⁶), —S(O)R¹⁵, —S(O)₂R¹⁵, —C(═NOR¹⁵)R¹⁶, and —P(O)(OR¹⁵)(OR¹⁶);

R⁴ is independently selected from the group consisting of H, alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl-, wherein each of said alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl- can be unsubstituted or optionally independently substituted with 1-5 substituents which can be the same or different, each substituent being independently selected from the group consisting of consisting of the moieties shown below;

R⁷ is selected from the group consisting of H, alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl-, wherein each of said alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl- can be unsubstituted or optionally independently substituted with 1-5 substituents which can be the same or different, each substituent being independently selected from the group consisting of consisting of the moieties shown below;

R⁸ is selected from the group consisting of H, alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl-, with each of said alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl- being unsubstituted or optionally independently substituted with 1-3 substituents which can be the same or different, each substituent being independently selected from the group consisting of the moieties shown below;

R⁹ is selected from the group consisting of alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl-, wherein each of said alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl- can be unsubstituted or optionally independently substituted with 1-3 substituents which can be the same or different, each substituent being independently selected from the group consisting of the moieties shown below,

R¹⁰ is selected from the group consisting of a bond, alkyl-, alkenyl-, alkynyl-, aryl-, arylalkyl-, alkylaryl-, cycloalkyl-, cycloalkylalkyl-, heteroaryl-, heteroarylalkyl-, heterocyclyl-, heterocyclyalkyl- and the moieties:

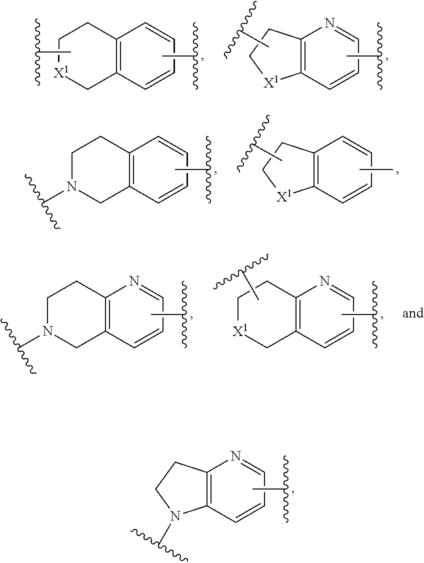

where X¹ is O, N(R¹⁴) or S;

wherein each of said alkyl-, alkenyl-, alkynyl-, aryl-, arylalkyl-, alkylaryl-, cycloalkyl-, cycloalkylalkyl-, heteroaryl-, heteroarylalkyl-, heterocyclyl- heterocyclyalkyl- and the above-noted moieties for R¹⁰ can be unsubstituted or optionally independently substituted with 1-3 substituents which can be the same or different, each being independently selected from the group consisting of the moieties shown below; and

R¹⁵, R¹⁶ and R¹⁷ are independently selected from the group consisting of H, alkyl, alkenyl, alkynyl, cycloalkyl, cycloalkylalkyl, heterocyclyl, heterocyclylalkyl, aryl, arylalkyl, heteroaryl, heteroarylalkyl, arylcycloalkyl, arylheterocyclyl, R¹⁸-alkyl, R¹⁸-cycloalkyl, R¹⁸-cycloalkylalkyl, R¹⁸-heterocyclyl, R¹⁸-heterocyclylalkyl, R¹⁸-aryl, R¹⁸-arylalkyl, R¹⁸-heteroaryl and R¹⁸-heteroarylalkyl;

R¹⁸ is 1-5 substituents independently selected from the group consisting of alkyl, alkenyl, alkynyl, aryl, arylalkyl, arylalkenyl, arylalkynyl, —NO₂, halo, heteroaryl, HO-alkyoxyalkyl, —CF₃, —CN, alkyl-CN, —C(O)R¹⁹, —C(O)OH, —C(O)OR¹⁹, —C(O)NHR²⁰, —C(O)NH₂, —C(O)NH₂—C(O)N(alkyl)₂, —C(O)N(alkyl)(aryl), —C(O)N(alkyl)(heteroaryl), —SR¹⁹, —S(O)₂R²⁰, —S(O)NH₂, —S(O)NH(alkyl), —S(O)N(alkyl)(alkyl), —S(O)NH(aryl), —S(O)₂NH₂, —S(O)₂NHR¹⁹, —S(O)₂NH(heterocyclyl), —S(O)₂N(alkyl)₂, —S(O)₂N(alkyl)(aryl), —OCF₃, —OH, —OR²⁰, —O-heterocyclyl, —O-cycloalkylalkyl, —O-heterocyclylalkyl, —NH₂, —NHR²⁰, —N(alkyl)₂, —N(arylalkyl)₂, —N(arylalkyl)-(heteroarylalkyl), —NHC(O)R²⁰, —NHC(O)NH₂, —NHC(O)NH(alkyl), —NHC(O)N(alkyl)(alkyl), —N(alkyl)C(O)NH(alkyl), —N(alkyl)C(O)N(alkyl)(alkyl), —NHS(O)₂R²⁰, —NHS(O)₂NH(alkyl), —NHS(O)₂N(alkyl)(alkyl), —N(alkyl)S(O)₂NH(alkyl) and —N(alkyl)S(O)₂N(alkyl)(alkyl);

or, alternately, two R¹⁸ moieties on adjacent carbons can be linked together to form:

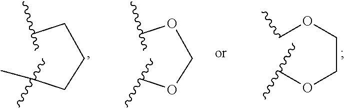

R¹⁹ is alkyl, cycloalkyl, aryl, arylalkyl or heteroarylalkyl;

R²⁰ is alkyl, cycloalkyl, aryl, halo substituted aryl, arylalkyl, heteroaryl or heteroarylalkyl;

wherein each of the alkyl, cycloalkyl, cycloalkylalkyl, heterocyclyl, heterocyclylalkyl, aryl, arylalkyl, alkylaryl, heteroaryl, heteroarylalkyl, alkenyl and alkynyl groups in R¹, R², R⁴, R⁵, R⁷, R⁸, R⁹, R¹⁰, R¹² and R¹⁴, a 5-14 membered aryl, 5-8 membered cycloalkyl, 5-8 membered cycloalkenyl, 5-14 membered heteroaryl, 5-8 membered heterocyclyl or 5-8 membered heterocyclenyl moiety formed from the joining of R³ and R⁶ are independently unsubstituted or substituted by 1 to 5 R²¹ groups independently selected from the group consisting of alkyl, alkenyl, alkynyl, cycloalkyl, cycloalkylalkyl, cycloalkenyl, heterocyclyl, heterocyclylalkyl, aryl, arylalkyl, heteroaryl, heteroarylalkyl, halo, —CN, —OR¹⁵, —C(O)R¹⁵, —C(O)OR¹⁵, —C(O)N(R¹⁵)(R¹⁶), —SR¹⁵, —S(O)N(R¹⁵)(R¹⁶), —CH(R¹⁵)(R¹⁶), —S(O)₂N(R¹⁵)(R¹⁶), —C(═NOR¹⁵)R¹⁶, —P(O)(OR¹⁵)(OR¹⁶), —N(R¹⁵)(R¹⁶), -alkyl-N(R¹⁵)(R¹⁶), —N(R¹⁵)C(O)R¹⁶, —CH₂—N(R¹⁵)C(O)R¹⁶, —CH₂—N(R¹⁵)C(O)N(R¹⁶)(R¹⁷), —CH₂—R¹⁵; —CH₂N(R¹⁵)(R¹⁶), —N(R¹⁵)S(O)R¹⁶, —N(R¹⁵)S(O)₂R¹⁶, —CH₂—N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —CH₂—N(R¹⁵)C(O)N(R¹⁶)(R¹⁷), —N(R¹⁵)C(O)OR¹⁶, —CH₂—N(R¹⁵)C(O)OR¹⁶, —S(O)R¹⁵, ═NOR¹⁵, —N₃, —NO₂ and —S(O)₂R¹⁵; and

wherein each of the alkyl, cycloalkenyl, cycloalkyl, cycloalkylalkyl, heterocyclyl, heterocyclylalkyl, aryl, arylalkyl, heteroaryl, heteroarylalkyl, alkenyl and alkynyl groups in R²¹ are independently unsubstituted or substituted by 1 to 5 R²² groups independently selected from the group consisting of alkyl, cycloalkyl, cycloalkenyl, heterocyclyl, aryl, heteroaryl, halo, —CF₃, —CN, —OR¹⁵, —C(O)R¹⁵, —C(O)OR¹⁵, -alkyl-C(O)OR¹⁵, C(O)N(R¹⁵)(R¹⁶), —SR¹⁵, —S(O)N(R¹⁵)(R¹⁶), —S(O)₂N(R¹⁵)(R¹⁶), —C(═NOR¹⁵)R¹⁶, —P(O)(OR¹⁵)(OR¹⁶), —N(R¹⁵)(R¹⁶), -alkyl-N(R¹⁵)(R¹⁶), —N(R¹⁵)C(O)R¹⁶, —CH₂—N(R¹⁵)C(O)R¹⁶, —N(R¹⁵)S(O)R¹⁶, —N(R¹⁵)S(O)₂R¹⁶, —CH₂—N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —CH₂—N(R¹⁵)C(O)N(R¹⁶)(R¹⁷), —N(R¹⁵)C(O)OR¹⁶, —CH₂—N(R¹⁵)C(O)OR¹⁶, —N₃, ═NOR¹⁵, —NO₂, —S(O)R¹⁵ and —S(O)₂R¹⁵.

In another embodiment, the present application discloses a compound, or pharmaceutically acceptable salts, solvates, esters or prodrugs of said compound, said compound having the general structure shown in Formula:

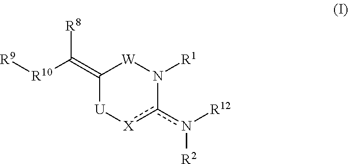

wherein:
R¹⁴ and R⁵ are joined together to form a 5-14 membered heteroaryl, 5-8 membered heterocyclyl or 5-8 membered heterocyclenyl moiety, with each of said heteroaryl, heterocyclyl or heterocyclenyl moiety being unsubstituted or optionally independently being substituted with 1-5 substituents which can be the same or different, each substituent being independently selected from the group consisting of the moieties shown below;
W is —S(O)—, —S(O)₂— or —C(O)—;
U is —N(R⁵)—;
X is —N(R¹⁴)—;
each dashed line of

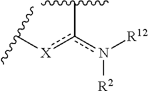

along with its adjoining single bond, together represents an optional double bond with the proviso that only one such double bond [symbol] is present at any given time, and further such that when the nitrogen of N(R²)(R¹²) is double bonded to the adjacent carbon between said nitrogen and X by the optional double bond, then R¹² is absent;

R¹ is selected from the group consisting of H, alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl-, wherein each of said alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl- can be unsubstituted or optionally independently substituted with 1-5 substituents which can be the same or different, each substituent being independently selected from the group consisting of the moieties shown below;

R² is selected from the group consisting of H, alkyl, alkenyl, alkynyl, cycloalkyl, cycloalkylalkyl, cycloalkenyl, heterocyclyl, heterocyclylalkyl, aryl, arylalkyl, heteroaryl, heteroarylalkyl, —CN, —C(O)R¹⁵, —C(O)OR¹⁵, —C(O)N(R¹⁵)(R¹⁶), —S(O)N(R¹⁵)(R¹⁶), —S(O)₂N(R¹⁵)(R¹⁶), —S(O)R¹⁵, —C(═NOR¹⁵)R¹⁶ and —P(O)(OR¹⁵)(OR¹⁶);

R¹² is independently selected from the group consisting of H, alkyl, alkenyl, alkynyl, cycloalkyl, cycloalkylalkyl, cycloalkenyl, heterocyclyl, heterocyclylalkyl, aryl, arylalkyl, heteroaryl, heteroarylalkyl, —CN, —C(O)R¹⁵, —C(O)OR¹⁵, —C(O)N(R¹⁵)(R¹⁶), —S(O)N(R¹⁵)(R¹⁶), —S(O)₂N(R¹⁵)(R¹⁶), —S(O)R¹⁵, —S(O)₂R¹⁶, —C(═NOR¹⁵)R¹⁶ and —P(O)(OR¹⁵)(OR¹⁶);

each R¹⁴ (when R¹⁴ is not joined to R⁵) can be the same or different, each being independently selected from the group consisting of H, alkyl, alkenyl, alkynyl, cycloalkyl, cycloalkylalkyl, cycloalkenyl, heterocyclyl, heterocyclylalkyl, aryl, arylalkyl, heteroaryl, heteroarylalkyl, —CN, —C(O)R¹⁵, —C(O)OR¹⁵, —C(O)N(R¹⁵)(R¹⁶), —S(O)N(R¹⁵)(R¹⁶), —S(O)₂N(R¹⁵)(R¹⁶), —S(O)R¹⁵, —S(O)₂R¹⁵, —C(═NOR¹⁵)R¹⁶, and —P(O)(OR¹⁵)(OR¹⁶);

R³ is independently selected from the group consisting of H, alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl-, wherein each of said alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl- can be unsubstituted or optionally independently substituted with 1-5 substituents which can be the same or different, each substituent being independently selected from the group consisting of consisting of the moieties shown below;

R⁴ is independently selected from the group consisting of H, alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl-, wherein each of said alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl- can be unsubstituted or optionally independently substituted with 1-5 substituents which can be the same or different, each substituent being independently selected from the group consisting of consisting of the moieties shown below;

R⁶ is selected from the group consisting of H, alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl-, wherein each of said alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl- can be unsubstituted or optionally independently substituted with 1-5 substituents which can be the same or different, each substituent being independently selected from the group consisting of consisting of the moieties shown below;

R⁷ is selected from the group consisting of H, alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl-, wherein each of said alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl- can be unsubstituted or optionally independently substituted with 1-5 substituents which can be the same or different, each substituent being independently selected from the group consisting of consisting of the moieties shown below;

R⁸ is selected from the group consisting of H, alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl-, with each of said alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl- being unsubstituted or optionally independently substituted with 1-3 substituents which can be the same or different, each substituent being independently selected from the group consisting of the moieties shown below;

R⁹ is selected from the group consisting of alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl-, wherein each of said alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl- can be unsubstituted or optionally independently substituted with 1-3 substituents which can be the same or different, each substituent being independently selected from the group consisting of the moieties shown below,

R¹⁰ is selected from the group consisting of a bond, alkyl-, alkenyl-, alkynyl-, aryl-, arylalkyl-, alkylaryl-, cycloalkyl-, cycloalkylalkyl-, heteroaryl-, heteroarylalkyl-, heterocyclyl-, heterocyclyalkyl- and the moieties:

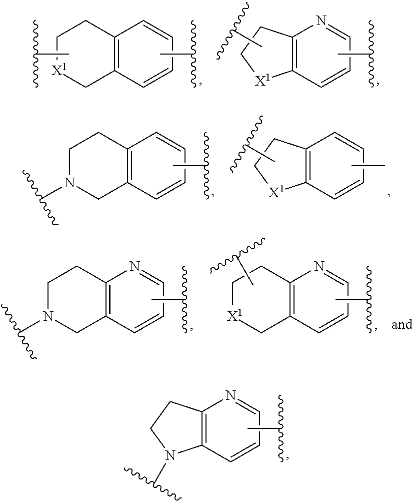

where X¹ is O, N(R¹⁴) or S;

wherein each of said alkyl-, alkenyl-, alkynyl-, aryl-, arylalkyl-, alkylaryl-, cycloalkyl-, cycloalkylalkyl-, heteroaryl-, heteroarylalkyl-, heterocyclyl- heterocyclyalkyl- and the above-noted moieties for R¹⁰ can be unsubstituted or optionally independently substituted with 1-3 substituents which can be the same or different, each being independently selected from the group consisting of the moieties shown below; and

R¹⁵, R¹⁶ and R¹⁷ are independently selected from the group consisting of H, alkyl, alkenyl, alkynyl, cycloalkyl, cycloalkylalkyl, heterocyclyl, heterocyclylalkyl, aryl, arylalkyl, heteroaryl, heteroarylalkyl, arylcycloalkyl, arylheterocyclyl, R¹⁸-alkyl, R¹⁸-cycloalkyl, R¹⁸-cycloalkylalkyl, R¹⁸-heterocyclyl, R¹⁸-heterocyclylalkyl, R¹⁸-aryl, R¹⁸-arylalkyl, R¹⁸-heteroaryl and R¹⁸-heteroarylalkyl;

R¹⁸ is 1-5 substituents independently selected from the group consisting of alkyl, alkenyl, alkynyl, aryl, arylalkyl, arylalkenyl, arylalkynyl, —NO₂, halo, heteroaryl, HO-alkyoxyalkyl, —CF₃, —CN, alkyl-CN, —C(O)R¹⁹, —C(O)OH, —C(O)OR¹⁹, —C(O)NHR²⁰, —C(O)NH₂, —C(O)NH₂—C(O)N(alkyl)₂, —C(O)N(alkyl)(aryl), —C(O)N(alkyl)(heteroaryl), —SR¹⁹, —S(O)₂R²⁰, —S(O)NH₂, —S(O)NH(alkyl), —S(O)N(alkyl)(alkyl), —S(O)NH(aryl), —S(O)₂NH₂, —S(O)₂NHR¹⁹, —S(O)₂NH(heterocyclyl), —S(O)₂N(alkyl)₂, —S(O)₂N(alkyl)(aryl), —OCF₃, —OH, —OR²⁰, —O-heterocyclyl, —O-cycloalkylalkyl, —O-heterocyclylalkyl, —NH₂, —NHR²⁰, —N(alkyl)₂, —N(arylalkyl)₂, —N(arylalkyl)-(heteroarylalkyl), —NHC(O)R²⁰, —NHC(O)NH₂, —NHC(O)NH(alkyl), —NHC(O)N(alkyl)(alkyl), —N(alkyl)C(O)NH(alkyl), —N(alkyl)C(O)N(alkyl)(alkyl), —NHS(O)₂R²⁰, —NHS(O)₂NH(alkyl), —NHS(O)₂N(alkyl)(alkyl), —N(alkyl)S(O)₂NH(alkyl) and —N(alkyl)S(O)₂N(alkyl)(alkyl);

or, alternately, two R¹⁸ moieties on adjacent carbons can be linked together to form:

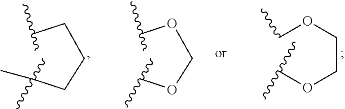

R¹⁹ is alkyl, cycloalkyl, aryl, arylalkyl or heteroarylalkyl;

R²⁰ is alkyl, cycloalkyl, aryl, halo substituted aryl, arylalkyl, heteroaryl or heteroarylalkyl;

wherein each of the alkyl, cycloalkyl, cycloalkylalkyl, heterocyclyl, heterocyclylalkyl, aryl, arylalkyl, alkylaryl, heteroaryl, heteroarylalkyl, alkenyl and alkynyl groups in R¹, R², R³, R⁴, R⁶, R⁷, R⁸, R⁹, R¹⁰, R¹² and R¹⁴, the 5-14 membered heteroaryl, 5-8 membered heterocyclyl or 5-8 membered heterocyclenyl moiety formed from the joining of R¹⁴ and R⁵ are independently unsubstituted or substituted by 1 to 5 R²¹ groups independently selected from the group consisting of alkyl, alkenyl, alkynyl, cycloalkyl, cycloalkylalkyl, cycloalkenyl, heterocyclyl, heterocyclylalkyl, aryl, arylalkyl, heteroaryl, heteroarylalkyl, halo, —CN, —OR¹⁵, —C(O)R¹⁵, —C(O)OR¹⁵, —C(O)N(R¹⁵)(R¹⁶), —SR'^{s}, —S(O)N(R¹⁵)(R¹⁶), —CH(R¹⁵)(R¹⁶), —S(O)₂N(R¹⁵)(R¹⁶), —C(═NOR¹⁵)R¹⁶, —P(O)(OR¹⁵)(OR¹⁶), —N(R¹⁵)(R¹⁶), -alkyl-N(R¹⁵)(R¹⁶), —N(R¹⁵)C(O)R¹⁶, —CH₂—N(R¹⁵)C(O)R¹⁶, —CH₂—N(R¹⁵)C(O)N(R¹⁶)(R¹⁷), —CH₂—R¹⁵; —CH₂N(R¹⁵)(R¹⁶), —N(R¹⁵)S(O)R¹⁶, —N(R¹⁵)S(O)₂R¹⁶, —CH₂—N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —CH₂—N(R¹⁵)C(O)N(R¹⁶)(R¹⁷), —N(R¹⁵)C(O)OR¹⁶, —CH₂—N(R¹⁵)C(O)OR¹⁶, —S(O)R¹⁵, ═NOR¹⁵, —N₃, —NO₂ and —S(O)₂R¹⁵; and

wherein each of the alkyl, cycloalkenyl, cycloalkyl, cycloalkylalkyl, heterocyclyl, heterocyclylalkyl, aryl, arylalkyl, heteroaryl, heteroarylalkyl, alkenyl and alkynyl groups in R²¹ are independently unsubstituted or substituted by 1 to 5 R²² groups independently selected from the group consisting of alkyl, cycloalkyl, cycloalkenyl, heterocyclyl, aryl, heteroaryl, halo, —CF₃, —CN, —OR¹⁵, —C(O)R¹⁵, —C(O)OR¹⁵, -alkyl-C(O)OR¹⁵, C(O)N(R¹⁵)(R¹⁶), —SR¹⁵, —S(O)N(R¹⁵)(R¹⁶), —S(O)₂N(R¹⁵)(R¹⁶), —C(═NOR¹⁵)R¹⁶, —P(O)(OR¹⁵)(OR¹⁶), —N(R¹⁵)(R¹⁶), -alkyl-N(R¹⁵)(R¹⁶), —N(R¹⁵)C(O)R¹⁶, —CH₂—N(R¹⁵)C(O)R¹⁶, —N(R¹⁵)S(O)R¹⁶, —N(R¹⁵)S(O)₂R¹⁶, —CH₂—N(R¹⁵)S(O)₂R¹⁶, —N(R¹⁵)S(O)₂N(R¹⁵)(R¹⁷), —N(R¹⁵)S(O)N(R¹⁶)(R¹⁷), —N(R¹⁵)C(O)N(R¹⁶)(R¹⁷), —CH₂—N(R¹⁵)C(O)N(R¹⁶)(R¹⁷), —N(R¹⁵)C(O)OR¹⁶, —CH₂—N(R¹⁵)C(O)OR¹⁶, —N₃, ═NOR¹⁵, —NO₂, —S(O)R¹⁵ and —S(O)₂R¹⁵.

In another embodiment of this invention, R¹ and R² are joined together to form a 5-14 membered heteroaryl moiety.

In another embodiment of this invention, R¹ and R² are joined together to form a 5-14 membered heteroaryl moiety optionally substituted with 1 to 5 independently selected R²¹ groups.

In another embodiment of this invention, R¹ and R² are joined together to form a 5-14 membered heteroaryl moiety substituted with 1 to 5 independently selected R²¹ groups.

In another embodiment of this invention, R¹ and R² are joined together to form a 5-14 membered heteroaryl moiety optionally substituted with 1 to 5 independently selected R²¹ groups, and said heteroaryl moiety is optionally fused with an aryl or heteroaryl ring, and the ring moiety resulting from the fusion is optionally substituted with 1 to 5 independently selected R²¹ groups.

In another embodiment of this invention, R¹ and R² are joined together to form a 5-14 membered heteroaryl moiety optionally substituted with 1 to 5 independently selected R²¹ groups, and said heteroaryl moiety is optionally fused with an aryl ring, and the ring moiety resulting from the fusion is optionally substituted with 1 to 5 independently selected R²¹ groups.

In another embodiment of this invention, R¹ and R² are joined together to form a 5-14 membered heteroaryl moiety optionally substituted with 1 to 5 independently selected R²¹ groups, and said heteroaryl moiety is optionally fused with a heteroaryl ring, and the ring moiety resulting from the fusion is optionally substituted with 1 to 5 independently selected R²¹ groups.

In another embodiment of this invention, R¹ and R² are joined together to form a 5-8 membered heterocyclyl moiety.

In another embodiment of this invention, R¹ and R² are joined together to form a 5-8 membered heterocyclyl moiety optionally substituted with 1 to 5 independently selected R²¹ groups.

In another embodiment of this invention, R¹ and R² are joined together to form 5-8 membered heterocyclyl moiety substituted with 1 to 5 independently selected R²¹ groups.

In another embodiment of this invention, R¹ and R² are joined together to form a 5-8 membered heterocyclyl moiety optionally substituted with 1 to 5 independently selected R²¹ groups, and said heteroaryl moiety is optionally fused with an aryl or heteroaryl ring, and the ring moiety resulting from the fusion is optionally substituted with 1 to 5 independently selected R²¹ groups.

In another embodiment of this invention, R¹ and R² are joined together to form a 5-8 membered heterocyclyl moiety optionally substituted with 1 to 5 independently selected R²¹ groups, and said heteroaryl moiety is optionally fused with an aryl ring, and the ring moiety resulting from the fusion is optionally substituted with 1 to 5 independently selected R²¹ groups.

In another embodiment of this invention, R¹ and R² are joined together to form a 5-8 membered heterocyclyl moiety optionally substituted with 1 to 5 independently selected R²¹ groups, and said heteroaryl moiety is optionally fused with a heteroaryl ring, and the ring moiety resulting from the fusion is optionally substituted with 1 to 5 independently selected R²¹ groups.

In another embodiment of this invention, R¹ and R² are joined together to form a 5-8 membered heterocyclenyl moiety.

In another embodiment of this invention, R¹ and R² are joined together to form a 5-8 membered heterocyclenyl moiety optionally substituted with 1 to 5 independently selected R²¹ groups.

In another embodiment of this invention, R¹ and R² are joined together to form 5-8 membered heterocyclenyl moiety substituted with 1 to 5 independently selected R²¹ groups.

In another embodiment of this invention, R¹ and R² are joined together to form a 5-8 membered heterocyclenyl moiety optionally substituted with 1 to 5 independently selected R²¹ groups, and said heteroaryl moiety is optionally fused with an aryl or heteroaryl ring, and the ring moiety resulting from the fusion is optionally substituted with 1 to 5 independently selected R²¹ groups.

In another embodiment of this invention, R¹ and R² are joined together to form a 5-8 membered heterocyclenyl moiety optionally substituted with 1 to 5 independently selected R²¹ groups, and said heteroaryl moiety is optionally fused with an aryl ring, and the ring moiety resulting from the fusion is optionally substituted with 1 to 5 independently selected R²¹ groups.

In another embodiment of this invention, R¹ and R² are joined together to form a 5-8 membered heterocyclenyl moiety optionally substituted with 1 to 5 independently selected R²¹ groups, and said heteroaryl moiety is optionally fused with a heteroaryl ring, and the ring moiety resulting from the fusion is optionally substituted with 1 to 5 independently selected R²¹ groups.

In another embodiment of this invention, R² and R⁶ are joined together to form a 5-14 membered heteroaryl moiety.

In another embodiment of this invention, R² and R⁶ are joined together to form a 5-14 membered heteroaryl moiety optionally substituted with 1 to 5 independently selected R²¹ groups.

In another embodiment of this invention, R² and R⁶ are joined together to form a 5-14 membered heteroaryl moiety substituted with 1 to 5 independently selected R²¹ groups.

In another embodiment of this invention, R² and R⁶ are joined together to form a 5-14 membered heteroaryl moiety optionally substituted with 1 to 5 independently selected R²¹ groups, and said heteroaryl moiety is optionally fused with an aryl or heteroaryl ring, and the ring moiety resulting from the fusion is optionally substituted with 1 to 5 independently selected R²¹ groups.

In another embodiment of this invention, R² and R⁶ are joined together to form a 5-14 membered heteroaryl moiety optionally substituted with 1 to 5 independently selected R²¹ groups, and said heteroaryl moiety is optionally fused with an aryl ring, and the ring moiety resulting from the fusion is optionally substituted with 1 to 5 independently selected R²¹ groups.

In another embodiment of this invention, R² and R⁶ are joined together to form a 5-14 membered heteroaryl moiety optionally substituted with 1 to 5 independently selected R²¹ groups, and said heteroaryl moiety is optionally fused with a heteroaryl ring, and the ring moiety resulting from the fusion is optionally substituted with 1 to 5 independently selected R²¹ groups.

In another embodiment of this invention, R² and R⁶ are joined together to form a 5-8 membered heterocyclyl moiety.

In another embodiment of this invention, R² and R⁶ are joined together to form a 5-8 membered heterocyclyl moiety optionally substituted with 1 to 5 independently selected R²¹ groups.

In another embodiment of this invention, R² and R⁶ are joined together to form 5-8 membered heterocyclyl moiety substituted with 1 to 5 independently selected R²¹ groups.

In another embodiment of this invention, R² and R⁶ are joined together to form a 5-8 membered heterocyclyl moiety optionally substituted with 1 to 5 independently selected R²¹ groups, and said heteroaryl moiety is optionally fused with an aryl or heteroaryl ring, and the ring moiety resulting from the fusion is optionally substituted with 1 to 5 independently selected R²¹ groups.

In another embodiment of this invention, R² and R⁶ are joined together to form a 5-8 membered heterocyclyl moiety optionally substituted with 1 to 5 independently selected R²¹ groups, and said heteroaryl moiety is optionally fused with an aryl ring, and the ring moiety resulting from the fusion is optionally substituted with 1 to 5 independently selected R²¹ groups.

In another embodiment of this invention, R² and R⁶ are joined together to form a 5-8 membered heterocyclyl moiety optionally substituted with 1 to 5 independently selected R²¹ groups, and said heteroaryl moiety is optionally fused with a heteroaryl ring, and the ring moiety resulting from the fusion is optionally substituted with 1 to 5 independently selected R²¹ groups.

In another embodiment of this invention, R² and R⁶ are joined together to form a 5-8 membered heterocyclenyl moiety.

In another embodiment of this invention, R² and R⁶ are joined together to form a 5-8 membered heterocyclenyl moiety optionally substituted with 1 to 5 independently selected R²¹ groups.

In another embodiment of this invention R² and R⁶ are joined together to form 5-8 membered heterocyclenyl moiety substituted with 1 to 5 independently selected R²¹ groups.

In another embodiment of this invention, R² and R⁶ are joined together to form a 5-8 membered heterocyclenyl moiety optionally substituted with 1 to 5 independently selected R²¹ groups, and said heteroaryl moiety is optionally fused with an aryl or heteroaryl ring, and the ring moiety resulting from the fusion is optionally substituted with 1 to 5 independently selected R²¹ groups.

In another embodiment of this invention, R² and R⁶ are joined together to form a 5-8 membered heterocyclenyl moiety optionally substituted with 1 to 5 independently selected R²¹ groups, and said heteroaryl moiety is optionally fused with an aryl ring, and the ring moiety resulting from the fusion is optionally substituted with 1 to 5 independently selected R²¹ groups.

In another embodiment of this invention, R² and R⁶ are joined together to form a 5-8 membered heterocyclenyl moiety optionally substituted with 1 to 5 independently selected R²¹ groups, and said heteroaryl moiety is optionally fused with a heteroaryl ring, and the ring moiety resulting from the fusion is optionally substituted with 1 to 5 independently selected R²¹ groups.

In another embodiment of this invention, R⁵ and R⁶ are joined together to form a 5-14 membered heteroaryl moiety.

In another embodiment of this invention, R⁵ and R⁶ are joined together to form a 5-14 membered heteroaryl moiety optionally substituted with 1 to 5 independently selected R²¹ groups.

In another embodiment of this invention, R⁵ and R⁶ are joined together to form a 5-14 membered heteroaryl moiety substituted with 1 to 5 independently selected R²¹ groups.

In another embodiment of this invention, R⁵ and R⁶ are joined together to form a 5-14 membered heteroaryl moiety optionally substituted with 1 to 5 independently selected R²¹ groups, and said heteroaryl moiety is optionally fused with an aryl or heteroaryl ring, and the ring moiety resulting from the fusion is optionally substituted with 1 to 5 independently selected R²¹ groups.

In another embodiment of this invention, R⁵ and R⁶ are joined together to form a 5-14 membered heteroaryl moiety optionally substituted with 1 to 5 independently selected R²¹ groups, and said heteroaryl moiety is optionally fused with an aryl ring, and the ring moiety resulting from the fusion is optionally substituted with 1 to 5 independently selected R²¹ groups.

In another embodiment of this invention, R⁵ and R⁶ are joined together to form a 5-14 membered heteroaryl moiety optionally substituted with 1 to 5 independently selected R²¹ groups, and said heteroaryl moiety is optionally fused with a heteroaryl ring, and the ring moiety resulting from the fusion is optionally substituted with 1 to 5 independently selected R²¹ groups.

In another embodiment of this invention, R⁵ and R⁶ are joined together to form a 5-8 membered heterocyclyl moiety.

In another embodiment of this invention, R⁵ and R⁶ are joined together to form a 5-8 membered heterocyclyl moiety optionally substituted with 1 to 5 independently selected R²¹ groups.

In another embodiment of this invention, R⁵ and R⁶ are joined together to form 5-8 membered heterocyclyl moiety substituted with 1 to 5 independently selected R²¹ groups.

In another embodiment of this invention, R⁵ and R⁶ are joined together to form a 5-8 membered heterocyclyl moiety optionally substituted with 1 to 5 independently selected R²¹ groups, and said heteroaryl moiety is optionally fused with an aryl or heteroaryl ring, and the ring moiety resulting from the fusion is optionally substituted with 1 to 5 independently selected R²¹ groups.

In another embodiment of this invention, R⁵ and R⁶ are joined together to form a 5-8 membered heterocyclyl moiety optionally substituted with 1 to 5 independently selected R²¹ groups, and said heteroaryl moiety is optionally fused with an aryl ring, and the ring moiety resulting from the fusion is optionally substituted with 1 to 5 independently selected R²¹ groups.

In another embodiment of this invention, R⁵ and R⁶ are joined together to form a 5-8 membered heterocyclyl moiety optionally substituted with 1 to 5 independently selected R²¹ groups, and said heteroaryl moiety is optionally fused with a heteroaryl ring, and the ring moiety resulting from the fusion is optionally substituted with 1 to 5 independently selected R²¹ groups.

In another embodiment of this invention, R⁵ and R⁶ are joined together to form a 5-8 membered heterocyclenyl moiety.

In another embodiment of this invention, R⁵ and R⁶ are joined together to form a 5-8 membered heterocyclenyl moiety optionally substituted with 1 to 5 independently selected R²¹ groups.

In another embodiment of this invention, R⁵ and R⁶ are joined together to form 5-8 membered heterocyclenyl moiety substituted with 1 to 5 independently selected R²¹ groups.

In another embodiment of this invention, R⁵ and R⁶ are joined together to form a 5-8 membered heterocyclenyl moiety optionally substituted with 1 to 5 independently selected R²¹ groups, and said heteroaryl moiety is optionally fused with an aryl or heteroaryl ring, and the ring moiety resulting from the fusion is optionally substituted with 1 to 5 independently selected R²¹ groups.

In another embodiment of this invention, R⁵ and R⁶ are joined together to form a 5-8 membered heterocyclenyl moiety optionally substituted with 1 to 5 independently selected R²¹ groups, and said heteroaryl moiety is optionally fused with an aryl ring, and the ring moiety resulting from the fusion is optionally substituted with 1 to 5 independently selected R²¹ groups.

In another embodiment of this invention, R⁵ and R⁶ are joined together to form a 5-8 membered heterocyclenyl moiety optionally substituted with 1 to 5 independently selected R²¹ groups, and said heteroaryl moiety is optionally fused with a heteroaryl ring, and the ring moiety resulting from the fusion is optionally substituted with 1 to 5 independently selected R²¹ groups.

In another embodiment of this invention, R² and R¹⁴ are joined together to form a 5-14 membered heteroaryl moiety.

In another embodiment of this invention, R² and R¹⁴ are joined together to form a 5-14 membered heteroaryl moiety optionally substituted with 1 to 5 independently selected R²¹ groups.

In another embodiment of this invention, R² and R¹⁴ are joined together to form a 5-14 membered heteroaryl moiety substituted with 1 to 5 independently selected R²¹ groups.

In another embodiment of this invention, R² and R¹⁴ are joined together to form a 5-14 membered heteroaryl moiety optionally substituted with 1 to 5 independently selected R²¹ groups, and said heteroaryl moiety is optionally fused with an aryl or heteroaryl ring, and the ring moiety resulting from the fusion is optionally substituted with 1 to 5 independently selected R²¹ groups.

In another embodiment of this invention, R² and R¹⁴ are joined together to form a 5-14 membered heteroaryl moiety optionally substituted with 1 to 5 independently selected R²¹ groups, and said heteroaryl moiety is optionally fused with an aryl ring, and the ring moiety resulting from the fusion is optionally substituted with 1 to 5 independently selected R²¹ groups.

In another embodiment of this invention, R² and R¹⁴ are joined together to form a 5-14 membered heteroaryl moiety optionally substituted with 1 to 5 independently selected R²¹ groups, and said heteroaryl moiety is optionally fused with a heteroaryl ring, and the ring moiety resulting from the fusion is optionally substituted with 1 to 5 independently selected R²¹ groups.

In another embodiment of this invention, R² and R¹⁴ are joined together to form a 5-8 membered heterocyclyl moiety.

In another embodiment of this invention, R² and R¹⁴ are joined together to form a 5-8 membered heterocyclyl moiety optionally substituted with 1 to 5 independently selected R²¹ groups.

In another embodiment of this invention, R² and R¹⁴ are joined together to form 5-8 membered heterocyclyl moiety substituted with 1 to 5 independently selected R²¹ groups.

In another embodiment of this invention, R² and R¹⁴ are joined together to form a 5-8 membered heterocyclyl moiety optionally substituted with 1 to 5 independently selected R²¹ groups, and said heteroaryl moiety is optionally fused with an aryl or heteroaryl ring, and the ring moiety resulting from the fusion is optionally substituted with 1 to 5 independently selected R²¹ groups.

In another embodiment of this invention, R² and R¹⁴ are joined together to form a 5-8 membered heterocyclyl moiety optionally substituted with 1 to 5 independently selected R²¹ groups, and said heteroaryl moiety is optionally fused with an aryl ring, and the ring moiety resulting from the fusion is optionally substituted with 1 to 5 independently selected R²¹ groups.

In another embodiment of this invention, R² and R¹⁴ are joined together to form a 5-8 membered heterocyclyl moiety optionally substituted with 1 to 5 independently selected R²¹ groups, and said heteroaryl moiety is optionally fused with a heteroaryl ring, and the ring moiety resulting from the fusion is optionally substituted with 1 to 5 independently selected R²¹ groups.

In another embodiment of this invention, R² and R¹⁴ are joined together to form a 5-8 membered heterocyclenyl moiety.

In another embodiment of this invention, R² and R¹⁴ are joined together to form a 5-8 membered heterocyclenyl moiety optionally substituted with 1 to 5 independently selected R²¹ groups.

In another embodiment of this invention, R² and R¹⁴ are joined together to form 5-8 membered heterocyclenyl moiety substituted with 1 to 5 independently selected R²¹ groups.

In another embodiment of this invention, R² and R¹⁴ are joined together to form a 5-8 membered heterocyclenyl moiety optionally substituted with 1 to 5 independently selected R²¹ groups, and said heteroaryl moiety is optionally fused with an aryl or heteroaryl ring, and the ring moiety resulting from the fusion is optionally substituted with 1 to 5 independently selected R²¹ groups.

In another embodiment of this invention, R² and R¹⁴ are joined together to form a 5-8 membered heterocyclenyl moiety optionally substituted with 1 to 5 independently selected R²¹ groups, and said heteroaryl moiety is optionally fused with an aryl ring, and the ring moiety resulting from the fusion is optionally substituted with 1 to 5 independently selected R²¹ groups.

In another embodiment of this invention, R² and R¹⁴ are joined together to form a 5-8 membered heterocyclenyl moiety optionally substituted with 1 to 5 independently selected R²¹ groups, and said heteroaryl moiety is optionally fused with a heteroaryl ring, and the ring moiety resulting from the fusion is optionally substituted with 1 to 5 independently selected R²¹ groups.

In another embodiment of this invention, R³ and R¹⁴ are joined together to form a 5-14 membered heteroaryl moiety.

In another embodiment of this invention, R³ and R¹⁴ are joined together to form a 5-14 membered heteroaryl moiety optionally substituted with 1 to 5 independently selected R²¹ groups.

In another embodiment of this invention, R³ and R¹⁴ are joined together to form a 5-14 membered heteroaryl moiety substituted with 1 to 5 independently selected R²¹ groups.

In another embodiment of this invention, R³ and R¹⁴ are joined together to form a 5-14 membered heteroaryl moiety optionally substituted with 1 to 5 independently selected R²¹ groups, and said heteroaryl moiety is optionally fused with an aryl or heteroaryl ring, and the ring moiety resulting from the fusion is optionally substituted with 1 to 5 independently selected R²¹ groups.

In another embodiment of this invention, R³ and R¹⁴ are joined together to form a 5-14 membered heteroaryl moiety optionally substituted with 1 to 5 independently selected R²¹ groups, and said heteroaryl moiety is optionally fused with an aryl ring, and the ring moiety resulting from the fusion is optionally substituted with 1 to 5 independently selected R²¹ groups.

In another embodiment of this invention, R³ and R¹⁴ are joined together to form a 5-14 membered heteroaryl moiety optionally substituted with 1 to 5 independently selected R²¹ groups, and said heteroaryl moiety is optionally fused with a heteroaryl ring, and the ring moiety resulting from the fusion is optionally substituted with 1 to 5 independently selected R²¹ groups.

In another embodiment of this invention, R³ and R¹⁴ are joined together to form a 5-8 membered heterocyclyl moiety.

In another embodiment of this invention, R³ and R¹⁴ are joined together to form a 5-8 membered heterocyclyl moiety optionally substituted with 1 to 5 independently selected R²¹ groups.

In another embodiment of this invention, R³ and R¹⁴ are joined together to form 5-8 membered heterocyclyl moiety substituted with 1 to 5 independently selected R²¹ groups.

In another embodiment of this invention, R³ and R¹⁴ are joined together to form a 5-8 membered heterocyclyl moiety optionally substituted with 1 to 5 independently selected R²¹ groups, and said heteroaryl moiety is optionally fused with an aryl or heteroaryl ring, and the ring moiety resulting from the fusion is optionally substituted with 1 to 5 independently selected R²¹ groups.

In another embodiment of this invention, R³ and R¹⁴ are joined together to form a 5-8 membered heterocyclyl moiety optionally substituted with 1 to 5 independently selected R²¹ groups, and said heteroaryl moiety is optionally fused with an aryl ring, and the ring moiety resulting from the fusion is optionally substituted with 1 to 5 independently selected R²¹ groups.

In another embodiment of this invention, R³ and R¹⁴ are joined together to form a 5-8 membered heterocyclyl moiety optionally substituted with 1 to 5 independently selected R²¹ groups, and said heteroaryl moiety is optionally fused with a heteroaryl ring, and the ring moiety resulting from the fusion is optionally substituted with 1 to 5 independently selected R²¹ groups.

In another embodiment of this invention, R³ and R¹⁴ are joined together to form a 5-8 membered heterocyclenyl moiety.

In another embodiment of this invention, R³ and R¹⁴ are joined together to form a 5-8 membered heterocyclenyl moiety optionally substituted with 1 to 5 independently selected R²¹ groups.

In another embodiment of this invention, R³ and R¹⁴ are joined together to form 5-8 membered heterocyclenyl moiety substituted with 1 to 5 independently selected R²¹ groups.

In another embodiment of this invention, R³ and R¹⁴ are joined together to form a 5-8 membered heterocyclenyl moiety optionally substituted with 1 to 5 independently selected R²¹ groups, and said heteroaryl moiety is optionally fused with an aryl or heteroaryl ring, and the ring moiety resulting from the fusion is optionally substituted with 1 to 5 independently selected R²¹ groups.

In another embodiment of this invention, R³ and R¹⁴ are joined together to form a 5-8 membered heterocyclenyl moiety optionally substituted with 1 to 5 independently selected R²¹ groups, and said heteroaryl moiety is optionally fused with an aryl ring, and the ring moiety resulting from the fusion is optionally substituted with 1 to 5 independently selected R²¹ groups.

In another embodiment of this invention, R³ and R¹⁴ are joined together to form a 5-8 membered heterocyclenyl moiety optionally substituted with 1 to 5 independently selected R²¹ groups, and said heteroaryl moiety is optionally fused with a heteroaryl ring, and the ring moiety resulting from the fusion is optionally substituted with 1 to 5 independently selected R²¹ groups.

In another embodiment of this invention, R³ and R⁶ are joined together to form a 5-14 membered aryl moiety.

In another embodiment of this invention, R³ and R⁶ are joined together to form a 5-14 membered aryl moiety optionally substituted with 1 to 5 independently selected R²¹ groups.

In another embodiment of this invention, R³ and R⁶ are joined together to form a 5-14 membered aryl moiety substituted with 1 to 5 independently selected R²¹ groups.

In another embodiment of this invention, R³ and R⁶ are joined together to form a 5-14 membered aryl moiety optionally substituted with 1 to 5 independently selected R²¹ groups, and said heteroaryl moiety is optionally fused with an aryl or heteroaryl ring, and the ring moiety resulting from the fusion is optionally substituted with 1 to 5 independently selected R²¹ groups.

In another embodiment of this invention, R³ and R⁶ are joined together to form a 5-14 membered aryl moiety optionally substituted with 1 to 5 independently selected R²¹ groups, and said heteroaryl moiety is optionally fused with an aryl ring, and the ring moiety resulting from the fusion is optionally substituted with 1 to 5 independently selected R²¹ groups.

In another embodiment of this invention, R³ and R⁶ are joined together to form a 5-14 membered aryl moiety optionally substituted with 1 to 5 independently selected R²¹ groups, and said heteroaryl moiety is optionally fused with a heteroaryl ring, and the ring moiety resulting from the fusion is optionally substituted with 1 to 5 independently selected R²¹ groups.

In another embodiment of this invention, R³ and R⁶ are joined together to form a 5-8 membered cycloalkyl moiety.

In another embodiment of this invention, R³ and R⁶ are joined together to form a 5-8 membered cycloalkyl moiety optionally substituted with 1 to 5 independently selected R²¹ groups.

In another embodiment of this invention, R³ and R⁶ are joined together to form a 5-8 membered cycloalkyl moiety substituted with 1 to 5 independently selected R²¹ groups.

In another embodiment of this invention, R³ and R⁶ are joined together to form a 5-8 membered cycloalkyl moiety optionally substituted with 1 to 5 independently selected R²¹ groups, and said heteroaryl moiety is optionally fused with an aryl or heteroaryl ring, and the ring moiety resulting from the fusion is optionally substituted with 1 to 5 independently selected R²¹ groups.

In another embodiment of this invention, R³ and R⁶ are joined together to form a 5-8 membered cycloalkyl moiety optionally substituted with 1 to 5 independently selected R²¹ groups, and said heteroaryl moiety is optionally fused with an aryl ring, and the ring moiety resulting from the fusion is optionally substituted with 1 to 5 independently selected R²¹ groups.

In another embodiment of this invention, R³ and R⁶ are joined together to form a 5-8 membered cycloalkyl moiety optionally substituted with 1 to 5 independently selected R²¹ groups, and said heteroaryl moiety is optionally fused with a heteroaryl ring, and the ring moiety resulting from the fusion is optionally substituted with 1 to 5 independently selected R²¹ groups.

In another embodiment of this invention, R³ and R⁶ are joined together to form a 5-8 membered cycloalkenyl moiety.

In another embodiment of this invention, R³ and R⁶ are joined together to form a 5-8 membered cycloalkenyl moiety optionally substituted with 1 to 5 independently selected R²¹ groups.

In another embodiment of this invention, R³ and R⁶ are joined together to form a 5-8 membered cycloalkenyl moiety substituted with 1 to 5 independently selected R²¹ groups.

In another embodiment of this invention, R³ and R⁶ are joined together to form a 5-8 membered cycloalkenyl moiety optionally substituted with 1 to 5 independently selected R²¹ groups, and said heteroaryl moiety is optionally fused with an aryl or heteroaryl ring, and the ring moiety resulting from the fusion is optionally substituted with 1 to 5 independently selected R²¹ groups.

In another embodiment of this invention, R³ and R⁶ are joined together to form a 5-8 membered cycloalkenyl moiety optionally substituted with 1 to 5 independently selected R²¹ groups, and said heteroaryl moiety is optionally fused with an aryl ring, and the ring moiety resulting from the fusion is optionally substituted with 1 to 5 independently selected R²¹ groups.

In another embodiment of this invention, R³ and R⁶ are joined together to form a 5-8 membered cycloalkenyl moiety optionally substituted with 1 to 5 independently selected R²¹ groups, and said heteroaryl moiety is optionally fused with a heteroaryl ring, and the ring moiety resulting from the fusion is optionally substituted with 1 to 5 independently selected R²¹ groups.

In another embodiment of this invention, R³ and R⁶ are joined together to form a 5-14 membered heteroaryl moiety.

In another embodiment of this invention, R³ and R⁶ are joined together to form a 5-14 membered heteroaryl moiety optionally substituted with 1 to 5 independently selected R²¹ groups.

In another embodiment of this invention, R³ and R⁶ are joined together to form a 5-14 membered heteroaryl moiety substituted with 1 to 5 independently selected R²¹ groups.

In another embodiment of this invention, R³ and R⁶ are joined together to form a 5-14 membered heteroaryl moiety optionally substituted with 1 to 5 independently selected R²¹ groups, and said heteroaryl moiety is optionally fused with an aryl or heteroaryl ring, and the ring moiety resulting from the fusion is optionally substituted with 1 to 5 independently selected R²¹ groups.

In another embodiment of this invention, R³ and R⁶ are joined together to form a 5-14 membered heteroaryl moiety optionally substituted with 1 to 5 independently selected R²¹ groups, and said heteroaryl moiety is optionally fused with an aryl ring, and the ring moiety resulting from the fusion is optionally substituted with 1 to 5 independently selected R²¹ groups.

In another embodiment of this invention, R³ and R⁶ are joined together to form a 5-14 membered heteroaryl moiety optionally substituted with 1 to 5 independently selected R²¹ groups, and said heteroaryl moiety is optionally fused with a heteroaryl ring, and the ring moiety resulting from the fusion is optionally substituted with 1 to 5 independently selected R²¹ groups.

In another embodiment of this invention, R³ and R⁶ are joined together to form a 5-8 membered heterocyclyl moiety.

In another embodiment of this invention, R³ and R⁶ are joined together to form a 5-8 membered heterocyclyl moiety optionally substituted with 1 to 5 independently selected R²¹ groups.

In another embodiment of this invention, R³ and R⁶ are joined together to form 5-8 membered heterocyclyl moiety substituted with 1 to 5 independently selected R²¹ groups.

In another embodiment of this invention, R³ and R⁶ are joined together to form a 5-8 membered heterocyclyl moiety optionally substituted with 1 to 5 independently selected R²¹ groups, and said heteroaryl moiety is optionally fused with an aryl or heteroaryl ring, and the ring moiety resulting from the fusion is optionally substituted with 1 to 5 independently selected R²¹ groups.

In another embodiment of this invention, R³ and R⁶ are joined together to form a 5-8 membered heterocyclyl moiety optionally substituted with 1 to 5 independently selected R²¹ groups, and said heteroaryl moiety is optionally fused with an aryl ring, and the ring moiety resulting from the fusion is optionally substituted with 1 to 5 independently selected R²¹ groups.

In another embodiment of this invention, R³ and R⁶ are joined together to form a 5-8 membered heterocyclyl moiety optionally substituted with 1 to 5 independently selected R²¹ groups, and said heteroaryl moiety is optionally fused with a heteroaryl ring, and the ring moiety resulting from the fusion is optionally substituted with 1 to 5 independently selected R²¹ groups.

In another embodiment of this invention, R³ and R⁶ are joined together to form a 5-8 membered heterocyclenyl moiety.

In another embodiment of this invention, R³ and R⁶ are joined together to form a 5-8 membered heterocyclenyl moiety optionally substituted with 1 to 5 independently selected R²¹ groups.

In another embodiment of this invention, R³ and R⁶ are joined together to form 5-8 membered heterocyclenyl moiety substituted with 1 to 5 independently selected R²¹ groups.

In another embodiment of this invention, R³ and R⁶ are joined together to form a 5-8 membered heterocyclenyl moiety optionally substituted with 1 to 5 independently selected R²¹ groups, and said heteroaryl moiety is optionally fused with an aryl or heteroaryl ring, and the ring moiety resulting from the fusion is optionally substituted with 1 to 5 independently selected R²¹ groups.

In another embodiment of this invention, R³ and R⁶ are joined together to form a 5-8 membered heterocyclenyl moiety optionally substituted with 1 to 5 independently selected R²¹ groups, and said heteroaryl moiety is optionally fused with an aryl ring, and the ring moiety resulting from the fusion is optionally substituted with 1 to 5 independently selected R²¹ groups.

In another embodiment of this invention, R³ and R⁶ are joined together to form a 5-8 membered heterocyclenyl moiety optionally substituted with 1 to 5 independently selected R²¹ groups, and said heteroaryl moiety is optionally fused with a heteroaryl ring, and the ring moiety resulting from the fusion is optionally substituted with 1 to 5 independently selected R²¹ groups.

In another embodiment of this invention, R⁵ and R¹⁴ are joined together to form a 5-14 membered heteroaryl moiety.

In another embodiment of this invention, R⁵ and R¹⁴ are joined together to form a 5-14 membered heteroaryl moiety optionally substituted with 1 to 5 independently selected R²¹ groups.

In another embodiment of this invention, R⁵ and R¹⁴ are joined together to form a 5-14 membered heteroaryl moiety substituted with 1 to 5 independently selected R²¹ groups.

In another embodiment of this invention, R⁵ and R¹⁴ are joined together to form a 5-14 membered heteroaryl moiety optionally substituted with 1 to 5 independently selected R²¹ groups, and said heteroaryl moiety is optionally fused with an aryl or heteroaryl ring, and the ring moiety resulting from the fusion is optionally substituted with 1 to 5 independently selected R²¹ groups.

In another embodiment of this invention, R⁵ and R¹⁴ are joined together to form a 5-14 membered heteroaryl moiety optionally substituted with 1 to 5 independently selected R²¹ groups, and said heteroaryl moiety is optionally fused with an aryl ring, and the ring moiety resulting from the fusion is optionally substituted with 1 to 5 independently selected R²¹ groups.

In another embodiment of this invention, R⁵ and R¹⁴ are joined together to form a 5-14 membered heteroaryl moiety optionally substituted with 1 to 5 independently selected R²¹ groups, and said heteroaryl moiety is optionally fused with a heteroaryl ring, and the ring moiety resulting from the fusion is optionally substituted with 1 to 5 independently selected R²¹ groups.

In another embodiment of this invention, R⁵ and R¹⁴ are joined together to form a 5-8 membered heterocyclyl moiety.

In another embodiment of this invention, R⁵ and R¹⁴ are joined together to form a 5-8 membered heterocyclyl moiety optionally substituted with 1 to 5 independently selected R²¹ groups.

In another embodiment of this invention, R⁵ and R¹⁴ are joined together to form 5-8 membered heterocyclyl moiety substituted with 1 to 5 independently selected R²¹ groups.

In another embodiment of this invention, R⁵ and R¹⁴ are joined together to form a 5-8 membered heterocyclyl moiety optionally substituted with 1 to 5 independently selected R²¹ groups, and said heteroaryl moiety is optionally fused with an aryl or heteroaryl ring, and the ring moiety resulting from the fusion is optionally substituted with 1 to 5 independently selected R²¹ groups.

In another embodiment of this invention, R⁵ and R¹⁴ are joined together to form a 5-8 membered heterocyclyl moiety optionally substituted with 1 to 5 independently selected R²¹ groups, and said heteroaryl moiety is optionally fused with an aryl ring, and the ring moiety resulting from the fusion is optionally substituted with 1 to 5 independently selected R²¹ groups.

In another embodiment of this invention, R⁵ and R¹⁴ are joined together to form a 5-8 membered heterocyclyl moiety optionally substituted with 1 to 5 independently selected R²¹ groups, and said heteroaryl moiety is optionally fused with a heteroaryl ring, and the ring moiety resulting from the fusion is optionally substituted with 1 to 5 independently selected R²¹ groups.

In another embodiment of this invention, R⁵ and R¹⁴ are joined together to form a 5-8 membered heterocyclenyl moiety.

In another embodiment of this invention, R⁵ and R¹⁴ are joined together to form a 5-8 membered heterocyclenyl moiety optionally substituted with 1 to 5 independently selected R²¹ groups.

In another embodiment of this invention, R⁵ and R¹⁴ are joined together to form 5-8 membered heterocyclenyl moiety substituted with 1 to 5 independently selected R²¹ groups.

In another embodiment of this invention, R⁵ and R¹⁴ are joined together to form a 5-8 membered heterocyclenyl moiety optionally substituted with 1 to 5 independently selected R²¹ groups, and said heteroaryl moiety is optionally fused with an aryl or heteroaryl ring, and the ring moiety resulting from the fusion is optionally substituted with 1 to 5 independently selected R²¹ groups.

In another embodiment of this invention, R⁵ and R¹⁴ are joined together to form a 5-8 membered heterocyclenyl moiety optionally substituted with 1 to 5 independently selected R²¹ groups, and said heteroaryl moiety is optionally fused with an aryl ring, and the ring moiety resulting from the fusion is optionally substituted with 1 to 5 independently selected R²¹ groups.

In another embodiment of this invention, R⁵ and R¹⁴ are joined together to form a 5-8 membered heterocyclenyl moiety optionally substituted with 1 to 5 independently selected R²¹ groups, and said heteroaryl moiety is optionally fused with a heteroaryl ring, and the ring moiety resulting from the fusion is optionally substituted with 1 to 5 independently selected R²¹ groups.

In another embodiment, W is —C(O)—.

In another embodiment, X is —N(R¹⁴)—.

In another embodiment, U is a bond.

In another embodiment, R⁸ is H.

In another embodiment, R⁸ is alkyl.

In another embodiment, R⁸ is methyl.

In another embodiment R¹ and R² are joined together to form a moiety selected from the group consisting of:

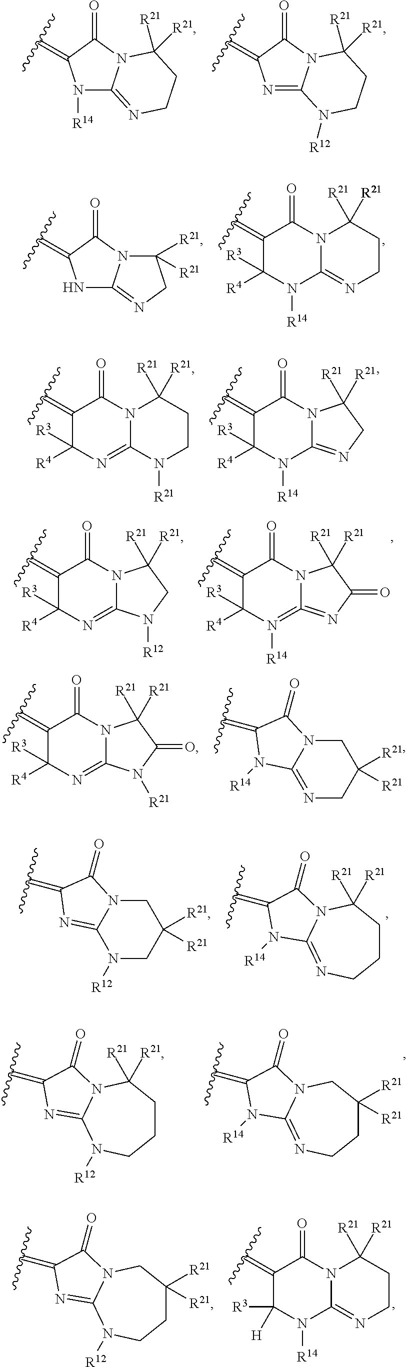

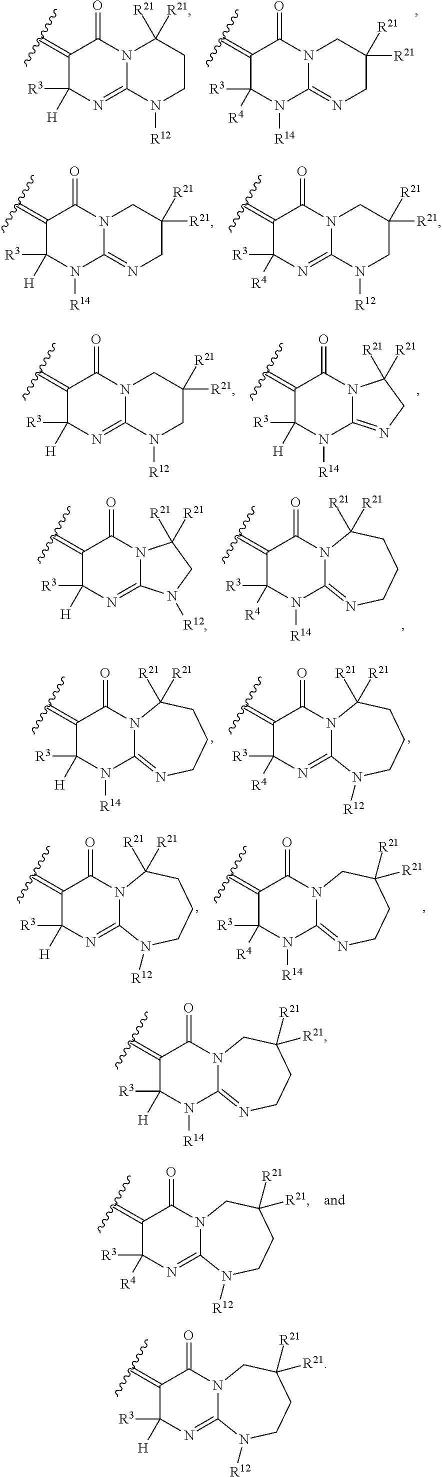

In another embodiment R² and R¹⁴ are joined together to form a moiety selected from the group consisting of:

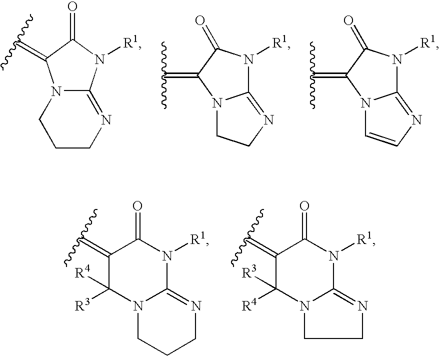

In another embodiment R³ and R¹⁴ are joined together to form

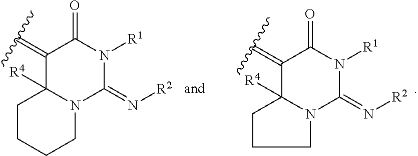

In another embodiment, R¹ and R² are joined together to form

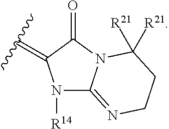

In another embodiment, R¹ and R² are joined together to form

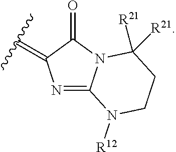

In another embodiment, R¹ and R² are joined together to form

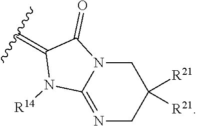

In another embodiment, R¹ and R² are joined together to form

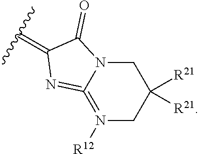

In another embodiment, R¹ and R² are joined together to form

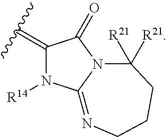

In another embodiment, R¹ and R² are joined together to form

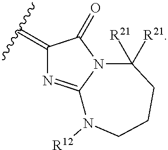

In another embodiment, R¹ and R² are joined together to form

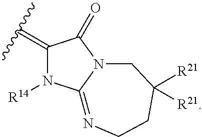

In another embodiment, R¹ and R² are joined together to form

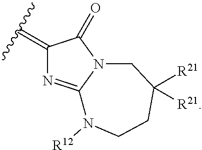

In another embodiment, R² and R¹⁴ are joined together to form

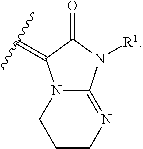

In another embodiment, R¹ and R² are joined together to form

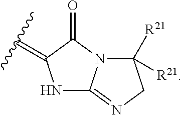

In another embodiment, R² and R¹⁴ are joined together to form

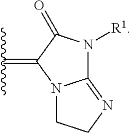

In another embodiment, R² and R¹⁴ are joined together to form

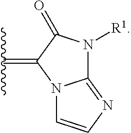

In another embodiment, R² and R¹⁴ are joined together to form

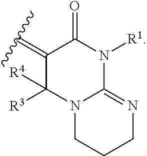

In another embodiment, R³ and R¹⁴ are joined together to form

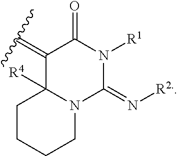

In another embodiment, R³ and R¹⁴ are joined together to form

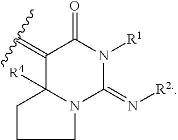

In another embodiment, R² and R¹⁴ are joined together to form

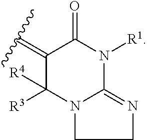

In another embodiment, R³ and R⁶ are joined together to form

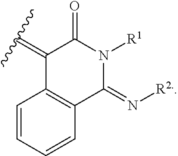

In another embodiment, R¹ and R² are joined together to form

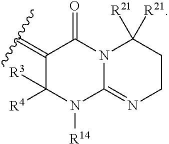

In another embodiment, R⁴ is H and R¹ and R² are joined together to form

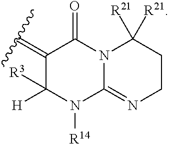

which can, for example, tautomerize to:

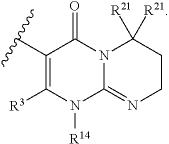

In another embodiment, R¹ and R² are joined together to form

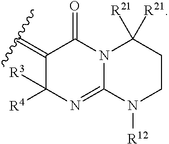

In another embodiment, R⁴ is H and R¹ and R² are joined together to form

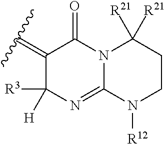

which can, for example, tautomerize to:

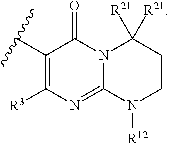

In another embodiment, R¹ and R² are joined together to form

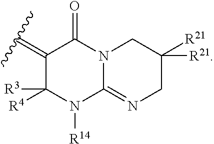

In another embodiment, R⁴ is H and R¹ and R² are joined together to form

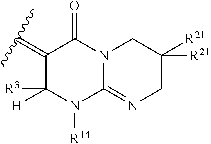

which can, for example, tautomerize to:

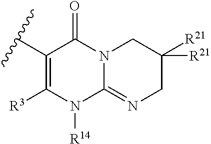

In another embodiment, R¹ and R² are joined together to form

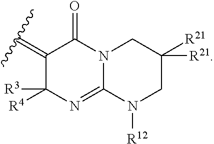

In another embodiment, R⁴ is H and R¹ and R² are joined together to form

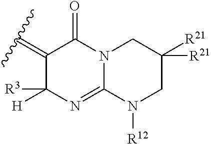

which can, for example, tautomerize to:

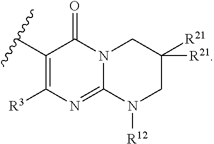

In another embodiment, R¹ and R² are joined together to form

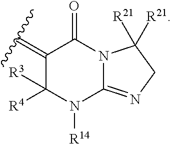

In another embodiment, R⁴ is H and R¹ and R² are joined together to form

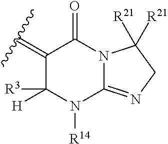

which can, for example, tautomerize to:

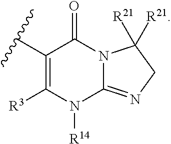

In another embodiment, R¹ and R² are joined together to form

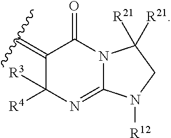

In another embodiment, R⁴ is H and R¹ and R² are joined together to form

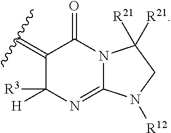

which can, for example, tautomerize to:

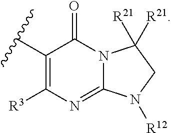

In another embodiment, R¹ and R² are joined together to form

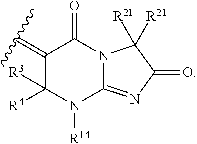

In another embodiment, R¹ and R² are joined together to form

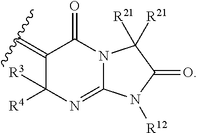

In another embodiment, R¹ and R² are joined together to form

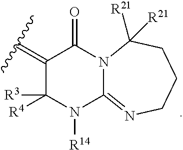

In another embodiment, R⁴ is H and R¹ and R² are joined together to form

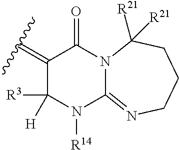

which can, for example, tautomerize to:

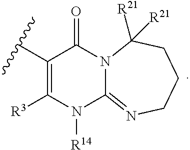

In another embodiment, R¹ and R² are joined together to form

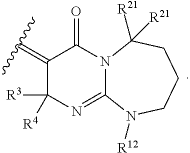

In another embodiment, R⁴ is H and R¹ and R² are joined together to form

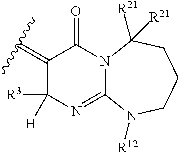

which can, for example, tautomerize to:

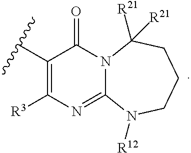

In another embodiment, R¹ and R² are joined together to form

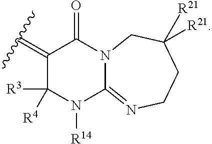

In another embodiment, R⁴ is H and R¹ and R² are joined together to form

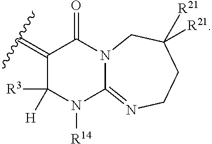

which can, for example, tautomerize to:

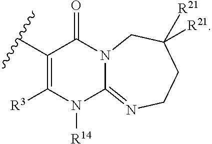

In another embodiment, R¹ and R² are joined together to form

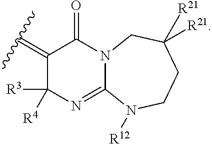

In another embodiment, R⁴ is H and R¹ and R² are joined together to form

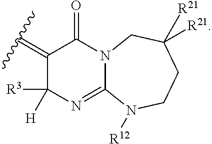

which can, for example, tautomerize to:

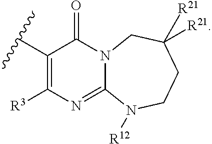

Thus another embodiment of this invention is directed to the tautomers of:

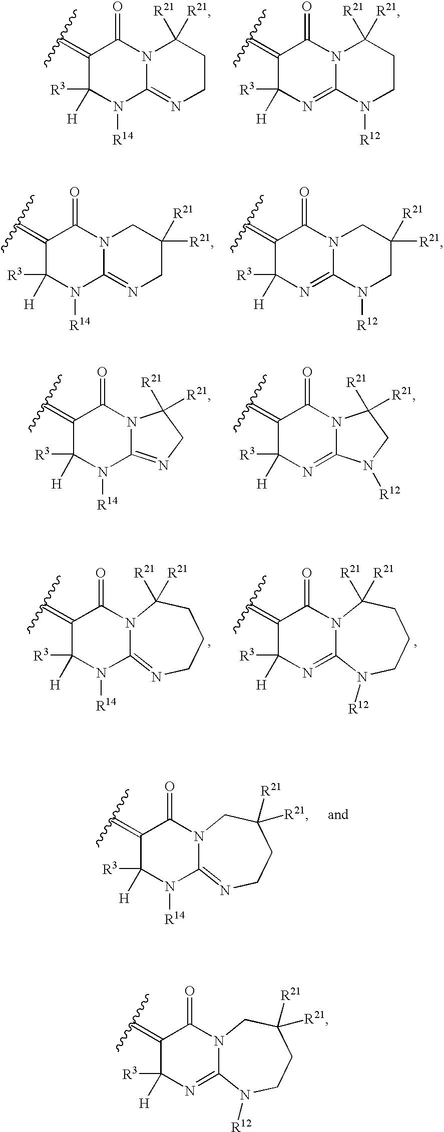

said tautomers being selected from the group consisting of:

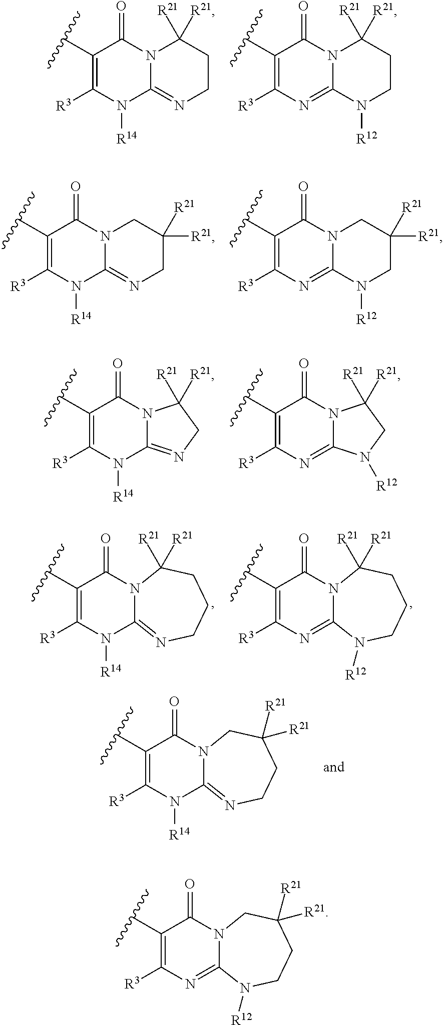

In another embodiment, R¹⁰ is aryl- and said aryl- is unsubstituted.

In another embodiment, R¹⁰ is

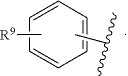

In another embodiment, R¹⁰ is aryl- and said aryl- is substituted with 1-3 substitutents, which can be the same or different, each being independently selected from the group consisting of halo, alkyl, —CN, —NH₂, —NH(alkyl), —N(alkyl)₂, hydroxy and alkoxy groups.

In another embodiment, R¹⁰ is

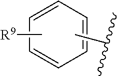

and R¹⁰ is substituted with 1-3 substitutents, which can be the same or different, each being independently selected from the group consisting of halo, alkyl, CN, NH₂, NH(alkyl), N(alkyl)₂, hydroxy and alkoxy groups.

In another embodiment R¹⁰ is aryl substituted with 1 to 3 independently selected R²¹ moieties.

In another embodiment R¹⁰ is aryl substituted with 1 to 3 R²¹ moieties, wherein each R²¹ moiety is the same or different —OR¹⁵ group.

In another embodiment R¹⁰ is aryl substituted with 1 R²¹ moiety.

In another embodiment R¹⁰ is aryl substituted with one R²¹ moiety, and said R²¹ moiety is —OR¹⁵.

In another embodiment R¹⁰ is phenyl substituted with 1 to 3 independently selected R²¹ moieties.

In another embodiment R¹⁰ is phenyl substituted with 1 to 3 R²¹ moieties, wherein each R²¹ moiety is the same or different —OR¹⁵ group.

In another embodiment R¹⁰ is phenyl substituted with 1 R²¹ moiety.

In another embodiment R¹⁰ is phenyl substituted with one R²¹ moiety, and said R²¹ moiety is —OR¹⁵.

In another embodiment R¹⁰ is phenyl substituted with one R²¹ moiety, said R²¹ moiety is —OR¹⁵, and said R¹⁵ is alkyl.

In another embodiment R¹⁰ is:

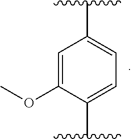

In another embodiment R¹⁰ is:

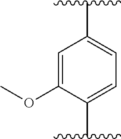

wherein the —R¹⁰-R⁹ moiety is:

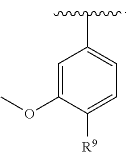

In another embodiment R¹⁰ is aryl substituted with 1 to 3 R²¹ moieties, wherein each R²¹ moiety is the same or different halo.

In another embodiment R¹⁰ is aryl substituted with 1 to 3 R²¹ moieties, wherein each R²¹ moiety is F.

In another embodiment R¹⁰ is aryl substituted with one R²¹ moiety, and said R²¹ moiety is halo.

In another embodiment R¹⁰ is aryl substituted with one R²¹ moiety, said R²¹ moiety is -halo, and said halo is F.

In another embodiment R¹⁰ is phenyl substituted with 1 to 3 R²¹ moieties, wherein each R²¹ moiety is the same or different halo.

In another embodiment R¹⁰ is phenyl substituted with 1 to 3 R²¹ moieties, wherein each R²¹ moiety is F.

In another embodiment R¹⁰ is phenyl substituted with one R²¹ moiety, and said R²¹ moiety is halo.

In another embodiment R¹⁰ is phenyl substituted with one R²¹ moiety, said R²¹ moiety is -halo, and said halo is F.

In another embodiment R¹⁰ is:

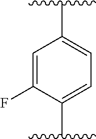

In another embodiment R¹⁰ is:

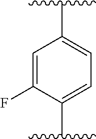

wherein the —R¹⁰-R⁹ moiety is:

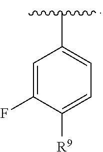

In another embodiment R¹⁰ is selected from the group consisting of:

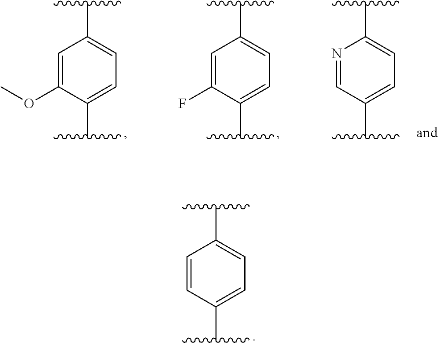

In another embodiment, R¹⁰ is unsubstituted heteroaryl.

In another embodiment, R¹⁰ is heteroaryl substituted with 1-3 substitutents, which can be the same or different, each being independently selected from the group consisting of halo, alkyl, CN, NH₂, NH(alkyl), N(alkyl)₂, hydroxy and alkoxy groups.

In another embodiment R¹⁰ is unsubstituted heteroaryl wherein said heteroaryl is pyridyl.

In another embodiment R¹⁰ is:

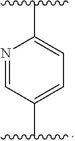

In another embodiment R¹⁰ is:

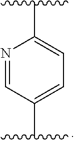

wherein the —R¹⁰-R⁹ moiety is:

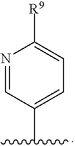

In another embodiment, R¹⁰ is aryl- and said aryl- is substituted with 1-3 substitutents, which can be the same or different, each being an alkoxy group.

In another embodiment, R¹⁰ is

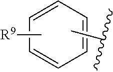

and R¹⁰ is substituted with 1-3 substitutents, which can be the same or different, each being an alkoxy group.

In another embodiment, R¹⁰ is aryl- is substituted with methoxy.

In another embodiment, R¹⁰ is

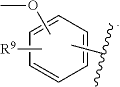

In another embodiment, R⁹ is unsubstituted heteroaryl.

In another embodiment, R⁹ is heteroaryl which is substituted with 1-3 substituents which can be the same or different, each substituent being independently selected from the group consisting of halo, alkyl, CN, NH₂, NH(alkyl), N(alkyl)₂, hydroxy and alkoxy groups.

In another embodiment of this invention R⁹ is selected from the group consisting of heteroaryl and heteroaryl substituted with 1-3 R²¹ groups, and wherein each R²¹ is independently selected.

In another embodiment, R⁹ is heteroaryl which is substituted with 1-3 substituents which can be the same or different, each substituent being independently selected from the group consisting of halo, alkyl, CN, NH₂, NH(alkyl), N(alkyl)₂, hydroxyl, alkoxy, alkyl substituted with halo (e.g., alkyl substituted with F, such as, for example, —CH₂F), and alkyl substituted with —OR¹⁵ (such as, for example, alkyl substituted with —OR¹⁵ wherein R¹⁵ is H, that is, —CH₂OH).

In another embodiment of this invention R⁹ is selected from the group consisting of imidazolyl and imidazolyl substituted with 1-3 R²¹ groups, and wherein each R²¹ is independently selected.

In another embodiment of this invention R⁹ is imidazolyl substituted with 1-3 R²¹ groups, and wherein each R²¹ is independently selected.

In another embodiment, R⁹ is imidazolyl which is substituted with 1-3 substituents which can be the same or different, each substituent being independently selected from the group consisting of halo, alkyl, CN, NH₂, NH(alkyl), N(alkyl)₂, hydroxyl, alkoxy, alkyl substituted with halo (e.g., alkyl substituted with F, such as, for example, —CH₂F), and alkyl substituted with —OR¹⁵ (such as, for example, alkyl substituted with —OR¹⁵ wherein R¹⁵ is H, that is, —CH₂OH).

In another embodiment, R⁹ is imidazolyl substituted with 1-3 substituents independently selected from the group consisting of halo, alkyl, CN, NH₂, NH(alkyl), N(alkyl)₂, hydroxy and alkoxy groups.

In another embodiment, R⁹ is imidazol-1-yl.

In another embodiment, R⁹ is 4-methyl-imidazol-1-yl.

In another embodiment, R⁹ is:

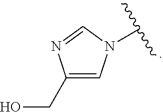

In another embodiment, R⁹ is:

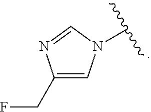

In another embodiment R¹⁰ is selected from the group consisting of aryl and aryl substituted with one or more R²¹ groups, and R⁹ is selected from the group consisting of heteroaryl and heteroaryl substituted with one or more R²¹ groups, and wherein each R²¹ is independently selected.

In another embodiment R¹⁰ is selected from the group consisting of phenyl and phenyl substituted with 1-3 independently selected R²¹ groups, and R⁹ is selected from the group consisting of imidazolyl and imidazolyl substituted with 1-3 independently selected R²¹ groups.

In another embodiment R¹⁰ is phenyl substituted with 1-3 independently selected R²¹ groups, and R⁹ is selected from the group consisting of imidazolyl and imidazolyl substituted with 1-3 independently selected R²¹ groups.

In another embodiment R¹⁰ is selected from the group consisting of heteroaryl and heteroaryl substituted with 1-3 R²¹ groups, and the R⁹ group is selected from the group consisting of heteroaryl and heteroaryl substituted with 1-3 R²¹ groups, and wherein each R²¹ is independently selected.

In another embodiment R¹⁰ is selected from the group consisting of pyridyl and pyridyl substituted with 1-3 R²¹ groups, and the R⁹ group is selected from the group consisting of imidazolyl and imidazolyl substituted with 1-3 R²¹ groups, and wherein each R²¹ is independently selected.

In another embodiment R¹⁰ is pyridyl, and the R⁹ group is imidazolyl substituted with 1-3 R²¹ groups, and wherein each R²¹ is independently selected.

In another embodiment the R⁹-R¹⁰— moiety is:

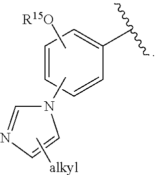

In another embodiment the R⁹-R¹⁰— moiety is:

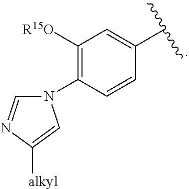

In another embodiment the R⁹-R¹⁰— moiety is:

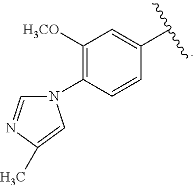

In another embodiment the R⁹-R¹⁰— moiety is:

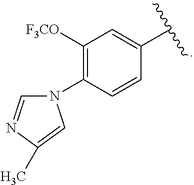

In another embodiment the R⁹-R¹⁰— moiety is:

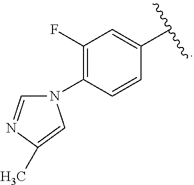

In another embodiment R⁹-R¹⁰— moiety is:

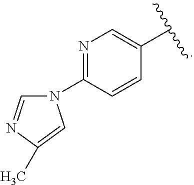

In another embodiment R⁹-R¹⁰— moiety is:

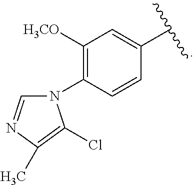

In another embodiment, R²¹ is independently selected from the group consisting of alkyl, alkyl-OH, unsubstituted arylalkyl-, arylalkyl wherein said aryl-portion of arylalkyl- is substituted with 1-3 halogen, unsubstituted aryl- and aryl wherein said aryl- is substituted with 1-3 halogen.

In another embodiment, R²¹ is independently selected from the group consisting of alkyl, alkyl-OH,

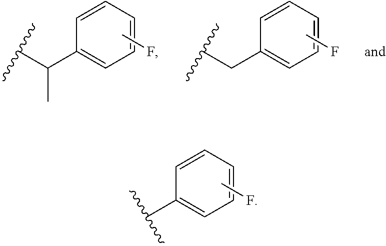

In another embodiment, R³, R⁴, R⁶ and R⁷ can be the same or different, each being independently selected from the group consisting of H and alkyl.

In another embodiment, R³, R⁴, R⁶ and R⁷ can be the same or different, each being independently selected from the group consisting of H and methyl.

In another embodiment R²¹ is alkyl.

In another embodiment R²¹ is -alkyl-OH.

In another embodiment R²¹ is

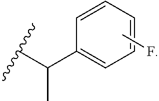

In another embodiment R²¹ is

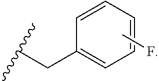

In another embodiment R²¹ is

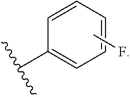

In another embodiment, the present application discloses a compound, or pharmaceutically acceptable salts, solvates, esters or prodrugs of said compound, said compound having the general structure shown in Formula:

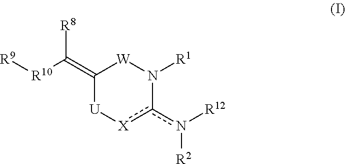

wherein:

W is —C(O)—;

U is a bond or —C(R³)(R⁴)—;

X is —N(R¹⁴)—;

each dashed line of

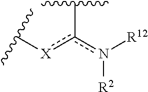

along with its adjoining single bond, together represents an optional double bond with the proviso that only one such double bond [symbol] is present at any given time, and further such that when the nitrogen of N(R²)(R¹²) is double bonded to the adjacent carbon between said nitrogen and X by the optional double bond, then R¹² is absent;

R¹ and R² are joined together to form a 5-14 membered heteroaryl, 5-8 membered heterocyclyl or 5-8 membered heterocyclenyl moiety, with each of said heteroaryl, heterocyclyl or heterocyclenyl moiety being unsubstituted or optionally independently being substituted with 1-5 R²¹ groups which can be the same or different;

R³ is independently selected from the group consisting of H, alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl-, wherein each of said alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl-;

R⁴ is independently selected from the group consisting of H, alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl-, wherein each of said alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl-;

R¹² is independently selected from the group consisting of H, alkyl, alkenyl, alkynyl, cycloalkyl, cycloalkylalkyl, cycloalkenyl, heterocyclyl, heterocyclylalkyl, aryl, arylalkyl, heteroaryl and heteroarylalkyl;

each R¹⁴ can be the same or different, each being independently selected from the group consisting of H, alkyl, alkenyl, alkynyl, cycloalkyl, cycloalkylalkyl, cycloalkenyl, heterocyclyl, heterocyclylalkyl, aryl, arylalkyl, heteroaryl and heteroarylalkyl;

R²¹ is independently selected from the group consisting of alkyl, alkyl-OH,

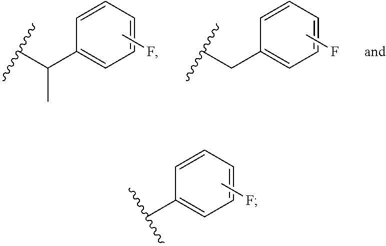

R⁹ is H or alkyl;

R¹⁰ is

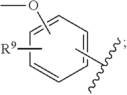

and

R⁹ is 4-methyl-imidazol-1-yl.

In another embodiment, the present application discloses a compound, or pharmaceutically acceptable salts, solvates, esters or prodrugs of said compound, said compound having the general structure shown in Formula:

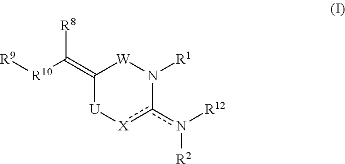

wherein:

W is —C(O)—;

U is a bond or —C(R³)(R⁴)—;

X is —N(R¹⁴)—;

each dashed line of

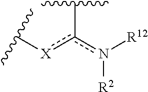

along with its adjoining single bond, together represents an optional double bond with the proviso that only one such double bond [symbol] is present at any given time, and further such that when the nitrogen of N(R²)(R¹²) is double bonded to the adjacent carbon between said nitrogen and X by the optional double bond, then R¹² is absent;

R¹ is selected from the group consisting of H, alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl-, wherein each of said alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl-being unsubstituted or optionally independently being substituted with 1-5 R²¹ groups which can be the same or different;

R² and R¹⁴ are joined together to form a 5-14 membered heteroaryl, 5-8 membered heterocyclyl or 5-8 membered heterocyclenyl moiety, with each of said heteroaryl, heterocyclyl or heterocyclenyl moiety being unsubstituted or optionally independently being substituted with 1-5 R²¹ groups which can be the same or different;

R³ is independently selected from the group consisting of H, alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl-, wherein each of said alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl-;

R⁴ is independently selected from the group consisting of H, alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl-, wherein each of said alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl-;

R¹² is independently selected from the group consisting of H, alkyl, alkenyl, alkynyl, cycloalkyl, cycloalkylalkyl, cycloalkenyl, heterocyclyl, heterocyclylalkyl, aryl, arylalkyl, heteroaryl and heteroarylalkyl;

each R¹⁴ (when not joined to R²) can be the same or different, each being independently selected from the group consisting of H, alkyl, alkenyl, alkynyl, cycloalkyl, cycloalkylalkyl, cycloalkenyl, heterocyclyl, heterocyclylalkyl, aryl, arylalkyl, heteroaryl and heteroarylalkyl;

. R²¹ is independently selected from the group consisting of alkyl, alkyl-OH,

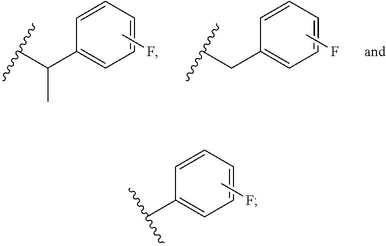

R⁸ is H or alkyl;

R¹⁰ is

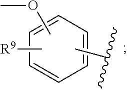

and

R⁹ is 4-methyl-imidazol-1-yl.

In another embodiment, the present application discloses a compound, or pharmaceutically acceptable salts, solvates, esters or prodrugs of said compound, said compound having the general structure shown in Formula:

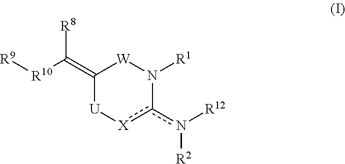

wherein:
R² and R⁶ are joined together to form a 5-14 membered heteroaryl, 5-8 membered heterocyclyl or 5-8 membered heterocyclenyl moiety, with each of said heteroaryl, heterocyclyl or heterocyclenyl moiety being unsubstituted or optionally independently being substituted with 1-5 substituents which can be the same or different, each substituent being independently selected from the group consisting of the moieties shown below; or R² and R¹⁴ are joined together to form a 5-14 membered heteroaryl, 5-8 membered heterocyclyl or 5-8 membered heterocyclenyl moiety, with each of said heteroaryl, heterocyclyl or heterocyclenyl moiety being unsubstituted or optionally independently being substituted with 1-5 substituents which can be the same or different, each substituent being independently selected from the group consisting of the moieties shown below;
W is —C(O)—;
U is a bond or —C(R³)(R⁴)—;
X is —C(R⁶)(R⁷)—;
each dashed line of

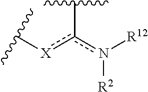

along with its adjoining single bond, together represents an optional double bond with the proviso that only one such double bond [symbol] is present at any given time, and further such that when the nitrogen of N(R²)(R¹²) is double bonded to the adjacent carbon between said nitrogen and X by the optional double bond, then R¹² is absent;

R¹ is independently selected from the group consisting of: alkyl, alkyl-OH,

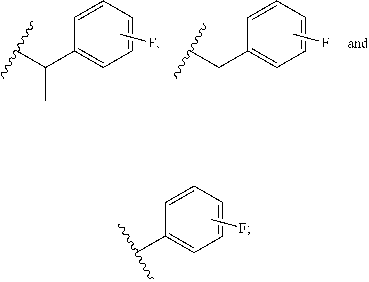

R⁵ is selected from the group consisting of H, alkyl, alkenyl, alkynyl, cycloalkyl, cycloalkylalkyl, cycloalkenyl, heterocyclyl, heterocyclylalkyl, aryl, arylalkyl, heteroaryl, heteroarylalkyl, —CN, —C(O)R¹⁵, —C(O)OR¹⁵, —C(O)N(R¹⁵)(R¹⁶), —S(O)N(R¹⁵)(R¹⁶), —S(O)₂N(R¹⁵)(R¹⁶), —S(O)R¹⁵, —S(O)₂R¹⁵, —C(═NOR¹⁵)R¹⁶ and —P(O)(OR¹⁵)(OR¹⁶);

R¹² is independently selected from the group consisting of H, alkyl, alkenyl, alkynyl, cycloalkyl, cycloalkylalkyl, cycloalkenyl, heterocyclyl, heterocyclylalkyl, aryl, arylalkyl, heteroaryl, heteroarylalkyl, —CN, —C(O)R¹⁵, —C(O)OR¹⁵, —C(O)N(R¹⁵)(R¹⁶), —S(O)N(R¹⁵)(R¹⁶), —S(O)₂N(R¹⁵)(R¹⁶)ₙ—S(O)R¹⁵, —S(O)₂R¹⁵, —C(═NOR¹⁵)R¹⁶ and —P(O)(OR¹⁵)(OR¹⁶);

each R¹⁴ (when R¹⁴ is not connected to R²) can be the same or different, each being independently selected from the group consisting of H, alkyl, alkenyl, alkynyl, cycloalkyl, cycloalkylalkyl, cycloalkenyl, heterocyclyl, heterocyclylalkyl, aryl, arylalkyl, heteroaryl, heteroarylalkyl, —CN, —C(O)R¹⁵, —C(O)OR¹⁶, —C(O)N(R¹⁵)(R¹⁶), —S(O)N(R¹⁵)(R¹⁶), —S(O)₂N(R¹⁵)(R¹⁶), —S(O)R¹⁵, —S(O)₂R¹⁵, —C(═NOR¹⁵)R¹⁶, and —P(O)(OR¹⁵)(OR¹⁶);

R³ is independently selected from the group consisting of H, alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl-, wherein each of said alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl- can be unsubstituted or optionally independently substituted with 1-5 substituents which can be the same or different, each substituent being independently selected from the group consisting of consisting of the moieties shown below;

R⁴ is independently selected from the group consisting of H, alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl-, wherein each of said alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl- can be unsubstituted or optionally independently substituted with 1-5 substituents which can be the same or different, each substituent being independently selected from the group consisting of consisting of the moieties shown below;

R⁶ (when R⁶ is not connected to R²) is selected from the group consisting of H, alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl-, wherein each of said alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl- can be unsubstituted or optionally independently substituted with 1-5 substituents which can be the same or different, each substituent being independently selected from the group consisting of consisting of the moieties shown below;

R⁷ is selected from the group consisting of H, alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl-, wherein each of said alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl- can be unsubstituted or optionally independently substituted with 1-5 substituents which can be the same or different, each substituent being independently selected from the group consisting of consisting of the moieties shown below;

R⁸ is H or alkyl;

R¹⁰ is

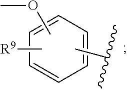

R⁹ is 4-methyl-imidazol-1-yl;

R¹⁵, R¹⁶ and R¹⁷ are independently selected from the group consisting of H, alkyl, alkenyl, alkynyl, cycloalkyl, cycloalkylalkyl, heterocyclyl, heterocyclylalkyl, aryl, arylalkyl, heteroaryl, heteroarylalkyl, arylcycloalkyl, arylheterocyclyl, R¹⁸-alkyl, R¹⁸-cycloalkyl, R¹⁸-cycloalkylalkyl, R¹⁸-heterocyclyl, R¹⁸-heterocyclylalkyl, R¹⁸-aryl, R¹⁸-arylalkyl, R¹⁸-heteroaryl and R¹⁸-heteroarylalkyl;

R¹⁸ is 1-5 substituents independently selected from the group consisting of alkyl, alkenyl, alkynyl, aryl, arylalkyl, arylalkenyl, arylalkynyl, —NO₂, halo, heteroaryl, HO-alkyoxyalkyl, —CF₃, —CN, alkyl-CN, —C(O)R¹⁹, —C(O)OH, —C(O)OR¹⁹, —C(O)NHR²⁰, —C(O)NH₂, —C(O)NH₂—C(O)N(alkyl)₂, —C(O)N(alkyl)(aryl), —C(O)N(alkyl)(heteroaryl), —SR¹⁹, —S(O)₂R²⁰, —S(O)NH₂, —S(O)NH(alkyl), —S(O)N(alkyl)(alkyl), —S(O)NH(aryl), —S(O)₂NH₂, —S(O)₂NHR¹⁹, —S(O)₂NH(heterocyclyl), —S(O)₂N(alkyl)₂, —S(O)₂N(alkyl)(aryl), —OCF₃, —OH, —OR²⁰, —O-heterocyclyl, —O-cycloalkylalkyl, —O-heterocyclylalkyl, —NH₂, —NHR²⁰, —N(alkyl)₂, —N(arylalkyl)₂, —N(arylalkyl)-(heteroarylalkyl), —NHC(O)R²⁰, —NHC(O)NH₂, —NHC(O)NH(alkyl), —NHC(O)N(alkyl)(alkyl), —N(alkyl)C(O)NH(alkyl), —N(alkyl)C(O)N(alkyl)(alkyl), —NHS(O)₂R²⁰, —NHS(O)₂NH(alkyl), —NHS(O)₂N(alkyl)(alkyl), —N(alkyl)S(O)₂NH(alkyl) and —N(alkyl)S(O)₂N(alkyl)(alkyl);

or, alternately, two R¹⁸ moieties on adjacent carbons can be linked together to form:

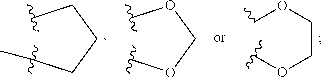

R¹⁹ is alkyl, cycloalkyl, aryl, arylalkyl or heteroarylalkyl;

R²⁰ is alkyl, cycloalkyl, aryl, halo substituted aryl, arylalkyl, heteroaryl or heteroarylalkyl;

wherein each of the alkyl, cycloalkyl, cycloalkylalkyl, heterocyclyl, heterocyclylalkyl, aryl, arylalkyl, alkylaryl, heteroaryl, heteroarylalkyl, alkenyl and alkynyl groups in R³, R⁴, R⁵, R⁶, R⁷, R¹² and R¹⁴, the 5-14 membered heteroaryl, 5-8 membered heterocyclyl or 5-8 membered heterocyclenyl moiety formed from the joining of R² and R¹⁴ or R² and R⁶ are independently unsubstituted or substituted by 1 to 5 R²¹ groups independently selected from the group consisting of alkyl, alkenyl, alkynyl, cycloalkyl, cycloalkylalkyl, cycloalkenyl, heterocyclyl, heterocyclylalkyl, aryl, arylalkyl, heteroaryl, heteroarylalkyl, halo, —CN, —OR¹⁵, —C(O)R¹⁵, —C(O)OR¹⁵, —C(O)N(R¹⁵)(R¹⁶), —SR¹⁵, —S(O)N(R¹⁵)(R¹⁶), —CH(R¹⁵)(R¹⁶), —S(O)₂N(R¹⁵)(R¹⁶), —C(═NOR¹⁵)R¹⁶, —P(O)(OR¹⁵)(OR¹⁶), —N(R¹⁵)(R¹⁶), -alkyl-N(R¹⁵)(R¹⁶), —N(R¹⁵)C(O)R¹⁶, —CH₂—N(R¹⁵)C(O)R¹⁶, —CH₂—N(R¹⁵)C(O)N(R¹⁶)(R¹⁷), —CH₂—R¹⁵; —CH₂N(R¹⁵)(R¹⁶), —N(R¹⁵)S(O)R¹⁶, —N(R¹⁵)S(O)₂R¹⁶, —CH₂—N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —CH₂—N(R¹⁵)C(O)N(R¹⁶)(R¹⁷), —N(R¹⁵)C(O)OR¹⁶, —CH₂—N(R¹⁵)C(O)OR¹⁶, —S(O)R¹⁵, ═NOR¹⁵, —N₃, —NO₂ and —S(O)₂R¹⁵; and

wherein each of the alkyl, cycloalkenyl, cycloalkyl, cycloalkylalkyl, heterocyclyl, heterocyclylalkyl, aryl, arylalkyl, heteroaryl, heteroarylalkyl, alkenyl and alkynyl groups in R²¹ are independently unsubstituted or substituted by 1 to 5 R²² groups independently selected from the group consisting of alkyl, cycloalkyl, cycloalkenyl, heterocyclyl, aryl, heteroaryl, halo, —CF₃, —CN, —OR¹⁵, —C(O)R¹⁵, —C(O)OR¹⁵, -alkyl-C(O)OR¹⁵, C(O)N(R¹⁵)(R¹⁶), —SR¹⁵, —S(O)N(R¹⁵)(R¹⁶), —S(O)₂N(R¹⁵)(R¹⁶), —C(═NOR¹⁵)R¹⁶, —P(O)(OR¹⁵)(OR¹⁶), —N(R¹⁵)(R¹⁶), -alkyl-N(R¹⁵)(R¹⁶), —N(R¹⁵)C(O)R¹⁶, —CH₂—N(R¹⁵)C(O)R¹⁶, —N(R¹⁵)S(O)R¹⁶, —N(R¹⁵)S(O)₂R¹⁶, —CH₂—N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —CH₂—N(R¹⁵)C(O)N(R¹⁶)(R¹⁷), —N(R¹⁵)C(O)OR¹⁶, —CH₂—N(R¹⁵)C(O)OR¹⁶, —N₃, ═NOR¹⁵, —NO₂, —S(O)R¹⁵ and —S(O)₂R¹⁵.

In another embodiment, the present application discloses a compound, or pharmaceutically acceptable salts, solvates, esters or prodrugs of said compound, said compound having the general structure shown in Formula:

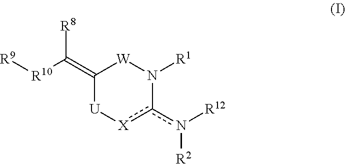

wherein:

W is —C(O)—;

U is —C(R³)(R⁴)—;

X is —C(R⁶)(R⁷)—;

each dashed line of

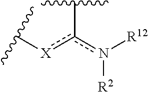

along with its adjoining single bond, together represents an optional double bond with the proviso that only one such double bond [symbol] is present at any given time, and further such that when the nitrogen of N(R²)(R¹²) is double bonded to the adjacent carbon between said nitrogen and X by the optional double bond, then R¹² is absent;

R¹ is independently selected from the group consisting of H, methyl,

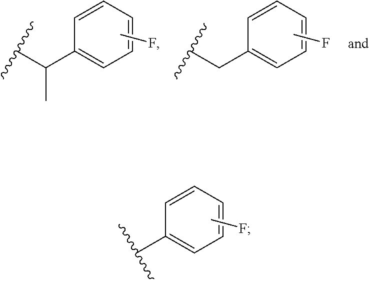

R³ and R⁶ are joined together to form a 5-14 membered aryl, 5-8 membered cycloalkyl, 5-8 membered cycloalkenyl, 5-14 membered heteroaryl, 5-8 membered heterocyclyl or 5-8 membered heterocyclenyl moiety, with each of said aryl, cycloalkyl, cycloalkenyl, heteroaryl, heterocyclyl or heterocyclenyl moiety being unsubstituted or optionally independently being substituted with 1-5 R²¹ groups which can be the same or different;

R⁴ is independently selected from the group consisting of H, alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl-, wherein each of said alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl-;

R¹² is independently selected from the group consisting of H, alkyl, alkenyl, alkynyl, cycloalkyl, cycloalkylalkyl, cycloalkenyl, heterocyclyl, heterocyclylalkyl, aryl, arylalkyl, heteroaryl and heteroarylalkyl;

each R¹⁴ can be the same or different, each being independently selected from the group consisting of H, alkyl, alkenyl, alkynyl, cycloalkyl, cycloalkylalkyl, cycloalkenyl, heterocyclyl, heterocyclylalkyl, aryl, arylalkyl, heteroaryl and heteroarylalkyl;

R²¹ is independently selected from the group consisting of alkyl, alkyl-OH,

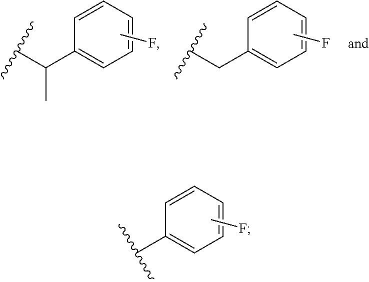

R⁸ is H or alkyl;

R¹⁰ is

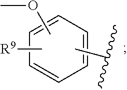

and

R⁹ is 4-methyl-imidazol-1-yl.

Another embodiment is directed to compounds of formula (I) wherein:
W is —C(O)—;
U is —C(R³)(R⁴)—;
X is —N(R¹⁴)—;
R¹ is alkyl, alkyl-OH,

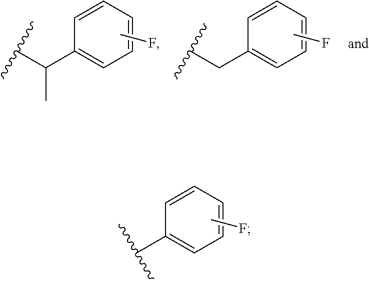

R²¹ is independently selected from the group consisting of alkyl, alkyl-OH,

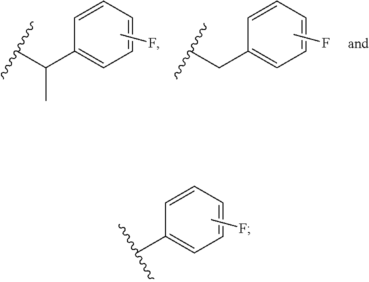

R⁸ is H or alkyl;
R¹⁰ is

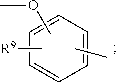

and
R⁹ is 4-methyl-imidazol-1-yl.

Another embodiment of this invention is directed to compounds of formula (I) wherein: (a) R¹ and R² are joined together to form a 5-14 membered heteroaryl moiety, (b) W is —C(O)—, (c) U is a bond or —C(R³)(R⁴)—, (d) X is —N(R¹⁴)—, (e) R⁸ is H or alkyl, (f) R¹⁰ is

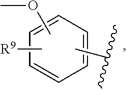

(g) R⁹ is 4-methyl-imidazol-1-yl, and (h) R²¹ is independently selected from the group consisting of alkyl, alkyl-OH,

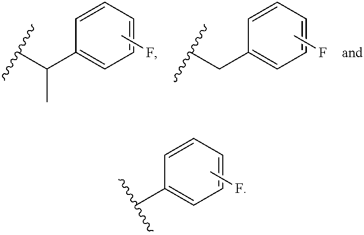

Another embodiment of this invention is directed to compounds of formula (I) wherein: (a) R¹ and R² are joined together to form a 5-14 membered heteroaryl moiety optionally substituted with 1 to 5 independently selected R²¹ groups, (b) W is —C(O)—, (c) U is a bond or —C(R³)(R⁴)—, (d) X is —N(R¹⁴)—, (e) R⁸ is H or alkyl, (f) R¹⁰ is

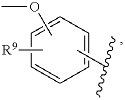

(g) R⁹ is 4-methyl-imidazol-1-yl, and (h) R²¹ is independently selected from the group consisting of alkyl, alkyl-OH,

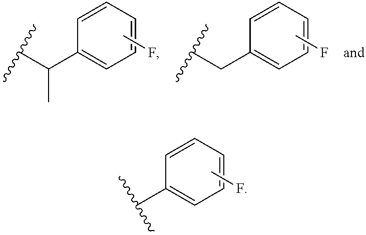

Another embodiment of this invention is directed to compounds of formula (I) wherein: (a) R¹ and R² are joined together to form a 5-14 membered heteroaryl moiety substituted with 1 to 5 independently selected R²¹ groups, (b) W is —C(O)—, (c) U is a bond or —C(R³)(R⁴)—, (d) X is —N(R¹⁴)—, (e) R⁸ is H or alkyl, (f) R¹⁰ is

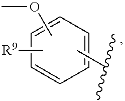

(g) R⁹ is 4-methyl-imidazol-1-yl, and (h) R²¹ is independently selected from the group consisting of alkyl, alkyl-OH,

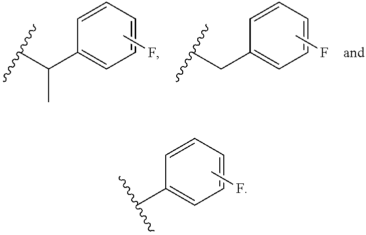

Another embodiment of this invention is directed to compounds of formula (I) wherein: (a) R¹ and R² are joined together to form a 5-14 membered heteroaryl moiety optionally substituted with 1 to 5 independently selected R²¹ groups, and said heteroaryl moiety is optionally fused with an aryl or heteroaryl ring, and the ring moiety resulting from the fusion is optionally substituted with 1 to 5 independently selected R²¹ groups, (b) W is —C(O)—, (c) U is a bond or —C(R³)(R⁴)—, (d) X is —N(R¹⁴)—, (e) R⁸ is H or alkyl, (f) R¹⁰ is

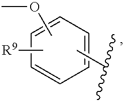

(g) R⁹ is 4-methyl-imidazol-1-yl, and (h) R²¹ is independently selected from the group consisting of alkyl, alkyl-OH,

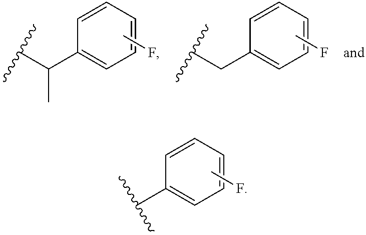

Another embodiment of this invention is directed to compounds of formula (I) wherein: (a) R¹ and R² are joined together to form a 5-14 membered heteroaryl moiety optionally substituted with 1 to 5 independently selected R²¹ groups, and said heteroaryl moiety is optionally fused with an aryl ring, and the ring moiety resulting from the fusion is optionally substituted with 1 to 5 independently selected R²¹ groups, (b) W is —C(O)—, (c) U is a bond or —C(R³)(R⁴)—, (d) X is —N(R¹⁴)—, (e) R⁸ is H or alkyl, (f) R¹⁰ is

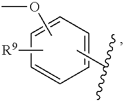

(g) R⁹ is 4-methyl-imidazol-1-yl, and (h) R²¹ is independently selected from the group consisting of alkyl, alkyl-OH,

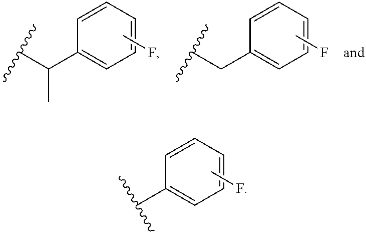

Another embodiment of this invention is directed to compounds of formula (I) wherein: (a) R¹ and R² are joined together to form a 5-14 membered heteroaryl moiety optionally substituted with 1 to 5 independently selected R²¹ groups, and said heteroaryl moiety is optionally fused with a heteroaryl ring, and the ring moiety resulting from the fusion is optionally substituted with 1 to 5 independently selected R²¹ groups, (b) W is —C(O)—, (c) U is a bond or —C(R³)(R⁴)—, (d) X is —N(R¹⁴)—, (e) R⁸ is H or alkyl, (f) R¹⁰ is

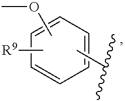

(g) R⁹ is 4-methyl-imidazol-1-yl, and (h) R²¹ is independently selected from the group consisting of alkyl, alkyl-OH,

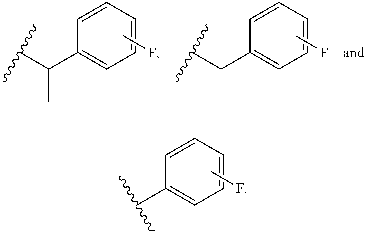

Another embodiment of this invention is directed to compounds of formula (I) wherein: (a) R¹ and R² are joined together to form a 5-8 membered heterocyclyl moiety, (b) W is —C(O)—, (c) U is a bond or —C(R³)(R⁴)—, (d) X is —N(R¹⁴)—, (e) R⁸ is H or alkyl, (f) R¹⁰ is

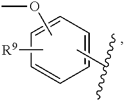

(g) R⁹ is 4-methyl-imidazol-1-yl, and (h) R²¹ is independently selected from the group consisting of alkyl, alkyl-OH,

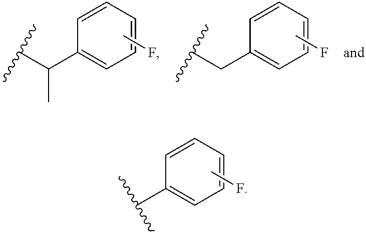

Another embodiment of this invention is directed to compounds of formula (I) wherein: (a) R¹ and R² are joined together to form a 5-8 membered heterocyclyl moiety optionally substituted with 1 to 5 independently selected R²¹ groups, (b) W is —C(O)—, (c) U is a bond or —C(R³)(R⁴)—, (d) X is —N(R¹⁴)—, (e) R⁸ is H or alkyl, (f) R¹⁰ is

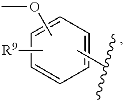

(g) R⁹ is 4-methyl-imidazol-1-yl, and (h) R²¹ is independently selected from the group consisting of alkyl, alkyl-OH,

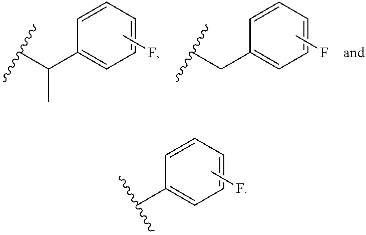

Another embodiment of this invention is directed to compounds of formula (I) wherein: (a) R¹ and R² are joined together to form a 5-8 membered heterocyclyl moiety substituted with 1 to 5 independently selected R²¹ groups, (b) W is —C(O)—, (c) U is a bond or —C(R³)(R⁴)—, (d) X is —N(R¹⁴)—, (e) R⁸ is H or alkyl, (f) R¹⁰ is

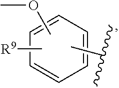

(g) R⁹ is 4-methyl-imidazol-1-yl, and (h) R²¹ is independently selected from the group consisting of alkyl, alkyl-OH,

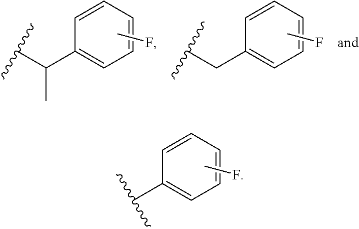

Another embodiment of this invention is directed to compounds of formula (I) wherein: (a) R¹ and R² are joined together to form a 5-8 membered heterocyclyl moiety optionally substituted with 1 to 5 independently selected R²¹ groups, and said heteroaryl moiety is optionally fused with an aryl or heteroaryl ring, and the ring moiety resulting from the fusion is optionally substituted with 1 to 5 independently selected R²¹ groups, (b) W is —C(O)—, (c) U is a bond or —C(R³)(R⁴)—, (d) X is —N(R¹⁴)—, (e) R⁸ is H or alkyl, (f) R¹⁰ is

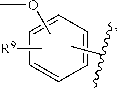

(g) R⁹ is 4-methyl-imidazol-1-yl, and (h) R²¹ is independently selected from the group consisting of alkyl, alkyl-OH,

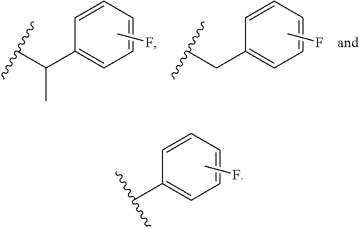

Another embodiment of this invention is directed to compounds of formula (I) wherein: (a) R¹ and R² are joined together to form a 5-8 membered heterocyclyl moiety optionally substituted with 1 to 5 independently selected R²¹ groups, and said heteroaryl moiety is optionally fused with an aryl ring, and the ring moiety resulting from the fusion is optionally substituted with 1 to 5 independently selected R²¹ groups, (b) W is —C(O)—, (c) U is a bond or —C(R³)(R⁴)—, (d) X is —N(R¹⁴)—, (e) R⁸ is H or alkyl, (f) R¹⁰ is

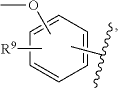

(g) R⁹ is 4-methyl-imidazol-1-yl, and (h) R²¹ is independently selected from the group consisting of alkyl, alkyl-OH,

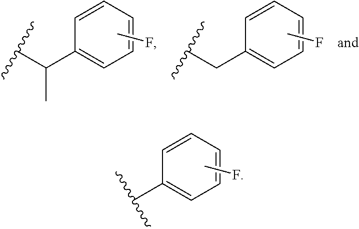

Another embodiment of this invention is directed to compounds of formula (I) wherein: (a) R¹ and R² are joined together to form a 5-8 membered heterocyclyl moiety optionally substituted with 1 to 5 independently selected R²¹ groups, and said heteroaryl moiety is optionally fused with a heteroaryl ring, and the ring moiety resulting from the fusion is optionally substituted with 1 to 5 independently selected R²¹ groups, (b) W is —C(O)—, (c) U is a bond or —C(R³)(R⁴)—, (d) X is —N(R¹⁴)—, (e) R⁸ is H or alkyl, (f) R¹⁰ is

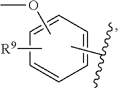

(g) R⁹ is 4-methyl-imidazol-1-yl, and (h) R²¹ is independently selected from the group consisting of alkyl, alkyl-OH,

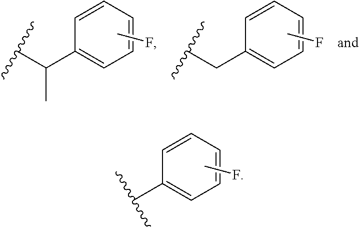

Another embodiment of this invention is directed to compounds of formula (I) wherein: (a) R¹ and R² are joined together to form a 5-8 membered heterocyclenyl moiety, (b) W is —C(O)—, (c) U is a bond or —C(R³)(R⁴)—, (d) X is (e) R⁸ is H or alkyl, (f) R¹⁰ is

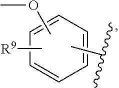

(g) R⁹ is 4-methyl-imidazol-1-yl, and (h) R²¹ is independently selected from the group consisting of alkyl, alkyl-OH,

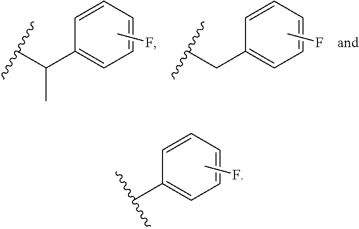

Another embodiment of this invention is directed to compounds of formula (I) wherein: (a) R¹ and R² are joined together to form a 5-8 membered heterocyclenyl moiety optionally substituted with 1 to 5 independently selected R²¹ groups, (b) W is —C(O)—, (c) U is a bond or —C(R³)(R⁴)—, (d) X is —N(R¹⁴)—, (e) R⁸ is H or alkyl, (f) R¹⁶ is

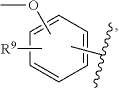

(g) R⁹ is 4-methyl-imidazol-1-yl, and (h) R²¹ is independently selected from the group consisting of alkyl, alkyl-OH,

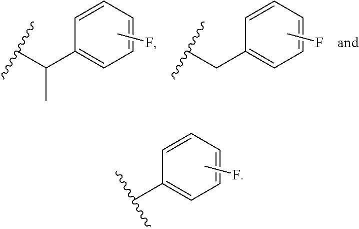

Another embodiment of this invention is directed to compounds of formula (I) wherein: (a) R¹ and R² are joined together to form a 5-8 membered heterocyclenyl moiety substituted with 1 to 5 independently selected R²¹ groups, (b) W is —C(O)—, (c) U is a bond or —C(R³)(R⁴)—, (d) X is —N(R¹⁴)—, (e) R⁸ is H or alkyl, (f) R¹⁶ is

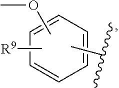

(g) R⁹ is 4-methyl-imidazol-1-yl, and (h) R²¹ is independently selected from the group consisting of alkyl, alkyl-OH,

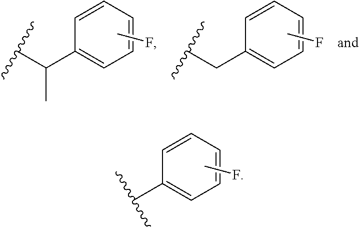

Another embodiment of this invention is directed to compounds of formula (I) wherein: (a) R¹ and R² are joined together to form a 5-8 membered heterocyclenyl moiety optionally substituted with 1 to 5 independently selected R²¹ groups, and said heteroaryl moiety is optionally fused with an aryl or heteroaryl ring, and the ring moiety resulting from the fusion is optionally substituted with 1 to 5 independently selected R²¹ groups, (b) W is —C(O)—, (c) U is a bond or —C(R³)(R⁴)—, (d) X is —N(R¹⁴)—, (e) R⁸ is H or alkyl, (f) R¹⁰ is

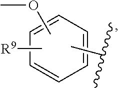

(g) R⁹ is 4-methyl-imidazol-1-yl, and (h) R²¹ is independently selected from the group consisting of alkyl, alkyl-OH,

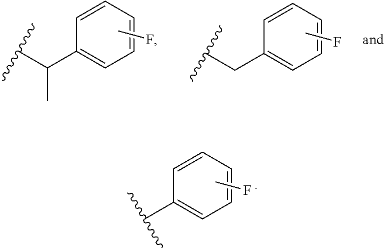

Another embodiment of this invention is directed to compounds of formula (I) wherein: (a) R¹ and R² are joined together to form a 5-8 membered heterocyclenyl moiety optionally substituted with 1 to 5 independently selected R²¹ groups, and said heteroaryl moiety is optionally fused with an aryl ring, and the ring moiety resulting from the fusion is optionally substituted with 1 to 5 independently selected R²¹ groups, (b) W is —C(O)—, (c) U is a bond or —C(R³)(R⁴)—, (d) X is —N(R¹⁴)—, (e) R⁸ is H or alkyl, (f) R¹⁰ is

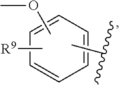

(g) R⁹ is 4-methyl-imidazol-1-yl, and (h) R²¹ is independently selected from the group consisting of alkyl, alkyl-OH,

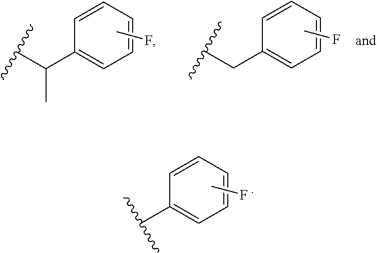

Another embodiment of this invention is directed to compounds of formula (I) wherein: (a) R¹ and R² are joined together to form a 5-8 membered heterocyclenyl moiety optionally substituted with 1 to 5 independently selected R²¹ groups, and said heteroaryl moiety is optionally fused with a heteroaryl ring, and the ring moiety resulting from the fusion is optionally substituted with 1 to 5 independently selected R²¹ groups, (b) W is —C(O)—, (c) U is a bond or —C(R³)(R⁴)—, (d) X is —N(R¹⁴)—, (e) R⁸ is H or alkyl, (f) R¹⁰ is

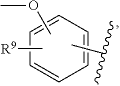

(g) R⁹ is 4-methyl-imidazol-1-yl, and (h) R²¹ is independently selected from the group consisting of alkyl, alkyl-OH,

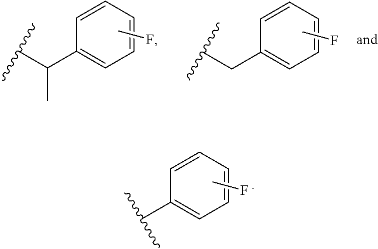

Another embodiment of this invention is directed to compounds of formula (I) wherein: (a) R² and R⁶ are joined together to form a 5-14 membered heteroaryl moiety, (b) W is —C(O)—, (c) U is a bond or —C(R³)(R⁴)—, (d) X is —C(R⁶)(R⁷)—, (e) R⁸ is H or alkyl, (f) R¹⁰ is

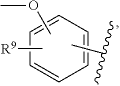

(g) R⁹ is 4-methyl-imidazol-1-yl, and (h) R²¹ is independently selected from the group consisting of alkyl, alkyl-OH,

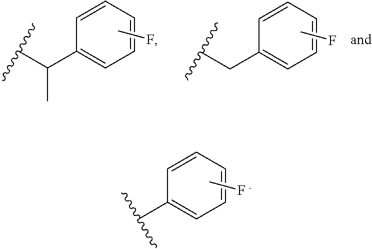

Another embodiment of this invention is directed to compounds of formula (I) wherein: (a) R² and R⁶ are joined together to form a 5-14 membered heteroaryl moiety optionally substituted with 1 to 5 independently selected R²¹ groups, (b) W is —C(O)—, (c) U is a bond or —C(R³)(R⁴)—, (d) X is —C(R⁶)(R⁷)—, (e) R⁸ is H or alkyl, (f) R¹⁰ is

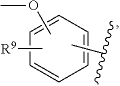

(g) R⁹ is 4-methyl-imidazol-1-yl, and (h) R²¹ is independently selected from the group consisting of alkyl, alkyl-OH,

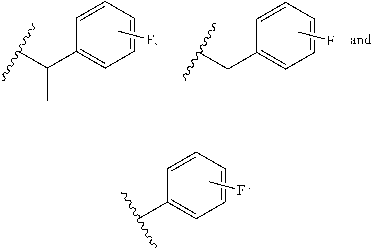

Another embodiment of this invention is directed to compounds of formula (I) wherein: (a) R² and R⁶ are joined together to form a 5-14 membered heteroaryl moiety substituted with 1 to 5 independently selected R²¹ groups, (b) W is —C(O)—, (c) U is a bond or —C(R³)(R⁴)—, (d) X is —C(R⁶)(R⁷)—, (e) R⁸ is H or alkyl, (f) R¹⁰ is

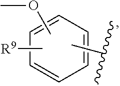

(g) R⁹ is 4-methyl-imidazol-1-yl, and (h) R²¹ is independently selected from the group consisting of alkyl, alkyl-OH,

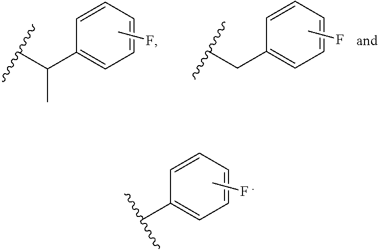

Another embodiment of this invention is directed to compounds of formula (I) wherein: (a) R² and R⁶ are joined together to form a 5-14 membered heteroaryl moiety optionally substituted with 1 to 5 independently selected R²¹ groups, and said heteroaryl moiety is optionally fused with an aryl or heteroaryl ring, and the ring moiety resulting from the fusion is optionally substituted with 1 to 5 independently selected R²¹ groups, (b) W is —C(O)—, (c) U is a bond or —C(R³)(R⁴)—, (d) X is —C(R⁶)(R⁷)—, (e) R⁸ is H or alkyl, (f) R¹⁰ is

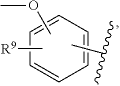

(g) R⁹ is 4-methyl-imidazol-1-yl, and (h) R²¹ is independently selected from the group consisting of alkyl, alkyl-OH,

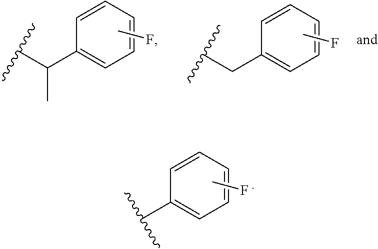

Another embodiment of this invention is directed to compounds of formula (I) wherein: (a) R² and R⁶ are joined together to form a 5-14 membered heteroaryl moiety optionally substituted with 1 to 5 independently selected R²¹ groups, and said heteroaryl moiety is optionally fused with an aryl ring, and the ring moiety resulting from the fusion is optionally substituted with 1 to 5 independently selected R²¹ groups, (b) W is —C(O)—, (c) U is a bond or —C(R³)(R⁴)—, (d) X is —C(R⁶)(R⁷)—, (e) R⁸ is H or alkyl, (f) R¹⁰ is

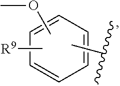

(g) R⁹ is 4-methyl-imidazol-1-yl, and (h) R²¹ is independently selected from the group consisting of alkyl, alkyl-OH,

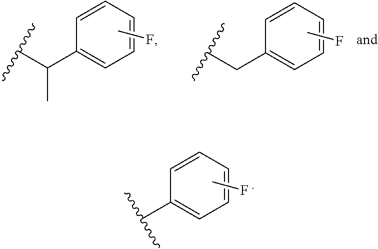

Another embodiment of this invention is directed to compounds of formula (I) wherein: (a) R² and R⁶ are joined together to form a 5-14 membered heteroaryl moiety optionally substituted with 1 to 5 independently selected R²¹ groups, and said heteroaryl moiety is optionally fused with a heteroaryl ring, and the ring moiety resulting from the fusion is optionally substituted with 1 to 5 independently selected R²¹ groups, (b) W is —C(O)—, (c) U is a bond or —C(R³)(R⁴)—, (d) X is —C(R⁶)(R⁷)—, (e) R⁸ is H or alkyl, (f) R¹⁰ is

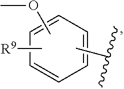

(g) R⁹ is 4-methyl-imidazol-1-yl, and (h) R²¹ is independently selected from the group consisting of alkyl, alkyl-OH,

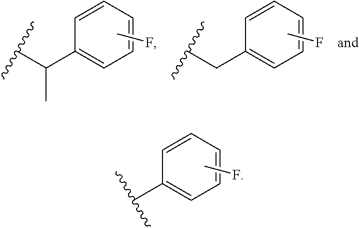

Another embodiment of this invention is directed to compounds of formula (I) wherein: (a) R² and R⁶ are joined together to form a 5-8 membered heterocyclyl moiety, (b) W is —C(O)—, (c) U is a bond or —C(R³)(R⁴)—, (d) X is —C(R⁶)(R⁷)—, (e) R⁸ is H or alkyl, (f) R¹⁰ is

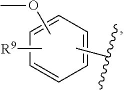

(g) R⁹ is 4-methyl-imidazol-1-yl, and (h) R²¹ is independently selected from the group consisting of alkyl, alkyl-OH,

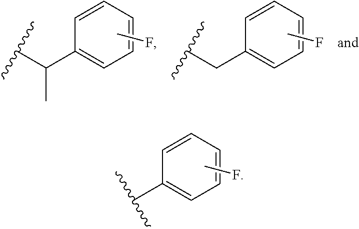

Another embodiment of this invention is directed to compounds of formula (I) wherein: (a) R² and R⁶ are joined together to form a 5-8 membered heterocyclyl moiety optionally substituted with 1 to 5 independently selected R²¹ groups, (b) W is —C(O)—, (c) U is a bond or —C(R³)(R⁴)—, (d) X is —C(R⁶)(R⁷)—, (e) R⁸ is H or alkyl, (f) R¹⁰ is

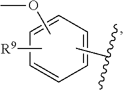

(g) R⁹ is 4-methyl-imidazol-1-yl, and (h) R²¹ is independently selected from the group consisting of alkyl, alkyl-OH,

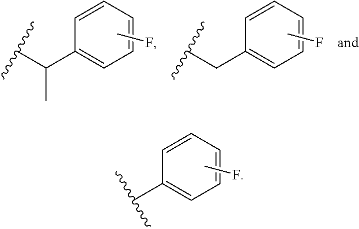

Another embodiment of this invention is directed to compounds of formula (I) wherein: (a) R² and R⁶ are joined together to form a 5-8 membered heterocyclyl moiety substituted with 1 to 5 independently selected R²¹ groups, (b) W is —C(O)—, (c) U is a bond or —C(R³)(R⁴)—, (d) X—C(R⁶)(R⁷)—, (e) R⁸ is H or alkyl, (f) R¹⁰ is

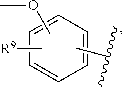

(g) R⁹ is 4-methyl-imidazol-1-yl, and (h) R²¹ is independently selected from the group consisting of alkyl, alkyl-OH,

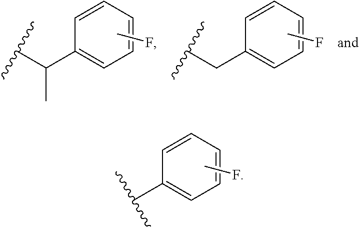

Another embodiment of this invention is directed to compounds of formula (I) wherein: (a) R² and R⁶ are joined together to form a 5-8 membered heterocyclyl moiety optionally substituted with 1 to 5 independently selected R²¹ groups, and said heteroaryl moiety is optionally fused with an aryl or heteroaryl ring, and the ring moiety resulting from the fusion is optionally substituted with 1 to 5 independently selected R²¹ groups, (b) W is —C(O)—, (c) U is a bond or —C(R³)(R⁴)—, (d) X is —C(R⁶)(R⁷)—, (e) R⁸ is H or alkyl, (f) R¹⁰ is

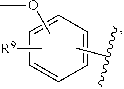

(g) R⁹ is 4-methyl-imidazol-1-yl, and (h) R²¹ is independently selected from the group consisting of alkyl, alkyl-OH,

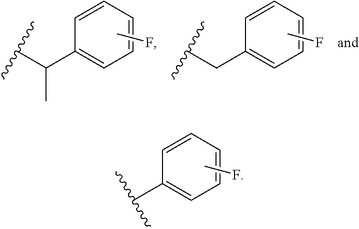

Another embodiment of this invention is directed to compounds of formula (I) wherein: (a) R² and R⁶ are joined together to form a 5-8 membered heterocyclyl moiety optionally substituted with 1 to 5 independently selected R²¹ groups, and said heteroaryl moiety is optionally fused with an aryl ring, and the ring moiety resulting from the fusion is optionally substituted with 1 to 5 independently selected R²¹ groups, (b) W is —C(O)—, (c) U is a bond or —C(R³)(R⁴)—, (d) X is —C(R⁶)(R⁷)—, (e) Fe is H or alkyl, (f) R¹⁰ is

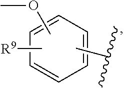

(g) R⁹ is 4-methyl-imidazol-1-yl, and (h) R²¹ is independently selected from the group consisting of alkyl, alkyl-OH,

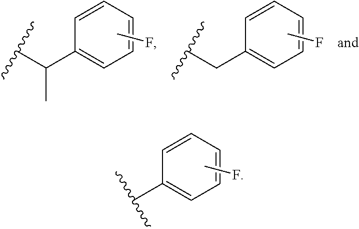

Another embodiment of this invention is directed to compounds of formula (I) wherein: (a) R² and R⁶ are joined together to form a 5-8 membered heterocyclyl moiety optionally substituted with 1 to 5 independently selected R²¹ groups, and said heteroaryl moiety is optionally fused with a heteroaryl ring, and the ring moiety resulting from the fusion is optionally substituted with 1 to 5 independently selected R²¹ groups, (b) W is —C(O)—, (c) U is a bond or —C(R³)(R⁴)—, (d) X is —C(R⁶)(R⁷)—, (e) R⁸ is H or alkyl, (f) R¹⁰ is

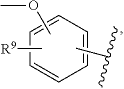

(g) R⁹ is 4-methyl-imidazol-1-yl, and (h) R²¹ is independently selected from the group consisting of alkyl, alkyl-OH,

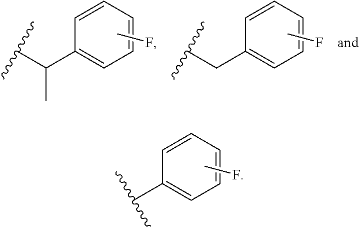

Another embodiment of this invention is directed to compounds of formula (I) wherein: (a) R² and R⁶ are joined together to form a 5-8 membered heterocyclenyl moiety, (b) W is —C(O)—, (c) U is a bond or —C(R³)(R⁴)—, (d) X is —C(R⁶)(R⁷)—, (e) R⁸ is H or alkyl, (f) R¹⁰ is

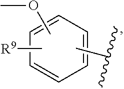

(g) R⁹ is 4-methyl-imidazol-1-yl, and (h) R²¹ is independently selected from the group consisting of alkyl, alkyl-OH,

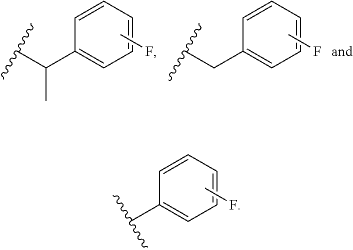

Another embodiment of this invention is directed to compounds of formula (I) wherein: (a) R² and R⁶ are joined together to form a 5-8 membered heterocyclenyl moiety optionally substituted with 1 to 5 independently selected R²¹ groups, (b) W is —C(O)—, (c) U is a bond or —C(R³)(R⁴)—, (d) X is —C(R⁶)(R⁷)—, (e) R⁸ is H or alkyl, (f) R¹⁰ is

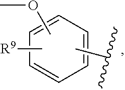

(g) R⁹ is 4-methyl-imidazol-1-yl, and (h) R²¹ is independently selected from the group consisting of alkyl, alkyl-OH,

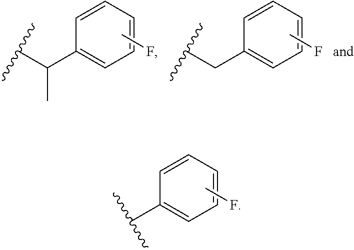

Another embodiment of this invention is directed to compounds of formula (I) wherein: (a) R² and R⁶ are joined together to form a 5-8 membered heterocyclenyl moiety substituted with 1 to 5 independently selected R²¹ groups, (b) W is —C(O)—, (c) U is a bond or —C(R³)(R⁴)—, (d) X is or —C(R⁶)(R⁷)—, (e) R⁸ is H or alkyl, (f) R¹⁰ is

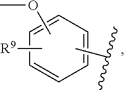

(g) R⁹ is 4-methyl-imidazol-1-yl, and (h) R²¹ is independently selected from the group consisting of alkyl, alkyl-OH,

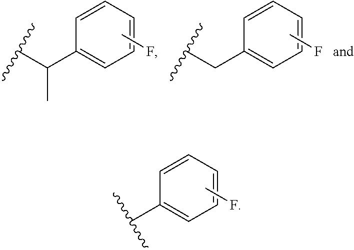

Another embodiment of this invention is directed to compounds of formula (I) wherein: (a) R² and R⁶ are joined together to form a 5-8 membered heterocyclenyl moiety optionally substituted with 1 to 5 independently selected R²¹ groups, and said heteroaryl moiety is optionally fused with an aryl or heteroaryl ring, and the ring moiety resulting from the fusion is optionally substituted with 1 to 5 independently selected R²¹ groups, (b) W is —C(O)—, (c) U is a bond or —C(R³)(R⁴)—, (d) X is —C(R⁶)(R⁷)—, (e) R⁸ is H or alkyl, (f) R¹⁰ is

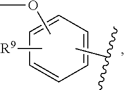

(g) R⁹ is 4-methyl-imidazol-1-yl, and (h) R²¹ is independently selected from the group consisting of alkyl, alkyl-OH,

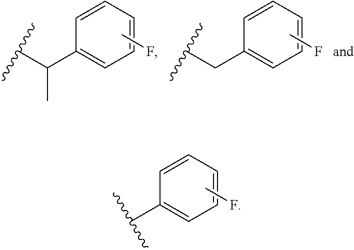

Another embodiment of this invention is directed to compounds of formula (I) wherein: (a) R² and R⁶ are joined together to form a 5-8 membered heterocyclenyl moiety optionally substituted with 1 to 5 independently selected R²¹ groups, and said heteroaryl moiety is optionally fused with an aryl ring, and the ring moiety resulting from the fusion is optionally substituted with 1 to 5 independently selected R²¹ groups, (b) W is —C(O)—, (c) U is a bond or —C(R³)(R⁴)—, (d) X is —C(R⁶)(R⁷)—, (e) R⁸ is H or alkyl, (f) R¹⁰ is

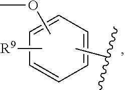

(g) R⁹ is 4-methyl-imidazol-1-yl, and (h) R²¹ is independently selected from the group consisting of alkyl, alkyl-OH,

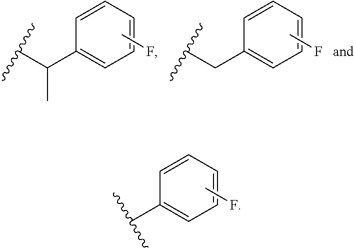

Another embodiment of this invention is directed to compounds of formula (I) wherein: (a) R² and R⁶ are joined together to form a 5-8 membered heterocyclenyl moiety optionally substituted with 1 to 5 independently selected R²¹ groups, and said heteroaryl moiety is optionally fused with a heteroaryl ring, and the ring moiety resulting from the fusion is optionally substituted with 1 to 5 independently selected R²¹ groups, (b) W is —C(O)—, (c) U is a bond or —C(R³)(R⁴)—, (d) X is —C(R⁶)(R⁷)—, (e) R⁸ is H or alkyl, (f) R¹⁰ is

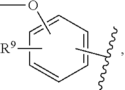

(g) R⁹ is 4-methyl-imidazol-1-yl, and (h) R²¹ is independently selected from the group consisting of alkyl, alkyl-OH,

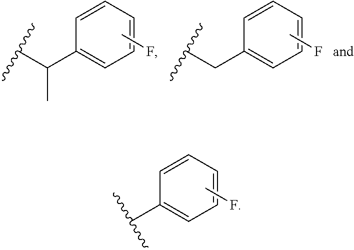

Another embodiment of this invention is directed to compounds of formula (I) wherein: (a) R² and R¹⁴ are joined together to form a 5-14 membered heteroaryl moiety, (b) W is —C(O)—, (c) U is a bond or —C(R³)(R⁴)—, (d) X is —N(R¹⁴)—, (e) R⁸ is H or alkyl, (f) R¹⁰ is

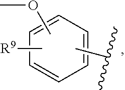

(g) R⁹ is 4-methyl-imidazol-1-yl, and (h) R²¹ is independently selected from the group consisting of alkyl, alkyl-OH,

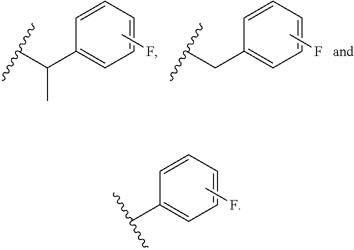

Another embodiment of this invention is directed to compounds of formula (I) wherein: (a) R² and R¹⁴ are joined together to form a 5-14 membered heteroaryl moiety optionally substituted with 1 to 5 independently selected R²¹ groups, (b) W is —C(O)—, (c) U is a bond or —C(R³)(R⁴)—, (d) X is —N(R¹⁴)—, (e) R⁸ is H or alkyl, (f) R¹⁰ is

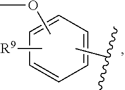

(g) R⁹ is 4-methyl-imidazol-1-yl, and (h) R²¹ is independently selected from the group consisting of alkyl, alkyl-OH,

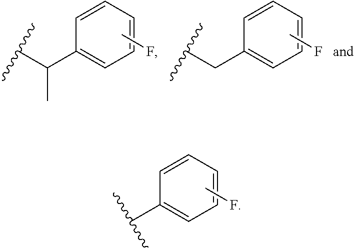

Another embodiment of this invention is directed to compounds of formula (I) wherein: (a) R² and R¹⁴ are joined together to form a 5-14 membered heteroaryl moiety substituted with 1 to 5 independently selected R²¹ groups, (b) W is —C(O)—, (c) U is a bond or —C(R³)(R⁴)—, (d) X is —N(R¹⁴)—, (e) R⁸ is H or alkyl, (f) R¹⁰ is

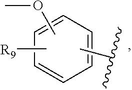

(g) R⁹ is 4-methyl-imidazol-1-yl, and (h) R²¹ is independently selected from the group consisting of alkyl, alkyl-OH,

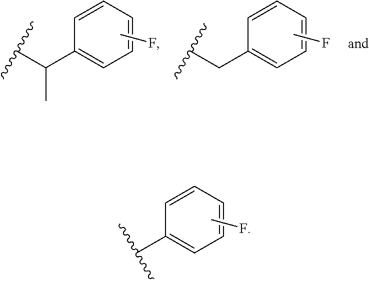

Another embodiment of this invention is directed to compounds of formula (I) wherein: (a) R² and R¹⁴ are joined together to form a 5-14 membered heteroaryl moiety optionally substituted with 1 to 5 independently selected R²¹ groups, and said heteroaryl moiety is optionally fused with an aryl or heteroaryl ring, and the ring moiety resulting from the fusion is optionally substituted with 1 to 5 independently selected R²¹ groups, (b) W is —C(O)—, (c) U is a bond or —C(R³)(R⁴)—, (d) X is —N(R¹⁴)—, (e) R⁸ is H or alkyl, (f) R¹⁰ is

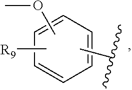

(g) R⁹ is 4-methyl-imidazol-1-yl, and (h) R²¹ is independently selected from the group consisting of alkyl, alkyl-OH,

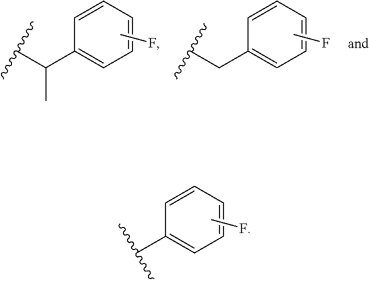

Another embodiment of this invention is directed to compounds of formula (I) wherein: (a) R² and R¹⁴ are joined together to form a 5-14 membered heteroaryl moiety optionally substituted with 1 to 5 independently selected R²¹ groups, and said heteroaryl moiety is optionally fused with an aryl ring; and the ring moiety resulting from the fusion is optionally substituted with 1 to 5 independently selected R²¹ groups, (b) W is —C(O)—, (c) U is a bond or —C(R³)(R⁴)—, (d) X is —N(R¹⁴)—, (e) R⁸ is H or alkyl, (f) R¹⁰ is

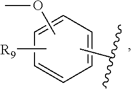

(g) R⁹ is 4-methyl-imidazol-1-yl, and (h) R²¹ is independently selected from the group consisting of alkyl, alkyl-OH,

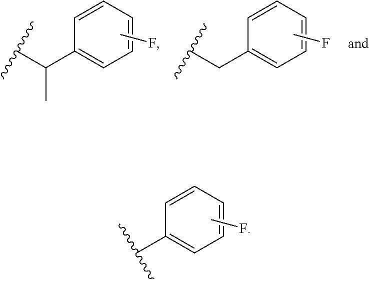

Another embodiment of this invention is directed to compounds of formula (I) wherein: (a) R² and R¹⁴ are joined together to form a 5-14 membered heteroaryl moiety optionally substituted with 1 to 5 independently selected R²¹ groups, and said heteroaryl moiety is optionally fused with a heteroaryl ring, and the ring moiety resulting from the fusion is optionally substituted with 1 to 5 independently selected R²¹ groups, (b) W is —C(O)—, (c) U is a bond or —C(R³)(R⁴)—, (d) X is —N(R¹⁴)—, (e) R⁸ is H or alkyl, (f) R¹⁰ is

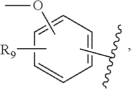

(g) R⁹ is 4-methyl-imidazol-1-yl, and (h) R²¹ is independently selected from the group consisting of alkyl, alkyl-OH,

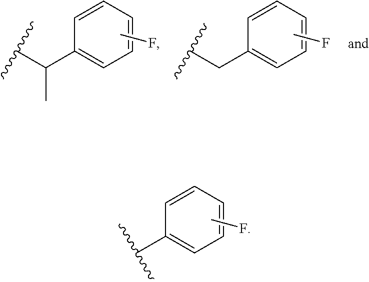

Another embodiment of this invention is directed to compounds of formula (I) wherein: (a) R² and R¹⁴ are joined together to form a 5-8 membered heterocyclyl moiety, (b) W is —C(O)—, (c) U is a bond or —C(R³)(R⁴)—, (d) X is —N(R¹⁴)—, (e) R⁸ is H or alkyl, (f) R¹⁰ is

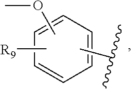

(g) R⁹ is 4-methyl-imidazol-1-yl, and (h) R²¹ is independently selected from the group consisting of alkyl, alkyl-OH,

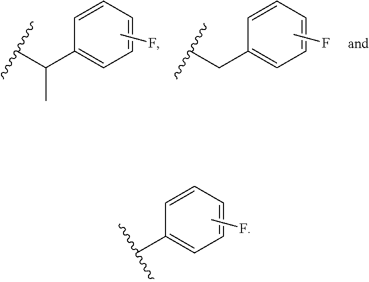

Another embodiment of this invention is directed to compounds of formula (I) wherein: (a) R² and R¹⁴ are joined together to form a 5-8 membered heterocyclyl moiety optionally substituted with 1 to 5 independently selected R²¹ groups, (b) W is —C(O)—, (c) U is a bond or —C(R³)(R⁴)—, (d) X is —N(R¹⁴)—, (e) R⁸ is H or alkyl, (f) R¹⁰ is

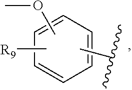

(g) R⁹ is 4-methyl-imidazol-1-yl, and (h) R²¹ is independently selected from the group consisting of alkyl, alkyl-OH,

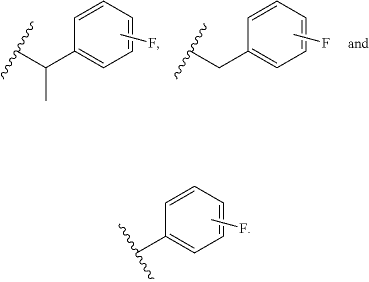

Another embodiment of this invention is directed to compounds of formula (I) wherein: (a) R² and R¹⁴ are joined together to form a 5-8 membered heterocyclyl moiety substituted with 1 to 5 independently selected R²¹ groups, (b) W is —C(O)—, (c) U is a bond or —C(R³)(R⁴)—, (d) X is —N(R¹⁴)—, (e) R⁸ is H or alkyl, (f) R¹⁰ is

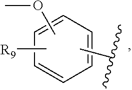

(g) R⁹ is 4-methyl-imidazol-1-yl, and (h) R²¹ is independently selected from the group consisting of alkyl, alkyl-OH,

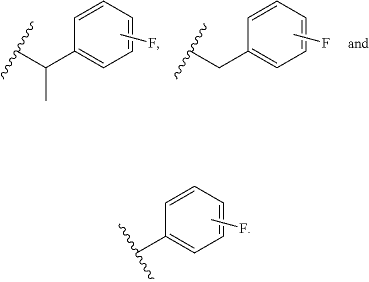

Another embodiment of this invention is directed to compounds of formula (I) wherein: (a) R² and R¹⁴ are joined together to form a 5-8 membered heterocyclyl moiety optionally substituted with 1 to 5 independently selected R²¹ groups, and said heteroaryl moiety is optionally fused with an aryl or heteroaryl ring, and the ring moiety resulting from the fusion is optionally substituted with 1 to 5 independently selected R²¹ groups, (b) W is —C(O)—, (c) U is a bond or —C(R³)(R⁴)—, (d) X is —N(R¹⁴)—, (e) R⁸ is H or alkyl, (f) R¹⁰ is

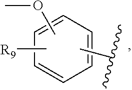

(g) R⁹ is 4-methyl-imidazol-1-yl, and (h) R²¹ is independently selected from the group consisting of alkyl, alkyl-OH,

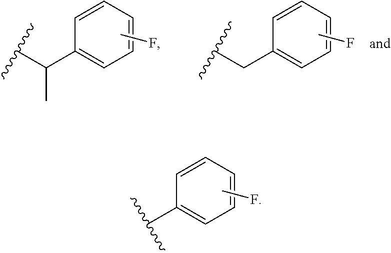

Another embodiment of this invention is directed to compounds of formula (I) wherein: (a) R² and R¹⁴ are joined together to form a 5-8 membered heterocyclyl moiety optionally substituted with 1 to 5 independently selected R²¹ groups, and said heteroaryl moiety is optionally fused with an aryl ring, and the ring moiety resulting from the fusion is optionally substituted with 1 to 5 independently selected R²¹ groups, (b) W is —C(O)—, (c) U is a bond or —C(R³)(R⁴)—, (d) X is —N(R¹⁴)—, (e) R⁸ is H or alkyl, (f) R¹⁰ is

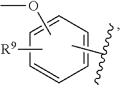

(g) R⁹ is 4-methyl-imidazol-1-yl, and (h) R²¹ is independently selected from the group consisting of alkyl, alkyl-OH,

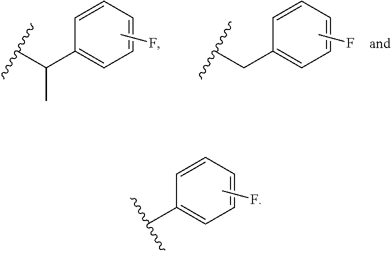

Another embodiment of this invention is directed to compounds of formula (I) wherein: (a) R² and R¹⁴ are joined together to form a 5-8 membered heterocyclyl moiety optionally substituted with 1 to 5 independently selected R²¹ groups, and said heteroaryl moiety is optionally fused with a heteroaryl ring, and the ring moiety resulting from the fusion is optionally substituted with 1 to 5 independently selected R²¹ groups, (b) W is —C(O)—, (c) U is a bond or —C(R³)(R⁴)—, (d) X is —N(R¹⁴)—, (e) R⁸ is H or alkyl, (f) R¹⁰ is

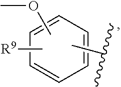

(g) R⁹ is 4-methyl-imidazol-1-yl, and (h) R²¹ is independently selected from the group consisting of alkyl, alkyl-OH,

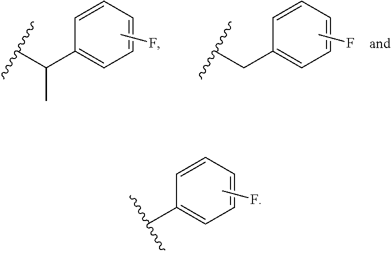

Another embodiment of this invention is directed to compounds of formula (I) wherein: (a) R² and R¹⁴ are joined together to form a 5-8 membered heterocyclenyl moiety, (b) W is —C(O)—, (c) U is a bond or —C(R³)(R⁴)—, (d) X is (e) R⁸ is H or alkyl, (f) R¹⁰ is

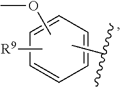

(g) R⁹ is 4-methyl-imidazol-1-yl, and (h) R²¹ is independently selected from the group consisting of alkyl, alkyl-OH,

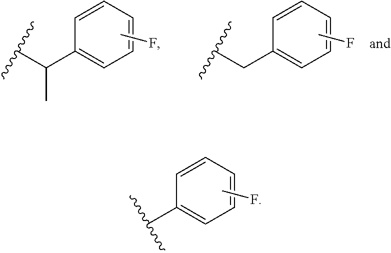

Another embodiment of this invention is directed to compounds of formula (I) wherein: (a) R² and R¹⁴ are joined together to form a 5-8 membered heterocyclenyl moiety optionally substituted with 1 to 5 independently selected R²¹ groups, (b) W is —C(O)—, (c) U is a bond or —C(R³)(R⁴)—, (d) X is —N(R¹⁴)—, (e) R⁸ is H or alkyl, (f) R¹⁰ is

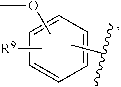

(g) R⁹ is 4-methyl-imidazol-1-yl, and (h) R²¹ is independently selected from the group consisting of alkyl, alkyl-OH,

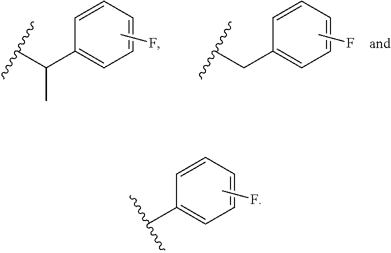

Another embodiment of this invention is directed to compounds of formula (I) wherein: (a) R² and R¹⁴ are joined together to form a 5-8 membered heterocyclenyl moiety substituted with 1 to 5 independently selected R²¹ groups, (b) W is —C(O)—, (c) U is a bond or —C(R³)(R⁴)—, (d) X is —N(R¹⁴)—, (e) R⁸ is H or alkyl, (f) R¹⁰ is

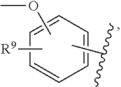

(g) R⁹ is 4-methyl-imidazol-1-yl, and (h) R²¹ is independently selected from the group consisting of alkyl, alkyl-OH,

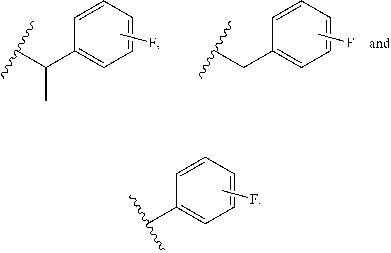

Another embodiment of this invention is directed to compounds of formula (I) wherein: (a) R² and R¹⁴ are joined together to form a 5-8 membered heterocyclenyl moiety optionally substituted with 1 to 5 independently selected R²¹ groups, and said heteroaryl moiety is optionally fused with an aryl or heteroaryl ring, and the ring moiety resulting from the fusion is optionally substituted with 1 to 5 independently selected R²¹ groups, (b) W is —C(O)—, (c) U is a bond or —C(R³)(R⁴)—, (d) X is —N(R¹⁴)—, (e) R⁸ is H or alkyl, (f) R¹⁰ is

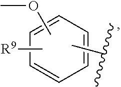

(g) R⁹ is 4-methyl-imidazol-1-yl, and (h) R²¹ is independently selected from the group consisting of alkyl, alkyl-OH,

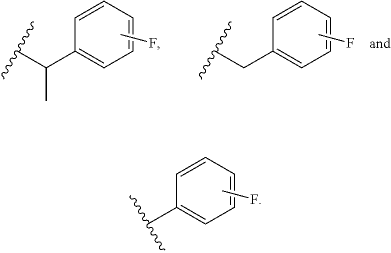

Another embodiment of this invention is directed to compounds of formula (I) wherein: (a) R² and R¹⁴ are joined together to form a 5-8 membered heterocyclenyl moiety optionally substituted with 1 to 5 independently selected R²¹ groups, and said heteroaryl moiety is optionally fused with an aryl ring, and the ring moiety resulting from the fusion is optionally substituted with 1 to 5 independently selected R²¹ groups, (b) W is —C(O)—, (c) U is a bond or —C(R³)(R⁴)—, (d) X is —N(R¹⁴)—, (e) R⁸ is H or alkyl, (f) R¹⁰ is

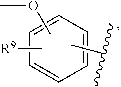

(g) R⁹ is 4-methyl-imidazol-1-yl, and (h) R²¹ is independently selected from the group consisting of alkyl, alkyl-OH,

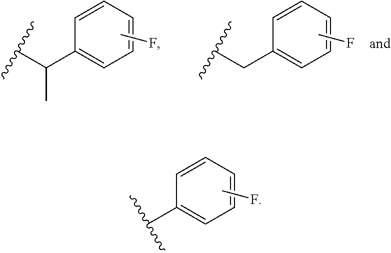

Another embodiment of this invention is directed to compounds of formula (I) wherein: (a) R² and R¹⁴ are joined together to form a 5-8 membered heterocyclenyl moiety optionally substituted with 1 to 5 independently selected R²¹ groups, and said heteroaryl moiety is optionally fused with a heteroaryl ring, and the ring moiety resulting from the fusion is optionally substituted with 1 to 5 independently selected R²¹ groups, (b) W is —C(O)—, (c) U is a bond or —C(R³)(R⁴)—, (d) X is —N(R¹⁴)—, (e) R⁸ is H or alkyl, (f) R¹⁰ is

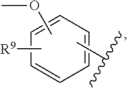

(g) R⁹ is 4-methyl-imidazol-1-yl, and (h) R²¹ is independently selected from the group consisting of alkyl, alkyl-OH,

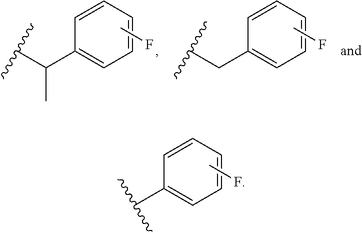

In another embodiment of this invention R¹ is

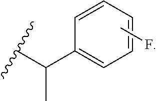

In another embodiment of this invention R¹ is

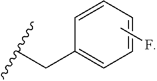

In another embodiment of this invention R¹ is

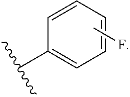

In another embodiment of this invention R¹ is

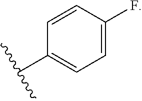

In another embodiment of this invention R¹ is H.

In another embodiment of this invention R¹ is alkyl.

In another embodiment, the present application discloses a compound, or pharmaceutically acceptable salts, solvates, esters or prodrugs of said compound, said compound having the general structures shown in the following formula:

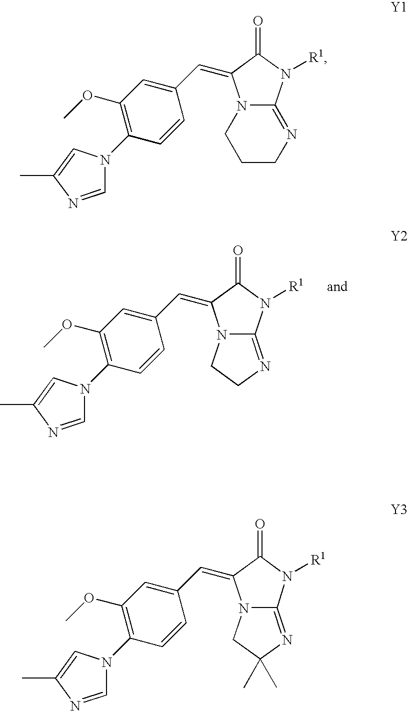

wherein R¹ is independently selected from the group consisting of H, alkyl,

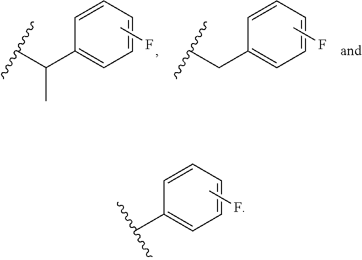

In another embodiment, R¹, for Y1, Y2 or Y3, is independently selected from the group consisting of

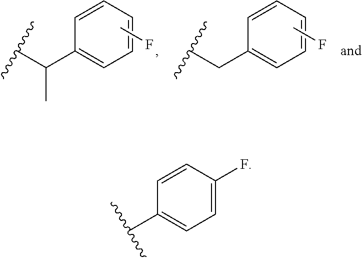

Another embodiment of this invention is directed to Y1 wherein R¹ is

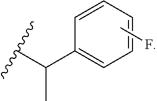

Another embodiment of this invention is directed to Y1 wherein R¹ is

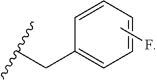

Another embodiment of this invention is directed to Y1 wherein R¹ is

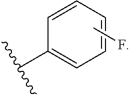

Another embodiment of this invention is directed to Y1 wherein R¹ is

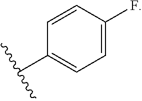

Another embodiment of this invention is directed to Y2 wherein R¹ is

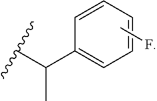

Another embodiment of this invention is directed to Y2 wherein R¹ is

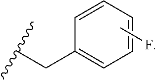

Another embodiment of this invention is directed to Y2 wherein R¹ is

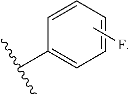

Another embodiment of this invention is directed to Y2 wherein R¹ is

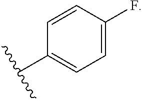

Another embodiment of this invention is directed to Y3 wherein R¹ is

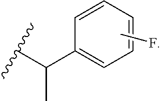

Another embodiment of this invention is directed to Y3 wherein R¹ is

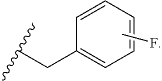

Another embodiment of this invention is directed to Y3 wherein R¹ is

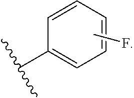

Another embodiment of this invention is directed to Y3 wherein R¹ is

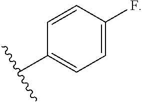

An illustrative group of compounds of the invention is shown in Table 1. This invention is also directed to a pharmaceutically acceptable salt, solvate, ester or prodrug of the compounds in Table 1.

TABLE 1

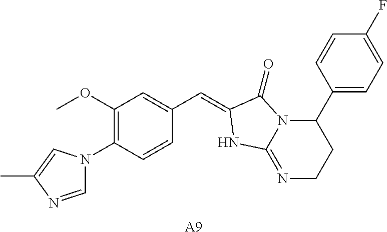

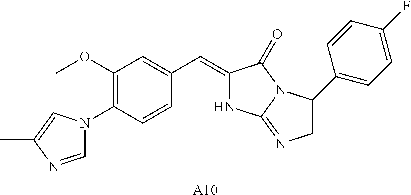

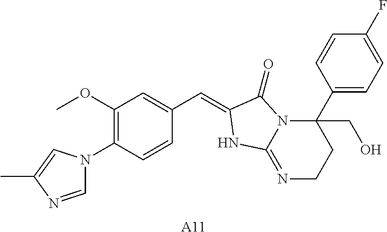

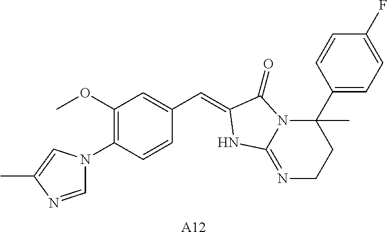

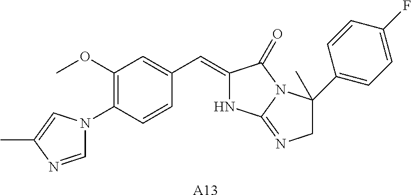

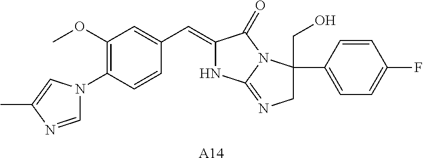

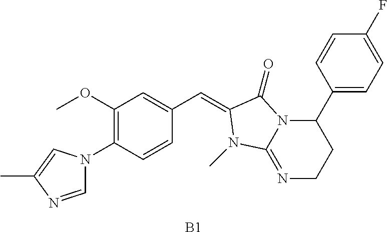

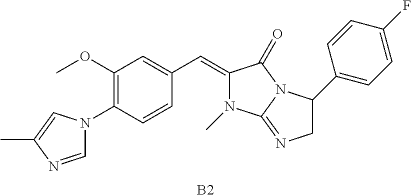

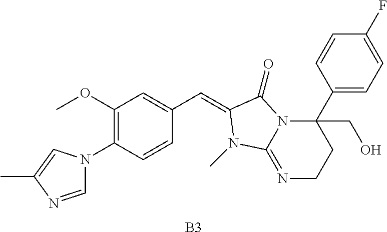

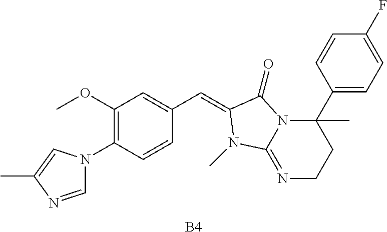

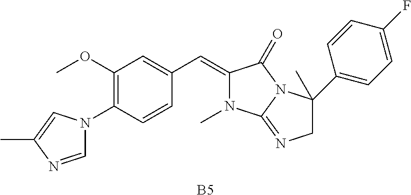

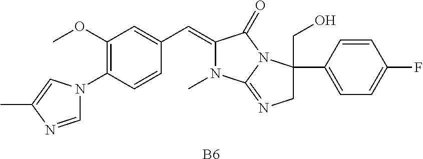

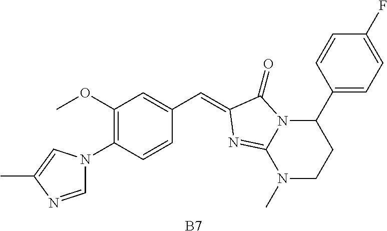

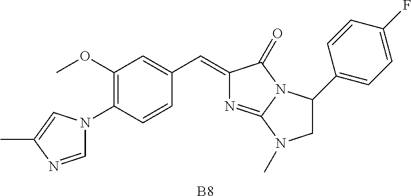

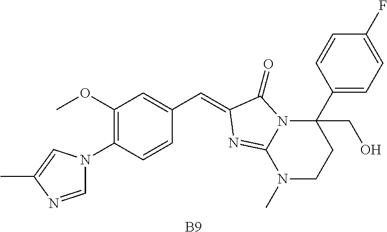

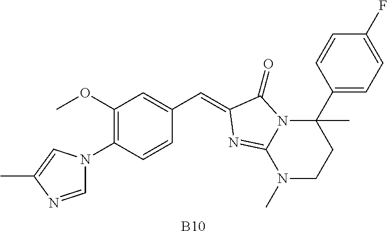

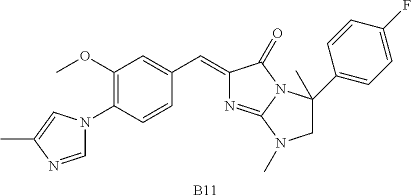

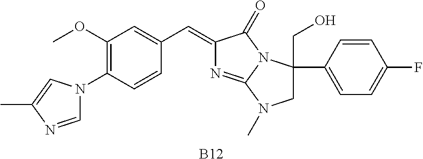

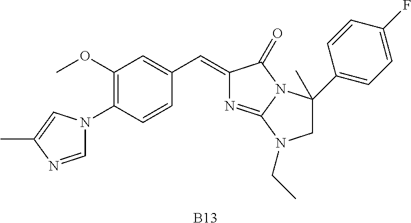

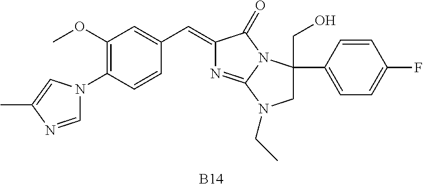

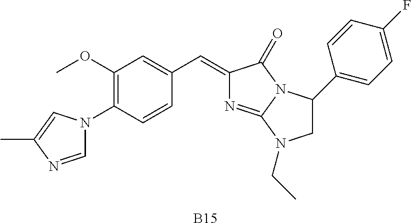

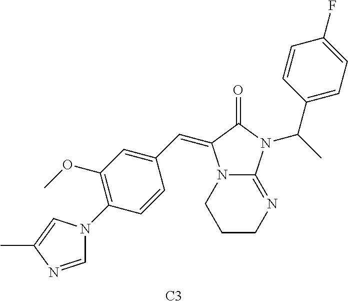

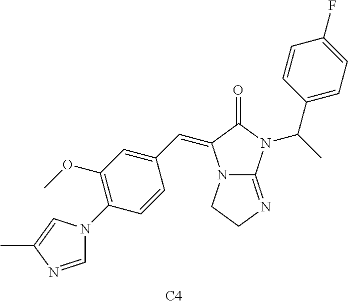

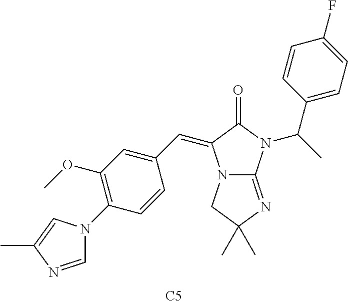

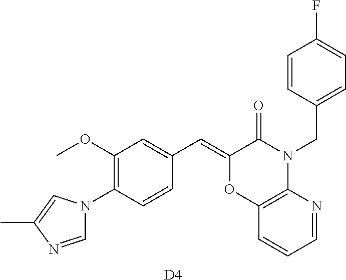

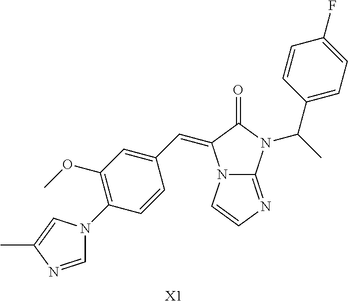

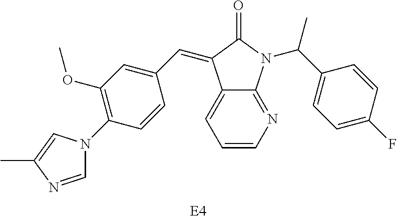

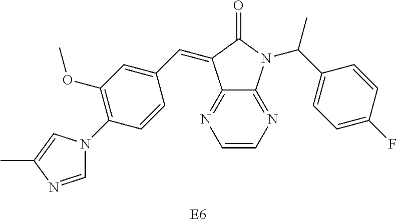

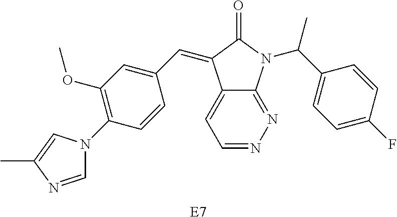

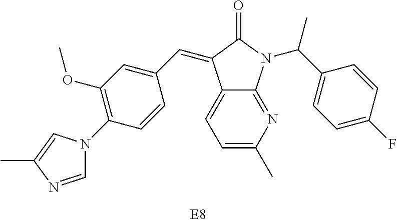

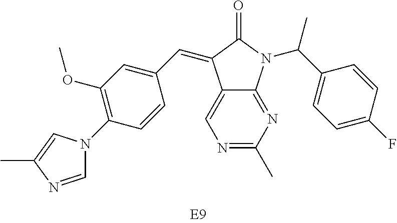

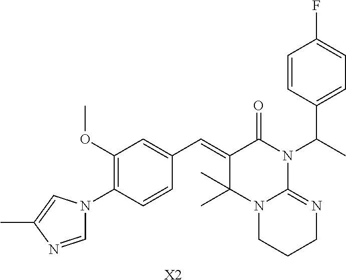

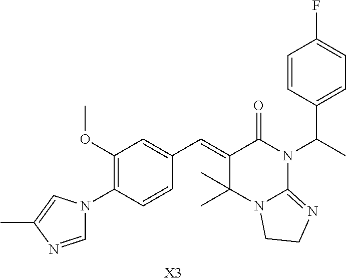

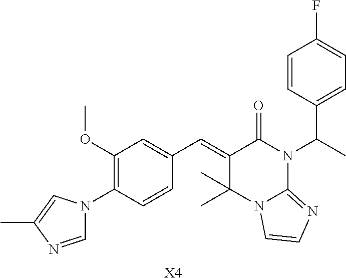

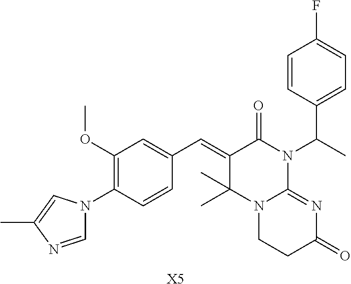

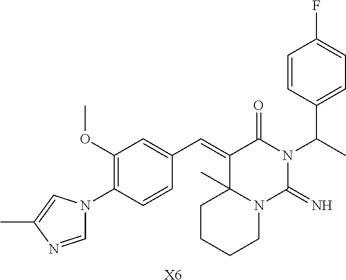

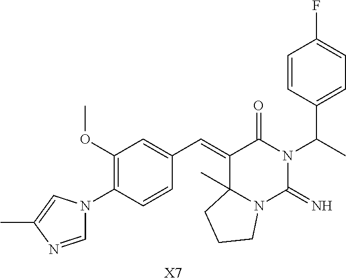

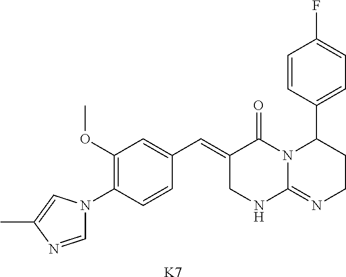

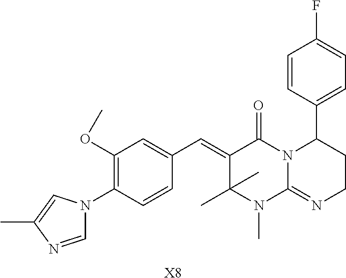

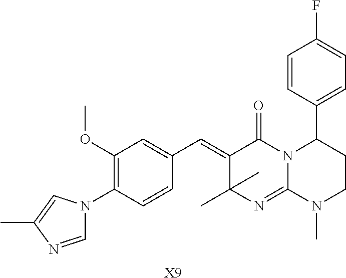

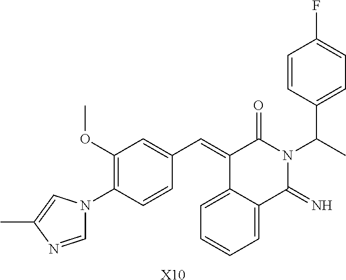

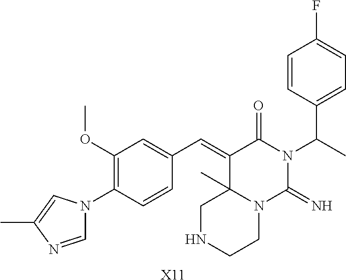

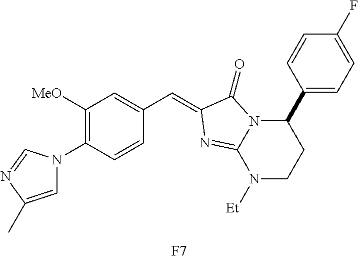

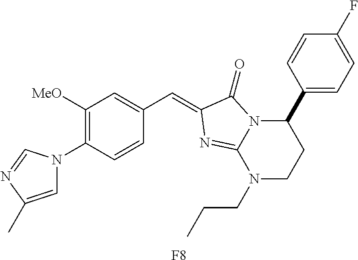

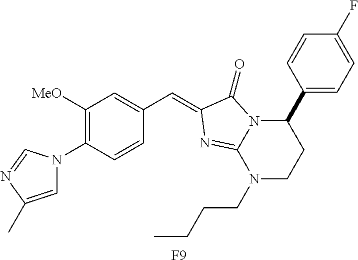

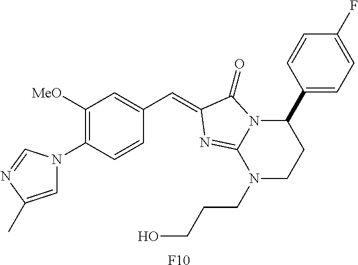

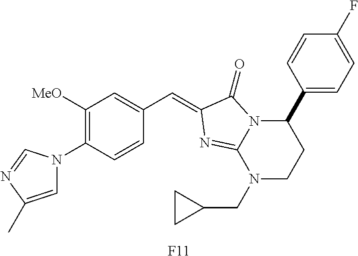

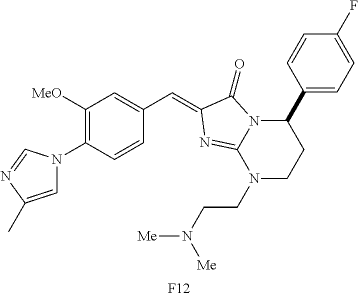

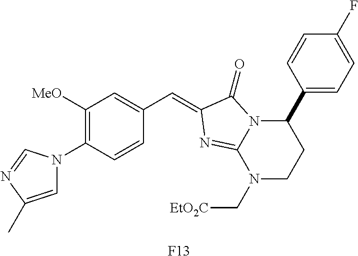

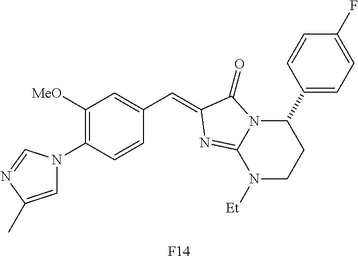

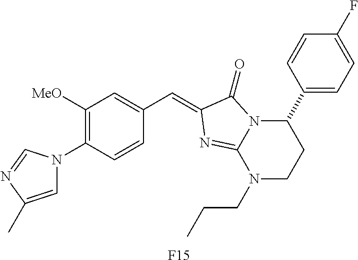

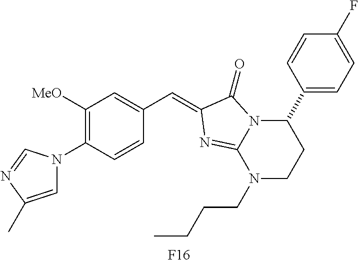

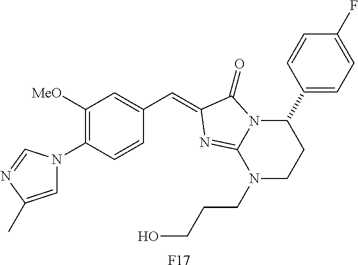

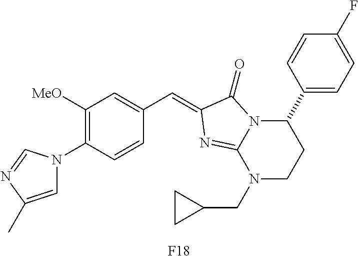

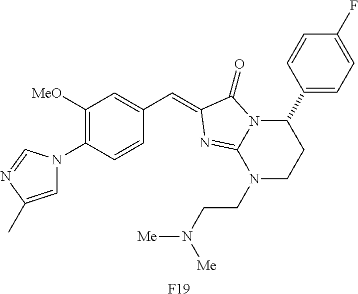

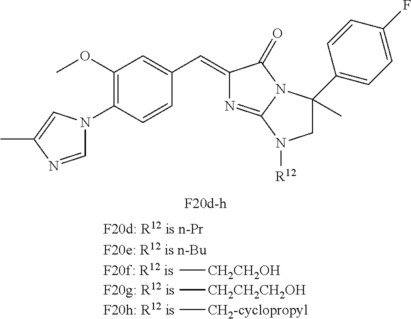

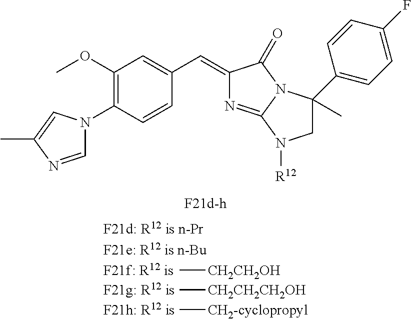

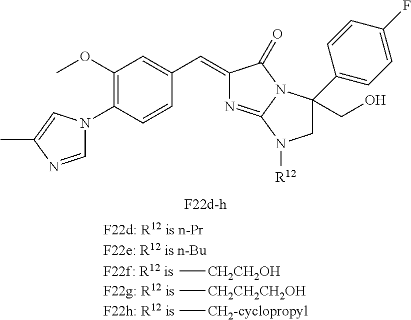

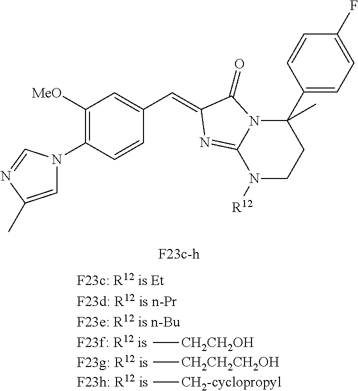

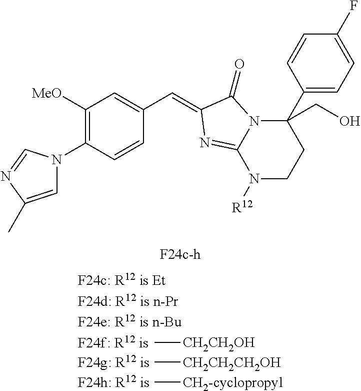

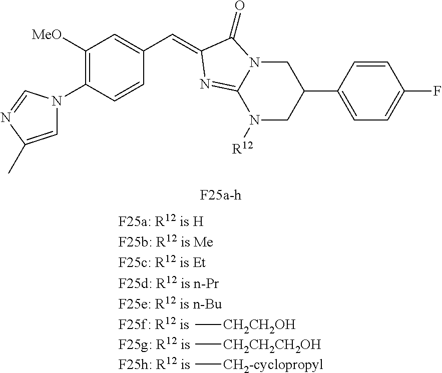

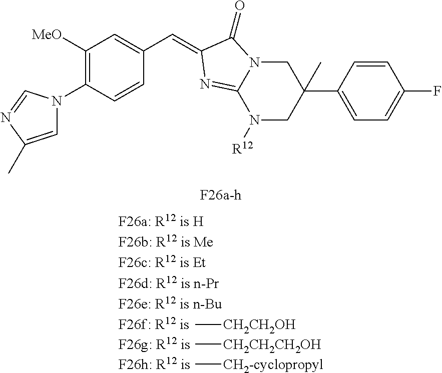

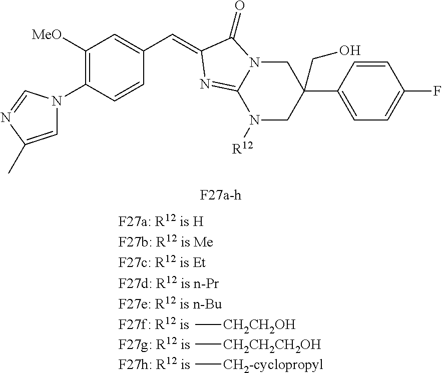

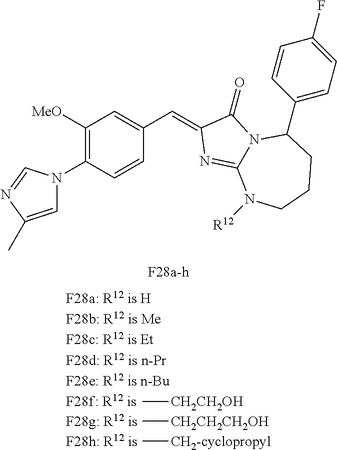

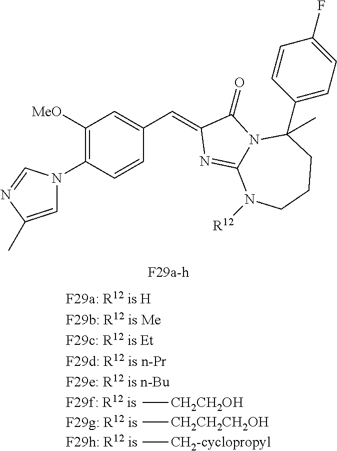

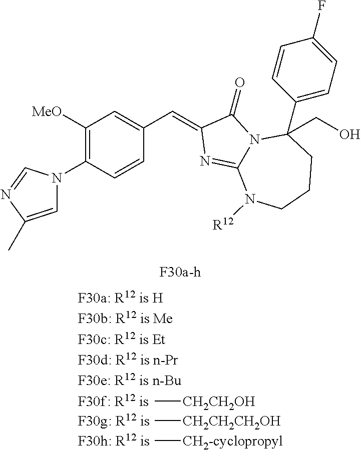

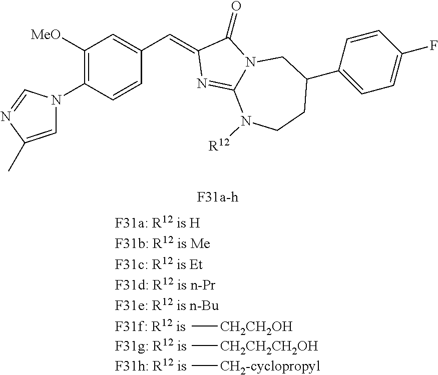

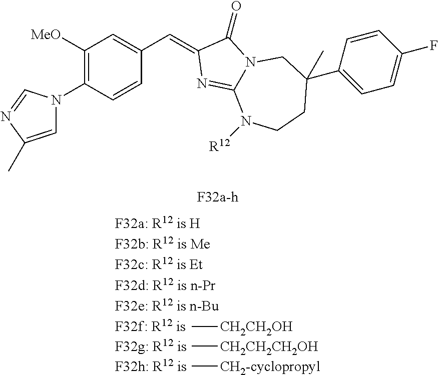

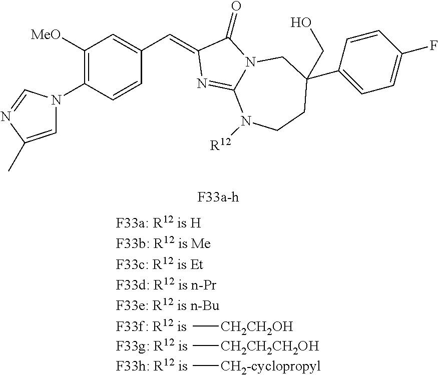

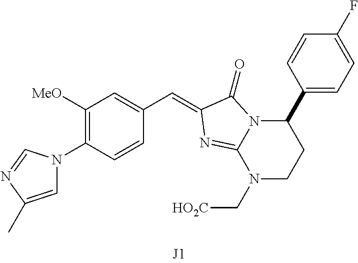

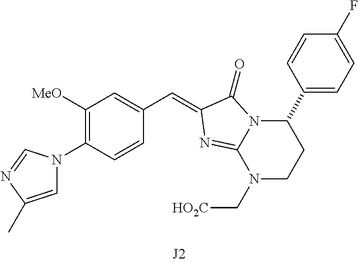

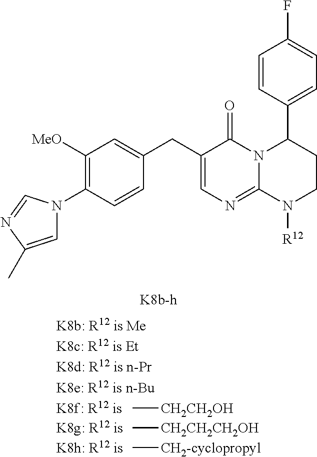

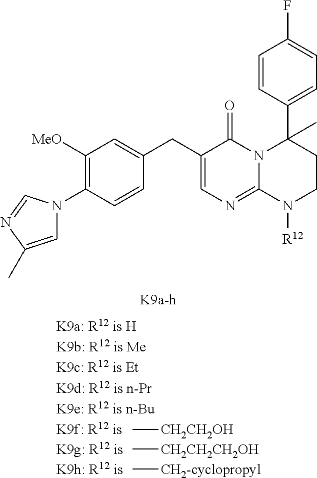

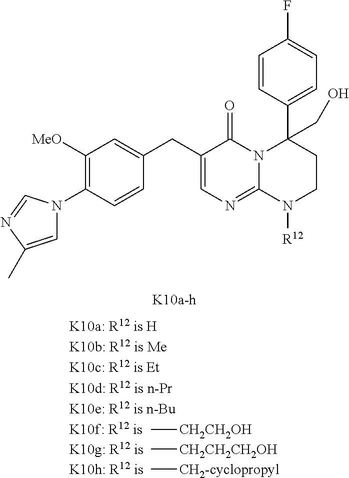

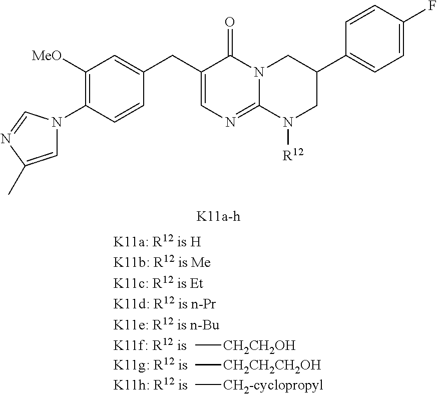

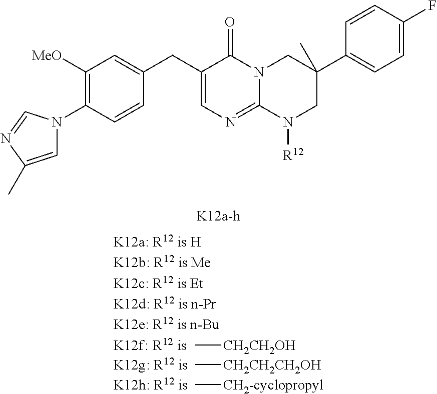

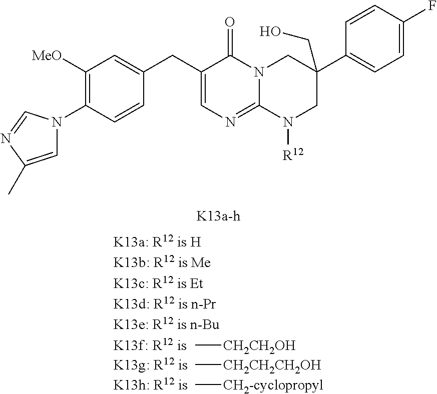

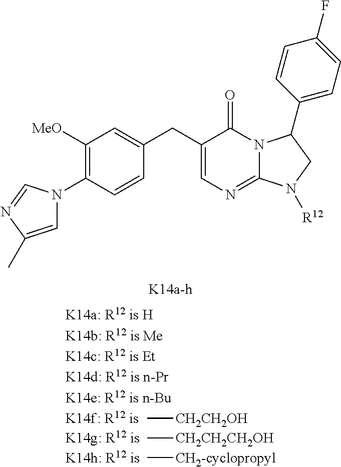

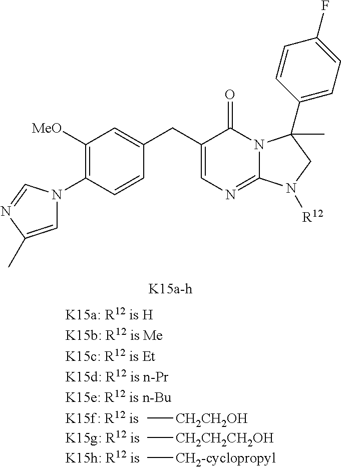

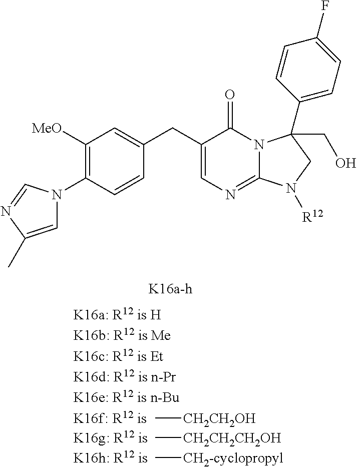

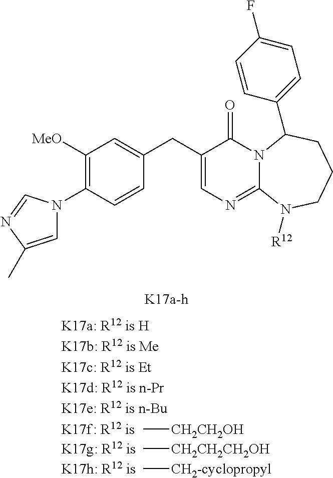

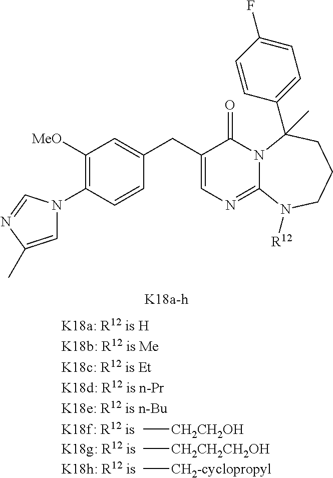

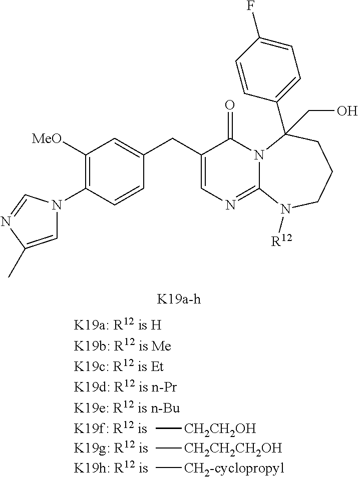

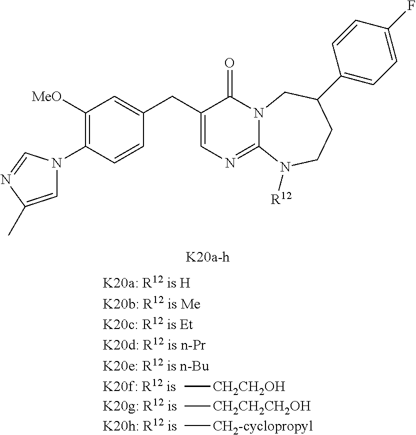

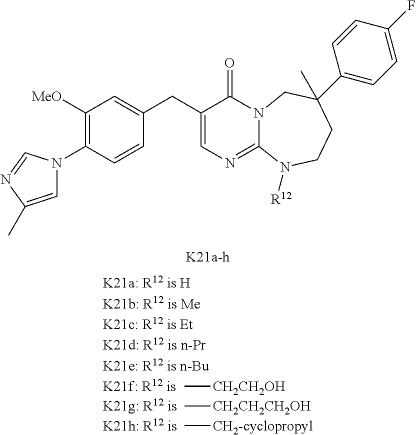

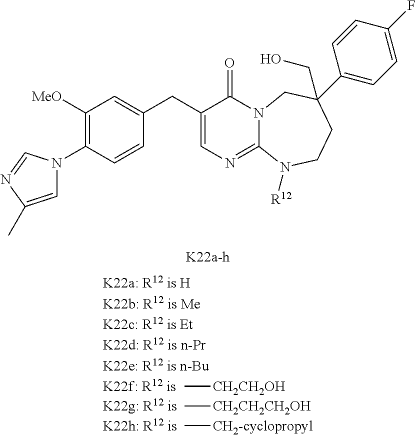

Another embodiment of this invention is directed to compounds of formula (I).

Another embodiment of this invention is directed to pharmaceutically acceptable salts of the compounds of formula (I).

Another embodiment of this invention is directed to pharmaceutically acceptable esters of the compounds of formula (I).

Another embodiment of this invention is directed to solvates of the compounds of formula (I).

Another embodiment of this invention is directed to compounds of formula (I) in pure form.

Another embodiment of this invention is directed to compounds of formula (I) in isolated form.

Another embodiment of this invention is directed to compounds of formula (I) in pure and isolated form.

Another embodiment of this invention is directed to a compounds of formula (I) selected from the group consisting of compounds of the formula: Y1, Y2, Y3, A9-A14, B1-B15, C3-C5, D4, E4, E6-E9, F7-F19, F20d-F20h, F21d-F21h, F22d-F22h, F23c-F23h, F24c-F24h, F25a-F25h, F26a-F26h, F27a-F27h, F28a-F28h, F29a-F29h, F30a-F30h, F31a-F31h, F32a-F32h, F33a-F33h, J1, J2, K7, K8b-K8h, K9a-K9h, K10a-K10h, K11a-K11h, K12a-K12h, K13a-K13h, K14a-K14h, K15a-K15h, K16a-K16h, K17a-K17h, K18a-K18h, K19a-K19h, K20a-K20h, K21a-K21h, K22a-K22h, and X1-X11.

Another embodiment of this invention is directed to a pharmaceutically acceptable salt of a compound of formula (I), wherein said compound of formula (I) is selected from the group consisting of the compounds of the formula: Y1, Y2, Y3, A9-A14, B1-B15, C3-C5, D4, E4, E6-E9, F7-F19, F20d-F20h, F21d-F21h, F22d-F22h, F23c-F23h, F24c-F24h, F25a-F25h, F26a-F26h, F27a-F27h, F28a-F28h, F29a-F29h, F30a-F30h, F31a-F31h, F32a-F32h, F33a-F33h, J1, J2, K7, K8b-K8h, K9a-K9h, K10a-K10h, K11a-K11h, K12a-K12h, K13a-K13h, K14a-K14h, K15a-K15h, K16a-K16h, K17a-K17h, K18a-K18h, K19a-K19h, K20a-K20h, K21a-K21h, K22a-K22h, and X1-X11.

Another embodiment of this invention is directed to a pharmaceutically acceptable ester of a compound of formula (I), wherein said compound of formula (I) is selected from the group consisting of the compounds of the formula: Y1, Y2, Y3, A9-A14, B1-B15, C3-C5, D4, E4, E6-E9, F7-F19, F20d-F20h, F21d-F21h, F22d-F22h, F23c-F23h, F24c-F24h, F25a-F25h, F26a-F26h, F27a-F27h, F28a-F28h, F29a-F29h, F30a-F30h, F31a-F31h, F32a-F32h, F33a-F33h, J1, J2, K7, K8b-K8h, K9a-K9h, K10a-K10h, K11a-K11h, K12a-K12h, K13a-K13h, K14a-K14h, K15a-K15h, K16a-K16h, K17a-K17h, K18a-K18h, K19a-K19h, K20a-K20h, K21a-K21h, K22a-K22h, and X1-X11.

Another embodiment of this invention is directed to a solvate of a compound of formula (I), wherein said compound of formula (I) is selected from the group consisting of the compounds of the formula: Y1, Y2, Y3, A9-A14, B1-B15, C3-C5, D4, E4, E6-E9, F7-F19, F20d-F20h, F21d-F21h, F22d-F22h, F23c-F23h, F24c-F24h, F25a-F25h, F26a-F26h, F27a-F27h, F28a-F28h, F29a-F29h, F30a-F30h, F31a-F31h, F32a-F32h, F33a-F33h, J1, J2, K7, K8b-F8h, K9a-K9h, K10a-K10h, K11a-K11h, K12a-K12h, K13a-K13h, K14a-K14h, K15a-K15h, K16a-K16h, K17a-K17h, K18a-K18h, K19a-K19h, K20a-K20h, K21a-K21h, K22a-K22h, and X1-X11.

Another embodiment of this invention is directed to a compounds of formula (I) in isolated form, wherein the compounds of formula (I) are selected from the group consisting of compounds of the formula: Y1, Y2, Y3, A9-A14, B1-B15, C3-C5, D4, E4, E6-E9, F7-F19, F20d-F20h, F21d-F21h, F22d-F22h, F23c-F23h, F24c-F24h, F25a-F25h, F26a-F26h, F27a-F27h, F28a-F28h, F29a-F29h, F30a-F30h, F31a-F31h, F32a-F32h, F33a-F33h, J1, J2, K7, K8b-K8h, K9a-K9h, K10a-K10h, K11a-K11h, K12a-K12h, K13a-K13h, K14a-K14h, K15a-K15h, K16a-K16h, K17a-K17h, K18a-K18h, K19a-K19h, K20a-K20h, K21a-K21h, K22a-K22h, and X1-X11.

Another embodiment of this invention is directed to a compounds of formula (I) in pure form, wherein the compounds of formula (I) are selected from the group consisting of compounds of the formula: Y1, Y2, Y3, A9-A14, B1-B15, C3-C5, D4, E4, E6-E9, F7-F19, F20d-F20h, F21d-F21h, F22d-F22h, F23c-F23h, F24c-F24h, F25a-F25h, F26a-F26h, F27a-F27h, F28a-F28h, F29a-F29h, F30a-F30h, F31a-F31h, F32a-F32h, F33a-F33h, J1, J2, K7, K8b-K8h, K9a-K9h, K10a-K10h, K11a-K11h, K12a-K12h, K13a-K13h, K14a-K14h, K15a-K15h, K16a-K16h, K17a-K17h, K18a-K18h, K19a-K19h, K20a-K20h, K21a-K21h, K22a-K22h, and X1-X11.

Another embodiment of this invention is directed to the compound of formula Y1.

Another embodiment of this invention is directed to the compound of formula Y2.

Another embodiment of this invention is directed to the compound of formula Y3.

Another embodiment of this invention is directed to the compound of formula A9 (e.g., (R)-A9 and (S)-A9).

Another embodiment of this invention is directed to the compound of formula (R)-A9.

Another embodiment of this invention is directed to the compound of formula (S)-A9.

Another embodiment of this invention is directed to the compound of formula A10.

Another embodiment of this invention is directed to the compound of formula A11.

Another embodiment of this invention is directed to the compound of formula A12.

Another embodiment of this invention is directed to the compound of formula A13.

Another embodiment of this invention is directed to the compound of formula A14.

Another embodiment of this invention is directed to the compound of formula B1.

Another embodiment of this invention is directed to the compound of formula B2.

Another embodiment of this invention is directed to the compound of formula B3.

Another embodiment of this invention is directed to the compound of formula B4.

Another embodiment of this invention is directed to the compound of formula B5.

Another embodiment of this invention is directed to the compound of formula B6.

Another embodiment of this invention is directed to the compound of formula B7 (e.g., (R)-B7 and (S)-B7).

Another embodiment of this invention is directed to the compound of formula (R)-B7.

Another embodiment of this invention is directed to the compound of formula (S)-B7.

Another embodiment of this invention is directed to the compound of formula B8.

Another embodiment of this invention is directed to the compound of formula B9.

Another embodiment of this invention is directed to the compound of formula B10.

Another embodiment of this invention is directed to the compound of formula B11.

Another embodiment of this invention is directed to the compound of formula B12.

Another embodiment of this invention is directed to the compound of formula B13.

Another embodiment of this invention is directed to the compound of formula B14.

Another embodiment of this invention is directed to the compound of formula B15.

Another embodiment of this invention is directed to the compound of formula C3.

Another embodiment of this invention is directed to the compound of formula C4.

Another embodiment of this invention is directed to the compound of formula C5.

Another embodiment of this invention is directed to the compound of formula D4.

Another embodiment of this invention is directed to the compound of formula E4.

Another embodiment of this invention is directed to the compound of formula E6.

Another embodiment of this invention is directed to the compound of formula E7.

Another embodiment of this invention is directed to the compound of formula E8.

Another embodiment of this invention is directed to the compound of formula E9.

Another embodiment of this invention is directed to the compound of formula F7.

Another embodiment of this invention is directed to the compound of formula F8.

Another embodiment of this invention is directed to the compound of formula F9.

Another embodiment of this invention is directed to the compound of formula F10.

Another embodiment of this invention is directed to the compound of formula F11.

Another embodiment of this invention is directed to the compound of formula F12.

Another embodiment of this invention is directed to the compound of formula F13.

Another embodiment of this invention is directed to the compound of formula F14.

Another embodiment of this invention is directed to the compound of formula F15.

Another embodiment of this invention is directed to the compound of formula F16.

Another embodiment of this invention is directed to the compound of formula F17.

Another embodiment of this invention is directed to the compound of formula F18.

Another embodiment of this invention is directed to the compound of formula F19.

Another embodiment of this invention is directed to the compound of formula F20d.

Another embodiment of this invention is directed to the compound of formula F20e.

Another embodiment of this invention is directed to the compound of formula F20f.

Another embodiment of this invention is directed to the compound of formula F20g.

Another embodiment of this invention is directed to the compound of formula F20h.

Another embodiment of this invention is directed to the compound of formula F21d.

Another embodiment of this invention is directed to the compound of formula F21e.

Another embodiment of this invention is directed to the compound of formula F21f.

Another embodiment of this invention is directed to the compound of formula F21g.

Another embodiment of this invention is directed to the compound of formula F21h.

Another embodiment of this invention is directed to the compound of formula F22d.

Another embodiment of this invention is directed to the compound of formula F22e.

Another embodiment of this invention is directed to the compound of formula F22f.

Another embodiment of this invention is directed to the compound of formula F22g.

Another embodiment of this invention is directed to the compound of formula F22h.

Another embodiment of this invention is directed to the compound of formula F23c.

Another embodiment of this invention is directed to the compound of formula F23d.

Another embodiment of this invention is directed to the compound of formula F23e.

Another embodiment of this invention is directed to the compound of formula F23f.

Another embodiment of this invention is directed to the compound of formula F23g.

Another embodiment of this invention is directed to the compound of formula F23h.

Another embodiment of this invention is directed to the compound of formula F24c.

Another embodiment of this invention is directed to the compound of formula F24d.

Another embodiment of this invention is directed to the compound of formula F24e.

Another embodiment of this invention is directed to the compound of formula F24f.

Another embodiment of this invention is directed to the compound of formula F24g.

Another embodiment of this invention is directed to the compound of formula F24h.

Another embodiment of this invention is directed to the compound of formula F25a.

Another embodiment of this invention is directed to the compound of formula F25b.

Another embodiment of this invention is directed to the compound of formula F25c.

Another embodiment of this invention is directed to the compound of formula F25d.

Another embodiment of this invention is directed to the compound of formula F25e.

Another embodiment of this invention is directed to the compound of formula F25f.

Another embodiment of this invention is directed to the compound of formula F25g.

Another embodiment of this invention is directed to the compound of formula F25h.

Another embodiment of this invention is directed to the compound of formula F26a.

Another embodiment of this invention is directed to the compound of formula F26b.

Another embodiment of this invention is directed to the compound of formula F26c.

Another embodiment of this invention is directed to the compound of formula F26d.

Another embodiment of this invention is directed to the compound of formula F26e.

Another embodiment of this invention is directed to the compound of formula F26f.

Another embodiment of this invention is directed to the compound of formula F26g.

Another embodiment of this invention is directed to the compound of formula F26h.

Another embodiment of this invention is directed to the compound of formula F27a.

Another embodiment of this invention is directed to the compound of formula F27b.

Another embodiment of this invention is directed to the compound of formula F27c.

Another embodiment of this invention is directed to the compound of formula F27d.

Another embodiment of this invention is directed to the compound of formula F27e.

Another embodiment of this invention is directed to the compound of formula F27f.

Another embodiment of this invention is directed to the compound of formula F27g.

Another embodiment of this invention is directed to the compound of formula F27h.

Another embodiment of this invention is directed to the compound of formula F28a.

Another embodiment of this invention is directed to the compound of formula F28b.

Another embodiment of this invention is directed to the compound of formula F28c.

Another embodiment of this invention is directed to the compound of formula F28d.

Another embodiment of this invention is directed to the compound of formula F28e.

Another embodiment of this invention is directed to the compound of formula F28f.

Another embodiment of this invention is directed to the compound of formula F28g.

Another embodiment of this invention is directed to the compound of formula F28h.

Another embodiment of this invention is directed to the compound of formula F29a.

Another embodiment of this invention is directed to the compound of formula F29b.

Another embodiment of this invention is directed to the compound of formula F29c.

Another embodiment of this invention is directed to the compound of formula F29d.

Another embodiment of this invention is directed to the compound of formula F29e.

Another embodiment of this invention is directed to the compound of formula F29f.

Another embodiment of this invention is directed to the compound of formula F29g.

Another embodiment of this invention is directed to the compound of formula F29h.

Another embodiment of this invention is directed to the compound of formula F30a.

Another embodiment of this invention is directed to the compound of formula F30b.

Another embodiment of this invention is directed to the compound of formula F30c.

Another embodiment of this invention is directed to the compound of formula F30d.

Another embodiment of this invention is directed to the compound of formula F30e.

Another embodiment of this invention is directed to the compound of formula F30f.

Another embodiment of this invention is directed to the compound of formula F30g.

Another embodiment of this invention is directed to the compound of formula F30h.

Another embodiment of this invention is directed to the compound of formula F31a.

Another embodiment of this invention is directed to the compound of formula F31b.

Another embodiment of this invention is directed to the compound of formula F31c.

Another embodiment of this invention is directed to the compound of formula F31d.

Another embodiment of this invention is directed to the compound of formula F31e.

Another embodiment of this invention is directed to the compound of formula F31f.

Another embodiment of this invention is directed to the compound of formula F31g.

Another embodiment of this invention is directed to the compound of formula F31h.

Another embodiment of this invention is directed to the compound of formula F32a.

Another embodiment of this invention is directed to the compound of formula F32b.

Another embodiment of this invention is directed to the compound of formula F32c.

Another embodiment of this invention is directed to the compound of formula F32d.

Another embodiment of this invention is directed to the compound of formula F32e.

Another embodiment of this invention is directed to the compound of formula F32f.

Another embodiment of this invention is directed to the compound of formula F32g.

Another embodiment of this invention is directed to the compound of formula F32h.

Another embodiment of this invention is directed to the compound of formula F33a.

Another embodiment of this invention is directed to the compound of formula F33b.

Another embodiment of this invention is directed to the compound of formula F33c.

Another embodiment of this invention is directed to the compound of formula F33d.

Another embodiment of this invention is directed to the compound of formula F33e.

Another embodiment of this invention is directed to the compound of formula F33f.

Another embodiment of this invention is directed to the compound of formula F33g.

Another embodiment of this invention is directed to the compound of formula F33h.

Another embodiment of this invention is directed to the compound of formula J1.

Another embodiment of this invention is directed to the compound of formula J2.

Another embodiment of this invention is directed to the compound of formula K7.

Another embodiment of this invention is directed to the compound of formula K8b.

Another embodiment of this invention is directed to the compound of formula K8c.

Another embodiment of this invention is directed to the compound of formula K8d.

Another embodiment of this invention is directed to the compound of formula K8e.

Another embodiment of this invention is directed to the compound of formula K8f.

Another embodiment of this invention is directed to the compound of formula K8g.

Another embodiment of this invention is directed to the compound of formula K8h.

Another embodiment of this invention is directed to the compound of formula K9a.

Another embodiment of this invention is directed to the compound of formula K9b.

Another embodiment of this invention is directed to the compound of formula K9c.

Another embodiment of this invention is directed to the compound of formula K9d.

Another embodiment of this invention is directed to the compound of formula K9e.

Another embodiment of this invention is directed to the compound of formula K9f.

Another embodiment of this invention is directed to the compound of formula K9g.

Another embodiment of this invention is directed to the compound of formula K9h.

Another embodiment of this invention is directed to the compound of formula K10a.

Another embodiment of this invention is directed to the compound of formula K10b.

Another embodiment of this invention is directed to the compound of formula K10c.

Another embodiment of this invention is directed to the compound of formula K10d.

Another embodiment of this invention is directed to the compound of formula K10e.

Another embodiment of this invention is directed to the compound of formula K10f.

Another embodiment of this invention is directed to the compound of formula K10g.

Another embodiment of this invention is directed to the compound of formula K10h.

Another embodiment of this invention is directed to the compound of formula K11a.

Another embodiment of this invention is directed to the compound of formula K11b.

Another embodiment of this invention is directed to the compound of formula K11c.

Another embodiment of this invention is directed to the compound of formula K11d.

Another embodiment of this invention is directed to the compound of formula K11e.

Another embodiment of this invention is directed to the compound of formula K11f.

Another embodiment of this invention is directed to the compound of formula K11g.

, Another embodiment of this invention is directed to the compound of formula K11h.

Another embodiment of this invention is directed to the compound of formula K12a.

Another embodiment of this invention is directed to the compound of formula K12b.

Another embodiment of this invention is directed to the compound of formula K12c.

Another embodiment of this invention is directed to the compound of formula K12d.

Another embodiment of this invention is directed to the compound of formula K12e.

Another embodiment of this invention is directed to the compound of formula K12f.

Another embodiment of this invention is directed to the compound of formula K12g.

Another embodiment of this invention is directed to the compound of formula K12h.

Another embodiment of this invention is directed to the compound of formula K13a.

Another embodiment of this invention is directed to the compound of formula K13b.

Another embodiment of this invention is directed to the compound of formula K13c.

Another embodiment of this invention is directed to the compound of formula K13d.

Another embodiment of this invention is directed to the compound of formula K13e.

Another embodiment of this invention is directed to the compound of formula K13f.

Another embodiment of this invention is directed to the compound of formula K13g.

Another embodiment of this invention is directed to the compound of formula K13h.

Another embodiment of this invention is directed to the compound of formula K14a.

Another embodiment of this invention is directed to the compound of formula K14b.

Another embodiment of this invention is directed to the compound of formula K14c.

Another embodiment of this invention is directed to the compound of formula K14d.

Another embodiment of this invention is directed to the compound of formula K14e.

Another embodiment of this invention is directed to the compound of formula K14f.

Another embodiment of this invention is directed to the compound of formula K14g.

Another embodiment of this invention is directed to the compound of formula K14h.

Another embodiment of this invention is directed to the compound of formula K15a.

Another embodiment of this invention is directed to the compound of formula K15b.

Another embodiment of this invention is directed to the compound of formula K15c.

Another embodiment of this invention is directed to the compound of formula K15d.

Another embodiment of this invention is directed to the compound of formula K15e.

Another embodiment of this invention is directed to the compound of formula K15f.

Another embodiment of this invention is directed to the compound of formula K15g.

Another embodiment of this invention is directed to the compound of formula K15h.

Another embodiment of this invention is directed to the compound of formula K16a.

Another embodiment of this invention is directed to the compound of formula K16b.

Another embodiment of this invention is directed to the compound of formula K16c.

Another embodiment of this invention is directed to the compound of formula K16d.

Another embodiment of this invention is directed to the compound of formula K16e.

Another embodiment of this invention is directed to the compound of formula K16f.

Another embodiment of this invention is directed to the compound of formula K16g.

Another embodiment of this invention is directed to the compound of formula K16h.

Another embodiment of this invention is directed to the compound of formula K17a.

Another embodiment of this invention is directed to the compound of formula K17b.

Another embodiment of this invention is directed to the compound of formula K17c.

Another embodiment of this invention is directed to the compound of formula K17d.

Another embodiment of this invention is directed to the compound of formula K17e.

Another embodiment of this invention is directed to the compound of formula K17f.

Another embodiment of this invention is directed to the compound of formula K17g.

Another embodiment of this invention is directed to the compound of formula K17h.

Another embodiment of this invention is directed to the compound of formula K18a.

Another embodiment of this invention is directed to the compound of formula K18b.

Another embodiment of this invention is directed to the compound of formula K18c.

Another embodiment of this invention is directed to the compound of formula K18d.

Another embodiment of this invention is directed to the compound of formula K18e.

Another embodiment of this invention is directed to the compound of formula K18f.

Another embodiment of this invention is directed to the compound of formula K18g.

Another embodiment of this invention is directed to the compound of formula K18h.

Another embodiment of this invention is directed to the compound of formula K19a.

Another embodiment of this invention is directed to the compound of formula K19b.

Another embodiment of this invention is directed to the compound of formula K19c.

Another embodiment of this invention is directed to the compound of formula K19d.

Another embodiment of this invention is directed to the compound of formula K19e.

Another embodiment of this invention is directed to the compound of formula K19f.

Another embodiment of this invention is directed to the compound of formula K19g.

Another embodiment of this invention is directed to the compound of formula K19h.

Another embodiment of this invention is directed to the compound of formula K20a.

Another embodiment of this invention is directed to the compound of formula K20b.

Another embodiment of this invention is directed to the compound of formula K20c.

Another embodiment of this invention is directed to the compound of formula K20d.

Another embodiment of this invention is directed to the compound of formula K20e.

Another embodiment of this invention is directed to the compound of formula K20f.

Another embodiment of this invention is directed to the compound of formula K20g.

Another embodiment of this invention is directed to the compound of formula K20h.

Another embodiment of this invention is directed to the compound of formula K21a.

Another embodiment of this invention is directed to the compound of formula K21b.

Another embodiment of this invention is directed to the compound of formula K21c.

Another embodiment of this invention is directed to the compound of formula K21d.

Another embodiment of this invention is directed to the compound of formula K21e.

Another embodiment of this invention is directed to the compound of formula K21f.

Another embodiment of this invention is directed to the compound of formula K21g.

Another embodiment of this invention is directed to the compound of formula K21h.

Another embodiment of this invention is directed to the compound of formula K22a.

Another embodiment of this invention is directed to the compound of formula K22b.

Another embodiment of this invention is directed to the compound of formula K22c.

Another embodiment of this invention is directed to the compound of formula K22d.

Another embodiment of this invention is directed to the compound of formula K22e.

Another embodiment of this invention is directed to the compound of formula K22f.

Another embodiment of this invention is directed to the compound of formula K22g.

Another embodiment of this invention is directed to the compound of formula K22h.

Another embodiment of this invention is directed to the compound of formula X1.

Another embodiment of this invention is directed to the compound of formula X2.

Another embodiment of this invention is directed to the compound of formula X3.

Another embodiment of this invention is directed to the compound of formula X4.

Another embodiment of this invention is directed to the compound of formula X5.

Another embodiment of this invention is directed to the compound of formula X6.

Another embodiment of this invention is directed to the compound of formula X7.

Another embodiment of this invention is directed to the compound of formula X8.

Another embodiment of this invention is directed to the compound of formula X9.

Another embodiment of this invention is directed to the compound of formula X10.

Another embodiment of this invention is directed to the compound of formula X11.

Another embodiment of this invention is directed to a solvate, a pharmaceutically acceptable ester, or a pharmaceutically acceptable salt of the compound of formula Y1.

Another embodiment of this invention is directed to a solvate, a pharmaceutically acceptable ester, or a pharmaceutically acceptable salt of the compound of formula Y2.

Another embodiment of this invention is directed to a solvate, a pharmaceutically acceptable ester, or a pharmaceutically acceptable salt of the compound of formula Y3.

Another embodiment of this invention is directed to a solvate, a pharmaceutically acceptable ester, or a pharmaceutically acceptable salt of the compound of formula A9.

Another embodiment of this invention is directed to a solvate, a pharmaceutically acceptable ester, or a pharmaceutically acceptable salt of the compound of formula A10.

Another embodiment of this invention is directed to a solvate, a pharmaceutically acceptable ester, or a pharmaceutically acceptable salt of the compound of formula A11.

Another embodiment of this invention is directed to a solvate, a pharmaceutically acceptable ester, or a pharmaceutically acceptable salt of the compound of formula A12.

Another embodiment of this invention is directed to a solvate, a pharmaceutically acceptable ester, or a pharmaceutically acceptable salt of the compound of formula A13.

Another embodiment of this invention is directed to a solvate, a pharmaceutically acceptable ester, or a pharmaceutically acceptable salt of the compound of formula A14.

Another embodiment of this invention is directed to a solvate, a pharmaceutically acceptable ester, or a pharmaceutically acceptable salt of the compound of formula B1.

Another embodiment of this invention is directed to a solvate, a pharmaceutically acceptable ester, or a pharmaceutically acceptable salt of the compound d of formula B2.

Another embodiment of this invention is directed to a solvate, a pharmaceutically acceptable ester, or a pharmaceutically acceptable salt of the compound of formula B3.

Another embodiment of this invention is directed to a solvate, a pharmaceutically acceptable ester, or a pharmaceutically acceptable salt of the compound of formula B4.

Another embodiment of this invention is directed to a solvate, a pharmaceutically acceptable ester, or a pharmaceutically acceptable salt of the compound of formula B5.

Another embodiment of this invention is directed to a solvate, a pharmaceutically acceptable ester, or a pharmaceutically acceptable salt of the compound of formula B6.

Another embodiment of this invention is directed to a solvate, a pharmaceutically acceptable ester, or a pharmaceutically acceptable salt of the compound of formula B7.

Another embodiment of this invention is directed to a solvate, a pharmaceutically acceptable ester, or a pharmaceutically acceptable salt of the compound of formula B8.

Another embodiment of this invention is directed to a solvate, a pharmaceutically acceptable ester, or a pharmaceutically acceptable salt of the compound of formula B9.

Another embodiment of this invention is directed to a solvate, a pharmaceutically acceptable ester, or a pharmaceutically acceptable salt of the compound of formula B10.

Another embodiment of this invention is directed to a solvate, a pharmaceutically acceptable ester, or a pharmaceutically acceptable salt of the compound of formula B11.

Another embodiment of this invention is directed to a solvate, a pharmaceutically acceptable ester, or a pharmaceutically acceptable salt of the compound of formula B12.

Another embodiment of this invention is directed to a solvate, a pharmaceutically acceptable ester, or a pharmaceutically acceptable salt of the compound of formula B13.

Another embodiment of this invention is directed to a solvate, a pharmaceutically acceptable ester, or a pharmaceutically acceptable salt of the compound of formula B14.

Another embodiment of this invention is directed to a solvate, a pharmaceutically acceptable ester, or a pharmaceutically acceptable salt of the compound of formula B15.

Another embodiment of this invention is directed to a solvate, a pharmaceutically acceptable ester, or a pharmaceutically acceptable salt of the compound of formula C3.

Another embodiment of this invention is directed to a solvate, a pharmaceutically acceptable ester, or a pharmaceutically acceptable salt of the compound of formula C4.

Another embodiment of this invention is directed to a solvate, a pharmaceutically acceptable ester, or a pharmaceutically acceptable salt of the compound of formula C5.

Another embodiment of this invention is directed to a solvate, a pharmaceutically acceptable ester, or a pharmaceutically acceptable salt of the compound of formula D4.

Another embodiment of this invention is directed to a solvate, a pharmaceutically acceptable ester, or a pharmaceutically acceptable salt of the compound of formula E4.

Another embodiment of this invention is directed to a solvate, a pharmaceutically acceptable ester, or a pharmaceutically acceptable salt of the compound of formula E6.

Another embodiment of this invention is directed to a solvate, a pharmaceutically acceptable ester, or a pharmaceutically acceptable salt of the compound d of formula E7.

Another embodiment of this invention is directed to a solvate, a pharmaceutically acceptable ester, or a pharmaceutically acceptable salt of the compound of formula E8.

Another embodiment of this invention is directed to a solvate, a pharmaceutically acceptable ester, or a pharmaceutically acceptable salt of the compound of formula E9.

Another embodiment of this invention is directed to a solvate, a pharmaceutically acceptable ester, or a pharmaceutically acceptable salt of the compound of formula F7.

Another embodiment of this invention is directed to the compound of formula F8.

Another embodiment of this invention is directed to a solvate, a pharmaceutically acceptable ester, or a pharmaceutically acceptable salt of the compound of formula F9.

Another embodiment of this invention is directed to the compound of formula F10.

Another embodiment of this invention is directed to a solvate, a pharmaceutically acceptable ester, or a pharmaceutically acceptable salt of the compound of formula F11.

Another embodiment of this invention is directed to a solvate, a pharmaceutically acceptable ester, or a pharmaceutically acceptable salt of the compound of formula F12.

Another embodiment of this invention is directed to a solvate, a pharmaceutically acceptable ester, or a pharmaceutically acceptable salt of the compound of formula F13.

Another embodiment of this invention is directed to a solvate, a pharmaceutically acceptable ester, or a pharmaceutically acceptable salt of the compound of formula F14.

Another embodiment of this invention is directed to a solvate, a pharmaceutically acceptable ester, or a pharmaceutically acceptable salt of the compound of formula F15.

Another embodiment of this invention is directed to a solvate, a pharmaceutically acceptable ester, or a pharmaceutically acceptable salt of the compound of formula F16.

Another embodiment of this invention is directed to a solvate, a pharmaceutically acceptable ester, or a pharmaceutically acceptable salt of the compound of formula F17.

Another embodiment of this invention is directed to a solvate, a pharmaceutically acceptable ester, or a pharmaceutically acceptable salt of the compound of formula F18.

Another embodiment of this invention is directed to a solvate, a pharmaceutically acceptable ester, or a pharmaceutically acceptable salt of the compound of formula F19.

Another embodiment of this invention is directed to a solvate, a pharmaceutically acceptable ester, or a pharmaceutically acceptable salt of the compound of formula F20d.

Another embodiment of this invention is directed to a solvate, a pharmaceutically acceptable ester, or a pharmaceutically acceptable salt of the compound of formula F20e.

Another embodiment of this invention is directed to a solvate, a pharmaceutically acceptable ester, or a pharmaceutically acceptable salt of the compound of formula F20f.

Another embodiment of this invention is directed to a solvate, a pharmaceutically acceptable ester, or a pharmaceutically acceptable salt of the compound of formula F20g.

Another embodiment of this invention is directed to a solvate, a pharmaceutically acceptable ester, or a pharmaceutically acceptable salt of the compound of formula F20h.

Another embodiment of this invention is directed to a solvate, a pharmaceutically acceptable ester, or a pharmaceutically acceptable salt of the compound of formula F21d.

Another embodiment of this invention is directed to a solvate, a pharmaceutically acceptable ester, or a pharmaceutically acceptable salt of the compound of formula F21e.

Another embodiment of this invention is directed to a solvate, a pharmaceutically acceptable ester, or a pharmaceutically acceptable salt of the compound of formula F21f.

Another embodiment of this invention is directed to a solvate, a pharmaceutically acceptable ester, or a pharmaceutically acceptable salt of the compound of formula F21g.

Another embodiment of this invention is directed to a solvate, a pharmaceutically acceptable ester, or a pharmaceutically acceptable salt of the compound of formula F21h.

Another embodiment of this invention is directed to a solvate, a pharmaceutically acceptable ester, or a pharmaceutically acceptable salt of the compound of formula F22d.

Another embodiment of this invention is directed to a solvate, a pharmaceutically acceptable ester, or a pharmaceutically acceptable salt of the compound of formula F22e.

Another embodiment of this invention is directed to a solvate, a pharmaceutically acceptable ester, or a pharmaceutically acceptable salt of the compound of formula F22f.

Another embodiment of this invention is directed to the compound of formula F22g.

Another embodiment of this invention is directed to a solvate, a pharmaceutically acceptable ester, or a pharmaceutically acceptable salt of the compound of formula F22h.

Another embodiment of this invention is directed to the compound of formula F23c.

Another embodiment of this invention is directed to a solvate, a pharmaceutically acceptable ester, or a pharmaceutically acceptable salt of the compound of formula F23d.

Another embodiment of this invention is directed to a solvate, a pharmaceutically acceptable ester, or a pharmaceutically acceptable salt of the compound of formula F23e.

Another embodiment of this invention is directed to a solvate, a pharmaceutically acceptable ester, or a pharmaceutically acceptable salt of the compound of formula F23f.

Another embodiment of this invention is directed to the compound of formula F23g.

Another embodiment of this invention is directed to a solvate, a pharmaceutically acceptable ester, or a pharmaceutically acceptable salt of the compound of formula F23h.

Another embodiment of this invention is directed to a solvate, a pharmaceutically acceptable ester, or a pharmaceutically acceptable salt of the compound of formula F24c.

Another embodiment of this invention is directed to a solvate, a pharmaceutically acceptable ester, or a pharmaceutically acceptable salt of the compound of formula F24d.

Another embodiment of this invention is directed to a solvate, a pharmaceutically acceptable ester, or a pharmaceutically acceptable salt of the compound of formula F24e.

Another embodiment of this invention is directed to a solvate, a pharmaceutically acceptable ester, or a pharmaceutically acceptable salt of the compound of formula F24f.

Another embodiment of this invention is directed to a solvate, a pharmaceutically acceptable ester, or a pharmaceutically acceptable salt of the compound of formula F24g.

Another embodiment of this invention is directed to a solvate, a pharmaceutically acceptable ester, or a pharmaceutically acceptable salt of the compound of formula F24h.

Another embodiment of this invention is directed to a solvate, a pharmaceutically acceptable ester, or a pharmaceutically acceptable salt of the compound of formula F25a.

Another embodiment of this invention is directed to a solvate, a pharmaceutically acceptable ester, or a pharmaceutically acceptable salt of the compound of formula F25b.

Another embodiment of this invention is directed to a solvate, a pharmaceutically acceptable ester, or a pharmaceutically acceptable salt of the compound of formula F25c.

Another embodiment of this invention is directed to a solvate, a pharmaceutically acceptable ester, or a pharmaceutically acceptable salt of the compound of formula F25d.

Another embodiment of this invention is directed to a solvate, a pharmaceutically acceptable ester, or a pharmaceutically acceptable salt of the compound of formula F25e.

Another embodiment of this invention is directed to a solvate, a pharmaceutically acceptable ester, or a pharmaceutically acceptable salt of the compound of formula F25f.

Another embodiment of this invention is directed to a solvate, a pharmaceutically acceptable ester, or a pharmaceutically acceptable salt of the compound of formula F25g.

Another embodiment of this invention is directed to a solvate, a pharmaceutically acceptable ester, or a pharmaceutically acceptable salt of the compound of formula F25h.

Another embodiment of this invention is directed to a solvate, a pharmaceutically acceptable ester, or a pharmaceutically acceptable salt of the compound of formula F26a.

Another embodiment of this invention is directed to a solvate, a pharmaceutically acceptable ester, or a pharmaceutically acceptable salt of the compound of formula F26b.

Another embodiment of this invention is directed to a solvate, a pharmaceutically acceptable ester, or a pharmaceutically acceptable salt of the compound of formula F26c.

Another embodiment of this invention is directed to a solvate, a pharmaceutically acceptable ester, or a pharmaceutically acceptable salt of the compound of formula F26d.

Another embodiment of this invention is directed to a solvate, a pharmaceutically acceptable ester, or a pharmaceutically acceptable salt of the compound of formula F26e.

Another embodiment of this invention is directed to a solvate, a pharmaceutically acceptable ester, or a pharmaceutically acceptable salt of the compound of formula F26f.

Another embodiment of this invention is directed to a solvate, a pharmaceutically acceptable ester, or a pharmaceutically acceptable salt of the compound of formula F26g.

Another embodiment of this invention is directed to a solvate, a pharmaceutically acceptable ester, or a pharmaceutically acceptable salt of the compound of formula F26h.

Another embodiment of this invention is directed to a solvate, a pharmaceutically acceptable ester, or a pharmaceutically acceptable salt of the compound of formula F27a.

Another embodiment of this invention is directed to a solvate, a pharmaceutically acceptable ester, or a pharmaceutically acceptable salt of the compound of formula F27b.

Another embodiment of this invention is directed to a solvate, a pharmaceutically acceptable ester, or a pharmaceutically acceptable salt of the compound of formula F27c.

Another embodiment of this invention is directed to a solvate, a pharmaceutically acceptable ester, or a pharmaceutically acceptable salt of the compound of formula F27d.

Another embodiment of this invention is directed to a solvate, a pharmaceutically acceptable ester, or a pharmaceutically acceptable salt of the compound of formula F27e.

Another embodiment of this invention is directed to a solvate, a pharmaceutically acceptable ester, or a pharmaceutically acceptable salt of the compound of formula F27f.

Another embodiment of this invention is directed to a solvate, a pharmaceutically acceptable ester, or a pharmaceutically acceptable salt of the compound of formula F27g.

Another embodiment of this invention is directed to a solvate, a pharmaceutically acceptable ester, or a pharmaceutically acceptable salt of the compound of formula F27h.

Another embodiment of this invention is directed to a solvate, a pharmaceutically acceptable ester, or a pharmaceutically acceptable salt of the compound of formula F28a.

Another embodiment of this invention is directed to a solvate, a pharmaceutically acceptable ester, or a pharmaceutically acceptable salt of the compound of formula F28b.

Another embodiment of this invention is directed to a solvate, a pharmaceutically acceptable ester, or a pharmaceutically acceptable salt of the compound of formula F28c.

Another embodiment of this invention is directed to a solvate, a pharmaceutically acceptable ester, or a pharmaceutically acceptable salt of the compound of formula F28d.

Another embodiment of this invention is directed to a solvate, a pharmaceutically acceptable ester, or a pharmaceutically acceptable salt of the compound of formula F28e.

Another embodiment of this invention is directed to a solvate, a pharmaceutically acceptable ester, or a pharmaceutically acceptable salt of the compound of formula F28f.

Another embodiment of this invention is directed to a solvate, a pharmaceutically acceptable ester, or a pharmaceutically acceptable salt of the compound of formula F28g.

Another embodiment of this invention is directed to a solvate, a pharmaceutically acceptable ester, or a pharmaceutically acceptable salt of the compound of formula F28h.

Another embodiment of this invention is directed to a solvate, a pharmaceutically acceptable ester, or a pharmaceutically acceptable salt of the compound of formula F29a.

Another embodiment of this invention is directed to a solvate, a pharmaceutically acceptable ester, or a pharmaceutically acceptable salt of the compound of formula F29b.

Another embodiment of this invention is directed to a solvate, a pharmaceutically acceptable ester, or a pharmaceutically acceptable salt of the compound of formula F29c.

Another embodiment of this invention is directed to a solvate, a pharmaceutically acceptable ester, or a pharmaceutically acceptable salt of the compound of formula F29d.

Another embodiment of this invention is directed to a solvate, a pharmaceutically acceptable ester, or a pharmaceutically acceptable salt of the compound of formula F29e.

Another embodiment of this invention is directed to a solvate, a pharmaceutically acceptable ester, or a pharmaceutically acceptable salt of the compound of formula F29f.

Another embodiment of this invention is directed to a solvate, a pharmaceutically acceptable ester, or a pharmaceutically acceptable salt of the compound of formula F29g.

Another embodiment of this invention is directed to a solvate, a pharmaceutically acceptable ester, or a pharmaceutically acceptable salt of the compound of formula F29h.

Another embodiment of this invention is directed to a solvate, a pharmaceutically acceptable ester, or a pharmaceutically acceptable salt of the compound of formula F30a.

Another embodiment of this invention is directed to a solvate, a pharmaceutically acceptable ester, or a pharmaceutically acceptable salt of the compound of formula F30b.

Another embodiment of this invention is directed to a solvate, a pharmaceutically acceptable ester, or a pharmaceutically acceptable salt of the compound of formula F30c.

Another embodiment of this invention is directed to a solvate, a pharmaceutically acceptable ester, or a pharmaceutically acceptable salt of the compound of formula F30d.

Another embodiment of this invention is directed to a solvate, a pharmaceutically acceptable ester, or a pharmaceutically acceptable salt of the compound of formula F30e.

Another embodiment of this invention is directed to a solvate, a pharmaceutically acceptable ester, or a pharmaceutically acceptable salt of the compound of formula F30f.

Another embodiment of this invention is directed to a solvate, a pharmaceutically acceptable ester, or a pharmaceutically acceptable salt of the compound of formula F30g.

Another embodiment of this invention is directed to a solvate, a pharmaceutically acceptable ester, or a pharmaceutically acceptable salt of the compound of formula F30h.

Another embodiment of this invention is directed to a solvate, a pharmaceutically acceptable ester, or a pharmaceutically acceptable salt of the compound of formula F31a.

Another embodiment of this invention is directed to a solvate, a pharmaceutically acceptable ester, or a pharmaceutically acceptable salt of the compound of formula F31b.

Another embodiment of this invention is directed to a solvate, a pharmaceutically acceptable ester, or a pharmaceutically acceptable salt of the compound of formula F31c.

Another embodiment of this invention is directed to a solvate, a pharmaceutically acceptable ester, or a pharmaceutically acceptable salt of the compound of formula F31d.

Another embodiment of this invention is directed to a solvate, a pharmaceutically acceptable ester, or a pharmaceutically acceptable salt of the compound of formula F31e.

Another embodiment of this invention is directed to a solvate, a pharmaceutically acceptable ester, or a pharmaceutically acceptable salt of the compound of formula F31f.

Another embodiment of this invention is directed to a solvate, a pharmaceutically acceptable ester, or a pharmaceutically acceptable salt of the compound of formula F31g.

Another embodiment of this invention is directed to a solvate, a pharmaceutically acceptable ester, or a pharmaceutically acceptable salt of the compound of formula F31h.

Another embodiment of this invention is directed to a solvate, a pharmaceutically acceptable ester, or a pharmaceutically acceptable salt of the compound of formula F32a.

Another embodiment of this invention is directed to a solvate, a pharmaceutically acceptable ester, or a pharmaceutically acceptable salt of the compound of formula F32b.

Another embodiment of this invention is directed to a solvate, a pharmaceutically acceptable ester, or a pharmaceutically acceptable salt of the compound of formula F32c.

Another embodiment of this invention is directed to a solvate, a pharmaceutically acceptable ester, or a pharmaceutically acceptable salt of the compound of formula F32d.

Another embodiment of this invention is directed to a solvate, a pharmaceutically acceptable ester, or a pharmaceutically acceptable salt of the compound of formula F32e.

Another embodiment of this invention is directed to a solvate, a pharmaceutically acceptable ester, or a pharmaceutically acceptable salt of the compound of formula F32f.

Another embodiment of this invention is directed to a solvate, a pharmaceutically acceptable ester, or a pharmaceutically acceptable salt of the compound of formula F32g.

Another embodiment of this invention is directed to a solvate, a pharmaceutically acceptable ester, or a pharmaceutically acceptable salt of the compound of formula F32h.

Another embodiment of this invention is directed to a solvate, a pharmaceutically acceptable ester, or a pharmaceutically acceptable salt of the compound of formula F33a.

Another embodiment of this invention is directed to a solvate, a pharmaceutically acceptable ester, or a pharmaceutically acceptable salt of the compound of formula F33b.

Another embodiment of this invention is directed to a solvate, a pharmaceutically acceptable ester, or a pharmaceutically acceptable salt of the compound of formula F33c.

Another embodiment of this invention is directed to a solvate, a pharmaceutically acceptable ester, or a pharmaceutically acceptable salt of the compound of formula F33d.

Another embodiment of this invention is directed to a solvate, a pharmaceutically acceptable ester, or a pharmaceutically acceptable salt of the compound of formula F33e.

Another embodiment of this invention is directed to a solvate, a pharmaceutically acceptable ester, or a pharmaceutically acceptable salt of the compound of formula F33f.

Another embodiment of this invention is directed to a solvate, a pharmaceutically acceptable ester, or a pharmaceutically acceptable salt of the compound of formula F33g.

Another embodiment of this invention is directed to a solvate, a pharmaceutically acceptable ester, or a pharmaceutically acceptable salt of the compound of formula F33h.

Another embodiment of this invention is directed to a solvate, a pharmaceutically acceptable ester, or a pharmaceutically acceptable salt of the compound of formula J1.

Another embodiment of this invention is directed to a solvate, a pharmaceutically acceptable ester, or a pharmaceutically acceptable salt of the compound of formula J2.

Another embodiment of this invention is directed to a solvate, a pharmaceutically acceptable ester, or a pharmaceutically acceptable salt of the compound of formula K7.

Another embodiment of this invention is directed to a solvate, a pharmaceutically acceptable ester, or a pharmaceutically acceptable salt of the compound of formula K8b.

Another embodiment of this invention is directed to a solvate, a pharmaceutically acceptable ester, or a pharmaceutically acceptable salt of the compound of formula K8c.

Another embodiment of this invention is directed to a solvate, a pharmaceutically acceptable ester, or a pharmaceutically acceptable salt of the compound of formula K8d.

Another embodiment of this invention is directed to a solvate, a pharmaceutically acceptable ester, or a pharmaceutically acceptable salt of the compound of formula K8e.

Another embodiment of this invention is directed to a solvate, a pharmaceutically acceptable ester, or a pharmaceutically acceptable salt of the compound of formula K8f.

Another embodiment of this invention is directed to a solvate, a pharmaceutically acceptable ester, or a pharmaceutically acceptable salt of the compound of formula K8g.

Another embodiment of this invention is directed to a solvate, a pharmaceutically acceptable ester, or a pharmaceutically acceptable salt of the compound of formula K8h.

Another embodiment of this invention is directed to a solvate, a pharmaceutically acceptable ester, or a pharmaceutically acceptable salt of the compound of formula K9a.

Another embodiment of this invention is directed to a solvate, a pharmaceutically acceptable ester, or a pharmaceutically acceptable salt of the compound of formula K9b.

Another embodiment of this invention is directed to a solvate, a pharmaceutically acceptable ester, or a pharmaceutically acceptable salt of the compound of formula K9c.

Another embodiment of this invention is directed to a solvate, a pharmaceutically acceptable ester, or a pharmaceutically acceptable salt of the compound of formula K9d.

Another embodiment of this invention is directed to a solvate, a pharmaceutically acceptable ester, or a pharmaceutically acceptable salt of the compound of formula K9e.

Another embodiment of this invention is directed to a solvate, a pharmaceutically acceptable ester, or a pharmaceutically acceptable salt of the compound of formula K9f.

Another embodiment of this invention is directed to a solvate, a pharmaceutically acceptable ester, or a pharmaceutically acceptable salt of the compound of formula K9g.

Another embodiment of this invention is directed to a solvate, a pharmaceutically acceptable ester, or a pharmaceutically acceptable salt of the compound of formula K9h.

Another embodiment of this invention is directed to a solvate, a pharmaceutically acceptable ester, or a pharmaceutically acceptable salt of the compound of formula K10a.

Another embodiment of this invention is directed to a solvate, a pharmaceutically acceptable ester, or a pharmaceutically acceptable salt of the compound of formula K10b.

Another embodiment of this invention is directed to a solvate, a pharmaceutically acceptable ester, or a pharmaceutically acceptable salt of the compound of formula K10c.

Another embodiment of this invention directed to a solvate, a pharmaceutically acceptable ester, or a pharmaceutically acceptable salt of the compound of formula K10d.

Another embodiment of this invention is directed to a solvate, a pharmaceutically acceptable ester, or a pharmaceutically acceptable salt of the compound of formula K10e.

Another embodiment of this invention is directed to a solvate, a pharmaceutically acceptable ester, or a pharmaceutically acceptable salt of the compound of formula K10f.

Another embodiment of this invention is directed to a solvate, a pharmaceutically acceptable ester, or a pharmaceutically acceptable salt of the compound of formula K10g.

Another embodiment of this invention is directed to a solvate, a pharmaceutically acceptable ester, or a pharmaceutically acceptable salt of the compound of formula K10h.

Another embodiment of this invention is directed to a solvate, a pharmaceutically acceptable ester, or a pharmaceutically acceptable salt of the compound of formula K11a.

Another embodiment of this invention is directed to a solvate, a pharmaceutically acceptable ester, or a pharmaceutically acceptable salt of the compound of formula K11b.

Another embodiment of this invention is directed to a solvate, a pharmaceutically acceptable ester, or a pharmaceutically acceptable salt of the compound of formula K11c.

Another embodiment of this invention is directed to a solvate, a pharmaceutically acceptable ester, or a pharmaceutically acceptable salt of the compound of formula K11d.

Another embodiment of this invention is directed to a solvate, a pharmaceutically acceptable ester, or a pharmaceutically acceptable salt of the compound of formula K11e.

Another embodiment of this invention is directed to a solvate, a pharmaceutically acceptable ester, or a pharmaceutically acceptable salt of the compound of formula K11f.

Another embodiment of this invention is directed to a solvate, a pharmaceutically acceptable ester, or a pharmaceutically acceptable salt of the compound of formula K11g.

Another embodiment of this invention is directed to a solvate, a pharmaceutically acceptable ester, or a pharmaceutically acceptable salt of the compound of formula K11h.

Another embodiment of this invention is directed to a solvate, a pharmaceutically acceptable ester, or a pharmaceutically acceptable salt of the compound of formula K12a.

Another embodiment of this invention is directed to a solvate, a pharmaceutically acceptable ester, or a pharmaceutically acceptable salt of the compound of formula K12b.

Another embodiment of this invention is directed to a solvate, a pharmaceutically acceptable ester, or a pharmaceutically acceptable salt of the compound of formula K12c.

Another embodiment of this invention is directed to a solvate, a pharmaceutically acceptable ester, or a pharmaceutically acceptable salt of the compound of formula K12d.

Another embodiment of this invention is directed to a solvate, a pharmaceutically acceptable ester, or a pharmaceutically acceptable salt of the compound of formula K12e.

Another embodiment of this invention is directed to a solvate, a pharmaceutically acceptable ester, or a pharmaceutically acceptable salt of the compound of formula K12f.

Another embodiment of this invention is directed to a solvate, a pharmaceutically acceptable ester, or a pharmaceutically acceptable salt of the compound of formula K12g.

Another embodiment of this invention is directed to a solvate, a pharmaceutically acceptable ester, or a pharmaceutically acceptable salt of the compound of formula K12h.

Another embodiment of this invention is directed to a solvate, a pharmaceutically acceptable ester, or a pharmaceutically acceptable salt of the compound of formula K13a.

Another embodiment of this invention is directed to a solvate, a pharmaceutically acceptable ester, or a pharmaceutically acceptable salt of the compound of formula K13b.

Another embodiment of this invention is directed to a solvate, a pharmaceutically acceptable ester, or a pharmaceutically acceptable salt of the compound of formula K13c.

Another embodiment of this invention is directed to a solvate, a pharmaceutically acceptable ester, or a pharmaceutically acceptable salt of the compound of formula K13d.

Another embodiment of this invention is directed to a solvate, a pharmaceutically acceptable ester, or a pharmaceutically acceptable salt of the compound of formula K13e.

Another embodiment of this invention is directed to a solvate, a pharmaceutically acceptable ester, or a pharmaceutically acceptable salt of the compound of formula K13f.

Another embodiment of this invention is directed to a solvate, a pharmaceutically acceptable ester, or a pharmaceutically acceptable salt of the compound of formula K13g.

Another embodiment of this invention is directed to a solvate, a pharmaceutically acceptable ester, or a pharmaceutically acceptable salt of the compound of formula K13h.

Another embodiment of this invention is directed to a solvate, a pharmaceutically acceptable ester, or a pharmaceutically acceptable salt of the compound of formula K14a.

Another embodiment of this invention is directed to a solvate, a pharmaceutically acceptable ester, or a pharmaceutically acceptable salt of the compound of formula K14b.

Another embodiment of this invention is directed to a solvate, a pharmaceutically acceptable ester, or a pharmaceutically acceptable salt of the compound of formula K14c.

Another embodiment of this invention is directed to a solvate, a pharmaceutically acceptable ester, or a pharmaceutically acceptable salt of the compound of formula K14d.

Another embodiment of this invention is directed to a solvate, a pharmaceutically acceptable ester, or a pharmaceutically acceptable salt of the compound of formula K14e.

Another embodiment of this invention is directed to a solvate, a pharmaceutically acceptable ester, or a pharmaceutically acceptable salt of the compound of formula K14f.

Another embodiment of this invention is directed to a solvate, a pharmaceutically acceptable ester, or a pharmaceutically acceptable salt of the compound of formula K14g.

Another embodiment of this invention is directed to a solvate, a pharmaceutically acceptable ester, or a pharmaceutically acceptable salt of the compound of formula K14h.

Another embodiment of this invention is directed to a solvate, a pharmaceutically acceptable ester, or a pharmaceutically acceptable salt of the compound of formula K15a.

Another embodiment of this invention is directed to a solvate, a pharmaceutically acceptable ester, or a pharmaceutically acceptable salt of the compound of formula K15b.

Another embodiment of this invention is directed to a solvate, a pharmaceutically acceptable ester, or a pharmaceutically acceptable salt of the compound of formula K15c.

Another embodiment of this invention is directed to a solvate, a pharmaceutically acceptable ester, or a pharmaceutically acceptable salt of the compound of formula K15d.

Another embodiment of this invention is directed to a solvate, a pharmaceutically acceptable ester, or a pharmaceutically acceptable salt of the compound of formula K15e.

Another embodiment of this invention is directed to a solvate, a pharmaceutically acceptable ester, or a pharmaceutically acceptable salt of the compound of formula K15f.

Another embodiment of this invention is directed to a solvate, a pharmaceutically acceptable ester, or a pharmaceutically acceptable salt of the compound of formula K15g.

Another embodiment of this invention is directed to a solvate, a pharmaceutically acceptable ester, or a pharmaceutically acceptable salt of the compound of formula K15h.

Another embodiment of this invention is directed to a solvate, a pharmaceutically acceptable ester, or a pharmaceutically acceptable salt of the compound of formula K16a.

Another embodiment of this invention is directed to a solvate, a pharmaceutically acceptable ester, or a pharmaceutically acceptable salt of the compound of formula K16b.

Another embodiment of this invention is directed to a solvate, a pharmaceutically acceptable ester, or a pharmaceutically acceptable salt of the compound of formula K16c.

Another embodiment of this invention is directed to a solvate, a pharmaceutically acceptable ester, or a pharmaceutically acceptable salt of the compound of formula K16d.

Another embodiment of this invention is directed to a solvate, a pharmaceutically acceptable ester, or a pharmaceutically acceptable salt of the compound of formula K16e.

Another embodiment of this invention is directed to a solvate, a pharmaceutically acceptable ester, or a pharmaceutically acceptable salt of the compound of formula K16f.

Another embodiment of this invention is directed to a solvate, a pharmaceutically acceptable ester, or a pharmaceutically acceptable salt of the compound of formula K16g.

Another embodiment of this invention is directed to a solvate, a pharmaceutically acceptable ester, or a pharmaceutically acceptable salt of the compound of formula K16h.

Another embodiment of this invention is directed to a solvate, a pharmaceutically acceptable ester, or a pharmaceutically acceptable salt of the compound of formula K17a.

Another embodiment of this invention is directed to a solvate, a pharmaceutically acceptable ester, or a pharmaceutically acceptable salt of the compound of formula K17b.

Another embodiment of this invention is directed to a solvate, a pharmaceutically acceptable ester, or a pharmaceutically acceptable salt of the compound of formula K17c.

Another embodiment of this invention is directed to a solvate, a pharmaceutically acceptable ester, or a pharmaceutically acceptable salt of the compound of formula K17d.

Another embodiment of this invention is directed to a solvate, a pharmaceutically acceptable ester, or a pharmaceutically acceptable salt of the compound of formula K17e.

Another embodiment of this invention is directed to a solvate, a pharmaceutically acceptable ester, or a pharmaceutically acceptable salt of the compound of formula K17f.

Another embodiment of this invention is directed to a solvate, a pharmaceutically acceptable ester, or a pharmaceutically acceptable salt of the compound of formula K17g.

Another embodiment of this invention is directed to a solvate, a pharmaceutically acceptable ester, or a pharmaceutically acceptable salt of the compound of formula K17h.

Another embodiment of this invention is directed to a solvate, a pharmaceutically acceptable ester, or a pharmaceutically acceptable salt of the compound of formula K18a.

Another embodiment of this invention is directed to a solvate, a pharmaceutically acceptable ester, or a pharmaceutically acceptable salt of the compound of formula K18b.

Another embodiment of this invention is directed to a solvate, a pharmaceutically acceptable ester, or a pharmaceutically acceptable salt of the compound of formula K18c.

Another embodiment of this invention is directed to a solvate, a pharmaceutically acceptable ester, or a pharmaceutically acceptable salt of the compound of formula K18d.

Another embodiment of this invention is directed to a solvate, a pharmaceutically acceptable ester, or a pharmaceutically acceptable salt of the compound of formula K18e.

Another embodiment of this invention is directed to a solvate, a pharmaceutically acceptable ester, or a pharmaceutically acceptable salt of the compound of formula K18f.

Another embodiment of this invention is directed to a solvate, a pharmaceutically acceptable ester, or a pharmaceutically acceptable salt of the compound of formula K18g.

Another embodiment of this invention is directed to a solvate, a pharmaceutically acceptable ester, or a pharmaceutically acceptable salt of the compound of formula K18h.

Another embodiment of this invention is directed to a solvate, a pharmaceutically acceptable ester, or a pharmaceutically acceptable salt of the compound of formula K19a.

Another embodiment of this invention is directed to a solvate, a pharmaceutically acceptable ester, or a pharmaceutically acceptable salt of the compound of formula K19b.

Another embodiment of this invention is directed to a solvate, a pharmaceutically acceptable ester, or a pharmaceutically acceptable salt of the compound of formula K19c.

Another embodiment of this invention is directed to a solvate, a pharmaceutically acceptable ester, or a pharmaceutically acceptable salt of the compound of formula K19d.

Another embodiment of this invention is directed to a solvate, a pharmaceutically acceptable ester, or a pharmaceutically acceptable salt of the compound of formula K19e.

Another embodiment of this invention is directed to a solvate, a pharmaceutically acceptable ester, or a pharmaceutically acceptable salt of the compound of formula K19f.

Another embodiment of this invention is directed to a solvate, a pharmaceutically acceptable ester, or a pharmaceutically acceptable salt of the compound of formula K19g.

Another embodiment of this invention is directed to a solvate, a pharmaceutically acceptable ester, or a pharmaceutically acceptable salt of the compound of formula K19h.

Another embodiment of this invention is directed to a solvate, a pharmaceutically acceptable ester, or a pharmaceutically acceptable salt of the compound of formula K20a.

Another embodiment of this invention is directed to a solvate, a pharmaceutically acceptable ester, or a pharmaceutically acceptable salt of the compound of formula K20b.

Another embodiment of this invention is directed to a solvate, a pharmaceutically acceptable ester, or a pharmaceutically acceptable salt of the compound of formula K20c.

Another embodiment of this invention is directed to a solvate, a pharmaceutically acceptable ester, or a pharmaceutically acceptable salt of the compound of formula K20d.

Another embodiment of this invention is directed to a solvate, a pharmaceutically acceptable ester, or a pharmaceutically acceptable salt of the compound of formula K20e.

Another embodiment of this invention is directed to a solvate, a pharmaceutically acceptable ester, or a pharmaceutically acceptable salt of the compound of formula K20f.

Another embodiment of this invention is directed to a solvate, a pharmaceutically acceptable ester, or a pharmaceutically acceptable salt of the compound of formula K20g.

Another embodiment of this invention is directed to a solvate, a pharmaceutically acceptable ester, or a pharmaceutically acceptable salt of the compound of formula K20h.

Another embodiment of this invention is directed to a solvate, a pharmaceutically acceptable ester, or a pharmaceutically acceptable salt of the compound of formula K21a.

Another embodiment of this invention is directed to a solvate, a pharmaceutically acceptable ester, or a pharmaceutically acceptable salt of the compound of formula K21b.

Another embodiment of this invention is directed to a solvate, a pharmaceutically acceptable ester, or a pharmaceutically acceptable salt of the compound of formula K21c.

Another embodiment of this invention is directed to a solvate, a pharmaceutically acceptable ester, or a pharmaceutically acceptable salt of the compound of formula K21d.

Another embodiment of this invention is directed to a solvate, a pharmaceutically acceptable ester, or a pharmaceutically acceptable salt of the compound of formula K21e.

Another embodiment of this invention is directed to a solvate, a pharmaceutically acceptable ester, or a pharmaceutically acceptable salt of the compound of formula K21f.

Another embodiment of this invention is directed to a solvate, a pharmaceutically acceptable ester, or a pharmaceutically acceptable salt of the compound of formula K21g.

Another embodiment of this invention is directed to a solvate, a pharmaceutically acceptable ester, or a pharmaceutically acceptable salt of the compound of formula K21 h.

Another embodiment of this invention is directed to a solvate, a pharmaceutically acceptable ester, or a pharmaceutically acceptable salt of the compound of formula K22a.

Another embodiment of this invention is directed to a solvate, a pharmaceutically acceptable ester, or a pharmaceutically acceptable salt of the compound of formula K22b.

Another embodiment of this invention is directed to a solvate, a pharmaceutically acceptable ester, or a pharmaceutically acceptable salt of the compound of formula K22c.

Another embodiment of this invention is directed to a solvate, a pharmaceutically acceptable ester, or a pharmaceutically acceptable salt of the compound of formula K22d.

Another embodiment of this invention is directed to a solvate, a pharmaceutically acceptable ester, or a pharmaceutically acceptable salt of the compound of formula K22e.

Another embodiment of this invention is directed to a solvate, a pharmaceutically acceptable ester, or a pharmaceutically acceptable salt of the compound of formula K22f.

Another embodiment of this invention is directed to a solvate, a pharmaceutically acceptable ester, or a pharmaceutically acceptable salt of the compound of formula K22g.

Another embodiment of this invention is directed to a solvate, a pharmaceutically acceptable ester, or a pharmaceutically acceptable salt of the compound of formula K22h.

Another embodiment of this invention is directed to a solvate, a pharmaceutically acceptable ester, or a pharmaceutically acceptable salt of the compound of formula X1.

Another embodiment of this invention is directed to a solvate, a pharmaceutically acceptable ester, or a pharmaceutically acceptable salt of the compound of formula X2.

Another embodiment of this invention is directed to a solvate, a pharmaceutically acceptable ester, or a pharmaceutically acceptable salt of the compound of formula X3.

Another embodiment of this invention is directed to a solvate, a pharmaceutically acceptable ester, or a pharmaceutically acceptable salt of the compound of formula X4.

Another embodiment of this invention is directed to a solvate, a pharmaceutically acceptable ester, or a pharmaceutically acceptable salt of the compound of formula X5.

Another embodiment of this invention is directed to a solvate, a pharmaceutically acceptable ester, or a pharmaceutically acceptable salt of the compound of formula X6.

Another embodiment of this invention is directed to a solvate, a pharmaceutically acceptable ester, or a pharmaceutically acceptable salt of the compound of formula X7.

Another embodiment of this invention is directed to a solvate, a pharmaceutically acceptable ester, or a pharmaceutically acceptable salt of the compound of formula X8.

Another embodiment of this invention is directed to a solvate, a pharmaceutically acceptable ester, or a pharmaceutically acceptable salt of the compound of formula X9.

Another embodiment of this invention is directed to a solvate, a pharmaceutically acceptable ester, or a pharmaceutically acceptable salt of the compound of formula X10.

Another embodiment of this invention is directed to a solvate, a pharmaceutically acceptable ester, or a pharmaceutically acceptable salt of the compound of formula X11.

As used above, and throughout this disclosure, the following terms, unless otherwise indicated, shall be understood to have the following meanings:

“Boc” means a tert-butoxycarbonyl group.

“DCE” means 1,2-dichloroethane.

“DCM” means dichloromethane.

“DMAP” means 4-(dimethylamino)pyridine.

“DMF” means N,N-dimethylformamide.

“Et” means ethyl.

“Et₃N” means triethylamine.

“EtOAc” means ethyl acetate.

“EtOH” means ethanol.

“¹HNMR” means proton nuclear magnetic resonance spectrum.

“HOAc” means acetic acid.

“Me” means methyl.

“MeOH” means methanol.

“MS” means mass spectrum.

“Ms” means a methanesulfonyl group.

“MsCl” means methanesulfonyl chloride.

“n-Bu” means n-butyl.

“n-Pr” means n-propyl.

“TBAF” means tetra-n-butylammonium fluoride.

“TBDPS” means a tert-butyldiphenylsilyl group.

“TBDPSCl” means tert-butyldiphenylsilylchloride.

“TFA” means trifluoroacetic acid.

“THF” means tetrahydrofuran.

At least one” means that there is one and there can be more than one (e.g. (a) 1, 2 or 3, or (b) 1 or 2, or (c) 1).

“One or more” means that there is one and there can be more than one (e.g. (a) 1, 2 or 3, or (b) 1 or 2, or (c) 1).

“Effective Amount” as used in the methods of treatment and the pharmaceutical compositions means a therapeutically effective amount.

“Patient” includes both human and animals.

“Mammal” means humans and other mammalian animals.

It is noted that the carbons of formula (I) and other formulas herein may be replaced with 1 to 3 silicon atoms so long as all valency requirements are satisfied.

“Alkyl” means an aliphatic hydrocarbon group which may be straight or branched and comprising about 1 to about 20 carbon atoms in the chain. Preferred alkyl groups contain about 1 to about 12 carbon atoms in the chain. More preferred alkyl groups contain about 1 to about 6 carbon atoms in the chain. Branched means that one or more lower alkyl groups such as methyl, ethyl or propyl, are attached to a linear alkyl chain. “Lower alkyl” means a group having about 1 to about 6 carbon atoms in the chain which may be straight or branched. “Alkyl” may be unsubstituted or optionally substituted by one or more substituents which may be the same or different, each substituent being independently selected from the group consisting of halo, alkyl, aryl, cycloalkyl, cyano, hydroxy, alkoxy, alkylthio, amino, oxime (e.g., ═N—OH), —NH(alkyl), —NH(cycloalkyl), —N(alkyl)₂, —O—C(O)-alkyl, —O—C(O)-aryl, —O—C(O)-cycloalkyl, carboxy and —C(O)O-alkyl. Non-limiting examples of suitable alkyl groups include methyl, ethyl, n-propyl, isopropyl and t-butyl.

“Alkenyl” means an aliphatic hydrocarbon group containing at least one carbon-carbon double bond and which may be straight or branched and comprising about 2 to about 15 carbon atoms in the chain. Preferred alkenyl groups have about 2 to about 12 carbon atoms in the chain; and more preferably about 2 to about 6 carbon atoms in the chain. Branched means that one or more lower alkyl groups such as methyl, ethyl or propyl, are attached to a linear alkenyl chain. “Lower alkenyl” means about 2 to about 6 carbon atoms in the chain which may be straight or branched. “Alkenyl” may be unsubstituted or optionally substituted by one or more substituents which may be the same or different, each substituent being independently selected from the group consisting of halo, alkyl, aryl, cycloalkyl, cyano, alkoxy and —S(alkyl). Non-limiting examples of suitable alkenyl groups include ethenyl, propenyl, n-butenyl, 3-methylbut-2-enyl, n-pentenyl, octenyl and decenyl.

“Alkylene” means a difunctional group obtained by removal of a hydrogen atom from an alkyl group that is defined above. Non-limiting examples of alkylene include methylene, ethylene and propylene.

“Alkynyl” means an aliphatic hydrocarbon group containing at least one carbon-carbon triple bond and which may be straight or branched and comprising about 2 to about 15 carbon atoms in the chain. Preferred alkynyl groups have about 2 to about 12 carbon atoms in the chain; and more preferably about 2 to about 4 carbon atoms in the chain. Branched means that one or more lower alkyl groups such as methyl, ethyl or propyl, are attached to a linear alkynyl chain. “Lower alkynyl” means about 2 to about 6 carbon atoms in the chain which may be straight or branched. Non-limiting examples of suitable alkynyl groups include ethynyl, propynyl, 2-butynyl and 3-methylbutynyl. “Alkynyl” may be unsubstituted or optionally substituted by one or more substituents which may be the same or different, each substituent being independently selected from the group consisting of alkyl, aryl and cycloalkyl.

“Aryl” means an aromatic monocyclic or multicyclic ring system comprising about 6 to about 14 carbon atoms, preferably about 6 to about 10 carbon atoms. The aryl group can be optionally substituted with one or more “ring system substituents” which may be the same or different, and are as defined herein. Non-limiting examples of suitable aryl groups include phenyl and naphthyl.

“Heteroaryl” means an aromatic monocyclic or multicyclic ring system comprising about 5 to about 14 ring atoms, preferably about 5 to about 10 ring atoms, in which one or more of the ring atoms is an element other than carbon, for example nitrogen, oxygen or sulfur, alone or in combination. Preferred heteroaryls contain about 5 to about 6 ring atoms. The “heteroaryl” can be optionally substituted by one or more “ring system substituents” which may be the same or different, and are as defined herein. The prefix aza, oxa or thia before the heteroaryl root name means that at least a nitrogen, oxygen or sulfur atom respectively, is present as a ring atom. A nitrogen atom of a heteroaryl can be optionally oxidized to the corresponding N-oxide. “Heteroaryl” may also include a heteroaryl as defined above fused to an aryl as defined above. Non-limiting examples of suitable heteroaryls include pyridyl, pyrazinyl, furanyl, thienyl, pyrimidinyl, pyridone (including N-substituted pyridones), isoxazolyl, isothiazolyl, oxazolyl, thiazolyl, pyrazolyl, furazanyl, pyrrolyl, pyrazolyl, triazolyl, 1,2,4-thiadiazolyl, pyrazinyl, pyridazinyl, quinoxalinyl, phthalazinyl, oxindolyl, imidazo[1,2-a]pyridinyl, imidazo[2,1-b]thiazolyl, benzofurazanyl, indolyl, azaindolyl, benzimidazolyl, benzothienyl, quinolinyl, imidazolyl, thienopyridyl, quinazolinyl, thienopyrimidyl, pyrrolopyridyl, imidazopyridyl, isoquinolinyl, benzoazaindolyl, 1,2,4-triazinyl, benzothiazolyl and the like. The term “heteroaryl” also refers to partially saturated heteroaryl moieties such as, for example, tetrahydroisoquinolyl, tetrahydroquinolyl and the like.

“Aralkyl” or “arylalkyl” means an aryl-alkyl- group in which the aryl and alkyl are as previously described. Preferred aralkyls comprise a lower alkyl group. Non-limiting examples of suitable aralkyl groups include benzyl, 2-phenethyl and naphthalenylmethyl. The bond to the parent moiety is through the alkyl.

“Alkylaryl” means an alkyl-aryl- group in which the alkyl and aryl are as previously described. Preferred alkylaryls comprise a lower alkyl group. Non-limiting example of a suitable alkylaryl group is tolyl. The bond to the parent moiety is through the aryl.

“Cycloalkyl” means a non-aromatic mono- or multicyclic ring system comprising about 3 to about 10 carbon atoms, preferably about 5 to about 10 carbon atoms. Preferred cycloalkyl rings contain about 5 to about 7 ring atoms. The cycloalkyl can be optionally substituted with one or more “ring system substituents” which may be the same or different, and are as defined above. Non-limiting examples of suitable monocyclic cycloalkyls include cyclopropyl, cyclopentyl, cyclohexyl, cycloheptyl and the like. Non-limiting examples of suitable multicyclic cycloalkyls include 1-decalinyl, norbornyl, adamantyl and the like.

“Cycloalkylalkyl” means a cycloalkyl moiety as defined above linked via an alkyl moiety (defined above) to a parent core. Non-limiting examples of suitable cycloalkylalkyls include cyclohexylmethyl, adamantylmethyl and the like.

“Cycloalkenyl” means a non-aromatic mono or multicyclic ring system comprising about 3 to about 10 carbon atoms, preferably about 5 to about 10 carbon atoms which contains at least one carbon-carbon double bond. Preferred cycloalkenyl rings contain about 5 to about 7 ring atoms. The cycloalkenyl can be optionally substituted with one or more “ring system substituents” which may be the same or different, and are as defined above. Non-limiting examples of suitable monocyclic cycloalkenyls include cyclopentenyl, cyclohexenyl, cyclohepta-1,3-dienyl, and the like. Non-limiting example of a suitable multicyclic cycloalkenyl is norbornylenyl.

“Cycloalkenylalkyl” means a cycloalkenyl moiety as defined above linked via an alkyl moiety (defined above) to a parent core. Non-limiting examples of suitable cycloalkenylalkyls include cyclopentenylmethyl, cyclohexenylmethyl and the like.

“Halogen” means fluorine, chlorine, bromine, or iodine. Preferred are fluorine, chlorine and bromine. “Halo” refers to fluoro, chloro, bromo or iodo.

“Ring system substituent” means a substituent attached to an aromatic or non-aromatic ring system which, for example, replaces an available hydrogen on the ring system. Ring system substituents may be the same or different, each being independently selected from the group consisting of alkyl, alkenyl, alkynyl, aryl, heteroaryl, aralkyl, alkylaryl, heteroaralkyl, heteroarylalkenyl, heteroarylalkynyl, alkylheteroaryl, hydroxy, hydroxyalkyl, alkoxy, aryloxy, aralkoxy, acyl, aroyl, halo, nitro, cyano, carboxy, alkoxycarbonyl, aryloxycarbonyl, aralkoxycarbonyl, alkylsulfonyl, arylsulfonyl, heteroarylsulfonyl, alkylthio, arylthio, heteroarylthio, aralkylthio, heteroaralkylthio, cycloalkyl, heterocyclyl, —O—C(O)-alkyl, —O—C(O)-aryl, —O—C(O)-cycloalkyl, —C(═N—CN)—NH₂, —C(═NH)—NH₂, —C(═NH)—NH(alkyl), oxime (e.g., ═N—OH), Y₁Y₂N—, Y₁Y₂N-alkyl-, Y₁Y₂NC(O)—, Y₁Y₂NSO₂— and —SO₂NY₁Y₂, wherein Y₁ and Y₂ can be the same or different and are independently selected from the group consisting of hydrogen, alkyl, aryl, cycloalkyl, and aralkyl. “Ring system substituent” may also mean a single moiety which simultaneously replaces two available hydrogens on two adjacent carbon atoms (one H on each carbon) on a ring system. Examples of such moiety are methylene dioxy, ethylenedioxy, —C(CH₃)₂— and the like which form moieties such as, for example:

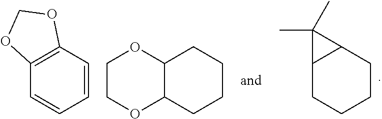

“Heteroarylalkyl” means a heteroaryl moiety as defined above linked via an alkyl moiety (defined above) to a parent core. Non-limiting examples of suitable heteroaryls include 2-pyridinylmethyl, quinolinylmethyl and the like.

“Heterocyclyl” or “heterocycloalkyl” means a non-aromatic saturated monocyclic or multicyclic ring system comprising about 3 to about 10 ring atoms, preferably about 5 to about 10 ring atoms, in which one or more of the atoms in the ring system is an element other than carbon, for example nitrogen, oxygen or sulfur, alone or in combination. There are no adjacent oxygen and/or sulfur atoms present in the ring system. Preferred heterocyclyls contain about 5 to about 6 ring atoms. The prefix aza, oxa or thia before the heterocyclyl root name means that at least a nitrogen, oxygen or sulfur atom respectively is present as a ring atom. Any —NH in a heterocyclyl ring may exist protected such as, for example, as an —N(Boc), —N(CBz), —N(Tos) group and the like; such protections are also considered part of this invention. The heterocyclyl can be optionally substituted by one or more “ring system substituents” which may be the same or different, and are as defined herein. The nitrogen or sulfur atom of the heterocyclyl can be optionally oxidized to the corresponding N-oxide, S-oxide or S,S-dioxide. Non-limiting examples of suitable monocyclic heterocyclyl rings include piperidyl, pyrrolidinyl, piperazinyl, morpholinyl, thiomorpholinyl, thiazolidinyl, 1,4-dioxanyl, tetrahydrofuranyl, tetrahydrothiophenyl, lactam, lactone, and the like. Heterocyclyl also includes rings wherein ═O replaces two available hydrogens on the same carbon atom (i.e., heterocyclyl includes rings having a carbonyl group in the ring). An example of such a heterocyclyl ring is pyrrolidone:

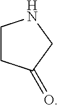

“Heterocyclylalkyl” or “heterocycloalkylalkyl” means a heterocyclyl moiety as defined above linked via an alkyl moiety (defined above) to a parent core. Non-limiting examples of suitable heterocyclylalkyls include piperidinylmethyl, piperazinylmethyl and the like.

“Heterocyclenyl” means a non-aromatic monocyclic or multicyclic ring system comprising about 3 to about 10 ring atoms, preferably about 5 to about 10 ring atoms, in which one or more of the atoms in the ring system is an element other than carbon, for example nitrogen, oxygen or sulfur atom, alone or in combination, and which contains at least one carbon-carbon double bond or carbon-nitrogen double bond. There are no adjacent oxygen and/or sulfur atoms present in the ring system. Preferred heterocyclenyl rings contain about 5 to about 6 ring atoms. The prefix aza, oxa or thia before the heterocyclenyl root name means that at least a nitrogen, oxygen or sulfur atom respectively is present as a ring atom. The heterocyclenyl can be optionally substituted by one or more ring system substituents, wherein “ring system substituent” is as defined above. The nitrogen or sulfur atom of the heterocyclenyl can be optionally oxidized to the corresponding N-oxide, S-oxide or S,S-dioxide. Non-limiting examples of suitable heterocyclenyl groups include 1,2,3,4-tetrahydropyridinyl, 1,2-dihydropyridinyl, 1,4-dihydropyridinyl, 1,2,3,6-tetrahydropyridinyl, 1,4,5,6-tetrahydropyrimidinyl, 2-pyrrolinyl, 3-pyrrolinyl, 2-imidazolinyl, 2-pyrazolinyl, dihydroimidazolyl, dihydrooxazolyl, dihydrooxadiazolyl, dihydrothiazolyl, 3,4-dihydro-2H-pyranyl, dihydrofuranyl, fluorodihydrofuranyl, 7-oxabicyclo[2.2.1]heptenyl, dihydrothiophenyl, dihydrothiopyranyl, and the like. “Heterocyclenyl” also includes rings wherein ═O replaces two available hydrogens on the same carbon atom (i.e., heterocyclenyl includes rings having a carbonyl group in the ring). An example of such a heterocyclenyl ring is pyrrolidinone:

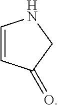

“Heterocyclenylalkyl” means a heterocyclenyl moiety as defined above linked via an alkyl moiety (defined above) to a parent core.

It should be noted that in hetero-atom containing ring systems of this invention, there are no hydroxyl groups on carbon atoms adjacent to a N, O or S, as well as there are no N or S groups on carbon adjacent to another heteroatom. Thus, for example, in the ring:

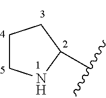

there is no —OH attached directly to carbons marked 2 and 5.

It should also be noted that tautomeric forms such as, for example, the moieties:

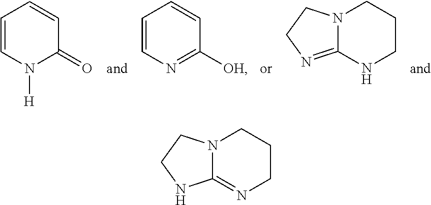

are considered equivalent in certain embodiments of this invention.

It should also be noted that isomeric forms such as, for example, the moieties:

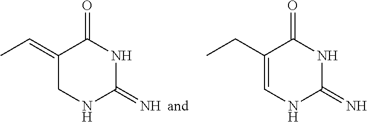

are considered equivalent in certain embodiments of this invention.

It should also be noted that isomeric forms such as, for example, the moieties:

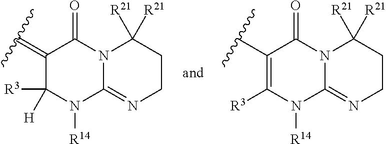

are considered equivalent in certain embodiments of this invention.

It should also be noted that isomeric forms such as, for example, the moieties:

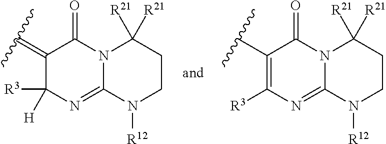

are considered equivalent in certain embodiments of this invention.

It should also be noted that isomeric forms such as, for example, the moieties:

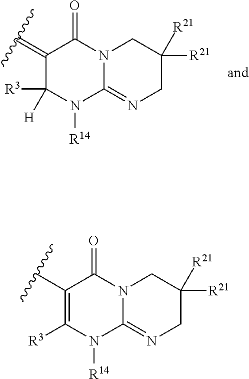

are considered equivalent in certain embodiments of this invention.

It should also be noted that isomeric forms such as, for example, the moieties:

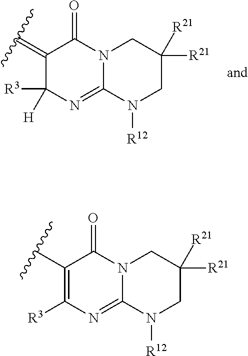

are considered equivalent in certain embodiments of this invention.

It should also be noted that isomeric forms such as, for example, the moieties:

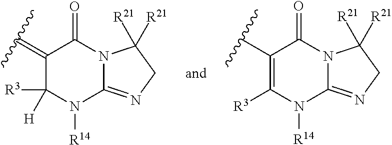

are considered equivalent in certain embodiments of this invention.

It should also be noted that isomeric forms such as, for example, the moieties:

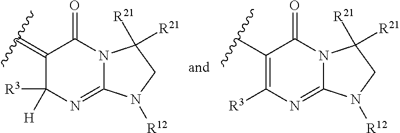

are considered equivalent in certain embodiments of this invention.

It should also be noted that isomeric forms such as, for example, the moieties:

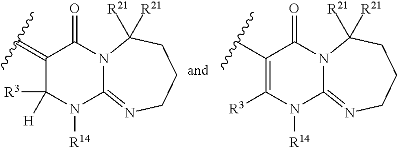

are considered equivalent in certain embodiments of this invention.

It should also be noted that isomeric forms such as, for example, the moieties:

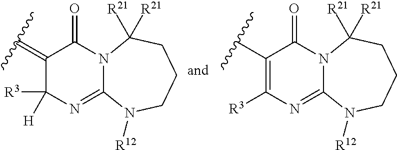

are considered equivalent in certain embodiments of this invention.

It should also be noted that isomeric forms such as, for example, the moieties:

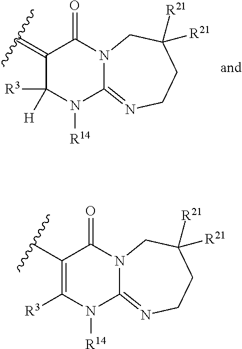

are considered equivalent in certain embodiments of this invention.

It should also be noted that isomeric forms such as, for example, the moieties:

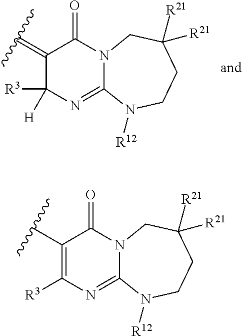

are considered equivalent in certain embodiments of this invention.

“Alkynylalkyl” means an alkynyl-alkyl- group in which the alkynyl and alkyl are as previously described. Preferred alkynylalkyls contain a lower alkynyl and a lower alkyl group. The bond to the parent moiety is through the alkyl. Non-limiting examples of suitable alkynylalkyl groups include propargylmethyl.

“Heteroaralkyl” means a heteroaryl-alkyl- group in which the heteroaryl and alkyl are as previously described. Preferred heteroaralkyls contain a lower alkyl group. Non-limiting examples of suitable aralkyl groups include pyridylmethyl, and quinolin-3-ylmethyl. The bond to the parent moiety is through the alkyl.

“Hydroxyalkyl” means a HO-alkyl- group in which alkyl is as previously defined. Preferred hydroxyalkyls contain lower alkyl. Non-limiting examples of suitable hydroxyalkyl groups include hydroxymethyl and 2-hydroxyethyl.

“Acyl” means an H—C(O)—, alkyl-C(O)— or cycloalkyl-C(O)—, group in which the various groups are as previously described. The bond to the parent moiety is through the carbonyl. Preferred acyls contain a lower alkyl. Non-limiting examples of suitable acyl groups include formyl, acetyl and propanoyl.

“Aroyl” means an aryl-C(O)— group in which the aryl group is as previously described. The bond to the parent moiety is through the carbonyl. Non-limiting examples of suitable groups include benzoyl and 1-naphthoyl.

“Alkoxy” means an alkyl-O— group in which the alkyl group is as previously described. Non-limiting examples of suitable alkoxy groups include methoxy, ethoxy, n-propoxy, isopropoxy and n-butoxy. The bond to the parent moiety is through the ether oxygen.

“Aryloxy” means an aryl-O— group in which the aryl group is as previously described. Non-limiting examples of suitable aryloxy groups include phenoxy and naphthoxy. The bond to the parent moiety is through the ether oxygen.

“Aralkyloxy” means an aralkyl-O— group in which the aralkyl group is as previously described. Non-limiting examples of suitable aralkyloxy groups include benzyloxy and 1- or 2-naphthalenemethoxy. The bond to the parent moiety is through the ether oxygen.

“Alkylthio” means an alkyl-S— group in which the alkyl group is as previously described. Non-limiting examples of suitable alkylthio groups include methylthio and ethylthio. The bond to the parent moiety is through the sulfur.

“Arylthio” means an aryl-S— group in which the aryl group is as previously described. Non-limiting examples of suitable arylthio groups include phenylthio and naphthylthio. The bond to the parent moiety is through the sulfur.

“Aralkylthio” means an aralkyl-S— group in which the aralkyl group is as previously described. Non-limiting example of a suitable aralkylthio group is benzylthio. The bond to the parent moiety is through the sulfur.

“Alkoxycarbonyl” means an alkyl-O—CO— group. Non-limiting examples of suitable alkoxycarbonyl groups include methoxycarbonyl and ethoxycarbonyl. The bond to the parent moiety is through the carbonyl.

“Aryloxycarbonyl” means an aryl-O—C(O)— group. Non-limiting examples of suitable aryloxycarbonyl groups include phenoxycarbonyl and naphthoxycarbonyl. The bond to the parent moiety is through the carbonyl.

“Aralkoxycarbonyl” means an aralkyl-O—C(O)— group. Non-limiting example of a suitable aralkoxycarbonyl group is benzyloxycarbonyl. The bond to the parent moiety is through the carbonyl.

“Alkylsulfonyl” means an alkyl-S(O₂)— group. Preferred groups are those in which the alkyl group is lower alkyl. The bond to the parent moiety is through the sulfonyl.

“Arylsulfonyl” means an aryl-S(O₂)— group. The bond to the parent moiety is through the sulfonyl.

The term “substituted” means that one or more hydrogens on the designated atom is replaced with a selection from the indicated group, provided that the designated atom's normal valency under the existing circumstances is not exceeded, and that the substitution results in a stable compound. Combinations of substituents and/or variables are permissible only if such combinations result in stable compounds. By “stable compound’ or “stable structure” is meant a compound that is sufficiently robust to survive isolation to a useful degree of purity from a reaction mixture, and formulation into an efficacious therapeutic agent.

The term “optionally substituted” means optional substitution with the specified groups, radicals or moieties.

The term “purified”, “in purified form” or “in isolated and purified form” for a compound refers to the physical state of said compound after being isolated from a synthetic process (e.g. from a reaction mixture), or natural source or combination thereof. Thus, the term “purified”, “in purified form” or “in isolated and purified form” for a compound refers to the physical state of said compound after being obtained from a purification process or processes described herein or well known to the skilled artisan (e.g., chromatography, recrystallization and the like) in sufficient purity to be characterizable by standard analytical techniques described herein or well known to the skilled artisan.

It should also be noted that any carbon as well as heteroatom with unsatisfied valences in the text, schemes, examples and Tables herein is assumed to have the sufficient number of hydrogen atom(s) to satisfy the valences.

When a functional group in a compound is termed “protected”, this means that the group is in modified form to preclude undesired side reactions at the protected site when the compound is subjected to a reaction. Suitable protecting groups will be recognized by those with ordinary skill in the art as well as by reference to standard textbooks such as, for example, T. W. Greene et al, Protective Groups in organic Synthesis (1991), Wiley, New York.

When any variable (e.g., aryl, heterocycle, R², etc.) occurs more than one time in any constituent or in Formula (I), its definition on each occurrence is independent of its definition at every other occurrence.

As used herein, the term “composition” is intended to encompass a product comprising the specified ingredients in the specified amounts, as well as any product which results, directly or indirectly, from combination of the specified ingredients in the specified amounts.

Prodrugs and solvates of the compounds of the invention are also contemplated herein. A discussion of prodrugs is provided in T. Higuchi and V. Stella, Pro-drugs as Novel Delivery Systems (1987) 14 of the A.C.S. Symposium Series, and in Bioreversible Carriers in Drug Design, (1987) Edward B. Roche, ed., American Pharmaceutical Association and Pergamon Press. The term “prodrug” means a compound (e.g., a drug precursor) that is transformed in vivo to yield a compound of Formula (I) or a pharmaceutically acceptable salt, hydrate or solvate of the compound. The transformation may occur by various mechanisms (e.g., by metabolic or chemical processes), such as, for example, through hydrolysis in blood. A discussion of the use of prodrugs is provided by T. Higuchi and W. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987.

For example, if a compound of Formula (I) or a pharmaceutically acceptable salt, hydrate or solvate of the compound contains a carboxylic acid functional group, a prodrug can comprise an ester formed by the replacement of the hydrogen atom of the acid group with a group such as, for example, (C₁-C₈)alkyl, (C₂-C₁₂)alkanoyloxymethyl, 1-(alkanoyloxy)ethyl having from 4 to 9 carbon atoms, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N—(C₁-C₂)alkylamino(C₂-C₃)alkyl (such as β-dimethylaminoethyl), carbamoyl-(C₁-C₂)alkyl, N,N-di(C₁-C₂)alkylcarbamoyl-(C₁-C₂)alkyl and piperidino-, pyrrolidino- or morpholino(C₂-C₃)alkyl, and the like.

Similarly, if a compound of Formula (I) contains an alcohol functional group, a prodrug can be formed by the replacement of the hydrogen atom of the alcohol group with a group such as, for example, (C₁-C₆)alkanoyloxymethyl, 1-((C₁-C₆)alkanoyloxy)ethyl, 1-methyl-1-((C₁-C₆)alkanoyloxy)ethyl, (C₁-C₆)alkoxycarbonyloxymethyl, N—(C₁-C₆)alkoxycarbonylaminomethyl, succinoyl, (C₁-C₆)alkanoyl, α-amino(C₁-C₄)alkanyl, arylacyl and α-aminoacyl, or α-aminoacyl-α-aminoacyl, where each α-aminoacyl group is independently selected from the naturally occurring L-amino acids, P(O)(OH)₂, —P(O)(O(C₁-C₆)alkyl)₂ or glycosyl (the radical resulting from the removal of a hydroxyl group of the hemiacetal form of a carbohydrate), and the like.

If a compound of Formula (I) incorporates an amine functional group, a prodrug can be formed by the replacement of a hydrogen atom in the amine group with a group such as, for example, R-carbonyl, RO-carbonyl, NRR′-carbonyl where R and R′ are each independently (C₁-C₁₀)alkyl, (C₃-C₇)cycloalkyl, benzyl, or R-carbonyl is a natural α-aminoacyl or natural α-aminoacyl, —C(OH)C(O)OY¹ wherein Y1 is H, (C₁-C₆)alkyl or benzyl, —C(OY²)Y³ wherein Y² is (C₁-C₄) alkyl and Y³ is (C₁-C₆)alkyl, carboxy(C₁-C₆)alkyl, amino(C₁-C₄)alkyl or mono-N— or di-N,N—(C₁-C₆)alkylaminoalkyl, —C(Y⁴)Y⁵ wherein Y⁴ is H or methyl and Y⁵ is mono-N— or di-N,N—(C₁-C₆)alkylamino morpholino, piperidin-1-yl or pyrrolidin-1-yl, and the like.

One or more compounds of the invention may exist in unsolvated as well as solvated forms with pharmaceutically acceptable solvents such as water, ethanol, and the like, and it is intended that the invention embrace both solvated and unsolvated forms. “Solvate” means a physical association of a compound of this invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. Non-limiting examples of suitable solvates include ethanolates, methanolates, and the like. “Hydrate” is a solvate wherein the solvent molecule is H₂O.

One or more compounds of the invention may optionally be converted to a solvate. Preparation of solvates is generally known. Thus, for example, M. Caira et al, J. Pharmaceutical Sci., 93(3), 601-611 (2004) describe the preparation of the solvates of the antifungal fluconazole in ethyl acetate as well as from water. Similar preparations of solvates, hemisolvate, hydrates and the like are described by E. C. van Tonder et al, AAPS Pharm Sci Tech., 5(1), article 12 (2004); and A. L. Bingham et al, Chem. Commun., 603-604 (2001). A typical, non-limiting, process involves dissolving the inventive compound in desired amounts of the desired solvent (organic or water or mixtures thereof) at a higher than ambient temperature, and cooling the solution at a rate sufficient to form crystals which are then isolated by standard methods. Analytical techniques such as, for example I. R. spectroscopy, show the presence of the solvent (or water) in the crystals as a solvate (or hydrate).

“Effective amount” or “therapeutically effective amount” is meant to describe an amount of compound or a composition of the present invention effective in inhibiting the above-noted diseases and thus producing the desired therapeutic, ameliorative, inhibitory or preventative effect.

The compounds of Formula (I) can form salts which are also within the scope of this invention. Reference to a compound of Formula (I) herein is understood to include reference to salts thereof, unless otherwise indicated. The term “salt(s)”, as employed herein, denotes acidic salts formed with inorganic and/or organic acids, as well as basic salts formed with inorganic and/or organic bases. In addition, when a compound of Formula (I) contains both a basic moiety, such as, but not limited to a pyridine or imidazole, and an acidic moiety, such as, but not limited to a carboxylic acid, zwitterions (“inner salts”) may be formed and are included within the term “salt(s)” as used herein. Pharmaceutically acceptable (i.e., non-toxic, physiologically acceptable) salts are preferred, although other salts are also useful. Salts of the compounds of the Formula (I) may be formed, for example, by reacting a compound of Formula (I) with an amount of acid or base, such as an equivalent amount, in a medium such as one in which the salt precipitates or in an aqueous medium followed by lyophilization.

Exemplary acid addition salts include acetates, ascorbates, benzoates, benzenesulfonates, bisulfates, borates, butyrates, citrates, camphorates, camphorsulfonates, fumarates, hydrochlorides, hydrobromides, hydroiodides, lactates, maleates, methanesulfonates, naphthalenesulfonates, nitrates, oxalates, phosphates, propionates, salicylates, succinates, sulfates, tartarates, thiocyanates, toluenesulfonates (also known as tosylates,) and the like. Additionally, acids which are generally considered suitable for the formation of pharmaceutically useful salts from basic pharmaceutical compounds are discussed, for example, by P. Stahl et al, Camille G. (eds.) Handbook of Pharmaceutical Salts. Properties, Selection and Use. (2002) Zurich: Wiley-VCH; S. Berge et al, Journal of Pharmaceutical Sciences (1977) 66(1) 1-19; P. Gould, International J. of Pharmaceutics (1986) 33 201-217; Anderson et al, The Practice of Medicinal Chemistry (1996), Academic Press, New York; and in The Orange Book (Food & Drug Administration, Washington, D.C. on their website). These disclosures are incorporated herein by reference thereto.

Exemplary basic salts include ammonium salts, alkali metal salts such as sodium, lithium, and potassium salts, alkaline earth metal salts such as calcium and magnesium salts, salts with organic bases (for example, organic amines) such as dicyclohexylamines, t-butyl amines, and salts with amino acids such as arginine, lysine and the like. Basic nitrogen-containing groups may be quarternized with agents such as lower alkyl halides (e.g. methyl, ethyl, and butyl chlorides, bromides and iodides), dialkyl sulfates (e.g. dimethyl, diethyl, and dibutyl sulfates), long chain halides (e.g. decyl, lauryl, and stearyl chlorides, bromides and iodides), aralkyl halides (e.g. benzyl and phenethyl bromides), and others.

All such acid salts and base salts are intended to be pharmaceutically acceptable salts within the scope of the invention and all acid and base salts are considered equivalent to the free forms of the corresponding compounds for purposes of the invention.

Pharmaceutically acceptable esters of the present compounds include the following groups: (1) carboxylic acid esters obtained by esterification of the hydroxy groups, in which the non-carbonyl moiety of the carboxylic acid portion of the ester grouping is selected from straight or branched chain alkyl (for example, acetyl, n-propyl, t-butyl, or n-butyl), alkoxyalkyl (for example, methoxymethyl), aralkyl (for example, benzyl), aryloxyalkyl (for example, phenoxymethyl), aryl (for example, phenyl optionally substituted with, for example, halogen, C₁₋₄alkyl, or C₁₋₄alkoxy or amino); (2) sulfonate esters, such as alkyl- or aralkylsulfonyl (for example, methanesulfonyl); (3) amino acid esters (for example, L-valyl or L-isoleucyl); (4) phosphonate esters and (5) mono-, di- or triphosphate esters. The phosphate esters may be further esterified by, for example, a C₁₋₂₀ alcohol or reactive derivative thereof, or by a 2,3-di(C₆₋₂₄)acyl glycerol.

Compounds of Formula (I), and salts, solvates, esters and prodrugs thereof, may exist in their tautomeric form (for example, as an amide, enol, keto or imino ether). All such tautomeric forms are contemplated herein as part of the present invention.

The compounds of Formula (I) may contain asymmetric or chiral centers, and, therefore, exist in different stereoisomeric forms. It is intended that all stereoisomeric forms of the compounds of Formula (I) as well as mixtures thereof, including racemic mixtures, form part of the present invention. In addition, the present invention embraces all geometric and positional isomers. For example, if a compound of Formula (I) incorporates a double bond or a fused ring, both the cis- and trans-forms, as well as mixtures, are embraced within the scope of the invention.

Diastereomeric mixtures can be separated into their individual diastereomers on the basis of their physical chemical differences by methods well known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Also, some of the compounds of Formula (I) may be atropisomers (e.g., substituted biaryls) and are considered as part of this invention. Enantiomers can also be separated by use of chiral HPLC column.

It is also possible that the compounds of Formula (I) may exist in different tautomeric forms, and all such forms are embraced within the scope of the invention. Also, for example, all keto-enol and imine-enamine forms of the compounds are included in the invention.

All stereoisomers (for example, geometric isomers, optical isomers and the like) of the present compounds (including those of the salts, solvates, esters and prodrugs of the compounds as well as the salts, solvates and esters of the prodrugs), such as those which may exist due to asymmetric carbons on various substituents, including enantiomeric forms (which may exist even in the absence of asymmetric carbons), rotameric forms, atropisomers, and diastereomeric forms, are contemplated within the scope of this invention, as are positional isomers (such as, for example, 4-pyridyl and 3-pyridyl). (For example, if a compound of Formula (I) incorporates a double bond or a fused ring, both the cis- and trans-forms, as well as mixtures, are embraced within the scope of the invention. Also, for example, all keto-enol and imine-enamine forms of the compounds are included in the invention.) Individual stereoisomers of the compounds of the invention may, for example, be substantially free of other isomers, or may be admixed, for example, as racemates or with all other, or other selected, stereoisomers. The chiral centers of the present invention can have the S or R configuration as defined by the IUPAC 1974 Recommendations. The use of the terms “salt”, “solvate”, “ester”, “prodrug” and the like, is intended to equally apply to the salt, solvate, ester and prodrug of enantiomers, stereoisomers, rotamers, tautomers, positional isomers, racemates or prodrugs of the inventive compounds.

The present invention also embraces isotopically-labelled compounds of the present invention which are identical to those recited herein, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorus, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively.

Certain isotopically-labelled compounds of Formula (I) (e.g., those labeled with ³H and ¹⁴C) are useful in compound and/or substrate tissue distribution assays. Tritiated (i.e., ³H) and carbon-14 (i.e., ¹⁴C) isotopes are particularly preferred for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Isotopically labelled compounds of Formula (I) can generally be prepared by following procedures analogous to those disclosed in the Schemes and/or in the Examples hereinbelow, by substituting an appropriate isotopically labelled reagent for a non-isotopically labelled reagent.

Polymorphic forms of the compounds of Formula (I), and of the salts, solvates, esters and prodrugs of the compounds of Formula (I), are intended to be included in the present invention.

The compounds according to the invention can have pharmacological properties; in particular, the compounds of Formula (I) can be modulators of gamma secretase (including inhibitors, antagonists and the like).

More specifically, the compounds of Formula (I) can be useful in the treatment of a variety of disorders of the central nervous system including, for example, including, but not limited to, Alzheimer's disease, AIDS-related dementia, Parkinson's disease, amyotrophic lateral sclerosis, retinitis pigmentosa, spinal muscular atrophy and cerebellar degeneration and the like.

Another aspect of this invention is a method of treating a mammal (e.g., human) having a disease or condition of the central nervous system by administering a therapeutically effective amount of at least one compound of Formula (I), or a pharmaceutically acceptable salt, solvate, ester or prodrug of said compound to the mammal.

A preferred dosage is about 0.001 to 500 mg/kg of body weight/day of the compound of Formula (I). An especially preferred dosage is about 0.01 to 25 mg/kg of body weight/day of a compound of Formula (I), or a pharmaceutically acceptable salt or solvate of said compound.

The compounds of this invention may also be useful in combination (administered together or sequentially) with one or more additional agents listed above.

The compounds of this invention may also be useful in combination (administered together or sequentially) with one or more compounds selected from the group consisting of Aβ antibody inhibitors, gamma secretase inhibitors and beta secretase inhibitors.

If formulated as a fixed dose, such combination products employ the compounds of this invention within the dosage range described herein and the other pharmaceutically active agent or treatment within its dosage range.

Accordingly, in an aspect, this invention includes combinations comprising an amount of at least one compound of Formula (I), or a pharmaceutically acceptable salt, solvate, ester or prodrug thereof, and an amount of one or more additional agents listed above wherein the amounts of the compounds/treatments result in desired therapeutic effect.

The pharmacological properties of the compounds of this invention may be confirmed by a number of pharmacological assays. Certain assays are exemplified later in this document.

This invention is also directed to pharmaceutical compositions which comprise at least one compound of Formula (I), or a pharmaceutically acceptable salt, solvate, ester or prodrug of said compound and at least one pharmaceutically acceptable carrier.

Another embodiment of this invention is directed to a pharmaceutical composition comprising an effective amount of one or more (e.g., one) compounds of formula (I) and a pharmaceutically acceptable carrier.

Another embodiment of this invention is directed to a pharmaceutical composition comprising an effective amount of a pharmaceutically acceptable salt of one or more (e.g., one) compounds of formula (I) and a pharmaceutically acceptable carrier.

Another embodiment of this invention is directed to a pharmaceutical composition comprising an effective amount of a pharmaceutically acceptable ester of one or more (e.g., one) compounds of formula (I) and a pharmaceutically acceptable carrier.

Another embodiment of this invention is directed to a pharmaceutical composition comprising an effective amount of a solvate of one or more (e.g., one) compounds of formula (I) and a pharmaceutically acceptable carrier.

Another embodiment of this invention is directed to a pharmaceutical composition comprising an effective amount of one or more (e.g., one) compounds of formula (I), and an effective amount of one or more (e.g., one) other pharmaceutically active ingredients (e.g., drugs), and a pharmaceutically acceptable carrier. Examples of the other pharmaceutically active ingredients include, but are not limited to drugs selected form the group consisting of: (a) drugs useful for the treatment of Alzheimer's disease, (b) drugs useful for inhibiting the deposition of amyloid protein (e.g., amyloid beta protein) in, on or around neurological tissue (e.g., the brain), (c) drugs useful for treating neurodegenerative diseases, and (d) drugs useful for inhibiting gamma-secretase.

As used herein, “other pharmaceutically active ingredients” includes, for example, pharmaceutically active ingredients selected from the group consisting of: BACE inhibitors (beta secretase inhibitors); muscarinic antagonists (e.g., m₁ agonists or m₂ antagonists); cholinesterase inhibitors (e.g., acetyl- and/or butyrylchlolinesterase inhibitors); gamma secretase inhibitors; gamma secretase modulators; HMG-CoA reductase inhibitors; non-steroidal anti-inflammatory agents; N-methyl-D-aspartate receptor antagonists; anti-amyloid antibodies; vitamin E; nicotinic acetylcholine receptor agonists; CB1 receptor inverse agonists or CB1 receptor antagonists; an antibiotic; growth hormone secretagogues; histamine H3 antagonists; AMPA agonists; PDE4 inhibitors; GABA_{A} inverse agonists; inhibitors of amyloid aggregation; glycogen synthase kinase beta inhibitors; promoters of alpha secretase activity; PDE-10 inhibitors; Exelon (rivastigmine); Cognex (tacrine); Tau kinase inhibitors (e.g., GSK3beta inhibitors, cdk5 inhibitors, or ERK inhibitors); anti-Abeta vaccine; APP ligands; agents that upregulate insulin cholesterol lowering agents (for example, statins such as Atorvastatin, Fluvastatin, Lovastatin; Mevastatin, Pitavastatin, Pravastatin, Rosuvastatin, Simvastatin); cholesterol absorption inhibitors (such as Ezetimibe); fibrates (such as, for example, for example, clofibrate, Clofibride, Etofibrate, and Aluminium Clofibrate); LXR agonists; LRP mimics; nicotinic receptor agonists; H3 receptor antagonists; histone deacetylase inhibitors; hsp90 inhibitors; ml muscarinic receptor agonists; 5-HT6 receptor antagonists; mGluR1; mGluR5; positive allosteric modulators or agonists; mGluR2/3 antagonists; anti-inflammatory agents that can reduce neuroinflammation; Prostaglandin EP2 receptor antagonists; PAI-1 inhibitors; and agents that can induce Abeta efflux such as gelsolin.

Another embodiment of this invention is directed to a pharmaceutical composition comprising an effective amount of one or more (e.g., one) compounds of formula (I), and effective amount of one or more BACE inhibitors, and a pharmaceutically acceptable carrier.

Another embodiment of this invention is directed to a pharmaceutical composition comprising an effective amount of one or more (e.g., one) compounds of formula (I), and effective amount of one or more cholinesterase inhibitors (e.g., acetyl- and/or butyrylchlolinesterase inhibitors), and a pharmaceutically acceptable carrier.

Another embodiment of this invention is directed to a pharmaceutical composition comprising an effective amount of one or more (e.g., one) compounds of formula (I), and effective amount of one or more muscarinic antagonists (e.g., m₁ agonists or m₂ antagonists), and a pharmaceutically acceptable carrier.

Another embodiment of this invention is directed to a pharmaceutical composition comprising an effective amount of one or more (e.g., one) compounds of formula (I), and effective amount of Exelon (rivastigmine), and a pharmaceutically acceptable carrier.

Another embodiment of this invention is directed to a pharmaceutical composition comprising an effective amount of one or more (e.g., one) compounds of formula (I), and effective amount of Cognex (tacrine), and a pharmaceutically acceptable carrier.

Another embodiment of this invention is directed to a pharmaceutical composition comprising an effective amount of one or more (e.g., one) compounds of formula (I), and effective amount of a Tau kinase inhibitor, and a pharmaceutically acceptable carrier.

Another embodiment of this invention is directed to a pharmaceutical composition comprising an effective amount of one or more (e.g., one) compounds of formula (I), and effective amount of one or more Tau kinase inhibitor (e.g., GSK3beta inhibitor, cdk5 inhibitor, ERK inhibitor), and a pharmaceutically acceptable carrier.

Another embodiment of this invention is directed to a pharmaceutical composition comprising an effective amount of one or more (e.g., one) compounds of formula (I), and effective amount of one anti-Abeta vaccine (active immunization), and a pharmaceutically acceptable carrier.

Another embodiment of this invention is directed to a pharmaceutical composition comprising an effective amount of one or more (e.g., one) compounds of formula (I), and effective amount of one or more APP ligands, and a pharmaceutically acceptable carrier.

Another embodiment of this invention is directed to a pharmaceutical composition comprising an effective amount of one or more (e.g., one) compounds of formula (I), and effective amount of one or more agents that upregulate insulin degrading enzyme and/or neprilysin, and a pharmaceutically acceptable carrier.

Another embodiment of this invention is directed to a pharmaceutical composition comprising an effective amount of one or more (e.g., one) compounds of formula (I), and effective amount of one or more cholesterol lowering agents (for example, statins such as Atorvastatin, Fluvastatin, Lovastatin, Mevastatin, Pitavastatin, Pravastatin, Rosuvastatin, Simvastatin, and cholesterol absorption inhibitor such as Ezetimibe), and a pharmaceutically acceptable carrier.

Another embodiment of this invention is directed to a pharmaceutical composition comprising an effective amount of one or more (e.g., one) compounds of formula (I), and effective amount of one or more fibrates (for example, clofibrate, Clofibride, Etofibrate, Aluminium Clofibrate), and a pharmaceutically acceptable carrier.

Another embodiment of this invention is directed to a pharmaceutical composition comprising an effective amount of one or more (e.g., one) compounds of formula (I), and effective amount of one or more LXR agonists, and a pharmaceutically acceptable carrier.

Another embodiment of this invention is directed to a pharmaceutical composition comprising an effective amount of one or more (e.g., one) compounds of formula (I), and effective amount of one or more LRP mimics, and a pharmaceutically acceptable carrier.

Another embodiment of this invention is directed to a pharmaceutical composition comprising an effective amount of one or more (e.g., one) compounds of formula (I), and effective amount of one or more 5-HT6 receptor antagonists, and a pharmaceutically acceptable carrier.

Another embodiment of this invention is directed to a pharmaceutical composition comprising an effective amount of one or more (e.g., one) compounds of formula (I), and effective amount of one or more nicotinic receptor agonists, and a pharmaceutically acceptable carrier.

Another embodiment of this invention is directed to a pharmaceutical composition comprising an effective amount of one or more (e.g., one) compounds of formula (I), and effective amount of one or more H3 receptor antagonists, and a pharmaceutically acceptable carrier.

Another embodiment of this invention is directed to a pharmaceutical composition comprising an effective amount of one or more (e.g., one) compounds of formula (I), and effective amount of one or more histone deacetylase inhibitors, and a pharmaceutically acceptable carrier.

Another embodiment of this invention is directed to a pharmaceutical composition comprising an effective amount of one or more (e.g., one) compounds of formula (I), and effective amount of one or more hsp90 inhibitors, and a pharmaceutically acceptable carrier.

Another embodiment of this invention is directed to a pharmaceutical composition comprising an effective amount of one or more (e.g., one) compounds of formula (I), and effective amount of one or more m1 muscarinic receptor agonists, and a pharmaceutically acceptable carrier.

Another embodiment of this invention is directed to a pharmaceutical composition comprising an effective amount of one or more (e.g., one) compounds of formula (I), and effective amount of one or more 5-HT6 receptor antagonists mGluR1 or mGluR5 positive allosteric modulators or agonists, and a pharmaceutically acceptable carrier.

Another embodiment of this invention is directed to a pharmaceutical composition comprising an effective amount of one or more (e.g., one) compounds of formula (I), and effective amount of one or more one mGluR2/3 antagonists, and a pharmaceutically acceptable carrier.

Another embodiment of this invention is directed to a pharmaceutical composition comprising an effective amount of one or more (e.g., one) compounds of formula (I), and effective amount of one or more anti-inflammatory agents that can reduce neuroinflammation, and a pharmaceutically acceptable carrier.

Another embodiment of this invention is directed to a pharmaceutical composition comprising an effective amount of one or more (e.g., one) compounds of formula (I), and effective amount of one or more Prostaglandin EP2 receptor antagonists, and a pharmaceutically acceptable carrier.

Another embodiment of this invention is directed to a pharmaceutical composition comprising an effective amount of one or more (e.g., one) compounds of formula (I), and effective amount of one or more PAI-1 inhibitors, and a pharmaceutically acceptable carrier.

Another embodiment of this invention is directed to a pharmaceutical composition comprising an effective amount of one or more (e.g., one) compounds of formula (I), and effective amount of one or more agents that can induce Abeta efflux such as gelsolin, and a pharmaceutically acceptable carrier.

Other embodiments of this invention are directed to any one of the above pharmaceutical compositions wherein the compounds of formula (I) are selected from the group consisting of compounds of the formula: Y1, Y2, Y3, A9-A14, B1-B15, C3-C5, D4, E4, E6-E9, F7-F19, F20d-F20h, F21d-F21h, F22d-F22h, F23c-F23h, F24c-F24h, F25a-F25h, F26a-F26h, F27a-F27h, F28a-F28h, F29a-F29h, F30a-F30h, F31a-F31h, F32a-F32h, F33a-F33h, J1, J2, K7, K8b-F8h, K9a-F9h, K10a-F10h, K11a-F11h, K12a-F12h, K13a-F13h, K14a-F14h, K15a-F15h, K16a-F16h, K17a-F17h, K18a-F18h, K19a-F19h, K20a-F20h, K21a-F21h, K22a-F22h, and X1-X11.

Another embodiment of this invention is directed to a pharmaceutical composition comprising an effective amount of at least one (e.g., one) compound of formula (I) selected from the group consisting of compounds of the formula: Y1, Y², Y3, A9-A14, B1-B15, C3-C5, D4, E4, E6-E9, F7-F19, F20d-F20h, F21d-F21h, F22d-F22h, F23c-F23h, F24c-F24h, F25a-F25h, F26a-F26h, F27a-F27h, F28a-F28h, F29a-F29h, F30a-F30h, F31a-F31h, F32a-F32h, F33a-F33h, J1, J2, K7, K8b-F8h, K9a-F9h, K10a-F10h, K11a-F11h, K12a-F12h, K13a-F13h, K14a-F14h, K15a-F15h, K16a-F16h, K17a-F17h, K18a-F18h, K19a-F19h, K20a-F20h, K21a-F21h, K22a-F22h, and X1-X11.

Another embodiment of this invention is directed to a pharmaceutical composition comprising an effective amount of a compound of formula (I) selected from the group consisting of compounds of the formula: Y1, Y2, Y3, A9-A14, B1-B15, C3-C5, D4, E4, E6-E9, F7-F19, F20d-F20h, F21d-F21h, F22d-F22h, F23c-F23h, F24c-F24h, F25a-F25h, F26a-F26h, F27a-F27h, F28a-F28h, F29a-F29h, F30a-F30h, F31a-F31h, F32a-F32h, F33a-F33h, J1, J2, K7, K8b-F8h, K9a-F9h, K10a-F10h, K11a-F11h, K12a-F12h, K13a-F13h, K14a-F14h, K15a-F15h, K16a-F16h, K17a-F17h, K18a-F18h, K19a-F19h, K20a-F20h, K21a-F21h, K22a-F22h, and X1-X11.

Another embodiment of this invention is directed to a pharmaceutical composition comprising an effective amount of a pharmaceutically acceptable salt of at least one (e.g., one) compound of formula (I) selected from the group consisting of compounds of the formula: Y1, Y2, Y3, A9-A14, B1-B15, C3-C5, D4, E4, E6-E9, F7-F19, F20d-F20h, F21d-F21h, F22d-F22h, F23c-F23h, F24c-F24h, F25a-F25h, F26a-F26h, F27a-F27h, F28a-F28h, F29a-F29h, F30a-F30h, F31a-F31h, F32a-F32h, F33a-F33h, J1, J2, K7, K8b-F8h, K9a-F9h, K10a-F10h, K11a-F11h, K12a-F12h, K13a-F13h, K14a-F14h, K15a-F15h, K16a-F16h, K17a-F17h, K18a-F18h, K19a-F19h, K20a-F20h, K21a-F21h, K22a-F22h, and X1-X11.

Another embodiment of this invention is directed to a pharmaceutical composition comprising an effective amount of a pharmaceutically acceptable salt of a compound of formula (I) selected from the group consisting of compounds of the formula: Y1, Y2, Y3, A9-A14, B1-B15, C3-C5, D4, E4, E6-E9, F7-F19, F20d-F20h, F21d-F21h, F22d-F22h, F23c-F23h, F24c-F24h, F25a-F25h, F26a-F26h, F27a-F27h, F28a-F28h, F29a-F29h, F30a-F30h, F31a-F31h, F32a-F32h, F33a-F33h, J1, J2, K7, K8b-F8h, K9a-F9h, K10a-F10h, K11a-F11h, K12a-F12h, K13a-F13h, K14a-F14h, K15a-F15h, K16a-F16h, K17a-F17h, K18a-F18h, K19a-F19h, K20a-F20h, K21a-F21h, K22a-F22h, and X1-X11.

Another embodiment of this invention is directed to a pharmaceutical composition comprising an effective amount of a pharmaceutically acceptable ester of at least one (e.g., one) compound of formula (I) selected from the group consisting of compounds of the formula: Y1, Y2, Y3, A9-A14, B1-B15, C3-C5, D4; E4, E6-E9, F7-F19, F20d-F20h, F21d-F21h, F22d-F22h, F23c-F23h, F24c-F24h, F25a-F25h, F26a-F26h, F27a-F27h, F28a-F28h, F29a-F29h, F30a-F30h, F31a-F31h, F32a-F32h, F33a-F33h, J1, J2, K7, K8b-F8h, K9a-F9h, K10a-F10h, K11a-F11h, K12a-F12h, K13a-F13h, K14a-F14h, K15a-F15h, K16a-F16h, K17a-F17h, K18a-F18h, K19a-F19h, K20a-F20h, K21a-F21h, K22a-F22h, and X1-X11.

Another embodiment of this invention is directed to a pharmaceutical composition comprising an effective amount of a pharmaceutically acceptable ester of a compound of formula (I) selected from the group consisting of compounds of the formula: Y1, Y2, Y3, A9-A14, B1-B15, C3-C5, D4, E4, E6-E9, F7-F19, F20d-F20h, F21d-F21h, F22d-F22h, F23c-F23h, F24c-F24h, F25a-F25h, F26a-F26h, F27a-F27h, F28a-F28h, F29a-F29h, F30a-F30h, F31a-F31h, F32a-F32h, F33a-F33h, J1, J2, K7, K8b-F8h, K9a-F9h, K10a-F10h, K11a-F11h, K12a-F12h, K13a-F13h, K14a-F14h, K15a-F15h, K16a-F16h, K17a-F17h, K18a-F18h, K19a-F19h, K20a-F20h, K21a-F21h, K22a-F22h, and X1-X11.

Another embodiment of this invention is directed to a pharmaceutical composition comprising an effective amount of a solvate of at least one (e.g., one) compound of formula (I) selected from the group consisting of compounds of the formula: Y1, Y2, Y3, A9-A14, B1-B15, C3-C5, D4, E4, E6-E9, F7-F19, F20d-F20h, F21d-F21h, F22d-F22h, F23c-F23h, F24c-F24h, F25a-F25h, F26a-F26h, F27a-F27h, F28a-F28h, F29a-F29h, F30a-F30h, F31a-F31h, F32a-F32h, F33a-F33h, J1, J2, K7, K8b-F8h, K9a-F9h, K10a-F10h, K11a-F11h, K12a-F12h, K13a-F13h, K14a-F14h, K15a-F15h, K16a-F16h, K17a-F17h, K18a-F18h, K19a-F19h, K20a-F20h, K21a-F21h, K22a-F22h, and X1-X11.

Another embodiment of this invention is directed to a pharmaceutical composition comprising an effective amount of a solvate of a compound of formula (I) selected from the group consisting of compounds of the formula: Y1, Y2, Y3, A9-A14, B1-B15, C3-C5, D4, E4, E6-E9, F7-F19, F20d-F20h, F21d-F21h, F22d-F22h, F23c-F23h, F24c-F24h, F25a-F25h, F26a-F26h, F27a-F27h, F28a-F28h, F29a-F29h, F30a-F30h, F31a-F31h, F32a-F32h, F33a-F33h, J1, J2, K7, K8b-F8h, K9a-F9h, K10a-F10h, K11a-F11h, K12a-F12h, K13a-F13h, K14a-F14h, K15a-F15h, K16a-F16h, K17a-F17h, K18a-F18h, K19a-F19h, K20a-F20h, K21a-F21h, K22a-F22h, and X1-X11.

The compounds of formula (I) can be useful as gamma secretase modulators and can be useful in the treatment and prevention of diseases such as; for example, central nervous system disorders (such as Alzheimers disease and Downs Syndrome), and treating mild cognitive impairment, glaucoma, cerebral amyloid angiopathy, stroke, dementia, microgliosis, brain inflammation, and olfactory function loss.

Thus, another embodiment of this invention is directed to a method for modulating (including inhibiting, antagonizing and the like) gamma-secretase comprising administering an effective amount of one or more (e.g., one) compounds of formula (I) to a patient in need of such treatment.

Another embodiment of this invention is directed to a method for modulating (including inhibiting, antagonizing and the like) gamma-secretase, comprising administering an effective amount of a compound of formula (I) to a patient in need of treatment.

Another embodiment of this invention is directed to a method of treating one or more neurodegenerative diseases, comprising administering an effective amount of one or more (e.g., one) compounds of formula (I) to a patient in need of treatment.

Another embodiment of this invention is directed to a method of treating one or more neurodegenerative diseases, comprising administering an effective amount of a compound of formula (I) to a patient in need of treatment.

Another embodiment of this invention is directed to a method of inhibiting the deposition of amyloid protein (e.g., amyloid beta protein) in, on or around neurological tissue (e.g., the brain), comprising administering an effective amount of one or more (e.g., one) compounds of formula (I) to a patient in need of treatment.

Another embodiment of this invention is directed to a method of inhibiting the deposition of amyloid protein (e.g., amyloid beta protein) in, on or around neurological tissue (e.g., the brain), comprising administering an effective amount of a compound of formula (I) to a patient in need of treatment.

Another embodiment of this invention is directed to a method of treating Alzheimer's disease, comprising administering an effective amount of one or more (e.g., one) compounds of formula (I) to a patient in need of treatment.

Another embodiment of this invention is directed to a method of treating Alzheimer's disease, comprising administering an effective amount of a compound of formula (I) to a patient in need of treatment.

Another embodiment of this invention is directed to a method of treating Downs syndrome, comprising administering an effective amount of one or more (e.g., one) compounds of formula (I) to a patient in need of treatment.

Another embodiment of this invention is directed to a method of treating Downs syndrome, comprising administering an effective amount of a compound of formula (I) to a patient in need of treatment.

Another embodiment of this invention is directed to a method of treating mild cognitive impairment, glaucoma, cerebral amyloid angiopathy, stroke, dementia, microgliosis, brain inflammation, or olfactory function loss, comprising administering an effective (i.e., therapeutically effective) amount of one or more (e.g., one) compounds of formula (I) to a patient in need of treatment.

Another embodiment of this invention is directed to a method of treating mild cognitive impairment, glaucoma, cerebral amyloid angiopathy, stroke, dementia, microgliosis, brain inflammation, or olfactory function loss, comprising administering an effective (i.e., therapeutically effective) amount of a compound of formula (I) to a patient in need of treatment.

Another embodiment of this invention is directed to a method of treating mild cognitive impairment, comprising administering an effective amount of one or more (e.g., one) compounds of formula (I) to a patient in need of treatment.

Another embodiment of this invention is directed to a method of treating glaucoma, comprising administering an effective amount of one or more (e.g., one) compounds of formula (I) to a patient in need of treatment.

Another embodiment of this invention is directed to a method of treating cerebral amyloid angiopathy, comprising administering an effective amount of one or more (e.g., one) compounds of formula (I) to a patient in need of treatment.

Another embodiment of this invention is directed to a method of treating stroke, comprising administering an effective amount of one or more (e.g., one) compounds of formula (I) to a patient in need of treatment.

Another embodiment of this invention is directed to a method of treating dementia, comprising administering an effective amount of one or more (e.g., one) compounds of formula (I) to a patient in need of treatment.

Another embodiment of this invention is directed to a method of treating microgliosis, comprising administering an effective amount of one or more (e.g., one) compounds of formula (I) to a patient in need of treatment.

Another embodiment of this invention is directed to a method of treating brain inflammation, comprising administering an effective amount of one or more (e.g., one) compounds of formula (I) to a patient in need of treatment.

Another embodiment of this invention is directed to a method of treating olfactory function loss, comprising administering an effective amount of one or more (e.g., one) compounds of formula (I) to a patient in need of treatment.

This invention also provides combination therapies for (1) modulating gamma-secretase, or (2) treating one or more neurodegenerative diseases, or (3) inhibiting the deposition of amyloid protein (e.g., amyloid beta protein) in, on or around neurological tissue (e.g., the brain), or (4) treating Alzheimer's disease. The combination therapies are directed to methods comprising the administration of an effective amount of one or more (e.g. one) compounds of formula (I) and the administration of an effective amount of one or more (e.g., one) other pharmaceutical active ingredients (e.g., drugs). The compounds of formula (I) and the other drugs can be administered separately (i.e., each is in its own separate dosage form), or the compounds of formula (I) can be combined with the other drugs in the same dosage form.

Thus, other embodiments of this invention are directed to any one of the methods of treatment, or methods of inhibiting, described herein, wherein an effective amount of the compound of formula (I) is used in combination with an effective amount of one or more other pharmaceutically active ingredients selected from the group consisting of: BACE inhibitors (beta secretase inhibitors); muscarinic antagonists (e.g., m₁ agonists or m₂ antagonists); cholinesterase inhibitors (e.g., acetyl- and/or butyrylchlolinesterase inhibitors); gamma secretase inhibitors; gamma secretase modulators; HMG-CoA reductase inhibitors; non-steroidal anti-inflammatory agents; N-methyl-D-aspartate receptor antagonists; anti-amyloid antibodies; vitamin E; nicotinic acetylcholine receptor agonists; CB1 receptor inverse agonists or CB1 receptor antagonists; an antibiotic; growth hormone secretagogues; histamine H3 antagonists; AMPA agonists; PDE4 inhibitors; GABA_{A} inverse agonists; inhibitors of amyloid aggregation; glycogen synthase kinase beta inhibitors; promoters of alpha secretase activity; PDE-10 inhibitors; Exelon (rivastigmine); Cognex (tacrine); Tau kinase inhibitors (e.g., GSK3beta inhibitors, cdk5 inhibitors, or ERK inhibitors); anti-Abeta vaccine; APP ligands; agents that upregulate insulin cholesterol lowering agents (for example, statins such as Atorvastatin, Fluvastatin, Lovastatin, Mevastatin, Pitavastatin, Pravastatin, Rosuvastatin, Simvastatin); cholesterol absorption inhibitors (such as Ezetimibe); fibrates (such as, for example, for example, clofibrate, Clofibride, Etofibrate, and Aluminium Clofibrate); LXR agonists; LRP mimics; nicotinic receptor agonists; H3 receptor antagonists; histone deacetylase inhibitors; hsp90 inhibitors; ml muscarinic receptor agonists; 5-HT6 receptor antagonists; mGluR1; mGluR5; positive allosteric modulators or agonists; mGluR2/3 antagonists; anti-inflammatory agents that can reduce neuroinflammation; Prostaglandin EP2 receptor antagonists; PAI-1 inhibitors; and agents that can induce Abeta efflux such as gelsolin.

Other embodiments of this invention are directed to any one of the methods of treatment, or methods of inhibiting, described herein, wherein an effective amount of the compound of formula (I) is used in combination with an effective amount of one or more other pharmaceutically active ingredients selected from the group consisting of: BACE inhibitors (beta secretase inhibitors); muscarinic antagonists (e.g., m₁ agonists or m₂ antagonists); cholinesterase inhibitors (e.g., acetyl- and/or butyrylcholinesterase inhibitors); gamma secretase inhibitors; gamma secretase modulators; HMG-CoA reductase inhibitors; non-steroidal anti-inflammatory agents; N-methyl-D-aspartate receptor antagonists; anti-amyloid antibodies; vitamin E; nicotinic acetylcholine receptor agonists; CB1 receptor inverse agonists or CB1 receptor antagonists; an antibiotic; growth hormone secretagogues; histamine H3 antagonists; AMPA agonists; PDE4 inhibitors; GABA_{A} inverse agonists; inhibitors of amyloid aggregation; glycogen synthase kinase beta inhibitors; promoters of alpha secretase activity; and PDE-10 inhibitors.

Other embodiments of this invention are directed to any one of the methods of treatment, or methods of inhibiting, described herein, wherein an effective amount of the compound of formula (I) is used in combination with an effective amount of one or more other pharmaceutically active ingredients selected from the group consisting of: Exelon (rivastigmine); Cognex (tacrine); Tau kinase inhibitors (e.g., GSK3beta inhibitors, cdk5 inhibitors, or ERK inhibitors); anti-Abeta vaccine; APP ligands; agents that upregulate insulin cholesterol lowering agents (for example, statins such as Atorvastatin, Fluvastatin, Lovastatin, Mevastatin, Pitavastatin, Pravastatin, Rosuvastatin, Simvastatin); cholesterol absorption inhibitors (such as Ezetimibe); fibrates (such as, for example, for example, clofibrate, Clofibride, Etofibrate, and Aluminium Clofibrate); LXR agonists; LRP mimics; nicotinic receptor agonists; H3 receptor antagonists; histone deacetylase inhibitors; hsp90 inhibitors; ml muscarinic receptor agonists; 5-HT6 receptor antagonists; mGluR1; mGluR5; positive allosteric modulators or agonists; mGluR2/3 antagonists; anti-inflammatory agents that can reduce neuroinflammation; Prostaglandin EP2 receptor antagonists; PAI-1 inhibitors; and agents that can induce Abeta efflux such as gelsolin.

Another embodiment of this invention is directed to a method of treating Alzheimer's disease, comprising administering an effective amount of one or more (e.g., one) compounds of formula (I), in combination with an effective (i.e., therapeutically effective) amount of one or more cholinesterase inhibitors (such as, for example, (±)-2,3-dihydro-5,6-dimethoxy-2-[[1-(phenylmethyl)-4-piperidinyl]methyl]-1H-inden-1-one hydrochloride, i.e., donepezil hydrochloride, available as the Aricept® brand of donepezil hydrochloride), to a patient in need of treatment.

Another embodiment of this invention is directed to a method of treating Alzheimer's disease, comprising administering an effective amount of a compound of formula (I), in combination with an effective amount of one or more (e.g., one) cholinesterase inhibitors (such as, for example, (±)-2,3-dihydro-5,6-dimethoxy-2-[[1-(phenylmethyl)-4-piperidinyl]methyl]-1H-inden-1-one hydrochloride, i.e., donepezil hydrochloride, available as the Aricept® brand of donepezil hydrochloride), to a patient in need of treatment.

Another embodiment of this invention is directed to a method of treating Alzheimer's disease, comprising administering an effective amount of one or more (e.g., one) compounds of formula (I), in combination with an effective amount of one or more compounds selected from the group consisting of Aβ antibody inhibitors, gamma secretase inhibitors and beta secretase inhibitors.

Another embodiment of this invention is directed to a method of treating Alzheimer's disease, comprising administering an effective amount of one or more (e.g., one) compounds of formula (I), in combination with an effective amount of one or more BACE inhibitors.

Another embodiment of this invention is directed to a method of treating Alzheimer's disease, comprising administering an effective amount of one or more compounds of formula (I), in combination with an effective amount of Exelon (rivastigmine).

Another embodiment of this invention is directed to a method of treating Alzheimer's disease, comprising administering an effective amount of one or more compounds of formula (I), in combination with an effective amount of Cognex (tacrine).

Another embodiment of this invention is directed to a method of treating Alzheimer's disease, comprising administering an effective amount of one or more compounds of formula (I), in combination with an effective amount of a Tau kinase inhibitor.

Another embodiment of this invention is directed to a method of treating Alzheimer's disease, comprising administering an effective amount of one or more compounds of formula (I), in combination with an effective amount of one or more Tau kinase inhibitor (e.g., GSK3beta inhibitor, cdk5 inhibitor, ERK inhibitor).

Another embodiment of this invention is directed to a method of treating Alzheimer's disease, comprising administering an effective amount of one or more compounds of formula (I), in combination with an effective amount of one anti-Abeta vaccination (active immunization).

Another embodiment of this invention is directed to a method of treating Alzheimer's disease, comprising administering an effective amount of one or more compounds of formula (I), in combination with an effective amount of one or more APP ligands.

Another embodiment of this invention is directed to a method of treating Alzheimer's disease, comprising administering an effective amount of one or more compounds of formula (I), in combination with an effective amount of one or more agents that upregulate insulin degrading enzyme and/or neprilysin.

Another embodiment of this invention is directed to a method of treating Alzheimer's disease, comprising administering an effective amount of one or more compounds of formula (I), in combination with an effective amount of one or more cholesterol lowering agents (for example, statins such as Atorvastatin, Fluvastatin, Lovastatin, Mevastatin, Pitavastatin, Pravastatin, Rosuvastatin, Simvastatin, and cholesterol absorption inhibitor such as Ezetimibe).

Another embodiment of this invention is directed to a method of treating Alzheimer's disease, comprising administering an effective amount of one or more compounds of formula (I), in combination with an effective amount of one or more fibrates (for example, clofibrate, Clofibride, Etofibrate, Aluminium Clofibrate).

Another embodiment of this invention is directed to a method of treating Alzheimer's disease, comprising administering an effective amount of one or more compounds of formula (I), in combination with an effective amount of one or more LXR agonists.

Another embodiment of this invention is directed to a method of treating Alzheimer's disease, comprising administering an effective amount of one or more compounds of formula (I), in combination with an effective amount of one or more LRP mimics.

Another embodiment of this invention is directed to a method of treating Alzheimer's disease, comprising administering an effective amount of one or more compounds of formula (I), in combination with an effective amount of one or more 5-HT6 receptor antagonists.

Another embodiment of this invention is directed to a method of treating Alzheimer's disease, comprising administering an effective amount of one or more compounds of formula (I), in combination with an effective amount of one or more nicotinic receptor agonists.

Another embodiment of this invention is directed to a method of treating Alzheimer's disease, comprising administering an effective amount of one or more compounds of formula (I), in combination with an effective amount of one or more H3 receptor antagonists.

Another embodiment of this invention is directed to a method of treating Alzheimer's disease, comprising administering an effective amount of one or more compounds of formula (I), in combination with an effective amount of one or more histone deacetylase inhibitors.

Another embodiment of this invention is directed to a method of treating Alzheimer's disease, comprising administering an effective amount of one or more compounds of formula (I), in combination with an effective amount of one or more hsp90 inhibitors.

Another embodiment of this invention is directed to a method of treating Alzheimer's disease, comprising administering an effective amount of one or more compounds of formula (I), in combination with an effective amount of one or more m1 muscarinic receptor agonists.

Another embodiment of this invention is directed to a method of treating Alzheimer's disease, comprising administering an effective amount of one or more compounds of formula (I), in combination with an effective amount of one or more 5-HT6 receptor antagonists, mGluR1, mGluR5, or positive allosteric modulators or agonists.

Another embodiment of this invention is directed to a method of treating Alzheimer's disease, comprising administering an effective amount of one or more compounds of formula (I), in combination with an effective amount of one or more mGluR2/3 antagonists.

Another embodiment of this invention is directed to a method of treating Alzheimer's disease, comprising administering an effective amount of one or more compounds of formula (I), in combination with an effective amount of one or more anti-inflammatory agents that can reduce neuroinflammation.

Another embodiment of this invention is directed to a method of treating Alzheimer's disease, comprising administering an effective amount of one or more compounds of formula (I), in combination with an effective amount of one or more Prostaglandin EP2 receptor antagonists.

Another embodiment of this invention is directed to a method of treating Alzheimer's disease, comprising administering an effective amount of one or more compounds of formula (I), in combination with an effective amount of one or more PAI-1 inhibitors.

Another embodiment of this invention is directed to a method of treating Alzheimer's disease, comprising administering an effective amount of one or more compounds of formula (I), in combination with an effective amount of one or more agents that can induce Abeta efflux such as gelsolin.

Another embodiment of this invention is directed to a method of treating Downs syndrome, comprising administering an effective amount of one or more (e.g., one) compounds of formula (I), in combination with an effective amount of one or more cholinesterase inhibitors (such as, for example, (±)-2,3-dihydro-5,6-dimethoxy-2-[[1-(phenylmethyl)-4-piperidinyl]methyl]-1H-inden-1-one hydrochloride, i.e., donepezil hydrochloride, available as the Aricept® brand of donepezil hydrochloride), to a patient in need of treatment.

Another embodiment of this invention is directed to a method of treating Downs syndrome, comprising administering an effective amount of a compound of formula (I), in combination with an effective amount of one or more (e.g., one) cholinesterase inhibitors (such as, for example, (±)-2,3-dihydro-5,6-dimethoxy-2-[[1-(phenylmethyl)-4-piperidinyl]methyl]-1H-inden-1-one hydrochloride, i.e., donepezil hydrochloride, available as the Aricept® brand of donepezil hydrochloride), to a patient in need of treatment.

Other embodiments of this invention are directed to any one of the above method of treating embodiments wherein the compounds of formula (I) are selected from the group consisting of compounds of the formula: Y1, Y2, Y3, A9-A14, B1-B15, C3-C5, D4, E4, E6-E9, F7-F19, F20d-F20h, F21d-F21h, F22d-F22h, F23c-F23h, F24c-F24h, F25a-F25h, F26a-F26h, F27a-F27h, F28a-F28h, F29a-F29h, F30a-F30h, F31a-F31h, F32a-F32h, F33a-F33h, J1, J2, K7, K8b-F8h, K9a-F9h, K10a-F10h, K11a-F11h, K12a-F12h, K13a-F13h, K14a-F14h, K15a-F15h, K16a-F16h, K17a-F17h, K18a-F18h, K19a-F19h, K20a-F20h, K21a-F21h, K22a-F22h, and X1-X11.

Another embodiment of this invention is directed to combinations (i.e., pharmaceutical compositions) comprising an effective amount of one or more (e.g., one) compounds of formula (I), in combination with an effective amount of one or more compounds selected from the group consisting of cholinesterase inhibitors (such as, for example, (±)-2,3-dihydro-5,6-dimethoxy-2-[[1-(phenylmethyl)-4-piperidinyl]methyl]-1H-inden-1-one hydrochloride, i.e., donepezil hydrochloride, available as the Aricept® brand of donepezil hydrochloride), Aβ antibody inhibitors, gamma secretase inhibitors and beta secretase inhibitors. The pharmaceutical compositions also comprise a pharmaceutically acceptable carrier.

Another embodiment of this invention is directed to combinations (i.e., pharmaceutical compositions) comprising an effective amount of one or more (e.g., one) compounds of formula (I), in combination with an effective amount of one or more compounds selected from the group consisting of cholinesterase inhibitors (such as, for example, (±)-2,3-dihydro-5,6-dimethoxy-2-[[1-(phenylmethyl)-4-piperidinyl]methyl]-1H-inden-1-one hydrochloride, i.e., donepezil hydrochloride, available as the Aricept® brand of donepezil hydrochloride), Aβ antibody inhibitors, gamma secretase inhibitors and beta secretase inhibitors, wherein the compound of formula (I) is selected from the group consisting of the compounds of the formula: Y1, Y2, Y3, A9-A14, B1-B15, C3-C5, D4, E4, E6-E9, F7-F19, F20d-F20h, F21d-F21h, F22d-F22h, F23c-F23h, F24c-F24h, F25a-F25h, F26a-F26h, F27a-F27h, F28a-F28h, F29a-F29h, F30a-F30h, F31a-F31h, F32a-F32h, F33a-F33h, J1, J2, K7, K8b-F8h, K9a-F9h, K10a-F10h, K11a-F11h, K12a-F12h, K13a-F13h, K14a-F14h, K15a-F15h, K16a-F16h, K17a-F17h, K18a-F18h, K19a-F19h, K20a-F20h, K21a-F21 h, K22a-F22h, and X1-X11. The pharmaceutical compositions also comprise a pharmaceutically acceptable carrier.

Another embodiment of this invention is directed to a kit comprising, in separate containers, in a single package, pharmaceutical compositions for use in combination, wherein one container comprises an effective amount of a compound of formula (I) in a pharmaceutically acceptable carrier, and another container (i.e., a second container) comprises an effective amount of another pharmaceutically active ingredient (as described above), the combined quantities of the compound of formula (I) and the other pharmaceutically active ingredient being effective to: (a) treat Alzheimer's disease, or (b) inhibit the deposition of amyloid protein (e.g., amyloid beta protein) in, on or around neurological tissue (e.g., the brain), or (c) treat neurodegenerative diseases, or (d) modulate the activity of gamma-secretase.

Another embodiment of this invention is directed to a kit comprising, in separate containers, in a single package, pharmaceutical compositions for use in combination, wherein one container comprises an effective amount of a compound of formula (I) in a pharmaceutically acceptable carrier, and another container (i.e., a second container) comprises an effective amount of another pharmaceutically active ingredient (as described above), the combined quantities of the compound of formula (I) and the other pharmaceutically active ingredient being effective to: (a) treat Alzheimer's disease, or (b) inhibit the deposition of amyloid protein (e.g., amyloid beta protein) in, on or around neurological tissue (e.g., the brain), or (c) treat neurodegenerative diseases, or (d) modulate the activity of gamma-secretase, wherein the compound of formula (I) is selected from the group consisting of the compounds of the formula: Y1, Y2, Y3, A9-A14, B1-B15, C3-C5, D4, E4, E6-E9, F7-F19, F20d-F20h, F21d-F21h, F22d-F22h, F23c-F23h, F24c-F24h, F25a-F25h, F26a-F26h, F27a-F27h, F28a-F28h, F29a-F29h, F30a-F30h, F31a-F31h, F32a-F32h, F33a-F33h, J1, J2, K7, K8b-F8h, K9a-F9h, K10a-F10h, K11a-F11h, K12a-F12h, K13a-F13h, K14a-F14h, K15a-F15h, K16a-F16h, K17a-F17h, K18a-F18h, K19a-F19h, K20a-F20h, K21a-F21h, K22a-F22h, and X1-X11.

Examples of cholinesterase inhibitors are tacrine, donepezil, rivastigmine, galantamine, pyridostigmine and neostigmine, with tacrine, donepezil, rivastigmine and galantamine being preferred.

Examples of m₁ agonists are known in the art. Examples of m₂ antagonists are also known in the art; in particular, m₂ antagonists are disclosed in U.S. Pat. Nos. 5,883,096; 6,037,352; 5,889,006; 6,043,255; 5,952,349; 5,935,958; 6,066,636; 5,977,138; 6,294,554; 6,043,255; and 6,458,812; and in WO 03/031412, all of which are incorporated herein by reference.

Examples of BACE inhibitors include those described in: US2005/0119227 published Jun. 2, 2005 (see also WO2005/016876 published Feb. 24, 2005), US2005/0043290 published Feb. 24, 2005 (see also WO2005/014540 published Feb. 17, 2005), WO2005/058311 published Jun. 30, 2005 (see also US2007/0072852 published Mar. 29, 2007), US2006/0111370 published May 25, 2006 (see also WO2006/065277 published Jun. 22, 2006), U.S. application Ser. No. 11/710,582 filed Feb. 23, 2007, US2006/0040994 published Feb. 23, 2006 (see also WO2006/014762 published Feb. 9, 2006), WO2006/014944 published Feb. 9, 2006 (see also US2006/0040948 published Feb. 23, 2006), WO2006/138266 published Dec. 28, 2006 (see also US2007/0010667 published Jan. 11, 2007), WO2006/138265 published Dec. 28, 2006, WO2006/138230 published Dec. 28, 2006, WO2006/138195 published Dec. 28, 2006 (see also US2006/0281729 published Dec. 14, 2006), WO2006/138264 published Dec. 28, 2006 (see also US2007/0060575 published Mar. 15, 2007), WO2006/138192 published Dec. 28, 2006 (see also US2006/0281730 published Dec. 14, 2006), WO2006/138217 published Dec. 28, 2006 (see also US2006/0287294 published Dec. 21, 2006), US2007/0099898 published 05/03/200 (see also WO2007/050721 published May 3, 2007), WO2007/053506 published May 10, 2007 (see also US2007/099875 published May 3, 2007), U.S. application Ser. No. 11/759,336 filed Jun. 7, 2007, U.S. Application Ser. No. 60/874,362 filed Dec. 12, 2006, and U.S. Application Ser. No. 60/874,419 filed Dec. 12, 2006, the disclosures of each being incorporated herein by reference thereto.

For preparing pharmaceutical compositions from the compounds described by this invention, inert, pharmaceutically acceptable carriers can be either solid or liquid. Solid form preparations include powders, tablets, dispersible granules, capsules, cachets and suppositories. The powders and tablets may be comprised of from about 5 to about 95 percent active ingredient. Suitable solid carriers are known in the art, e.g., magnesium carbonate, magnesium stearate, talc, sugar or lactose. Tablets, powders, cachets and capsules can be used as solid dosage forms suitable for oral administration. Examples of pharmaceutically acceptable carriers and methods of manufacture for various compositions may be found in A. Gennaro (ed.), Remington's Pharmaceutical Sciences, 18^{th} Edition, (1990), Mack Publishing Co., Easton, Pa.

Liquid form preparations include solutions, suspensions and emulsions. As an example may be mentioned water or water-propylene glycol solutions for parenteral injection or addition of sweeteners and opacifiers for oral solutions, suspensions and emulsions. Liquid form preparations may also include solutions for intranasal administration.

Aerosol preparations suitable for inhalation may include solutions and solids in powder form, which may be in combination with a pharmaceutically acceptable carrier, such as an inert compressed gas, e.g. nitrogen.

Also included are solid form preparations that are intended to be converted, shortly before use, to liquid form preparations for either oral or parenteral administration. Such liquid forms include solutions, suspensions and emulsions.

The compounds of the invention may also be deliverable transdermally. The transdermal compositions can take the form of creams, lotions, aerosols and/or emulsions and can be included in a transdermal patch of the matrix or reservoir type as are conventional in the art for this purpose.

The compounds of this invention may also be delivered subcutaneously.

Preferably the compound is administered orally.

Preferably, the pharmaceutical preparation is in a unit dosage form. In such form, the preparation is subdivided into suitably sized unit doses containing appropriate quantities of the active component, e.g., an effective amount to achieve the desired purpose.

The quantity of active compound in a unit dose of preparation may be varied or adjusted from about 1 mg to about 100 mg, preferably from about 1 mg to about 50 mg, more preferably from about 1 mg to about 25 mg, according to the particular application.

The actual dosage employed may be varied depending upon the requirements of the patient and the severity of the condition being treated. Determination of the proper dosage regimen for a particular situation is within the skill of the art. For convenience, the total daily dosage may be divided and administered in portions during the day as required.

The amount and frequency of administration of the compounds of the invention and/or the pharmaceutically acceptable salts thereof will be regulated according to the judgment of the attending clinician considering such factors as age, condition and size of the patient as well as severity of the symptoms being treated. A typical recommended daily dosage regimen for oral administration can range from about 1 mg/day to about 500 mg/day, preferably 1 mg/day to 200 mg/day, in two to four divided doses.

Another aspect of this invention is a kit comprising a therapeutically effective amount of at least one compound of Formula (I), or a pharmaceutically acceptable salt, solvate, ester or prodrug of said compound and a pharmaceutically acceptable carrier, vehicle or diluent.

Yet another aspect of this invention is a kit comprising an amount of at least one compound of Formula (I), or a pharmaceutically acceptable salt, solvate, ester or prodrug of said compound and an amount of at least one additional agent listed above, wherein the amounts of the two or more ingredients result in desired therapeutic effect.

The invention disclosed herein is exemplified by the following illustrative example which should not be construed to limit the scope of the disclosure. Alternative mechanistic pathways and analogous structures will be apparent to those skilled in the art.

### ILLUSTRATIVE EXAMPLES

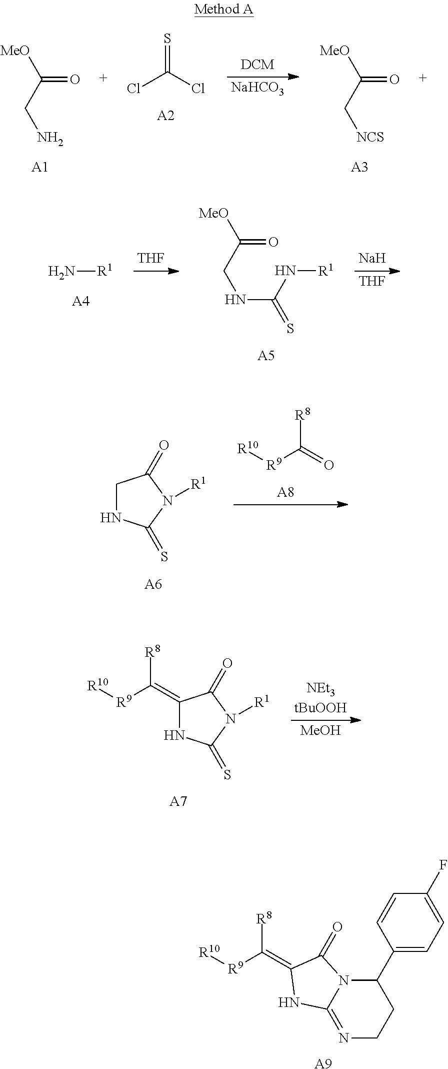

### Method A, Step 1

To a round bottom flask at 0° C. is added the hydrochloride salt of glycine methyl ester (2.00 g, 15.9 mmol), thiophosgene (2.67 mL, 35.0 mmol) in DCM (10 mL) and sat'd NaHCO₃ aq solution (10 mL). The reaction is stirred vigorously while warming to room temperature over 16h. The mixture is extracted with DCM and water. The organic portion is dried over sodium sulfate, filtered, and concentrated in vacuo to yield 0.42 g of methyl-2-isothiocyanatoacetate.

¹H NMR (CDCl₃) δ (ppm): 3.83 (s, 3H); 4.25 (s, 2H).

### Method A, Step 2

To a round bottom flask is added methyl-2-isothiocyanatoacetate (0.42 g, 3.2 mmol) and A4 (R¹=α-2-Boc-aminoethyl-p-fluoro-benzyl) in (0.48 mL, 3.5 mmol) in THF (5 mL) and stir at room until starting material is consumed. The mixture is extracted with EtOAc and water (2×), then 1M HCl aq (2×) then sat'd NaHCO₃ aq solution (2×). The organic portion is dried over sodium sulfate, filtered, and concentrated in vacuo to yield A5 (R¹=α-2-Boc-aminoethyl-p-fluoro-benzyl).

### Method A, Step 3

To a round bottom flask at 0° C. containing a solution of sodium hydride (114 mg, 2.85 mmol) in anhydrous THF (5 mL) will be slowly added a solution of A5 (R¹=α-2-Boc-aminoethyl-p-fluoro-benzyl, 2.0 mmol) in THF (10 mL). The reaction will be allowed to warm to room temperature, diluted with EtOAc, extracted with 1N HCl aq (2×30 mL), then brine (40 mL). The organic portion will be dried over sodium sulfate, filtered, and concentrated in vacuo to yield A6 (R¹=α-2-Boc-aminoethyl-p-fluoro-benzyl).

### Method A, Step 4

In a round bottom flask containing piperidine (0.41 mL, 4.2 mmol) will be added a solution of A8 (R⁸=H, R⁹=4-R¹⁰-3-Methoxyphenyl, R¹⁰=4-Methylimidazol-1-yl) (429 mg, 2.0 mmol) and A6 (R¹=α-2-Boc-aminoethyl-p-fluoro-benzyl, 1.9 mmol) in ethanol (20 mL). This mixture will be stirred at reflux temperature for 16 h. The reaction mixture will be cooled to room temperature, diluted with EtOAc, extracted with water then brine. The organic portion will be dried over sodium sulfate, filtered, and concentrated in vacuo to yield the crude product which will be chromatographed to produce A7 (R¹=α-2-Boc-aminoethyl-p-fluoro-benzyl, R⁸=H, R⁹=4—R¹⁰-3-Methoxyphenyl, R¹⁰=4-Methylimidazol-1-yl)

### Method A, Step 5

A7 (R¹=α-2-Boc-aminoethyl-p-fluoro-benzyl, R⁸=H, R⁹=4-R¹⁰-3-Methoxyphenyl, R¹⁰=4-Methylimidazol-1-yl) will be treated with 20% TFA/DCM until starting material disappears before the volatiles will be removed. The residue will be dissolved in DCM, and then this solution will be washed with then sat'd NaHCO₃ aq solution (2×). The organic portion will be dried over sodium sulfate, filtered, and concentrated in vacuo. The residue will be dissolved in MeOH (1 mL) and transferred into a sealed tube. To the sealed vial will be added triethylamine (0.25 mL) and a 70% solution of tert-butyl hydroperoxide in water (0.5 mL), and then this mixture will be agitated at room temperature for 16 h. The reaction mixture will be concentrated in vacuo and residue will be purified by reverse phase chromatography to produce A9 (R⁸=H, R⁹=4-R¹⁰-3-Methoxyphenyl, R¹⁰=4-Methylimidazol-1-yl).

The following compounds will be generated using method similar to Method A.

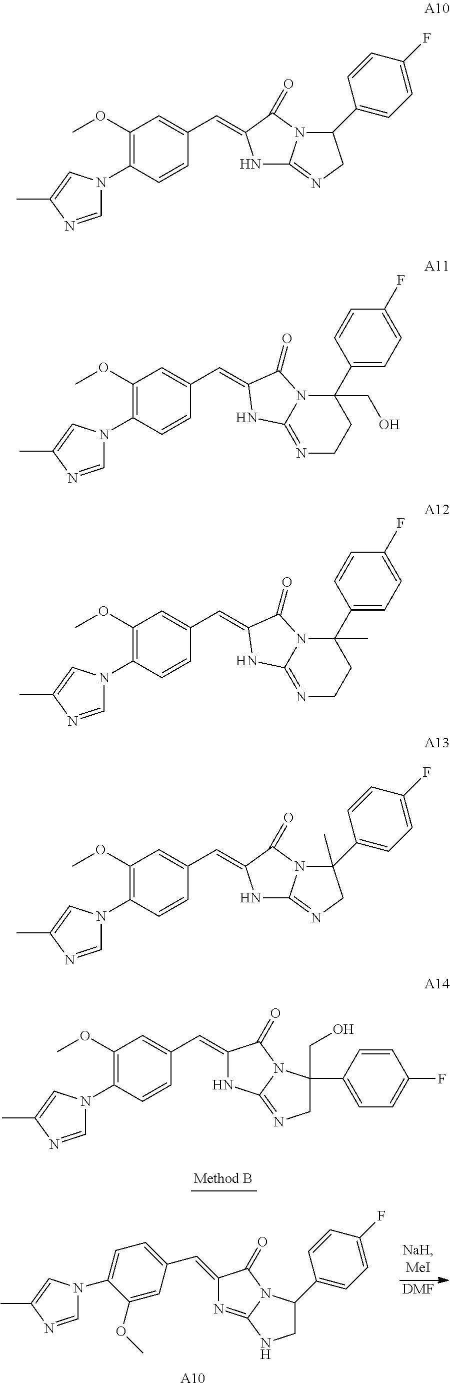

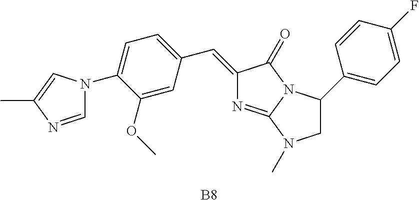

To a round bottom flask at 0° C. containing a mixture of a 60% sodium hydride dispersion (2.4 mg, 0.06 mmol) in DMF (2 mL) was added a solution of A10 (25 mg, 0.06 mmol) in DMF (1 mL). After this mixture was stirred for 0.33 h at 0° C., methyl iodide (3.7 μL, 0.6 mmol) was added and the mixture was removed from the ice-bath after 0.16 h. After 1 h the reaction was diluted with 75% ethyl acetate/hexanes (40 mL), washed with water (3×10 mL), washed with brine (1×10 mL), dried over Na₂SO₄, and concentrated in vacuo. The crude material was purified by silica gel chromatography with methanol/ammonium hydroxide/DCM to afford compound B8 (15 mg, 57%) as a yellow film. ¹HNMR (CDCl₃, 400 MHz) δ 7.99 (s, 1H), 7.75 (s, 1H, 7.58 (d, 1H), 7.31-7.23 (m, 3H), 7.09 (t, 2H), 6.95 (s, 1H), 6.60 (s, 1H), 5.29-5.27 (m, 1H), 4.31 (t, 1H), 3.91 (s, 3H), 3.76-3.72 (m, 1H), 3.16 (s, 3H), 2.30 (s, 3H); MS (M+1)⁺ m/z calcd for C₂₄H₂₂FN₅O₂⁺=432.4, found m/z=432.2.

The following compounds will be synthesized using method similar to Method B.

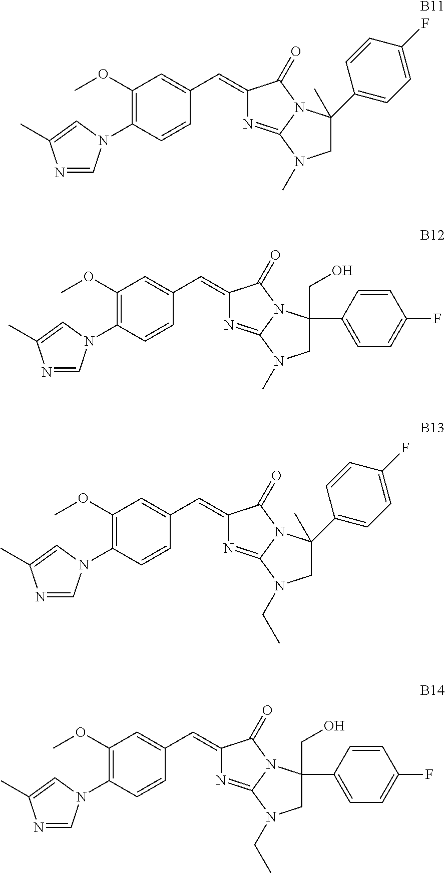

The following compound (“Cpd”) was made using a method similar to Method B:

| | | | m/z |
|---|---|---|---|
| | | Molecular | Found |
| Cpd | Structure | Weight | (M + 1)⁺ |
| B15 | 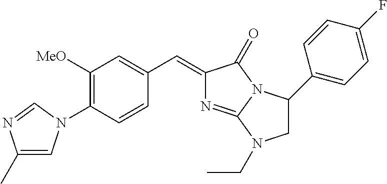 | 445.5 | 446.2 |

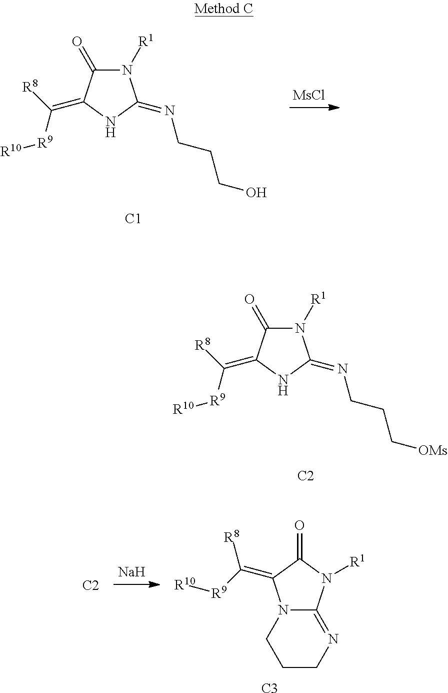

Into a vial was added C1 (R¹=α-methyl-p-fluoro-benzyl, R⁸=H, R⁹=4-R¹⁰-3-Methoxyphenyl, R¹⁰=4-Methylimidazol-1-yl) (15.1 mg, 0.0316 mmol) in tetrahydrofuran (1 mL), triethylamine (20 μL, 0.1 mmol) and methanesulfonyl chloride (10 μL, 0.1 mmol). The vial was sealed and agitated at room temperature for 3 h. This mixture was extracted with ethyl acetate and water, the organic portion was collected, dried over sodium sulfate, concentrated, and used in the next step without further purification. This residue was taken up in THF (1 mL) and a 60% dispersion of sodium hydride in mineral oil (1.90 mg, 0.0474 mmol) was added. This mixture was stirred for 2 h at room temperature, and then quenched with water. The mixture was concentrated in vacuo and purified by reverse phase chromatography to produce 1.8 mg of C3 (R¹=α-methyl-p-fluoro-benzyl, R⁸=H, R⁹=4-R¹⁰-3-Methoxyphenyl, R¹⁰=4-Methylimidazol-1-yl). ¹H NMR (CDCl₃) δ (ppm): 1.97 (d, 3H); 1.87 (t, 2H); 2.43 (s, 3H); 3.53 (d of t; 2H); 3.73 (t, 2H); 3.97 (s, 3H); 5.43 (q, 1H); 5.48 (s, 1H); 7.15 (t, 2H); 7.23 (t, 2H); 7.38 (s, 1H); 7.55 (d, 1H); 7.60 (br s, 1H); 7.61 (t, 1H); 9.16 (s, 1H). Mass Spec (m/z) M+H (ESI): 460.3

The following compounds (R¹=α-methyl-p-fluoro-benzyl) will be generated using method similar to Method C.

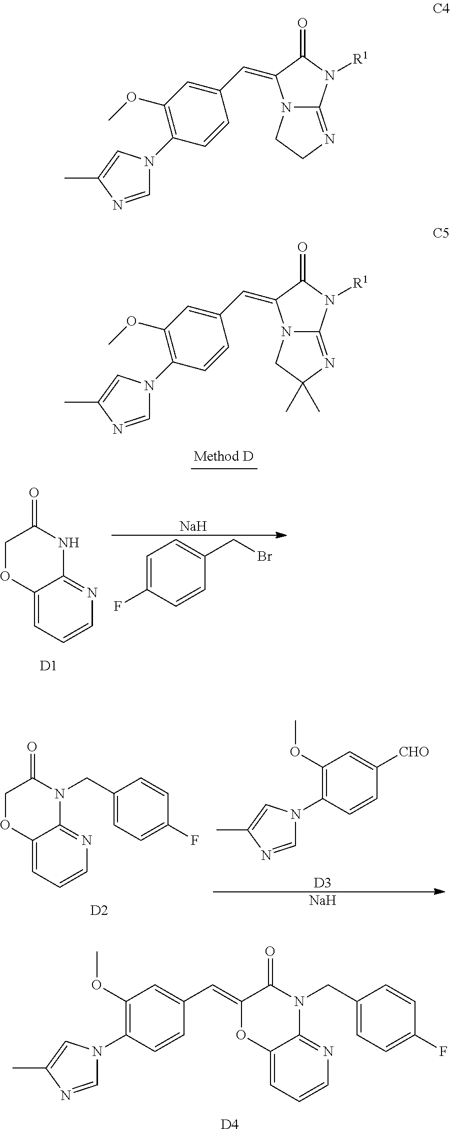

### Method D, Step 1

D1 was prepared according to Savelon et al, Bioorg. Med. Chem. 1998, 6, 133. To a slurry of D1 (301 mg, 2.0 mmol) in DMF (3 mL) was added NaH 60% in hexanes (92 mg, 2.30 mmol) followed by p-fluorobenzyl bromide (472 mg, 2.5 mmol). The reaction was stirred for 2 h at 120° C., then cooled and poured into brine and EtOAc. The organic layer was washed with brine, dried over sodium sulfate and concentrated. The residue was purified by chromatography over silica gel (eluted with Hexanes/EtOAc 99:1 to 50:50) to afford 372 mg (72%) of D2.

### Method D, Step 2

To a slurry of D2 (171 mg, 0.66 mmol) and D3 (134 mg, 0.62 mmol) in THF (3 mL) was added NaH 60% in oil (75 mg, 1.86 mmol) followed by DMF (0.2 mL). The reaction was stirred 3 h at 65° C., and then poured into dilute HCl. This mixture was neutralized with 1:1 saturated aq. sodium bicarbonate/brine and extracted with EtOAc. The organic layers were dried over sodium sulfate, filtered, and concentrated. The crude material was purified over silica gel (eluted with CH₂Cl₂/MeOH 9:1) to provide 54.1 mg of D4: ¹H NMR (CDCl₃ 400 MHz) δ 8.12 (m, 1H), 7.75 (s, 1H), 7.45-7.60 (m, 4H), 7.39 (d, J=8 Hz, 1H), 7.28 (d, J=8.8

Hz, 1H), 6.90-7.10 (m, 5H), 5.30 (s, 2H), 3.92 (s, 3H), 2.30 (s, 3H); LCMS (MH⁺)=457.2; retention time=2.10 min.

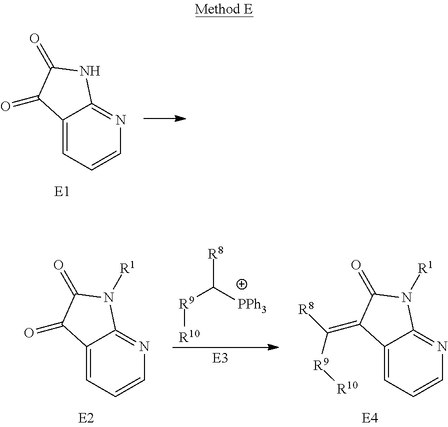

### Method E, Step 1

To a THF solution of E1 and R1-Br (R¹=α-methyl-p-fluorobenzyl) will be added NaH (1.1 eq) and the solution will be heated until the starting material disappears. After removal of volatiles, the residue will be chromatographed to give compound E2.

### Method E, Step 2.

To a THF solution of E2 (R¹=α-methyl-p-fluorobenzyl) will be added E3 (R⁸=H, R⁹=4-R¹⁰-3-Methoxyphenyl and R¹⁰=4-Methylimidazol-1-yl) and NaH (1.1 eq). The reaction mixture will be heated until the starting material disappears. After removal of volatiles, the residue will be chromatographed to give compound E4 (R¹=α-methyl-p-fluorobenzyl, R⁸=H, R⁹=4-R¹⁰-3-Methoxyphenyl and R¹⁰=4-Methylimidazol-1-yl).

The following compounds will be synthesized using method similar to Method E.

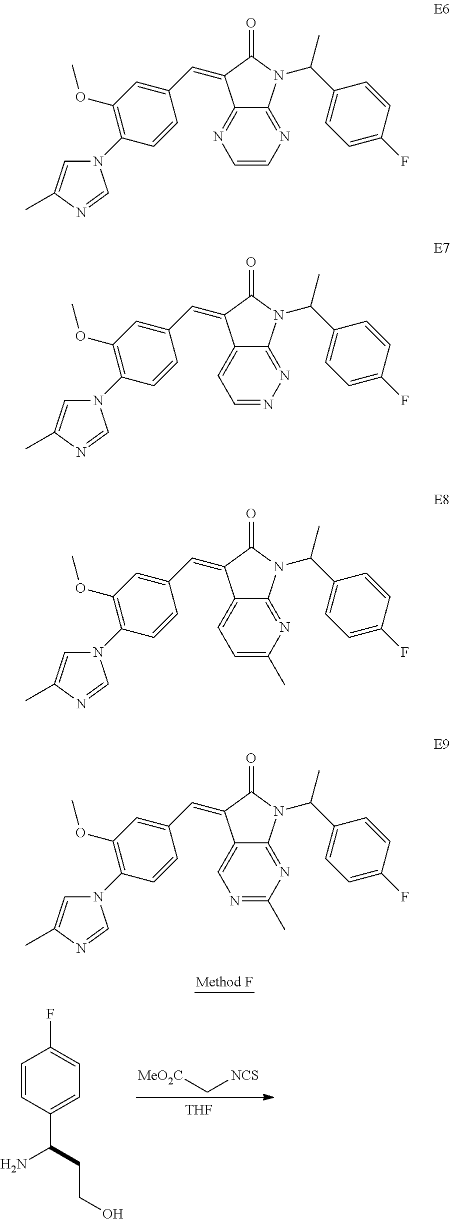

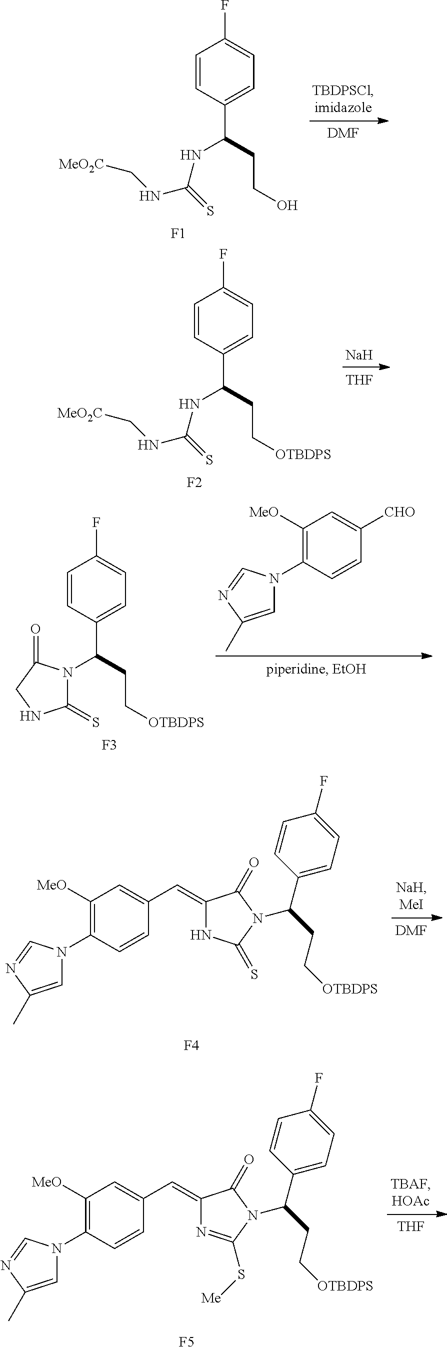

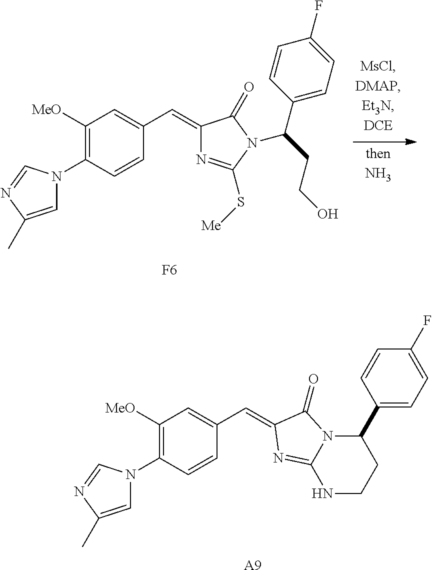

### Method F, Step 1

To a 0° C. solution of methyl 2-isothiocyanatoacetate (1.12 mL, 10.3 mmol) in tetrahydrofuran (30 mL) was added a solution of (R)-3-amino-3-(4-fluorophenyl)propan-1-ol (2.0 g) in tetrahydrofuran. After two minutes, the cooling bath was removed. After being stirred for 50 minutes, the reaction solution was diluted with ethyl acetate (150 mL). This solution was then washed twice with water, washed twice with aq. 1M HCl, washed once with saturated aq. NaHCO₃, washed once with brine, and dried over Na₂SO₄. This dried solution was filtered and concentrated to afford a brown oil that was used without purification.

### Method F, Step 2

The resulting, crude F1 was dissolved in dimethylformamide (20 mL), and then imidazole (1.4 g) and Pert-butylchlorodiphenylsilane (3.2 mL) were added to this room temperature solution. This solution was stirred at room temperature for 15 h and then used in the next step without additional manipulation.

### Method F, Step 3

To a 0° C. mixture of a 60% NaH oil dispersion (1.03 g) in tetrahydrofuran (20 mL) was added the crude, DMF reaction mixture from Step 2 containing F2, added dropwise over 20 minutes. This resulting reaction mixture was allowed to warm slowly as the cooling bath warmed. The reaction was quenched with water. This mixture was diluted with EtOAc/hexanes (3:1, 150 mL), and then washed twice with water. The combined aq. layers were extracted with EtOAc/hexanes (1:1, 20 mL). The combined organic layers were then washed twice with saturated aq. NH₄Cl, washed once with brine, and dried over Na₂SO₄ overnight. The dried organic layers were filtered, absorbed onto silica gel (17g), and chromatographed (EtOAc/hexanes) to afford compound F3 (4.30 g, 82% over three steps) as a yellow foam. ¹HNMR (CDCl₃, 400 MHz) δ 7.67-7.65 (m, 2H), 7.60-7.57 (m, 4H), 7.44-7.32 (m, 6H), 7.00 (t, 2H), 6.20 (t, 1H), 3.84 (s, 2H), 3.74-3.59 (m, 2H), 2.70-2.58 (m, 2H), 1.06 (s, 9H); MS (M+1)⁺ m/z calcd for C₂₈H₃₂FN₂O₂SSi⁺=507.2, found m/z=507.3.

### Method F, Step 4

A mixture of 3-methoxy-4-(4-methyl-1H-imidazol-1-yl)benzaldehyde (1.22 g, 5.64 mmol), F3 (4.30 g), piperidine (1.4 mL), and ethanol (23 mL) was heated to 85° C. for 3 h. The resulting solution was absorbed onto silica gel (18g) and chromatographed (MeOH/aq. NH₄OH/CH₂Cl₂) to afford slightly impure compound F4 (3.59 g) as a brown foam. MS (M+1)⁺ m/z calcd for C₄₀H₄₂FN₄O₃SSi⁺=705.3, found m/z=705.4.

### Method F, Step 5

To a 0° C. solution of F4 (3.59 g) in DMF (20 mL) was added a 60% NaH oil dispersion (0.22 g). After 15 minutes, methyl iodide (0.35 mL) was added to the 0° C. reaction mixture. This resulting reaction mixture was allowed to warm slowly as the cooling bath warmed. The reaction was quenched with water. This mixture was diluted with EtOAc/hexanes (3:1, 150 mL), washed twice with water, and then washed once with brine. The combined aq. layers were extracted with EtOAc/hexanes (1:1, 20 mL). The combined organic layers were dried over Na₂SO₄, filtered, and concentrated to afford crude compound F5 (3.66 g).

### Method F, Step 6

To a room temperature solution of F5 (3.66 g) in tetrahydrofuran (25 mL) was added acetic acid (0.44 mL), followed by a 1M solution of tetrabutylammonium fluoride in tetrahydrofuran (7.6 mL). After being stirred for 3 h at room temperature, the reaction solution was quenched with saturated aq. NaHCO₃. This mixture was then extracted three times with EtOAc. The combined organic layers were washed once with brine, and dried over Na₂SO₄, filtered, absorbed onto silica gel (15g), and chromatographed (MeOH/aq. NH₄OH/CH₂Cl₂) to afford compound F6 (1.69 g, 62% over three steps) as a dark yellow foam. ¹HNMR (CDCl₃, 500 MHz) δ 8.32 (s, 1H), 7.82 (s, 1H), 7.58 (dd, 1H), 7.52-7.49 (m, 2H), 7.32 (t, 1H), 7.10 (t, 2H), 7.02 (s, 1H), 6.97 (s, 1H), 5.48 (dd, 1H), 3.95 (s, 3H), 3.82 (ddd, 1H), 3.69 (ddd, 1H), 2.78 (m, 1H), 2.70 (s, 3H), 2.53 (m, 1H), 2.35 (s, 3H); MS (M+1)⁺ m/z calcd for C₂₅H₂₆FN₄O₃S⁺=481.2, found m/z=481.3.

### Method F, Step 7

To a room temperature solution of F6 (94 mg, 0.19 mmol), 4-dimethylaminopyridine (few small crystals), and triethylamine (82 μL) in 1,2-dichloroethane (2 mL) was added methanesulfonyl chloride (30 μL). After 20 minutes, a 0.5 M solution of NH₃ in 1,4-dioxane (4 mL) was added to the reaction mixture, which was then sealed and heated to 65° C. After 2h, a 7 M solution of NH₃ in methanol (1.1 mL) was added, and then heating was continued at 65° C. After being stirred 65° C. for 19 h, the reaction mixture was absorbed onto silica gel and chromatographed (MeOH/aq. NH₄OH/CH₂Cl₂) to provide impure (R)-A9. This material was further purified by reverse phase HPLC (water, acetonitrile, 0.1% trifluoroacetic acid) to afford (R)-A9 (R¹²=H) (34.1 mg, 40%) as a yellow solid. ¹HNMR (CD₃OD, 500 MHz) δ 9.20 (s, 1H), 7.65 (d, 1H), 7.62-7.61 (m, 2H), 7.48 (dd, 1H), 7.39-7.36 (m, 2H), 7.18 (t, 2H), 7.02 (s, 1H), 5.54 (dd, 1H), 4.07 (s, 3H), 3.68 (ddd, 1H), 3.35 (ddd, 1H), 2.51-2.44 (m, 1H), 2.46 (d, 3H), 2.30 (dddd, 1H); MS (M+1)⁺ m/z calcd for C₂₄H₂₃FN₅O₂⁺=432.2, found m/z=432.2.

The following compounds (“Cpd”) were made using a method similar to Method F:

| | | | m/z |
|---|---|---|---|
| | | Molecular | Found |
| Cpd | Structure | Weight | (M + 1)⁺ |
| (R)- B7 | 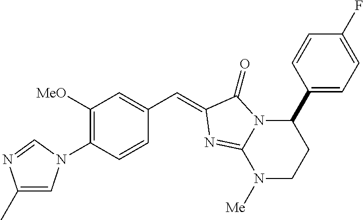 | 445.5 | 446.2 |
| F7 | 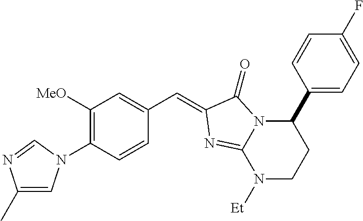 | 459.5 | 460.3 |
| F8 | 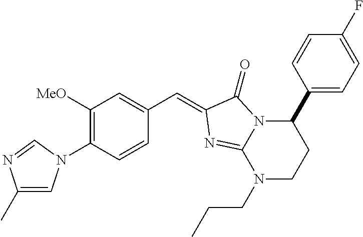 | 473.5 | 474.3 |
| F9 | 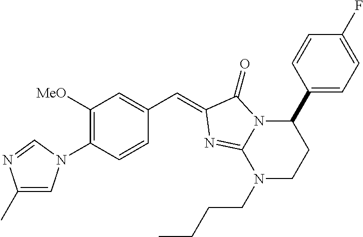 | 487.6 | 488.3 |
| F10 | 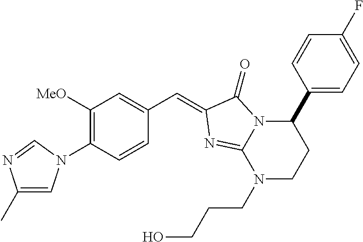 | 489.5 | 490.3 |
| F11 | 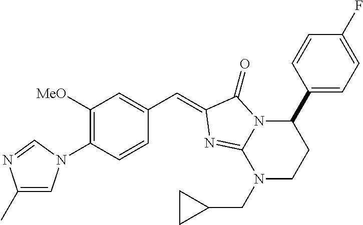 | 485.6 | 486.3 |
| F12 | 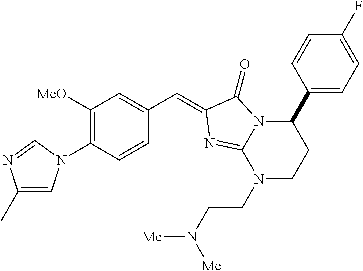 | 502.6 | 503.3 |
| F13 | 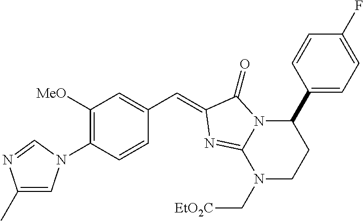 | 517.6 | 518.3 |
| (S)- A9 | 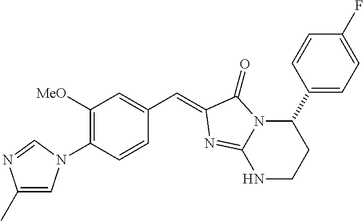 | 431.5 | 432.2 |
| (S)- B7 | 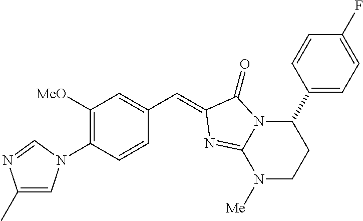 | 445.5 | 446.2 |
| F14 | 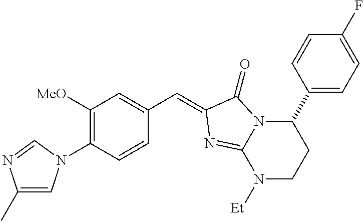 | 459.5 | 460.3 |
| F15 | 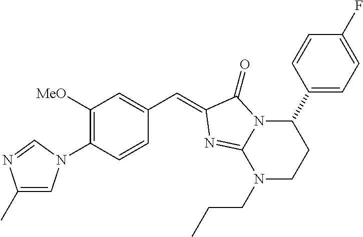 | 473.5 | 474.3 |
| F16 | 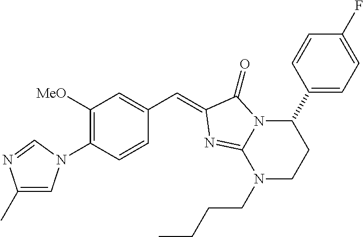 | 487.6 | 488.3 |
| F17 | 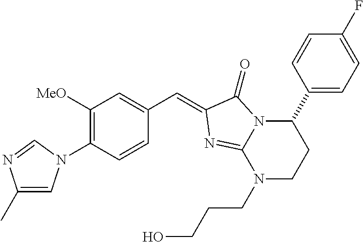 | 489.5 | 490.3 |
| F18 | 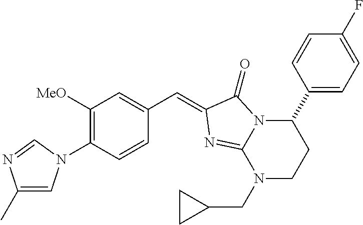 | 485.6 | 486.3 |
| F19 | 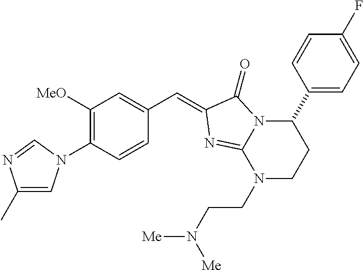 | 502.6 | 503.3 |

The following compounds will be synthesized using method similar to Method F.

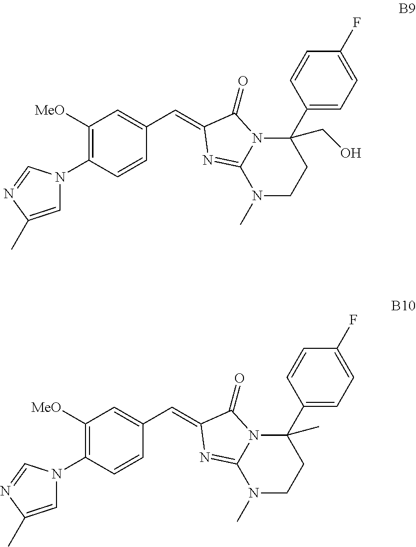

The following compounds (R¹²=n-Pr for d, n-Bu for e, —CH₂CH₂OH for f, —CH₂CH₂CH₂OH for g, —CH₂-cyclopropyl for h) will be synthesized using method similar to Method F.

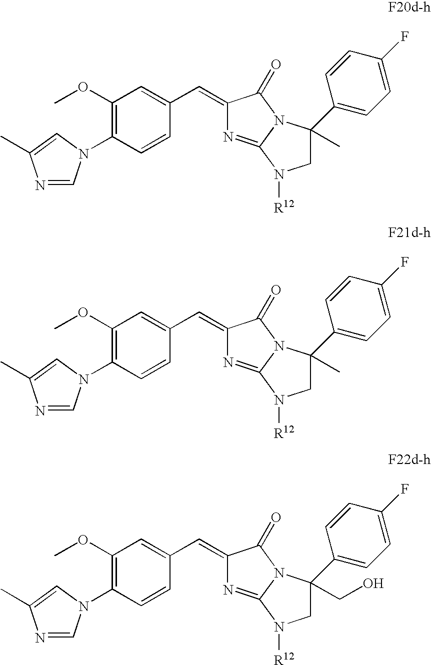

The following compounds (R¹²=Et for c, n-Pr for d, n-Bu for e, —CH₂CH₂OH for f, —CH₂CH₂CH₂OH for g, —CH₂-cyclopropyl for h) will be synthesized using method similar to Method F.

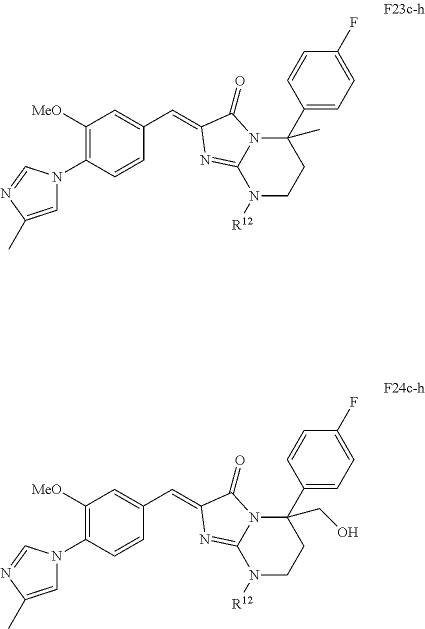

The following compounds (R¹²=H for a, Me for b, Et for c, n-Pr for d, n-Bu for e, —CH₂CH₂OH for f, —CH₂CH₂CH₂OH for g, —CH₂-cyclopropyl for h) will be synthesized using method similar to Method F.

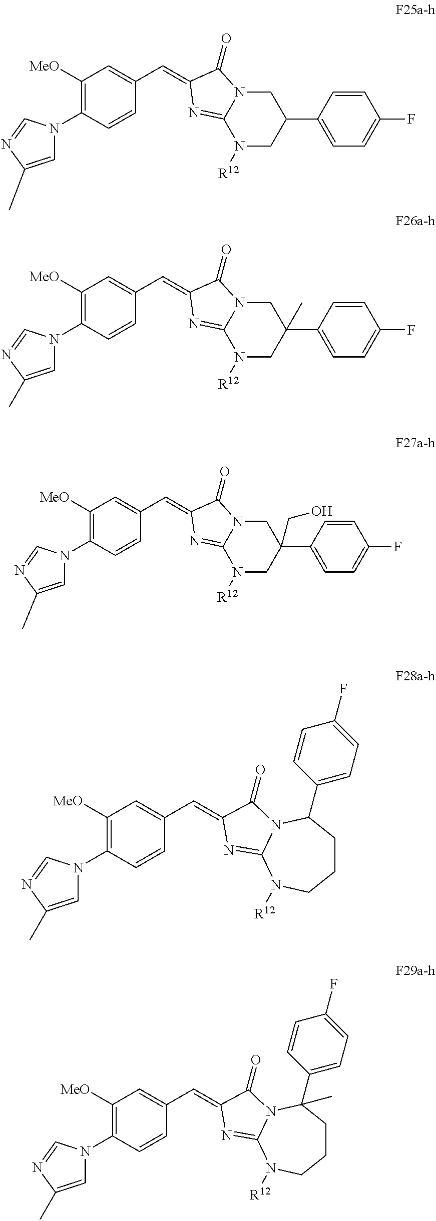

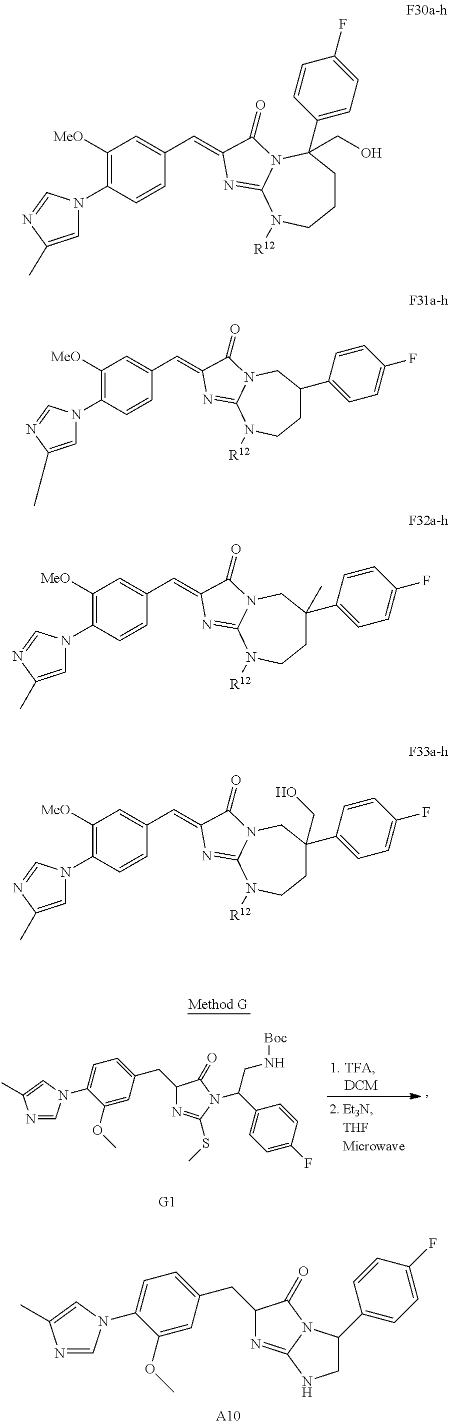

(Compound G1 was synthesized from tert-butyl 2-amino-2-(4-fluorophenyl)ethylcarbamate using a method similar to Method F, Steps 1 and 3-5.)

### Method G

To a round bottom flask at 0° C. containing a solution of G1 (191 mg, 0.34 mmol) in DCM (30 mL) was added trifluoroacetic acid (2.5 mL, 33.8 mmol). The reaction stirred for 0.25 h at 0° C. followed by warming to room temperature over 3 h. The reaction mixture was concentrated in vacuo and used crude in the next step. To a microwave vial containing a solution of the aforementioned crude material in THF (15 mL) was added triethylamine (4.7 mL, 33.8 mmol). The vial was then capped and heated in a microwave at 120° C. for 0.5 h on high absorption. The resulting mixture was concentrated in vacuo and purified by silica gel chromatography with methanol/ammonium hydroxide/DCM to afford compound A10 (R¹²=H) (101.5 mg, 72%) as a yellow solid. ¹HNMR (CDCl₃, 400 MHz) δ 7.82 (s, 1H), 7.66-7.62 (m, 2H), 7.34-7.31 (m, 2H), 7.23 (d, 1H), 7.10 (t, 2H), 7.07 (s, 1H), 6.62 (s, 1H), 5.33-5.30 (m, 1H), 4.45 (t, 1H), 3.85 (s, 4H), 2.29 (s, 3H); MS (M+1)⁺ m/z calcd for C₂₃H₂₀FN₅O₂⁺=418.4, found m/z=418.2.

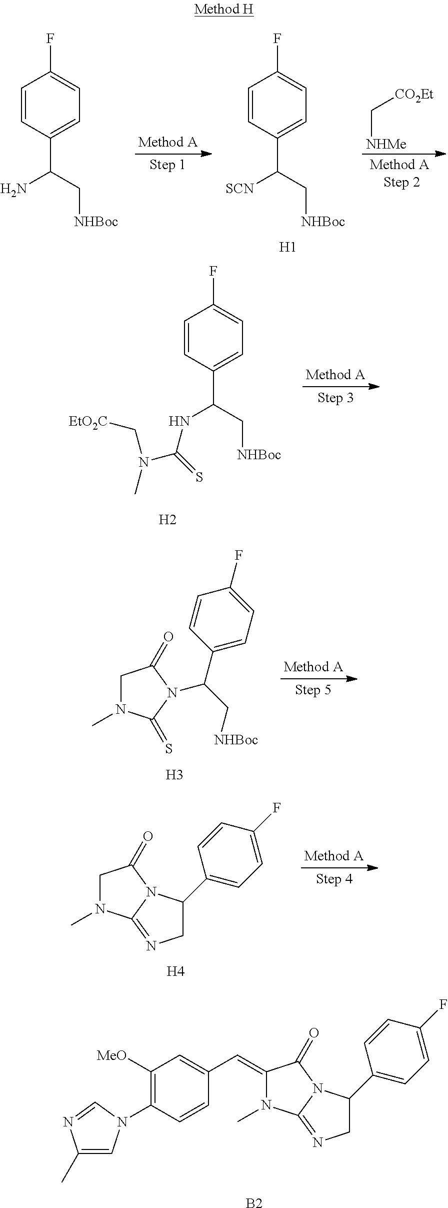

The following compounds will be synthesized using method similar to Method H.

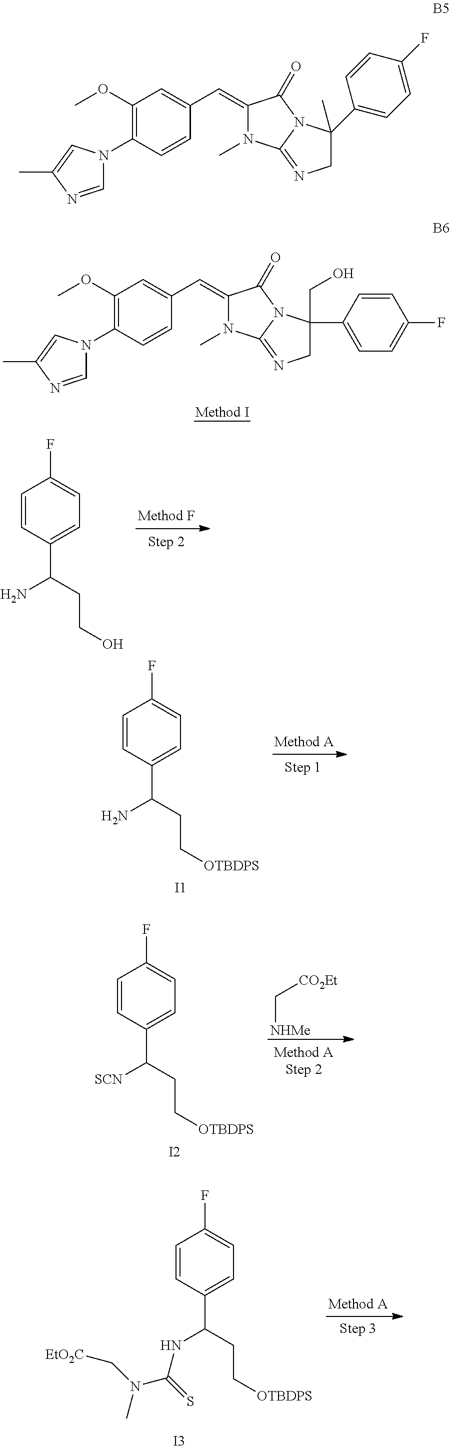

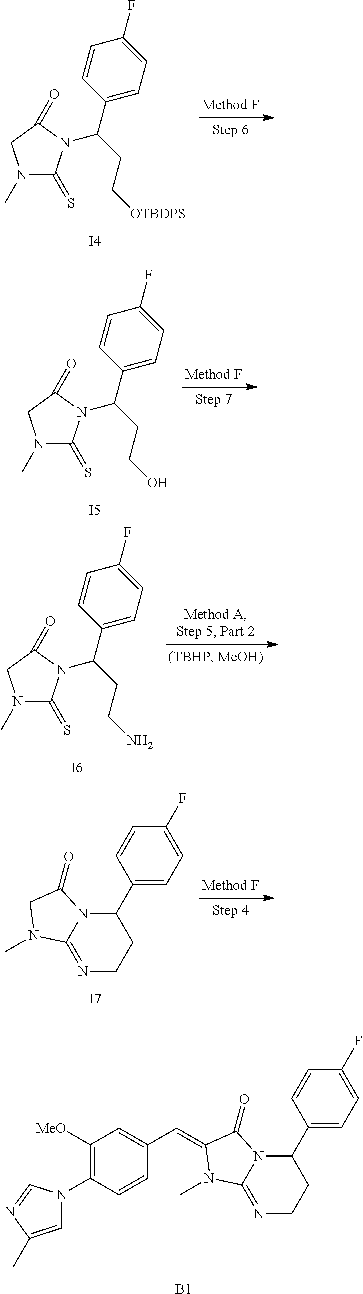

The following compounds will be synthesized using method similar to Method I.

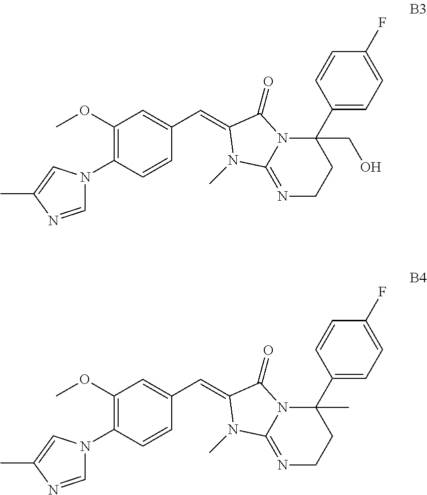

Starting from methyl 3-methyl-3-(methylamino)butanoate, the following compound will be synthesized using method similar to Method I:

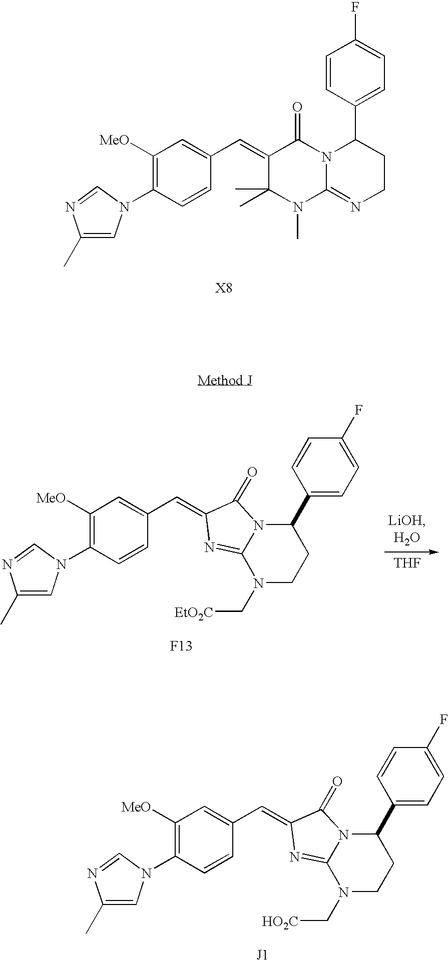

### Method J, Step 1

A mixture of F13 (10 mg, 19 μmol), LiOH—H₂O (4 mg), H₂O (0.5 mL), and tetrahydrofuran (1.5 mL) was sealed and heated to 65° C. After 1.5 h, the reaction mixture quenched with saturated aq. NH₄Cl. This mixture was then extracted three times with dichloromethane. The combined organic layers were washed once with brine, and dried over Na₂SO₄, filtered, and concentrated to provide 8.9 mg of a yellow residue. This crude material was purified by reverse phase HPLC (water, acetonitrile, 0.1% trifluoroacetic acid) to afford J1 (7.7 mg, 83%) as a yellow solid. ¹HNMR (CD₃CN, 500 MHz) δ 8.72 (d, 1H), 8.37 (d, 1H), 7.58 (dd, 2H), 7.43 (d, 1H), 7.39 (s, 1H), 7.32-7.30 (m, 2H), 7.16 (t, 2H), 6.57 (s, 1H), 5.35 (br d, 1H), 4.69 (d, 1H), 4.24 (d, 1H), 3.96 (s, 3H), 3.44 (ddd, 1H), 3.37 (ddd, 1H), 2.63-2.47 (m, 1H), 2.41 (d, 3H), 2.16 (dddd, 1H); MS (M+1)⁺ m/z calcd for C₂₆H₂₅FN₅O₄⁺=490.2, found m/z=490.3.

The following compound (“Cpd”) was made using a method similar to Method J:

| | | | m/z |
|---|---|---|---|
| | | Molecular | Found |
| Cpd | Structure | Weight | (M + 1)⁺ |
| J2 | 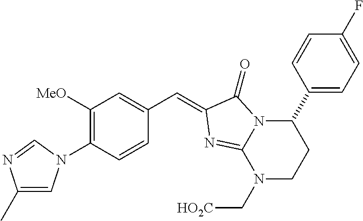 | 489.5 | 490.3 |

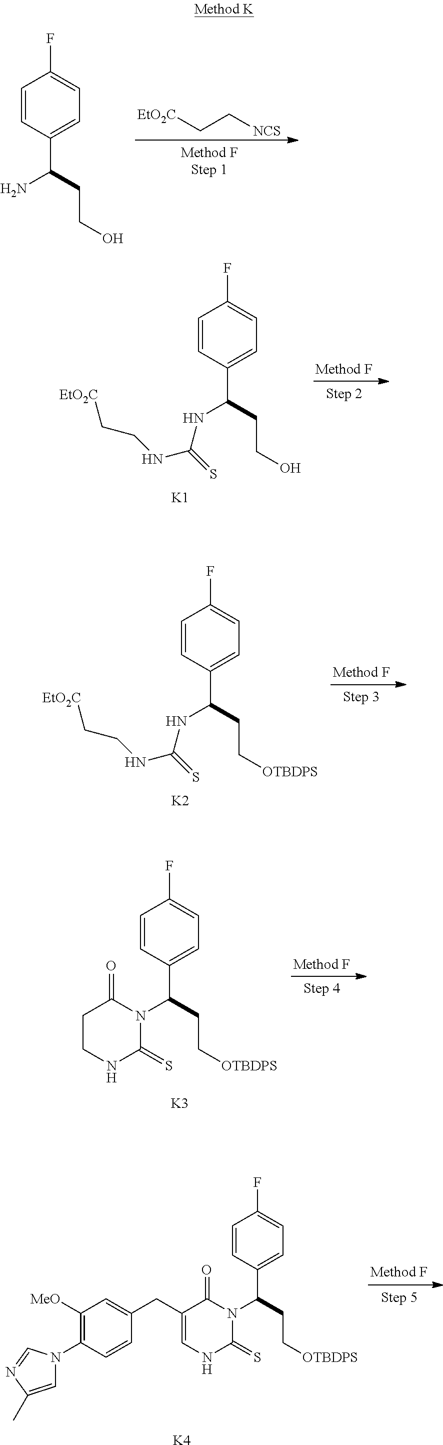

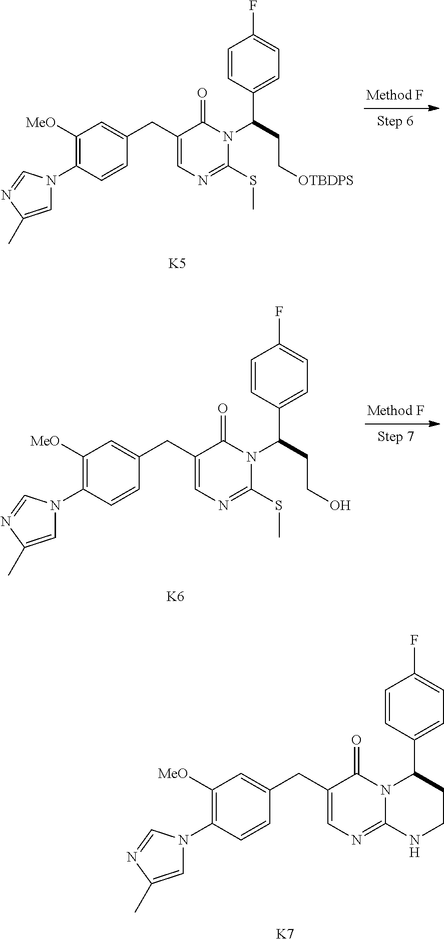

The following compounds (R¹²=Me for b, Et for c, n-Pr for d, n-Bu for e, —CH₂CH₂OH for f, —CH₂CH₂CH₂OH for g, —CH₂-cyclopropyl for h) will be synthesized using method similar to Method K.

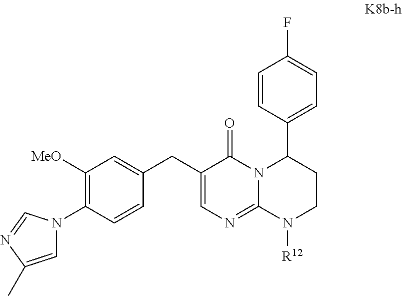

The following compounds (R¹²=H for a, Me for b, Et for c, n-Pr for d, n-Bu for e, —CH₂CH₂OH for f, —CH₂CH₂CH₂OH for g, —CH₂-cyclopropyl h) will be synthesized using method similar to Method K.

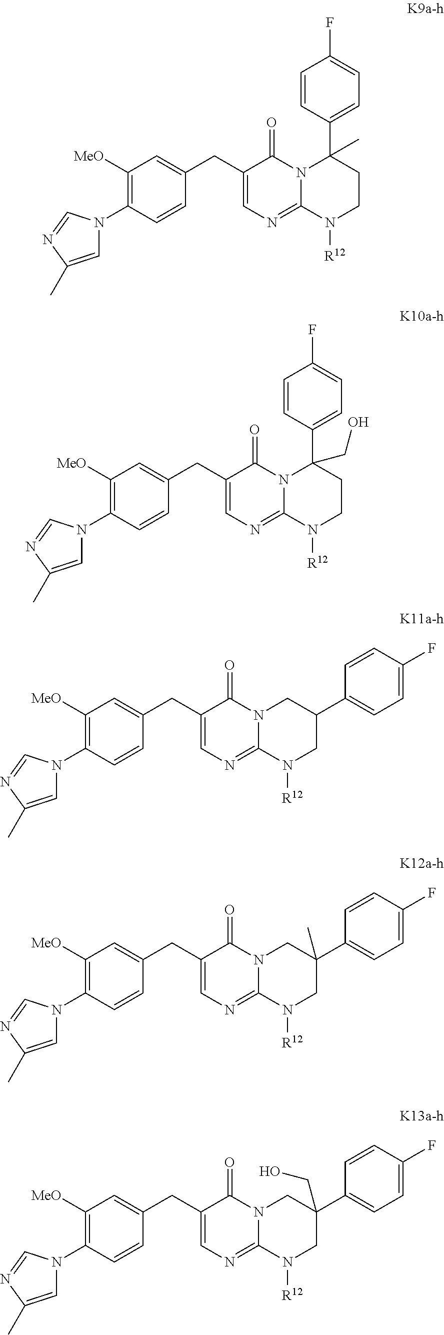

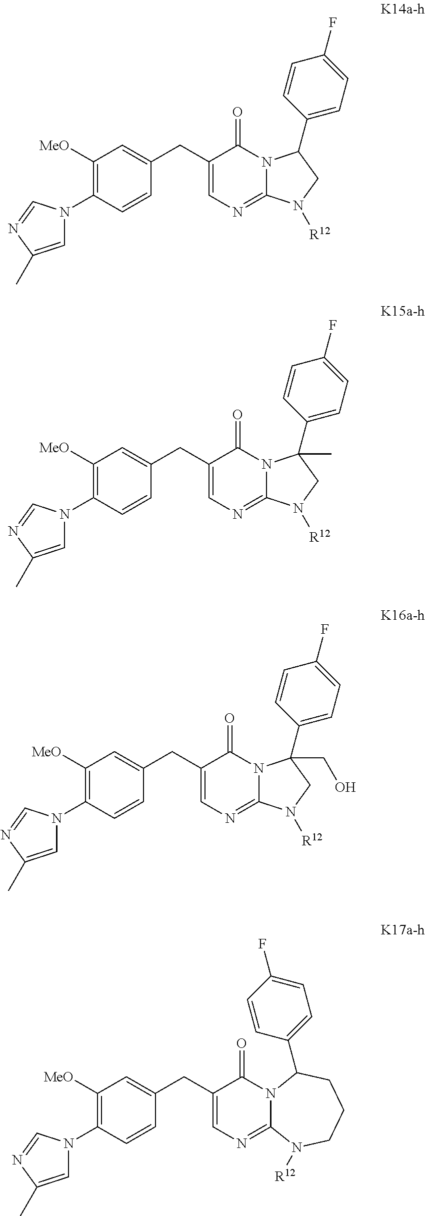

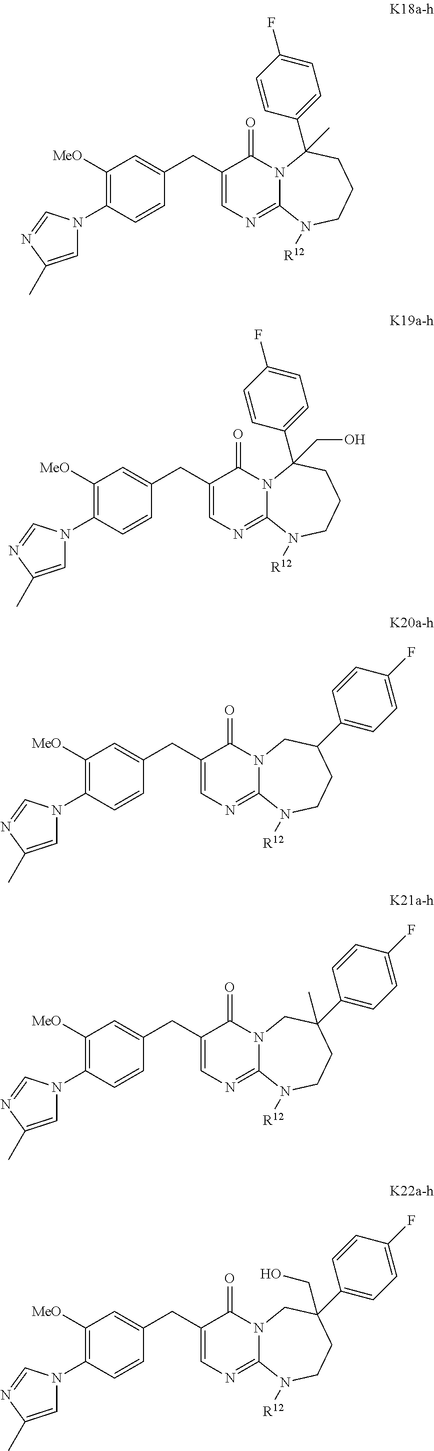

Starting from methyl 3-amino-3-methylbutanoate, the following compound will be synthesized using method similar to Method K.

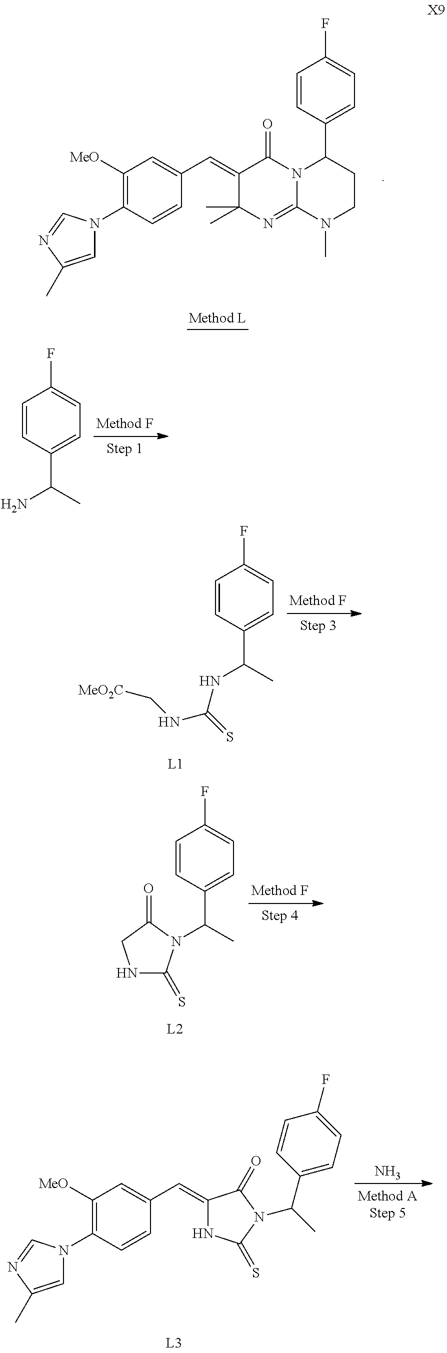

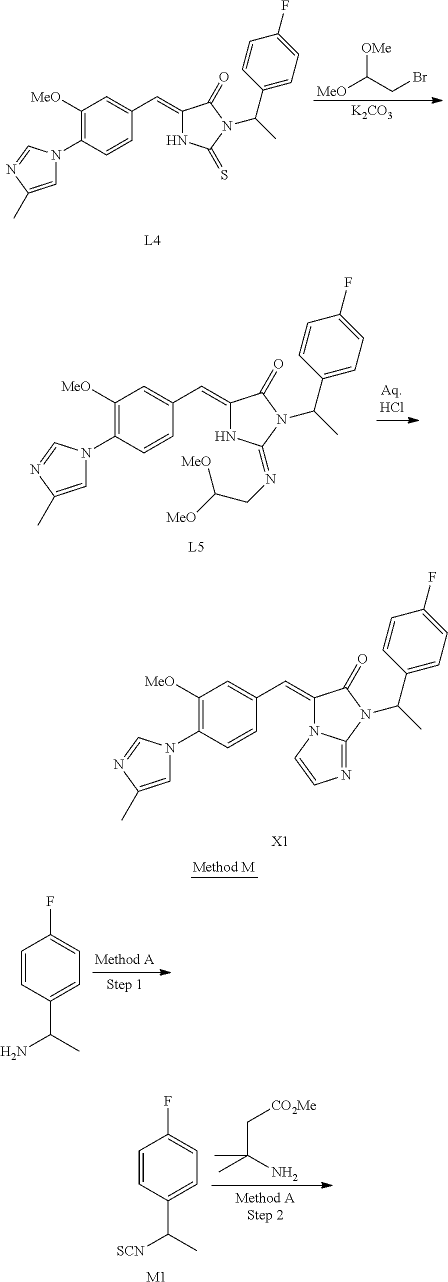

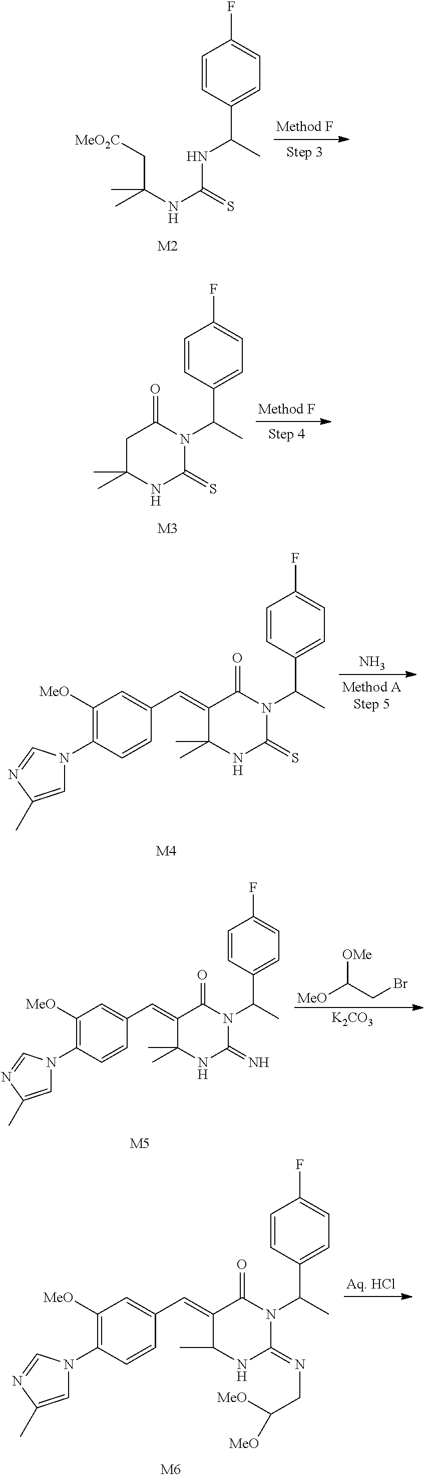

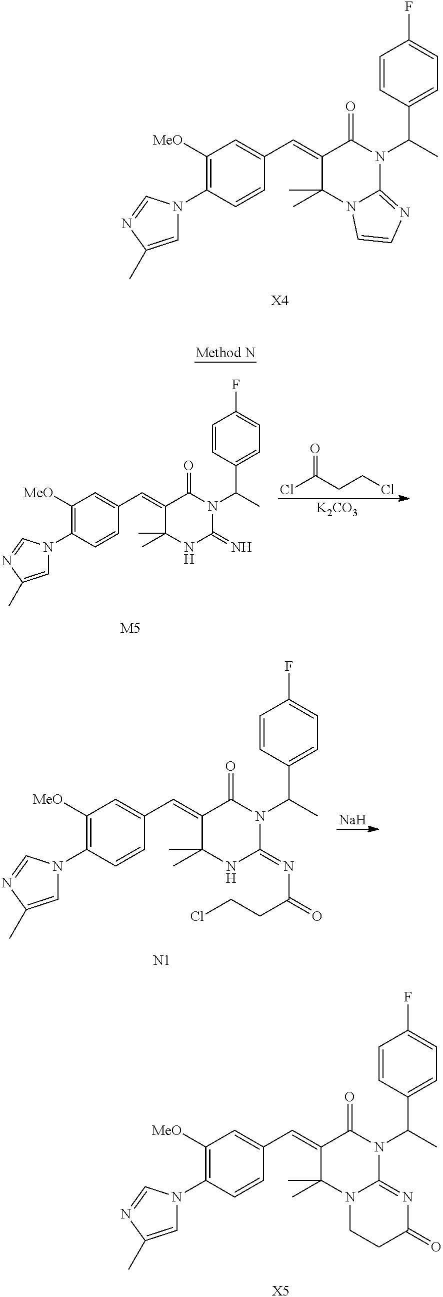

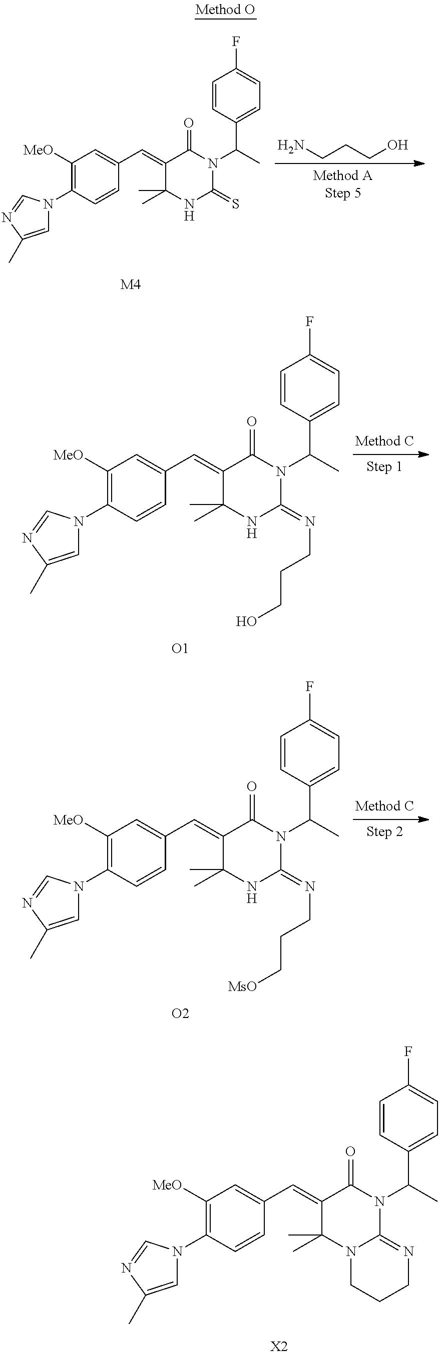

The following compound will be synthesized using method similar to Method O:

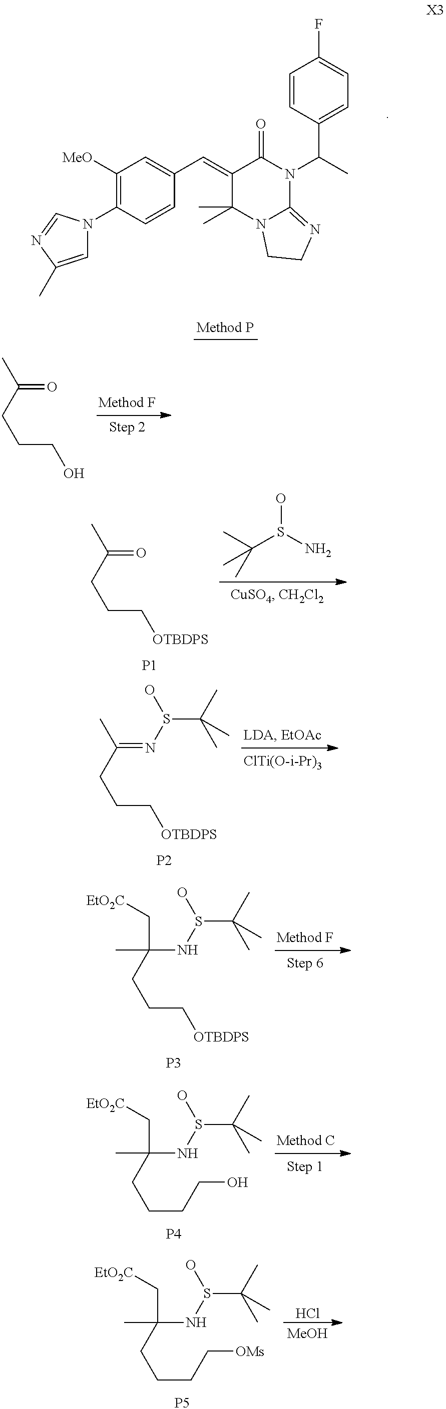

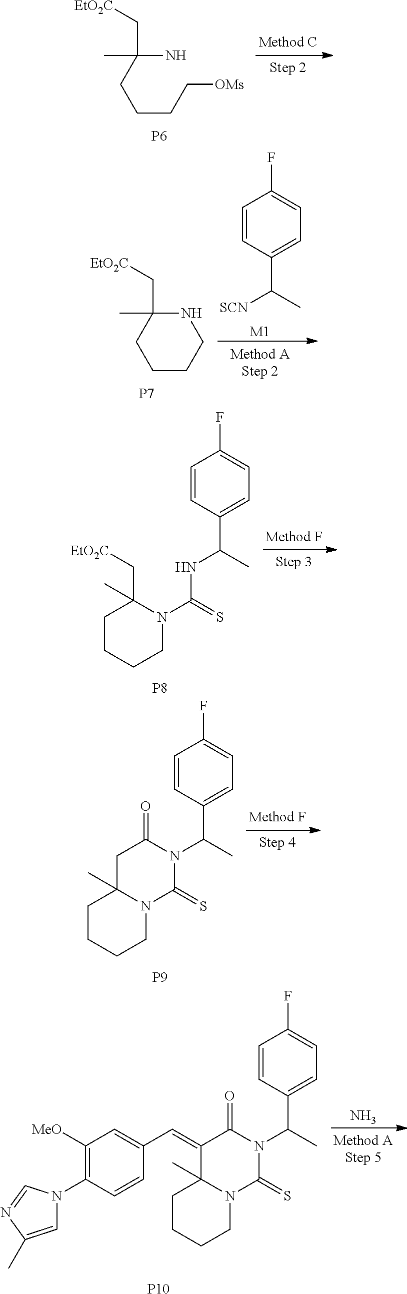

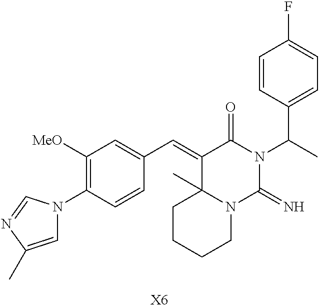

The following compound will be synthesized using method similar to Method P:

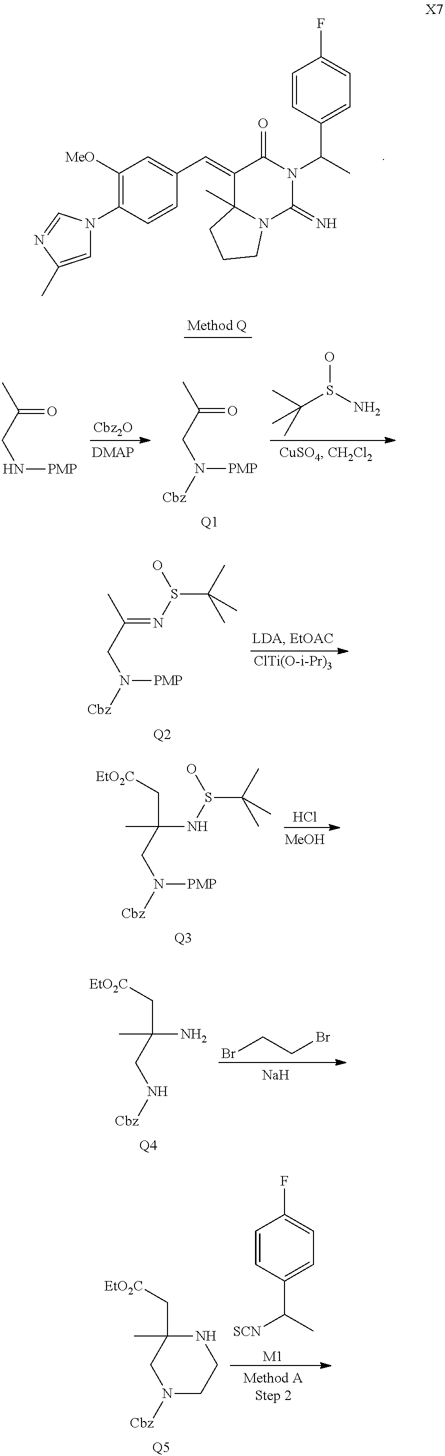

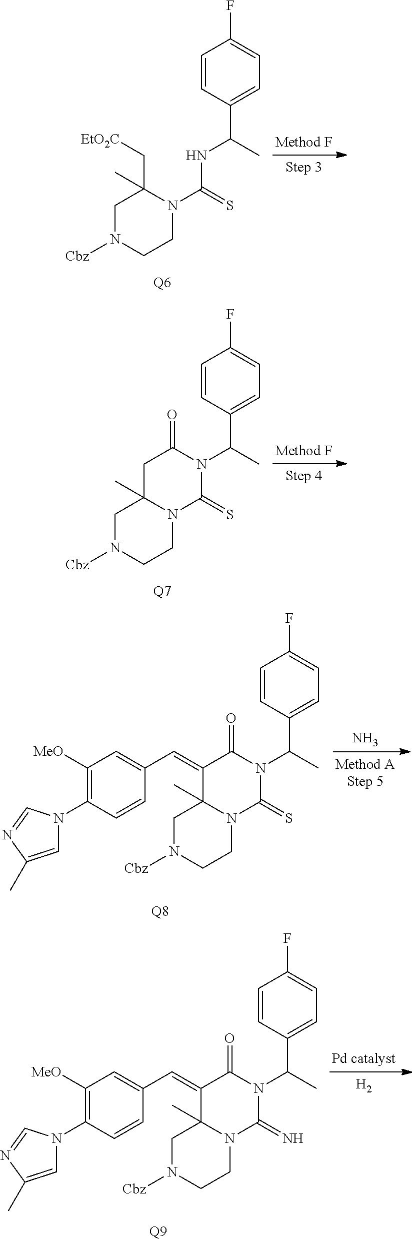

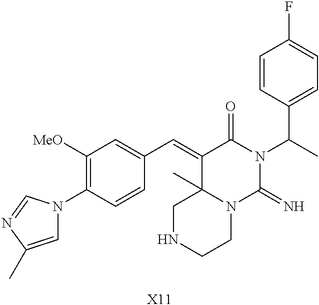

### Assay:

Secretase Reaction and Aβ Analysis in Whole Cells: HEK293 cells overexpressing APP with Swedish and London mutations were treated with the specified compounds for 5 hour at 37° C. in 100 ml of DMEM medium containing 10% fetal bovine serum. At the end of the incubation, total Aβ, Aβ40 and Aβ42 were measured using electrochemiluminescence (ECL) based sandwich immunoassays. Total Aβ was determined using a pair of antibodies TAG-WO2 and biotin-4G8, Aβ40 was identified with antibody pairs TAG-G2-10 and biotin-4G8, while Aβ42 was identified with TAG-G2-11 and biotin-4G8. The ECL signal was measured using Sector Imager 2400 (Meso Scale Discovery).

MS Analysis of Aβ Profile: Aβ profile in conditioned media was determined using surface enhanced laser desorption/ionization (SELDI) mass spectrometry. Conditioned media was incubated with antibody W02 coated PS20 ProteinChip array. Mass spectra of Aβ captured on the array were read on SELDI ProteinChip Reader (Bio-Rad) according to manufacture's instructions.

CSF Aβ Analysis: Aβ in rat CSF was determined using MSD technology as described above. Aβ40 was measured using antibody pair Tag-G2-10 and biotin-4G8, while Aβ342 was measured using Tag-anti Aβ42 (Meso Scale Discovery) and biotin-4G8. The ECL signal was measured using Sector Imager 2400 (Meso Scale Discovery).

Matrix-assisted laser desorption/ionization mass spectrometric (MALDI MS) analysis of Aβ is performed on a Voyager-DE STR mass spectrometer (ABI, Framingham, Mass.). The instrument is equipped with a pulsed nitrogen laser (337 nm). Mass spectra are acquired in the linear mode with an acceleration voltage of 20 kV. Each spectrum presented in this work represents an average of 256 laser shots. To prepare the sample-matrix solution, 1 μL of immunoprecipitated Aβ sample is mixed with 3 μL of saturated α-cyano-4-hydroxycinnamic acid solution in 0.1% TFA/acetonitrile. The sample-matrix solution is then applied to the sample plate and dried at ambient temperature prior to mass spectrometric analysis. All the spectra are externally calibrated with a mixture of bovine insulin and ACTH (18-39 clip).

The compounds (“Cpd”) in Table 2 had an Aβ 42 IC₅₀ in the range of about 85 nM to about 13807 nM. Compounds (R)-A9, (R)-B7, F7-F13, J1, (S)-A9, (S)-B7, F14-F19, J2, A10, B8, B15 and D3 in Table 2 had a Total Aβ/Aβ 42 in the range of 1.4 to 188.

TABLE 2

| | | | m/z |
|---|---|---|---|
| | | Molecular | Found |
| Cpd | Structure | Weight | (M + 1)⁺ |
| (R)-A9 | 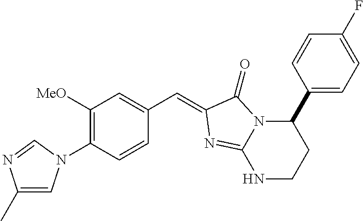 | 431.5 | 432.2 |
| (R)-B7 | 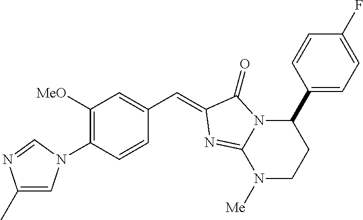 | 445.5 | 446.2 |
| F7 | 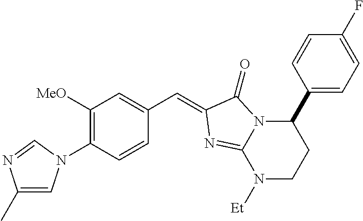 | 459.5 | 460.3 |
| F8 | 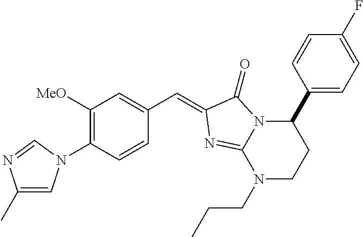 | 473.5 | 474.3 |
| F9 | 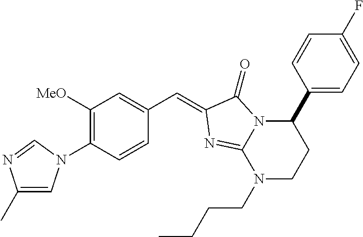 | 487.6 | 488.3 |
| F10 | 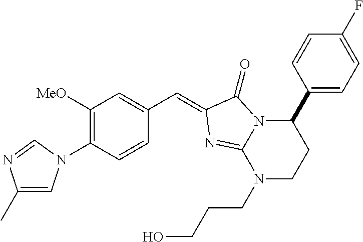 | 489.5 | 490.3 |
| F11 | 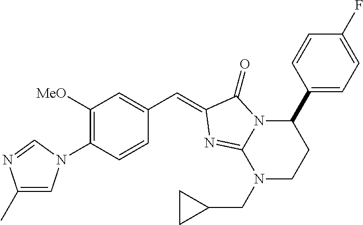 | 485.6 | 486.3 |
| F12 | 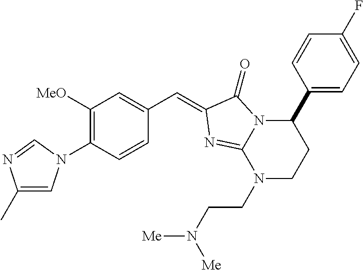 | 502.6 | 503.3 |
| F13 | 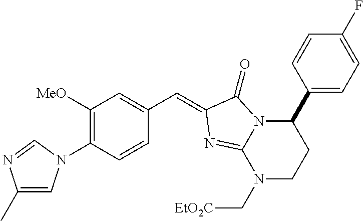 | 517.6 | 518.3 |
| J1 | 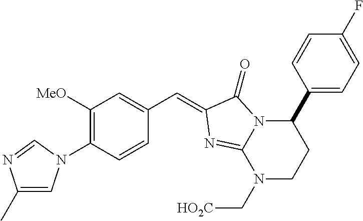 | 489.5 | 490.3 |
| (S)-A9 | 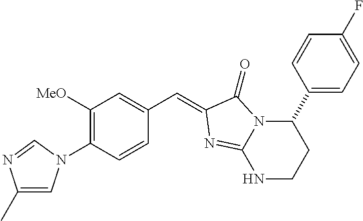 | 431.5 | 432.2 |
| (S)-B7 | 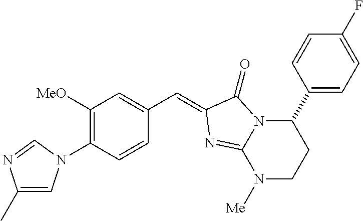 | 445.5 | 446.2 |
| F14 | 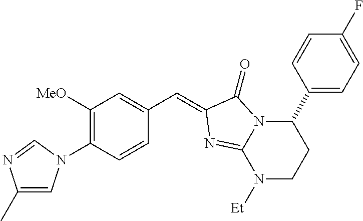 | 459.5 | 460.3 |
| F15 | 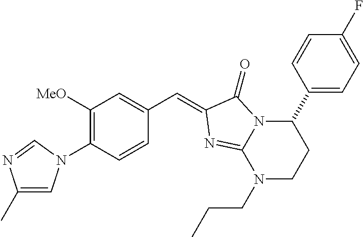 | 473.5 | 474.3 |
| F16 | 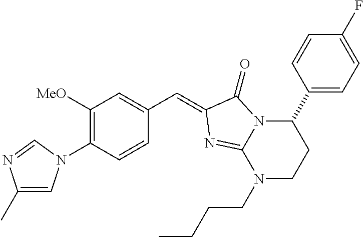 | 487.6 | 488.3 |
| F17 | 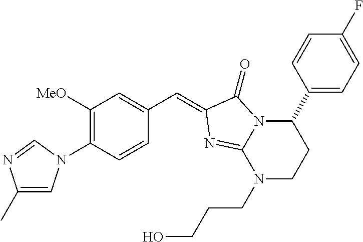 | 489.5 | 490.3 |
| F18 | 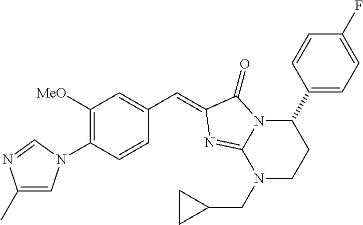 | 485.6 | 486.3 |
| F19 | 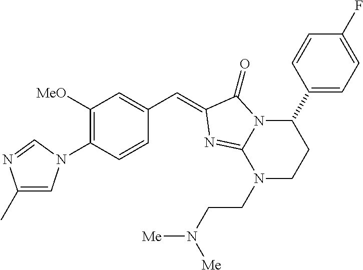 | 502.6 | 503.3 |
| J2 | 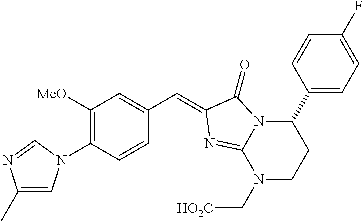 | 489.5 | 490.3 |
| A10 | 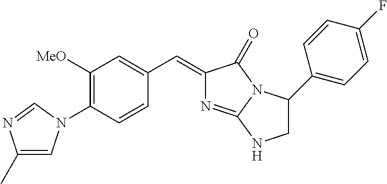 | 417.4 | 418.2 |
| B8 | 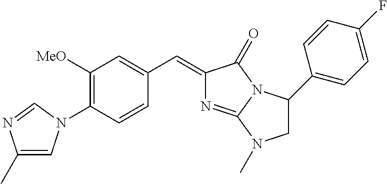 | 431.4 | 432.2 |
| B15 | 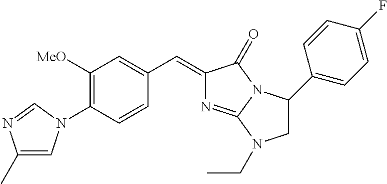 | 445.5 | 446.2 |
| C3 | 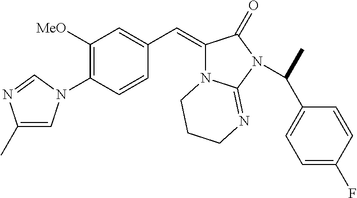 | 459.5 | 460.3 |
| D3 | 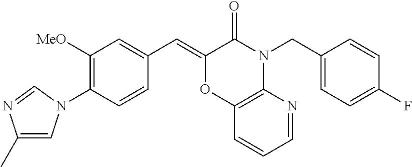 | 456.5 | 457.2 |

While the present invention has been described in conjunction with the specific embodiments set forth above, many alternatives, modifications and other variations thereof will be apparent to those of ordinary skill in the art. All such alternatives, modifications and variations are intended to fall within the spirit and scope of the present invention.

1. A compound of the formula: 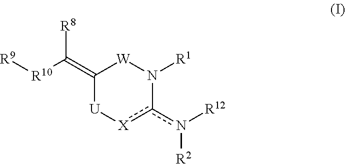
or a pharmaceutically acceptable salt thereof, wherein:
either
(i) R¹ and R² are joined together to form a 5-14 membered heteroaryl, 5-8 membered heterocyclyl or 5-8 membered heterocyclenyl moiety, wherein: (a) said heteroaryl moiety is optionally substituted with 1 to 5 independently selected R²¹ groups, (b) said heterocyclyl moiety is optionally substituted with 1 to 5 independently selected R²¹ groups, (c) said heterocyclenyl moiety is optionally substituted with 1 to 5 independently selected R²¹ groups, and (d) said heteroaryl, heterocyclyl or heterocyclenyl moiety is optionally fused with an aryl or heteroaryl ring, and the ring moiety resulting from the fusion is optionally substituted with 1 to 5 independently selected R²¹ groups; or
(ii) R² and R⁶ are joined together to form a 5-14 membered heteroaryl, 5-8 membered heterocyclyl or 5-8 membered heterocyclenyl moiety, wherein: (a) said heteroaryl moiety is optionally substituted with 1 to 5 independently selected R²¹ groups, (b) said heterocyclyl moiety is optionally substituted with 1 to 5 independently selected R²¹ groups, (c) said heterocyclenyl moiety is optionally substituted with 1 to 5 independently selected R²¹ groups, and (d) said heteroaryl, heterocyclyl or heterocyclenyl moiety is optionally fused with an aryl or heteroaryl ring, and the ring moiety resulting from the fusion is optionally substituted with 1 to 5 independently selected R²¹ groups; or
(iii) R⁵ and R⁶ are joined together to form a 5-14 membered heteroaryl, 5-8 membered heterocyclyl or 5-8 membered heterocyclenyl moiety, wherein: (a) said heteroaryl moiety is optionally substituted with 1 to 5 independently selected R²¹ groups, (b) said heterocycyl moiety is optionally substituted with 1 to 5 independently selected R²¹ groups, (c) said heterocyclenyl moiety is optionally substituted with 1 to 5 independently selected R²¹ groups, and (d) said heteroaryl, heterocyclyl or heterocyclenyl moiety is optionally fused with an aryl or heteroaryl ring, and the ring moiety resulting from the fusion is optionally substituted with 1 to 5 independently selected R²¹ groups; or
(iv) R² and R¹⁴ are joined together to form a 5-14 membered heteroaryl, 5-8 membered heterocyclyl or 5-8 membered heterocyclenyl moiety, wherein: (a) said heteroaryl moiety is optionally substituted with 1 to 5 independently selected R²¹ groups, (b) said heterocyclyl moiety is optionally substituted with 1 to 5 independently selected R²¹ groups, (c) said heterocyclenyl moiety is optionally substituted with 1 to 5 independently selected R²¹ groups, and (d) said heteroaryl, heterocyclyl or heterocyclenyl moiety is optionally fused with an aryl or heteroaryl ring, and the ring moiety resulting from the fusion is optionally substituted with 1 to 5 independently selected R²¹ groups; or
(v) R³ and R¹⁴ are joined together to form a 5-14 membered heteroaryl, 5-8 membered heterocyclyl or 5-8 membered heterocyclenyl moiety, wherein: (a) said heteroaryl moiety is optionally substituted with 1 to 5 independently selected R²¹ groups, (b) said heterocyclyl moiety is optionally substituted with 1 to 5 independently selected R²¹ groups, (c) said heterocyclenyl moiety is optionally substituted with 1 to 5 independently selected R²¹ groups, and (d) said heteroaryl, heterocyclyl or heterocyclenyl moiety is optionally fused with an aryl or heteroaryl ring, and the ring moiety resulting from the fusion is optionally substituted with 1 to 5 independently selected R²¹ groups; or
(vi) R³ and R⁶ are joined together to form a 5-14 membered aryl, 5-8 membered cycloakyl, 5-8 membered cycloalkenyl, 5-14 membered heteroaryl, 5-8 membered heterocyclyl or 5-8 membered heterocyclenyl moiety, wherein: (a) said aryl moiety is optionally substituted with 1 to 5 independently selected R²¹ groups, (b) said cycloalkyl moiety is optionally substituted with 1 to 5 independently selected R²¹ groups, (c) said cycloalkenyl moiety is optionally substituted with 1 to 5 independently selected R²¹ groups, (d) said heteroaryl moiety is optionally substituted with 1 to 5 independently selected R²¹ groups, (e) said heterocyclyl moiety is optionally substituted with 1 to 5 independently selected R²¹ groups, (f) said heterocyclenyl moiety is optionally substituted with 1 to 5 independently selected R²¹ groups, and (g) said aryl, cycloalkyl, cycloalkenyl, heteroaryl, heterocyclyl or heterocyclenyl moiety is optionally fused with an aryl or heteroaryl ring, and the ring moiety resulting from the fusion is optionally substituted with 1 to 5 independently selected R²¹ groups; or
(vii) R⁵ and R¹⁴ are joined together to form a 5-14 membered heteroaryl, 5-8 membered heterocyclyl or 5-8 membered heterocyclenyl moiety, wherein: (a) said heteroaryl moiety is optionally substituted with 1 to 5 independently selected R²¹ groups, (b) said heterocycyl moiety is optionally substituted with 1 to 5 independently selected R²¹ groups, (c) said heterocyclenyl moiety is optionally substituted with 1 to 5 independently selected R²¹ groups, and (d) said heteroaryl, heterocyclyl or heterocyclenyl moiety is optionally fused with an aryl or heteroaryl ring, and the ring moiety resulting from the fusion is optionally substituted with 1 to 5 independently selected R²¹ groups;
W is —S(O)—, —S(O)₂— or —C(O)—;
U is a bond, —C(O)—, —O—, —N(R⁵)— or —C(R³)(R⁴)—;
X is —N(R¹⁴)— or —C(R⁶)(R⁷)—;
The dashed lines [symbol] in formula (I) represent optional bonds provided that: (a) only one optional bond can be present, and (b) when the optional bond between the nitrogen (of the NR² moiety) and the ring carbon is present then R¹² is absent;
R¹ (when R¹ is not joined to R²) is independently selected from the group consisting of H, alkyl, alkenyl, alkynyl, aryl, arylalkyl-, alkylaryl-, cycloalkyl, cycloalkylalkyl-, heteroaryl, heteroarylalkyl-, heterocyclyl and heterocyclyalkyl-, wherein each of said alkyl, alkenyl, alkynyl, aryl, aryalkyl-, alkylaryl-, cycloalkyl, cycloalkylalkyl-, heteroaryl, heteroarylalkyl-, heterocyclyl and heterocyclyalkyl-R¹ group is optionally substituted with 1-5 independently selected R²¹ substituents;
R¹ (when R² is not joined to R¹, R⁶ or R¹⁴) is independently selected from the group consisting of H, alkyl, alkenyl, alkynyl, cycloalkyl, cycloalkylalkyl-, cycloalkenyl, heterocyclyl, heterocyclylalkyl-, aryl, arylalkyl-, heteroaryl, heteroarylalkyl-, —CN, —C(O)R¹⁵, —C(O)OR¹⁵, —C(O)N(R¹⁵)(R¹⁶), —S(O)N(R¹⁵)(R¹⁶), —S(O)₂N(R¹⁵)(R¹⁶), —S(O)R¹⁵, —S(O)R¹⁵, —C(═NOR¹⁵)R¹⁶ and —P(O)(OR¹⁵)(OR¹⁶), and wherein each of said alkyl, alkenyl, alkynyl, cycloalkyl, cycloalkylalkyl-, cycloalkenyl, heterocyclyl, heterocyclyalkyl-, aryl, arylalkyl-, heteroaryl, and heteroarylalkyl R² group is optionally substituted with 1-5 independently selected R²¹ groups;
R³ (when R³ is not joined to R⁵ or R¹⁴) is independently selected from the group consisting of H, alkyl, alkenyl, alkynyl, aryl, arylalkyl-, alkylaryl-, cycloalkyl, cycloalkylalkyl-, heteroaryl, heteroarylalkyl-, heterocyclyl and heterocyclyalkyl-, wherein each of said alkyl-, alkenyl, alkynyl-, aryl-, arylalkyl-, alkylaryl-, cycloalkyl-, cycloalkylalkyl-, heteroaryl-, heteroarylalkyl-, heterocyclyl and heterocyclyalkyl-R³ group is optionally substituted with 1-5 independently selected R²¹ groups;
R⁴ is independently selected from the group consisting of H, alkyl, alkenyl, alkynyl, aryl, arylalkyl-, alkylaryl-, cycloalkyl-, cycloalkylalkyl-, heteroaryl-, heteroarylalkyl-, heterocyclyl- and heterocyclyalkyl-, and wherein each of said alkyl, alkenyl, alkynyl, aryl, arylalkyl-, alkylaryl-, cycloalkyl, cycloalkylalkyl-, heteroaryl, heteroarylalkyl-, heterocyclyl and heterocyclyalkyl-R⁴ group is optionally substituted with 1-5 independently selected R²¹ substituents;
R⁵ (when R⁵ is not joined to R⁵ or R¹⁴) is independently selected from the group consisting of H, alkyl, alkenyl, alkynyl, cycloalkyl, cycloalkylalkyl, cycloalkenyl, heterocyclyl, heterocyclylalkyl, aryl, arylalkyl, heteroaryl, heteroarylalkyl, —CN, —C(O)R¹⁵, —C(O)OR¹⁵, —C(O)N(R¹⁵)(R¹⁶), —S(O)N(R¹⁵)(R¹⁶), —S(O)₂N(R¹⁵)(R¹⁶), —S(O)R¹⁵, —S(O)₂R¹⁵, —C(═NOR¹⁵)R¹⁶ and —P(O)(OR¹⁵)(OR¹⁶), and wherein each of said alkyl, alkenyl, alkynyl, cycloalkyl, cycloalkylalkyl, cycloalkenyl, heterocyclyl, heterocycylalkyl, aryl, arylalkyl, heteroaryl, and heteroaryalkyl R⁵ groups is optionally substituted with 1-5 independently selected R²¹ groups;
R⁶ (when R⁶ is not joined to R², R³ or R⁵) is independently selected from the group consisting of H, alkyl, alkenyl, alkynyl, aryl, arylalkyl-, alkylaryl-, cycloalkyl, cycloalkylalkyl-, heteroaryl, heteroarylalkyl-, heterocyclyl- and heterocyclyalkyl-, wherein each of said alkyl- alkenyl- alkynyl, aryl-, arylalkyl-, alkylaryl-, cycloalkyl-, cycloalkylalkyl-, heteroaryl, heteroarylalkyl-, heterocyclyl- and heterocyclyalkyl-R⁶ group is optionally substituted with 1-5 independently selected R²¹;
R⁷ is independently selected from the group consisting of H, alkyl, alkenyl, alkynyl, aryl, aryalkyl-, alkylaryl-, cycloalkyl, cycloalkylalkyl-, heteroaryl, heteroarylalkyl-, heterocyclyl and heterocyclyalkyl- and wherein each of said alkyl, alkenyl, alkynyl, aryl, arylalkyl-, alkylaryl-, cycloakyl, cycloalkylalkyl-, heteroaryl, heteroarylalkyl-, heterocyclyl and heterocyclyalkyl-R⁷ group is optionally substituted with 1-5 independently selected R²¹ substituents;
R⁸ is independently selected from the group consisting of H, alkyl, alkenyl, alkynyl, aryl, arylalkyl-, alkylaryl-, cycloalkyl, cycloalkylalkyl-, heteroaryl, heteroarylalkyl-, heterocyclyl and heterocyclyalkyl-, and wherein each of said alkyl, alkenyl, alkynyl, aryl, arylalkyl-, alkylaryl-, cycloalkyl, cycloalkylalkyl-, heteroaryl, heteroarylalkyl-, heterocyclyl and heterocyclyalkyl-R⁸ group is optionally substituted with 1-3 independently selected R²¹ substituents;
R⁹ is independently selected from the group consisting of alkyl, alkenyl alkynyl, aryl, arylalkyl-, alkylaryl-, cycloalkyl, cycloalkylalkyl-, heteroaryl, heteroarylalkyl-, heterocyclyl and heterocyclyalkyl-, and wherein each of said alkyl, alkenyl, alkynyl, aryl, arylalkyl-, alkylaryl-, cycloalkyl, cycloalkylalkyl-, heteroaryl, heteroarylalkyl-, heterocyclyl and heterocyclyalkyl-R⁹ group is optionally substituted with 1-3 independently selected R²¹ groups,
R¹⁰ is independently selected from the group consisting of: a bond, alkyl, alkenyl, alkynyl, aryl, arylalkyl-, alkylaryl-, cycloalkyl, cycloalkylalkyl-, heteroaryl, heteroarylalkyl-, heterocyclyl, heterocycylalkyl-, 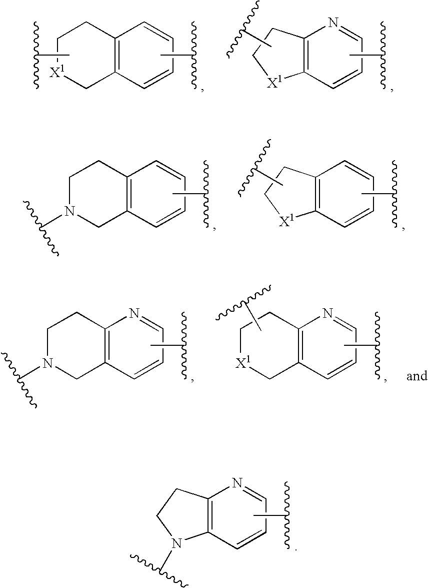
wherein X¹ is O, N(R¹⁴) or S; and wherein each of said R¹⁰ moieties (except for the bond) is optionally substituted with 1-3 independently selected R²¹ substituents;
R¹² is independently selected from the group consisting of H, alkyl, alkenyl, alkynyl, cycloalkyl, cycloalkylalkyl-, cycloalkenyl, heterocyclyl, heterocyclyalkyl-, aryl, arylalkyl-, heteroaryl, heteroarylalkyl-, —CN, —C(O)R¹⁵, —C(O)OR¹⁵, —C(O)N(R¹⁵)(R¹⁶), —S(O)N(R¹⁵)(R¹⁶), —S(O)₂N(R¹⁵)(R¹⁶), —S(O)R¹⁵, —S(O)₂R¹⁵, —C(═NOR¹⁵)R¹⁶ and —P(O)(OR¹⁵)(OR¹⁶), and wherein each of said alkyl, alkenyl, alkynyl, cycloalkyl, cycloalkylalkyl-, cycloalkenyl, heterocyclyl, heterocyclyalkyl-, aryl, arylalkyl-, heteroaryl, and heteroarylalkyl-R¹² group is optionally substituted with 1 to 5 independently selected R²¹ groups;
R¹⁴ (when R¹⁴ is not joined to R², R³ or R⁵) is independently selected from the group consisting of H, alkyl, alkenyl, alkynyl, cycloalkyl, cycloalkylalkyl-, cycloalkenyl, heterocyclyl, heterocyclylalkyl-, aryl, arylalkyl-, heteroaryl, heteroarylalkyl-, —CN, —C(O)R¹⁵, —C(O)OR¹⁵, —C(O)N(R¹⁵)(R¹⁶), —S(O)N(R¹⁵)(R¹⁶), —S(O)₂N(R¹⁵)(R¹⁶), —S(O)R¹⁵, —S(O)₂R¹⁵, —C(═NOR¹⁵)R¹⁶, and —P(O)(OR¹⁵)(OR¹⁶), and wherein each of said alkyl, alkenyl, alkynyl, cycloalkyl, cycloalkylalkyl-, cycloalkenyl, heterocyclyl, heterocyclylalkyl-, aryl, arylalkyl-, heteroaryl, and heteroarylalkyl-R¹⁴ group is optionally substituted with 1 to 5 independently selected R²¹ groups;
R¹⁵, R¹⁶ and R¹⁷ are each independently selected from the group consisting of H, alkyl, alkenyl, alkynyl, cycloalkyl, cycloalkylalkyl-, heterocyclyl, heterocyclylalkyl-, aryl, arylalkyl-, heteroaryl, heteroarylalkyl-, arylcycloalkyl-, arylheterocyclyl-, (R¹⁸)ₙ-alkyl-, (R¹⁸)ₙ-cycloalkyl-, (R¹⁸)ₙ-cycloalkylalkyl-, (R¹⁸)ₙ-heterocyclyl-, (R¹⁸)ₙ-heterocyclylalkyl-, (R¹⁸)ₙ-aryl-, (R¹⁸)ₙ-arylalkyl-, (R¹⁸)ₙ-heteroaryl- and (R¹⁸)ₙ-heteroarylalkyl, wherein n is 1 to 5;
Each R¹⁸ is independently selected from the group consisting of alkyl, alkenyl, alkynyl, aryl, arylalkyl-, arylalkenyl-, arylalkynyl-, —NO₂, halo, heteroaryl, HO-alkyoxyalkyl-, —CF, —CN, -alkyl-CN, —C(O)R¹⁹, —C(O)OH, —C(O)OR¹⁹, —C(O)NHR²⁰, —C(O)NH₂, —C(O)NH₂—C(O)N(alkyl)₂, —C(O)N(alkyl)(aryl), —C(O)N(alkyl)(heteroaryl), —SR¹⁹, —S(O)R²⁰, —S(O)NH₂, —S(O)NH(alkyl), —S(O)N(alkyl)(alkyl), —S(O)NH(aryl), —S(O)₂NH₂, —S(O)₂NHR¹⁹, —S(O)₂NH(heterocyclyl), —S(O)₂N(alkyl)₂, —S(O)₂N(alkyl)(aryl), —OCF₃, —OH, —OR²⁰, —O-heterocyclyl, —O-cycloalkylalkyl, —O-heterocyclyalkyl, —NH₂, —NHR²⁰, —N(alkyl)₂, —N(arylalkyl)₂, —N(arylalkyl)-(heteroarylalkyl)-NHC(O)R²⁰, —NHC(O)NH₂, —NHC(O)NH(alkyl), —NHC(O)N(alkyl)(alkyl), —N(alkyl)C(O)NH(alkyl), —N(alkyl)C(O)N(alkyl)(alkyl), —NHS(O)₂R²⁰, —NHS(O)₂NH(alkyl), —NHS(O)₂N(alkyl)(alkyl), —N(alkyl)S(O)₂NH(alkyl) and —N(alkyl)S(O)₂N(alkyl)(alkyl);
or, alternately, two R¹⁸ moieties on adjacent carbons can be linked together to form: 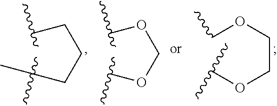
R¹⁹ is independently selected from the group consisting of: alkyl, cycloalkyl, aryl, arylalkyl- and heteroarylalkyl-;
R²⁰ is independently selected from the group consisting of: alkyl, cycloalkyl, aryl, halo substituted aryl, arylalkyl-, heteroaryl or heteroarylalkyl;
each R²¹ is independently selected from the group consisting of: alkyl, alkenyl, alkynyl, cycloalkyl, cycloalkylalkyl-, cycloalkenyl, heterocyclyl, heterocyclylalkyl-, aryl, arylalkyl-, heteroaryl, heteroarylalkyl-1, halo, —CN, —OR¹⁵, —C(O)R¹⁵, —C(O)OR¹⁵, —C(O)N(R¹⁵)(R¹⁶), —SR¹⁵, —S(O)N(R¹⁵)(R¹⁶), —CH(R¹⁵)(R¹⁶), —S(O)₂N(R¹⁵)(R¹⁶), —C(═NOR¹⁵)R¹⁶, —P(O)(OR¹⁵)(OR¹⁶), —N(R¹⁵)(R¹⁶), -alkyl-N(R¹⁵)(R¹⁶), —N(R¹⁵)C(O)R¹⁶, —CH₂—N(R¹⁵)C(O)R¹⁶, —CH₂—N(R¹⁵)C(O)N(R¹⁶)(R¹⁷), —CH₂—R¹⁵; —CH₂N(R¹⁵)(R¹⁶), —N(R¹⁵)S(O)R¹⁶, —N(R¹⁵)S(O)₂R¹⁶, —CH₂—N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —CH₂—N(R¹⁵)C(O)N(R¹⁶)(R¹⁷), —N(R¹⁵)C(O)OR¹⁶, —CH₂—N(R¹⁵)C(O)OR¹⁶, —S(O)R¹⁵, ═NOR¹⁵, —N₃, —NO₂ and —S(O)₂R¹⁵; and wherein each of the alkyl, cycloalkenyl, cycloalkyl, cycloalkylalkyl, heterocyclyl, heterocycylalkyl, aryl, arylalkyl, heteroaryl, heteroarylalkyl, alkenyl and alkynyl R²¹ groups is optionally substituted with 1 to 5 independently selected R²² groups;
Each R²² is independently selected from the group consisting of: alkyl, cycloalkyl, cycloalkenyl, heterocyclyl, aryl, heteroaryl, halo, —CF₃, —CN, —OR¹⁵, —C(O)R¹⁵, —C(O)OR¹⁵, -alkyl-C(O)OR¹⁵, C(O)N(R¹⁵)(R¹⁶), —SR¹⁵, —S(O)N(R¹⁵)(R¹⁶), —S(O)₂N(R¹⁵)(R¹⁶), —C(═NOR¹⁵)R¹⁶, —P((OR¹⁵)(OR¹⁶), —N(R¹⁵)(R¹⁶), -alkyl-N(R¹⁵)(R¹⁶), —N(R¹⁵)C(O)R¹⁶, —CH₂—N(R¹⁵)C(O)R¹⁶, —N(R¹⁵)S(O)R¹⁶, —N(R¹⁵)S(O)₂R¹⁶, —CH—N(R¹⁵)S(O)₂R¹⁶, —N(R¹⁵)S(O)₂N(R¹⁶)(R¹⁷), —N(R¹⁵)S(O)N(R¹⁶)(R¹⁷, —N(R¹⁵)C(O)N(R¹⁶)(R¹⁷), —CH₂—N(R¹⁵)C(O)N(R¹⁶)(R¹⁷), —N(R¹⁵)C(O)OR¹⁶, —CH₂—N(R¹⁵)C(O)OR¹⁶, —N₃, ═NOR¹⁵, —NO₂, —S(O)R¹⁵ and —S(O)₂R¹⁵.

2.-8. (canceled)

9. The compound of claim 1, wherein W is —C(O)—, X is —N(R¹⁴)—, and wherein U is a bond.

10.-11. (canceled)

12. The compound of claim 1, wherein R⁸ is H, or R⁸ is methyl.

13.-14. (canceled)

15. The compound of claim 1, wherein R¹⁰ is selected from the group consisting of: (1) unsubstituted aryl; (2) aryl substituted with 1 to 3 independently selected R²¹ moieties; and (3) is aryl substituted with 1-3 substitutents, which can be the same or different, each being independently selected from the group consisting of halo, alkyl, —CN, —NH₂, —NH(alkyl), —N(alkyl)₂, hydroxy and alkoxy groups.

16. The compound of claim 1, wherein R¹⁰ is selected from the group consisting of: 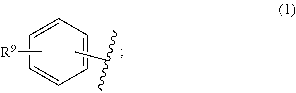
(2) phenyl substituted with 1 to 3 independently selected R²¹ moieties; and 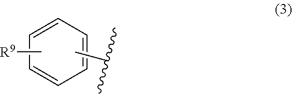
and said R¹⁰ is substituted with 1-3 substitutents, which can be the same or different each being independently selected from the group consisting of halo, alkyl, CN, NH₂, NH(alkyl), N(alkyl)₂, hydroxy and alkoxy groups.

17.-20. (canceled)

21. The compound of claim 1, wherein R¹⁰ is selected from the group consisting of: (1 unsubstituted heteroaryl; and (2) heteroaryl substituted with 1-3 substitutents, which can be the same or different, each being independently selected from the group consisting of halo, alkyl, CN, NH₂, NH(alkyl), N(alkyl)₂, hydroxy and alkoxy groups.

22. (canceled)

23. The compound of claim 1, wherein R¹⁰ is selected from the group consisting of: (1) aryl- and said aryl- is substituted with 1-3 substitutents, which can be the same or different, each being an alkoxy group; and (2) aryl- is substituted with methoxy.

24. The compound of claim 1, wherein R¹⁰ is selected from the group consisting of: 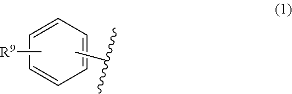
and said R¹⁰ is substituted with 1-3 substitutents, which can be the same or different, each being an alkoxy group;

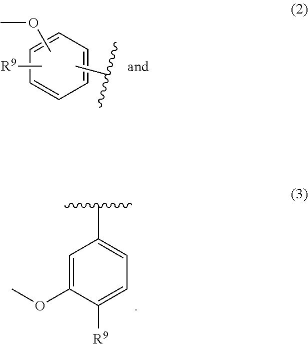

25.-27. (canceled)

28. The compound of claim 1, wherein R⁹ is selected from the group consisting of: (1) unsubstituted heteroaryl; and (2) heteroaryl which is substituted with 1-3 substituents which can be the same or different, each substituent being independently selected from the group consisting of halo, alkyl, CN, NH₂, NH(alkyl), N(alkyl)₂, hydroxy and alkoxy groups.

29. (canceled)

30. The compound of claim 1, wherein R⁹ is selected from the group consisting of: (1) imidazol-1-yl; and (2) 4-methyl-imidazol-1-yl.

31. (canceled)

32. The compound of claim 1, wherein R²¹ is independently selected from the group consisting of alkyl, alkyl-OH, unsubstituted arylalkyl-, arylalkyl wherein said aryl- portion of arylalkyl- is substituted with 1-3 halogen, unsubstituted aryl- and aryl wherein said aryl- is substituted with 1-3 halogen.

33. The compound of claim 1, wherein R²¹ is independently selected from the group consisting of alkyl, alkyl-OH,

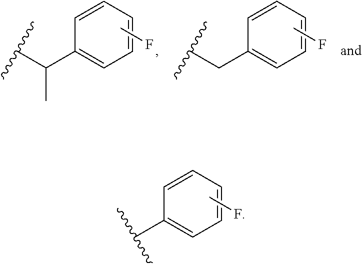

34. The compound of claim 1, wherein R³, R⁴, R⁶ and R⁷ can be the same or different, each being independently selected from the group consisting of H and alkyl.

35. The compound of claim 1, wherein R³, R⁴, R⁶ and R⁷ can be the same or different, each being independently selected from the group consisting of H and methyl.

36. The compound of claim 1 wherein the R⁹-R¹⁰-moiety is selected from the group consisting of:

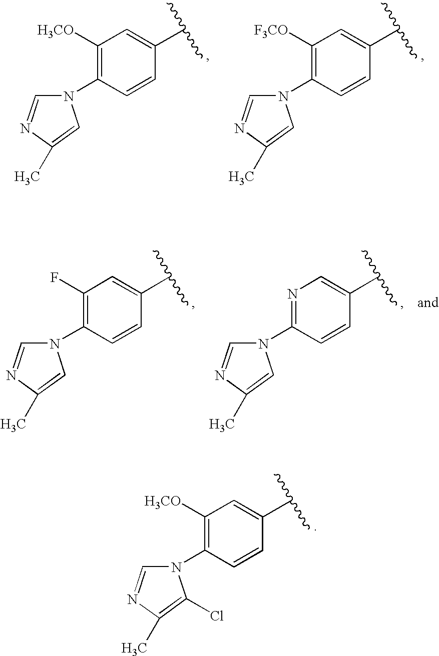

37. The compound of claim 1 wherein:
(1)
R¹ and R² are joined together as described in (i), or R² and R¹⁴ are joined together as described in (iv);
W is —C(O)—;
U is a bond or —C(R³)(R⁴)—;
X is —N(R¹⁴)—;
R²¹ is independently selected from the group consisting of alkyl, alkyl-OH, 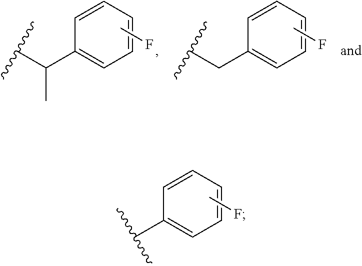
R⁸ is H or alkyl;
R¹⁰ is 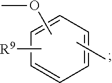
and
R⁹ is 4-methyl-imidazol-1-yl;
(2)
R² and R⁶ are joined together as described in (ii);
W is —C(O)—;
U is a bond or —C(R³)(R⁴)—;
X is —C(R⁶)(R⁷)—;
R¹ is alkyl, alkyl-OH, 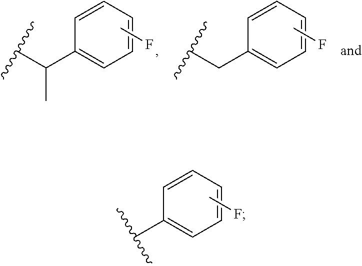
R⁸ is H or alkyl;
R¹⁰ is 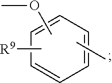
and
R⁹ is 4-methyl-imidazol-1-yl;
(3)
R³ and R¹⁴ are joined together as described in (v);
W is —C(O)—;
U is —C(R³)(R⁴)—;
X is —N(R¹⁴)—;
R¹ is alkyl, alkyl-OH; 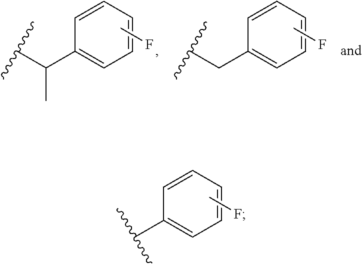
R²¹ is independently selected from the group consisting of alkyl, alkyl-OH, 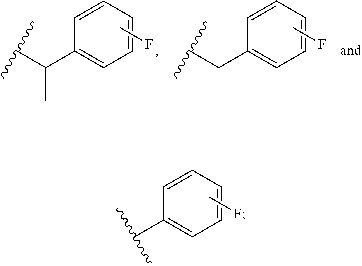
R⁸ is H or alkyl;
R¹⁰ is 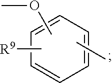
and
R⁹ is 4-methyl-imidazol-1-yl;
(4)
R³ and R⁶ are joined together as described in (vi);
W is —C(O)—;
U is —C(R³)(R⁴)—;
X is —C(R⁶)(R⁷)—;
R¹ is alkyl, alkyl-OH, 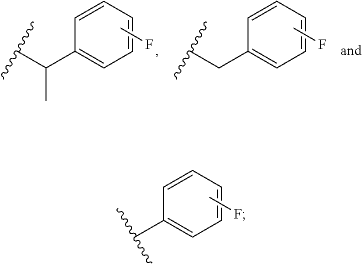
R²¹ is independently selected from the group consisting of alkyl, alkyl-OH, 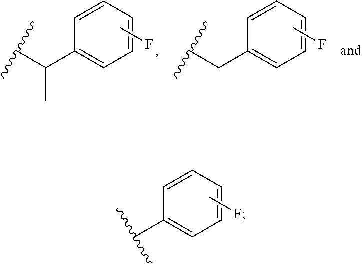
R⁸ is H or alkyl;
R¹⁰ is 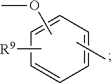
and
R⁹ is 4-methyl-imidazol-1-yl.

38.-41. (canceled)

42. The compound of claim 1, wherein:
(1) R¹ and R² are joined together to form a moiety selected from the group consisting of: 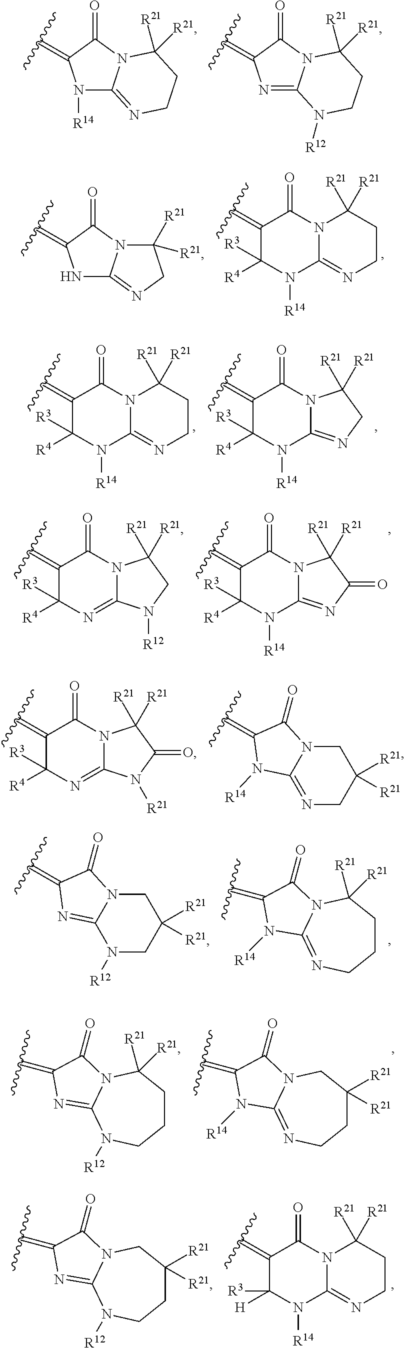 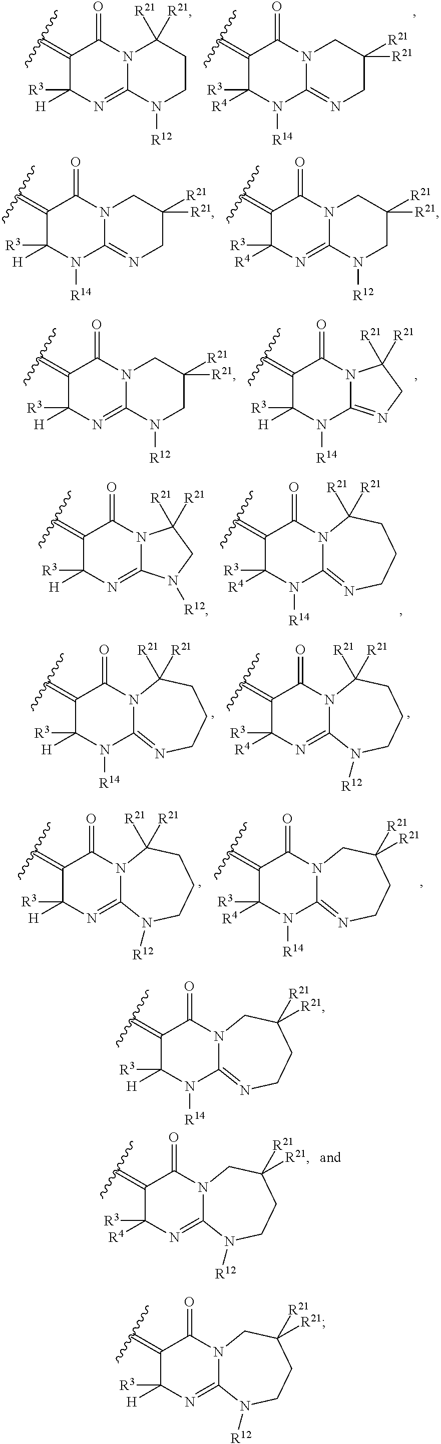
or
(2) R² and R¹⁴ are joined together to form a moiety selected from the group consisting of: 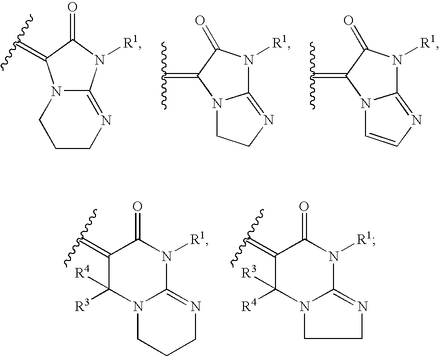
or
(3) R³ and R¹⁴ are joined together to form 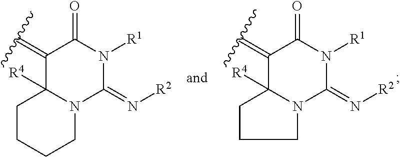
or
(4) R³ and R⁶ are joined to ether to form

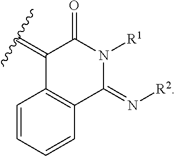

46. The compound of claim 37, wherein said compound is as described in (4), and wherein said compound is selected from the group of tautomers consisting of:

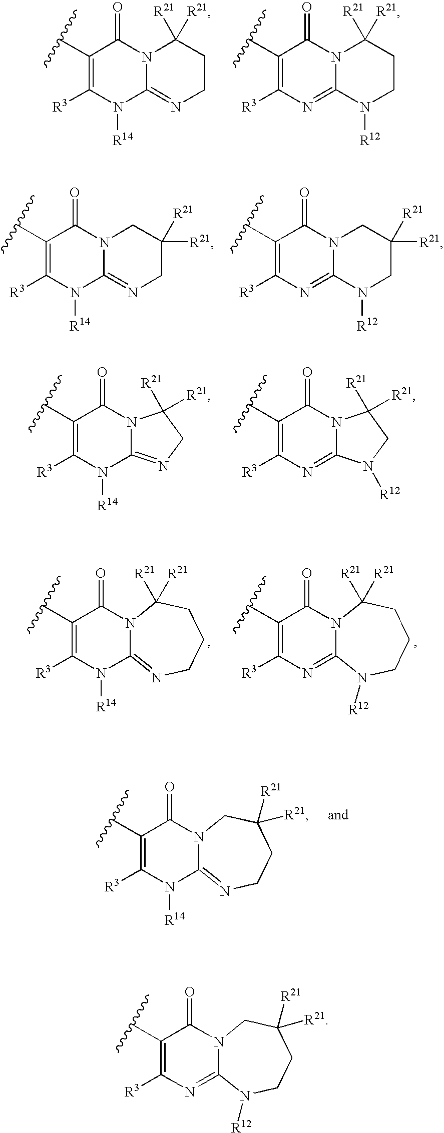

47. The compound of claim 1 selected from the group consisting of: 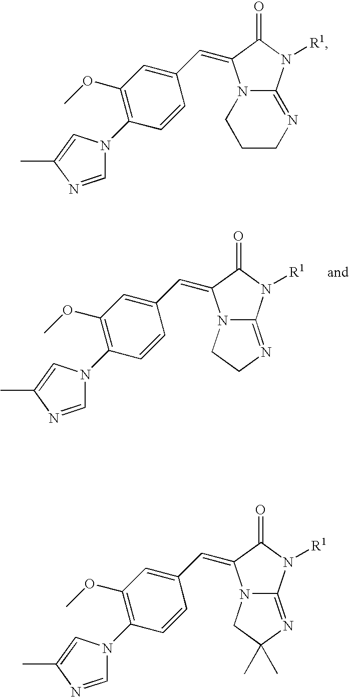
wherein R¹ is independently selected from the group consisting of H, alkyl,

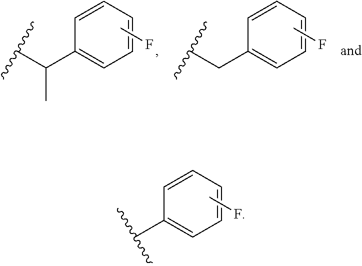

48. The compound of claim 47 wherein R¹ is independently selected from the group consisting of:

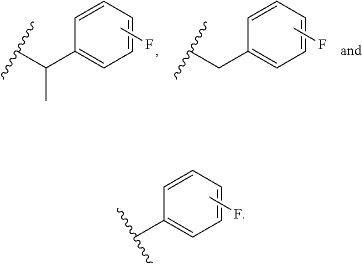

49. The compound of Claim 1 selected from the group consisting of: Y1, Y2, Y3, A9-A14, B1-B15, C3-C5, D4, E4, E6-E9, F7-F19, F20d-F20h, F21d-F21h, F22d-F22h, F23c-F23h, F24c-F24h, F25a-F25h. F26a-F26h, F27a-F27h, F28a-F28h, F29a-F29h, F30a-F30h. F31a-F31h, F32a-F32h, F33a-F33h, J1, J2, K7, K8b-K8h, K9a-K9h, K10a-K10h, K11a-K11h, K12a-K12h, K13a-K13h, K14a-K14h, K15a-K15h, K16a-K16h, K17a-K17h, K18a-K18h, K19a-K19h, K20a-K20h, K21a-K21h, K22a-K22h, and X1-X11.

50. The compound of Claim 1 selected from the group consisting of: (R)-A9, (R)-B7, F7-F13, J1, (S)-A9, (S)-B7, F4-F19, J2, A10, B8, B15 and D3.

51.-74. (canceled)

75. A pharmaceutical composition comprising:
(1) a therapeutically effective amount of one or more compounds of claim 1, and a pharmaceutically acceptable carrier; or
(2) an effective amount of one or more compounds of claim 1, and an effective amount of one or more other pharmaceutically active ingredients, and a pharmaceutically acceptable carrier, said other pharmaceutically active ingredients are selected form the group consisting of: (a) drugs useful for the treatment of Alzheimer's disease, (b) drugs useful for inhibiting the deposition of amyloid protein in, on or around neurological tissue, (c) drugs useful for treating neurodegenerative diseases, and (d) drugs useful for inhibiting gamma-secretase; or
(3) an effective amount of one or more compounds of claim 1, and an effective amount of one or more other pharmaceutically active ingredients, and a pharmaceutically acceptable carrier, wherein said other pharmaceutically active ingredients are selected from the group consisting of; BACE inhibitors; muscarinic antagonists; cholinesterase inhibitors; gamma secretase inhibitors; gamma secretase modulators; HMG-CoA reductase inhibitors; non-steroidal anti-inflammatory agents; N-methyl-D-aspartate receptor antagonists; anti-amyloid antibodies; vitamin E; nicotinic acetylcholine receptor antagonists; CB1 receptor inverse agonists or CB1 receptor antagonists; an antibiotic; growth hormone secretagogues; histamine H3 antagonists; AMPA agonists; PDE4 inhibitors; GABA_{A} inverse agonists; inhibitors of amyloid aggregation; glycogen synthase kinase beta inhibitors promoters of alpha secretase activity; PDE-10 inhibitors; Exelon; Cognex; Tau kinase inhibitors; anti-Abeta vaccine; APP ligands; agents that upregulate insulin cholesterol lowering agents; cholesterol absorption inhibitors; fibrates; LXR agonists; LRP mimics; nicotinic receptor agonists; H3 receptor antagonists; histone deacetylase inhibitors; hsp90 inhibitors; m1 muscarinic receptor agonists; 5-HT6 receptor antagonists; mGluR1; mGluR5; positive allosteric modulators or agonists; mGluR2/3 antagonists; anti-inflammatory agents that can reduce neuroinflammation; Prostaglandin EP2 receptor antagonists; PAI-1 inhibitors; and agents that can induce Abeta efflux such as gelsolin; or
(4) a therapeutically effective amount of one or more compounds of claim 1, and pharmaceutically acceptable carrier, said composition further comprising a therapeutically effective amount of one compounds selected from the group consisting of cholinesterase inhibitors, Aβ antibody inhibitors, gamma secretase inhibitors and beta secretase inhibitors; or
(5) the composition in said (4) above wherein said cholinesterase inhibitor is donepezil hydrochloride; or
(6) a therapeutically effective amount of one or more compounds of claim 1, and a pharmaceutically acceptable carrier, and an effective amount of one or more BACE inhibitors.

76.-85. (canceled)

86. A method of treating Alzheimers disease comprising administering a therapeutically effective amount of at least one compound of claim 1 to a patient in need of such treatment.

87.-89. (canceled)

90. A method of:
(1) modulating gamma-secretase comprising administering an effective amount of one or more compounds of claim 1 to a patient in need of such treatment; or
(2) treating one or more neurodegenerative diseases comprising administering an effective amount of one or more compounds of claim 1 to a patient in need of treatment; or
(3) inhibiting the deposition of amyloid protein in, on or around neurological tissue, comprising administering an effective amount of one or more compounds of claim 1 to a patient in need of treatment; or
(4) treating mild cognitive impairment comprising administering an effective amount of one or more compounds of claim 1 to a patient in need of treatment
(5) treating glaucoma comprising administering an effective amount of one or more compounds of claim 1 to a patient in need of treatment; or
(6) treating cerebral amyloid angiopathy comprising administering an effective amount of one or more compounds of claim 1 to a patient in need of treatment; or
(7) treating stroke comprising administering an effective amount of one or more compounds of claim 1 to a patient in need of treatment; or
(8) treating dementia comprising administering an effective amount of one or more compounds of claim 1 to a patient in need of treatment, or
(9) treating microgliosis comprising administering an effective amount of one or more compounds of claim 1 to a patient in need of treatment; or
(10) treating brain inflammation comprising administering an effective amount of one or more compounds of claim 1 to a patient in need of treatment; or
(11) treating olfactory function loss comprising administering an effective amount of one or more compounds of claim 1 to a patient in need of treatment; or
(12) treating a central nervous system disorder comprising administering a therapeutically effective amount of at least one compound of claim 1 to a patient in need of such treatment; or
(13) treating Downs syndrome comprising administering a therapeutically effective amount of at least one compound of claim 1 to a patient in need of such treatment; or
(14) treating down's syndrome comprising administering an effective amount of one or more compounds of claim 1, in combination with an effective amount of one or more cholinesterase inhibitors, to a patient in need of treatment; or
(15) treating down's syndrome comprising administering an effective amount of one or more compounds of claim 1, in combination with an effective amount of donepezil hydrochloride, to a patient in need of treatment.

91. (canceled)

92. A method of treating Alzheimer's disease comprising administering an effective amount of one or more compounds of claim 1 in combination with an effective amount of:
(1) one or more cholinesterase inhibitors; or
(2) donepezil hydrochloride; or
(3) one or more compounds selected from the group consisting of Aβ antibody inhibitors, gamma secretase inhibitors and beta secretase inhibitors; or
(4) one or more BACE inhibitors; or
(5) Exelon; or
(6) Cognex; or
(7) a Tau kinase inhibitor; or
(8) a Tau kinase inhibitor selected from the group consisting of: GSK3beta inhibitors, cdk5 inhibitors, and ERK inhibitors; or
(9) one anti-Abeta vaccination; or
(10) one or more APP ligands; or
(11) one or more agents that upregulate insulin degrading enzyme and/or neprilysin; or
(12) one or more cholesterol lowering agents; or
(13) one or more cholesterol lowering agents selected from the group consisting of statins and cholesterol absorption inhibitors; or
(14) one or more cholesterol lowering agents selected from the group consisting of: Atorvastatin, Fluvastatin, Lovastatin, Mevastatin, Pitavastatin, Pravastatin, Rosuvastatin, Simvastatin, and Ezetimibe; or
(15) one or more fibrates; or
(16) one or more fibrates selected from the group consisting of: clofibrate, Clofibride, Etofibrate, and Aluminium Clofibrate; or
(17) one or more LXR agonists; or
(18) one or more LRP mimics; or
(19) one or more 5-HT6 receptor antagonists; or
(20) one or more nicotinic receptor agonists; or
(21) one or more H3 receptor antagonists; or
(22) one or more histone deacetylase inhibitors; or
(23) one or more hsp90 inhibitors; or
(24) one or more m1 muscarinic receptor agonists; or
(25) one or more 5-HT6 receptor antagonists, mGluR1, mGluR5, or positive allosteric modulators or agonists; or
(26) one or more mGluR2/3 antagonists; or
(27) one or more anti-inflammatory agents that can reduce neuroinflammation; or
(28) one or more Prostaglandin EP2 receptor antagonists; or
(29) one or more PAI-1 inhibitors; or
(30) one or more agents that can induce Abeta efflux; or
(31) gelsolin.

93.-94. (canceled)

95. A kit comprising, in separate containers, in a single package, pharmaceutical compositions for use in combination, wherein one container comprises an effective amount of a compound of claim 1 in a pharmaceutically acceptable carrier, and another container comprises an effective amount of another pharmaceutically active ingredient, the combined quantities of the compound of claim 1 and the other pharmaceutically active ingredient being effective to: (a) treat Alzheimer's disease, or (b) inhibit the deposition of amyloid protein in, on or around neurological tissue, or (c) treat neurodegenerative diseases, or (d) modulate the activity of gamma-secretase.

96. (canceled)
